(19) United States
(12) Patent Application Publication
(10) Pub. No.: US 2021/0095283 A1
(43) Pub. Date: Apr. 1, 2021

(54) HETERODUPLEX NUCLEIC ACID MOLECULES AND USES THEREOF

(71) Applicant: Avidity Biosciences, Inc., La Jolla, CA (US)

(72) Inventors: Andrew John GEALL, La Jolla, CA (US); Venkata Ramana DOPPALAPUDI, La Jolla, CA (US); Rachel Elizabeth JOHNS, La Jolla, CA (US); Rob BURKE, La Jolla, CA (US); David Sai-Ho CHU, La Jolla, CA (US); Michael Caramian COCHRAN, La Jolla, CA (US); Michael David HOOD, La Jolla, CA (US); Hanhua HUANG, La Jolla, CA (US)

(21) Appl. No.: 16/960,543

(22) PCT Filed: Jan. 3, 2019
(86) PCT No.: PCT/US19/12223

§ 371 Date: Jul. 7, 2020

Related U.S. Application Data
(60) Provisional application No. 62/613,742, filed on Jan. 4, 2018.

(51) Int. Cl. C12N 15/113; C07K 16/28

(52) U.S. Cl. CPC C12N 15/113; C07K 16/2863; C12N 2310/14; C12N 2310/3513; C12N 2310/3181; C12N 2310/3233; C12N 2310/346; C12N 2310/31

(57) ABSTRACT

Disclosed herein are heteroduplex nucleic acid molecules, heteroduplex nucleic acid conjugates, and pharmaceutical compositions for modulating a protein expression. Also described herein include methods of treating a disease or indication which utilize a heteroduplex nucleic acid molecule, a heteroduplex nucleic acid conjugate, or a pharmaceutical composition that comprises a heteroduplex nucleic acid molecule.

### CROSS-REFERENCE

This application claims the benefit of U.S. Provisional Application No. 62/613,742, filed Jan. 4, 2018, which the applications is incorporated herein by reference in its entirety.

### BACKGROUND OF THE DISCLOSURE

Gene suppression by RNA-induced gene silencing provides several levels of control: transcription inactivation, small interfering RNA (siRNA)-induced mRNA degradation, and siRNA-induced transcriptional attenuation. In some instances, RNA interference (RNAi) provides long lasting effect over multiple cell divisions. As such, RNAi represents a viable method useful for drug target validation, gene function analysis, pathway analysis, and disease therapeutics.

### SUMMARY OF THE DISCLOSURE

Disclosed herein, in certain embodiments, are heteroduplex nucleic acid molecules, heteroduplex nucleic acid conjugates, and pharmaceutical compositions for modulating protein expression. In some embodiments, also described herein are heteroduplex nucleic acid molecules, heteroduplex nucleic acid conjugates, and pharmaceutical compositions with increased target tissue uptake and decreased hepatic clearance. In some embodiments, additionally described herein are heteroduplex nucleic acid molecules, heteroduplex nucleic acid conjugates, and pharmaceutical compositions for use in modulating protein expression in one or more diseases or conditions.

Disclosed herein is a molecule of Formula (I): A-(X¹—B)ₙ wherein A comprises a binding moiety; B consists of a hetero-duplex polynucleotide consisting of a guide strand and a passenger strand; X¹ consists of a bond or first non-polymeric linker; and n is an averaged value selected from 1-12; wherein the guide strand comprises at least one but no more than 10 phosphorothioate-modified non-natural nucleotides; wherein the passenger strand comprises a plurality of phosphorodiamidate morpholino oligomers or a plurality of peptide nucleic acid-modified non-natural nucleotides; and wherein the hetero-duplex polynucleotide has one of: a greater hepatocyte stability, reduced overall charge, reduced hepatocyte uptake, or extended pharmacokinetics, compare to analogous homoduplex nucleotide. In some embodiments, the passenger strand further comprises at least one inverted abasic moiety. In some embodiments, the guide strand further comprises at least one modified internucleotide linkage, at least one inverted abasic moiety, at least one 5′-vinylphosphonate modified non-natural nucleotide, or a combination thereof. In some embodiments, the guide strand comprises about 2, 3, 4, 5, 6, 7, 8, or 9 phosphorothioate-modified non-natural nucleotides. In some embodiments, the guide strand comprises 1 phosphorothioate-modified non-natural nucleotide. In some embodiments, the phosphorothioate modified non-natural nucleotide is located at an internucleotide linkage of the polynucleotide. In some embodiments, the at least one 5′-vinylphosphonate modified non-natural nucleotide is located at the 5′-terminus of the guide strand. In some embodiments, the at least one 5′-vinylphosphonate modified non-natural nucleotide is located about 1, 2, 3, 4, or 5 bases away from the 5′ terminus of the guide strand. In some embodiments, the at least one 5′-vinylphosphonate modified non-natural nucleotide is further modified at the 2′-position. In some embodiments, the 2′-modification is selected from 2′-O-methyl, 2′-O-methoxyethyl (2′-O-MOE), 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified nucleotide. In some embodiments, the at least one 5′-vinylphosphonate modified non-natural nucleotide is selected from:

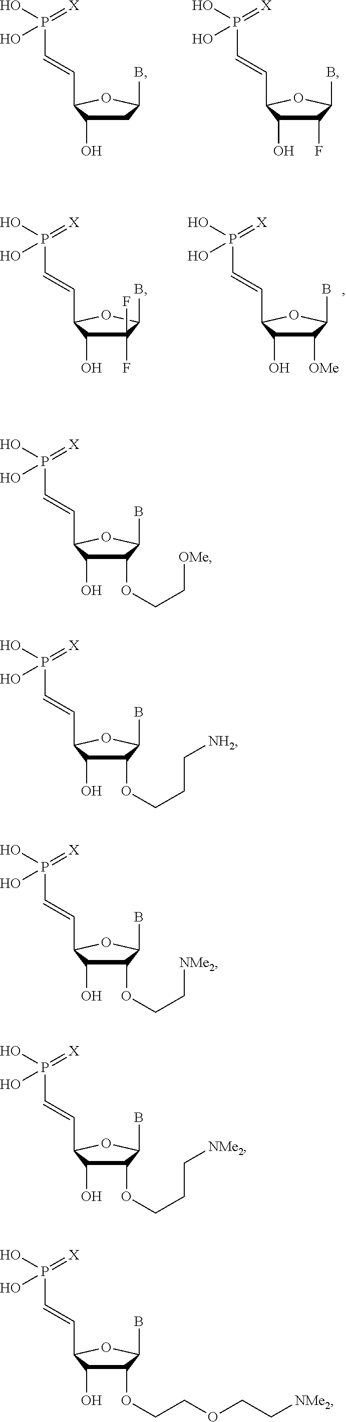

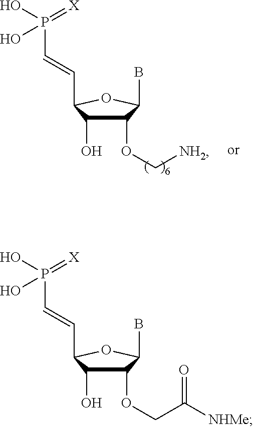

wherein X is O or S; and B is a heterocyclic base moiety. In some embodiments, the at least one 5′-vinylphosphonate modified non-natural nucleotide is selected from:

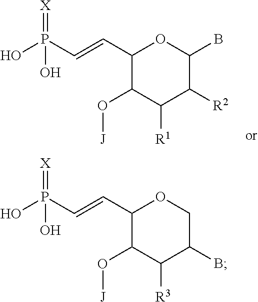

wherein X is O or S; B is a heterocyclic base moiety;

R¹, R², and R³ are independently selected from hydrogen, halogen, alkyl or alkoxy; and

J is an internucleotide linking group linking to the adjacent nucleotide of the polynucleotide. In some embodiments, the at least one 5′-vinylphosphonate modified non-natural nucleotide is selected from:

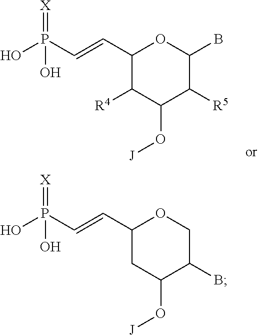

wherein X is O or S; B is a heterocyclic base moiety;

R⁴, and R⁵ are independently selected from hydrogen, halogen, alkyl or alkoxy; and

J is an internucleotide linking group linking to the adjacent nucleotide of the polynucleotide.
In some embodiments, the at least one 5′-vinylphosphonate modified non-natural nucleotide is selected from:

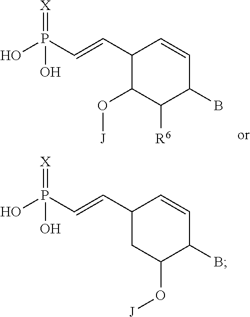

wherein X is O or S; B is a heterocyclic base moiety;

R⁶ is selected from hydrogen, halogen, alkyl or alkoxy; and

J is an internucleotide linking group linking to the adjacent nucleotide of the polynucleotide
In some embodiments, the at least one 5′-vinylphosphonate modified non-natural nucleotide is a locked nucleic acid (LNA). In some embodiments, the at least one 5′-vinylphosphonate modified non-natural nucleotide is a ethylene nucleic acid (ENA). In some embodiments, the at least one 5′-vinylphosphonate modified non-natural nucleotide is selected from:

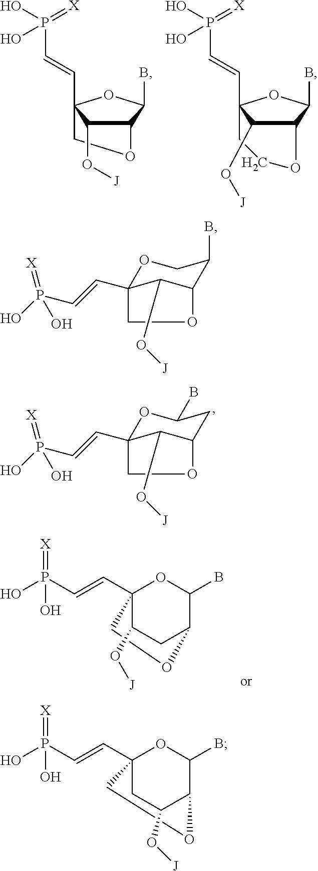

wherein X is O or S; B is a heterocyclic base moiety; and

J is an internucleotide linking group linking to the adjacent nucleotide of the polynucleotide.
In some embodiments, the at least one 5′-vinylphosphonate modified non-natural nucleotide is selected from:

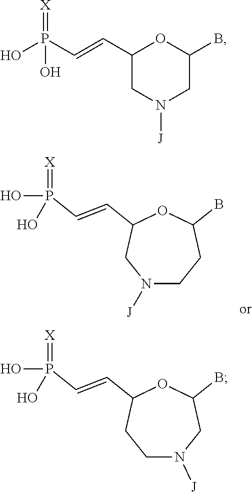

wherein X is O or S; B is a heterocyclic base moiety; and

J is an internucleotide linking group linking to the adjacent nucleotide of the polynucleotide.
In some embodiments, the at least one 5′-vinylphosphonate modified non-natural nucleotide is:

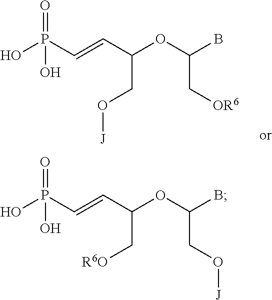

wherein X is O or S; B is a heterocyclic base moiety; R⁶ is selected from hydrogen, halogen, alkyl or alkoxy; and J is an internucleotide linking group linking to the adjacent nucleotide of the polynucleotide. In some embodiments, the at least one inverted abasic moiety is at at least one terminus. In some embodiments, the guide strand comprises RNA nucleotides. In some embodiments, the passenger strand comprises at least 6, 7, 8, 9, 10, 11, 12, 13, 14, 15, 16, 17, 18, 19, 20, or more phosphorodiamidate morpholino oligomer-modified non-natural nucleotides. In some embodiments, the passenger strand comprises 100% phosphorodiamidate morpholino oligomer-modified non-natural nucleotides. In some embodiments, the passenger strand is shorter in length than the guide strand, thereby generating a 5′ overhang, a 3′ overhang, or a combination thereof. In some embodiments, the passenger strand is equal in length to the guide strand, thereby generating a blunt end at each terminus of the hetero-duplex polynucleotide. In some embodiments, the passenger strand when hybridized to the guide strand further comprises at least one, two, three, four, or more mismatches, optionally comprising at least one, two, three, four, or more internal mismatches. In some embodiments, the hetero-duplex polynucleotide is a phosphorodiamidate morpholino oligomer/RNA hetero-duplex. In some embodiments, the passenger strand comprises at least 6, 7, 8, 9, 10, 11, 12, 13, 14, 15, 16, 17, 18, 19, 20, or more peptide nucleic acid-modified non-natural nucleotides. In some embodiments, the passenger strand comprises 100% peptide nucleic acid-modified non-natural nucleotides. In some embodiments, the passenger strand is shorter in length than the guide strand, thereby generating a 5′ overhang, a 3′ overhang, or a combination thereof. In some embodiments, the passenger strand is equal in length to the guide strand, thereby generating a blunt end at each terminus of the hetero-duplex polynucleotide. In some embodiments, the passenger strand when hybridized to the guide strand further comprises at least one, two, three, four, or more mismatches, optionally comprising at least one, two, three, four, or more internal mismatches. In some embodiments, the hetero-duplex polynucleotide is a peptide nucleic acid/RNA hetero-duplex. In some embodiments, the passenger strand is conjugated to A-X¹. In some embodiments, A-X¹ is conjugated to the 5′ end of the passenger strand. In some embodiments, A-X¹ is conjugated to the 3′ end of the passenger strand. In some embodiments, the guide strand comprises a sequence having at least 80%, 85%, 90%, 95%, 96%, 97%, 98%, 99%, or 100% sequence identity to SEQ ID NOs: 16-45, 422-1173, 1181-1184, or 1195-1242. In some embodiments, the passenger strand comprises a sequence having at least 80%, 85%, 90%, 95%, 96%, 97%, 98%, 99%, or 100% sequence identity to SEQ ID NOs: 16-45, 422-1173, 1181-1184, or 1195-1242. In some embodiments, the passenger strand comprises two or more polynucleotides, wherein each of the two or more polynucleotides hybridizes to a separate region on the guide strand, forming either a continuous strand without a gap between the termini of the two or more polynucleotides or a gap of about 1, 2, 3, or more bases between the termini of the two or more polynucleotides. In some embodiments, the two or more polynucleotides independently comprise at least 6, 7, 8, 9, 10, 11, 12, 13, 14, 15, 16, 17, 18, 19, 20, or more phosphorodiamidate morpholino oligomer-modified non-natural nucleotides or at least 6, 7, 8, 9, 10, 11, 12, 13, 14, 15, 16, 17, 18, 19, 20, or more peptide nucleic acid-modified non-natural nucleotides. In some embodiments, the two or more polynucleotides independently comprise 100% phosphorodiamidate morpholino oligomer-modified non-natural nucleotides or 100% peptide nucleic acid-modified non-natural nucleotides. In some embodiments, the overhang is about 1, 2, 3, 4, 5, 6, 7, 8, 9, 10, or more bases. In some embodiments, X¹ is a bond. In some embodiments, X¹ is a C₁-C₆ alkyl group. In some embodiments, X¹ is a homobifuctional linker or a heterobifunctional linker, optionally conjugated to a C₁-C₆ alkyl group. In some embodiments, the binding moiety comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some embodiments, the binding moiety comprises a peptide or small molecule. In some embodiments, n is an averaged value selected from 2-12, 4-12, 4-8, 6-8, or 8-12. In some embodiments, n is an averaged value of about 2, 4, 6, 8, 10, or 12. In some embodiments, n is an averaged value of about 2, 4, 6, or 8. In some embodiments, the molecule further comprises C. In some embodiments, C is polyethylene glycol. In some embodiments, C has a molecular weight of about 1000 Da, 2000 Da, or 5000 Da. In some embodiments, C is directly conjugated to B via X². In some embodiments, X² consists of a bond or second non-polymeric linker. In some embodiments, X² is a bond. In some embodiments, X² is a C₁-C₆ alkyl group. In some embodiments, X² is a homobifuctional linker or a heterobifunctional linker, optionally conjugated to a C₁-C₆ alkyl group. In some embodiments, the passenger strand is conjugated to A-X¹ and X²—C. In some embodiments, A-X¹ is conjugated to the 5′ end of the passenger strand and X²—C is conjugated to the 3′ end of the passenger strand. In some embodiments, X²—C is conjugated to the 5′ end of the passenger strand and A-X¹ is conjugated to the 3′ end of the passenger strand. In some embodiments, the molecule further comprises D. In some embodiments, D is an endosomolytic moiety. In some embodiments, the molecule has a reduced hepatic clearance rate compare to an analogous molecule comprising a homoduplex nucleotide. In some embodiments, the molecule has reduced uptake mediated by the Stabilin-1 or Stabilin-2 receptor relative to an analogous molecule comprising a homoduplex nucleotide. In some embodiments, the molecule has an increased plasma half-life relative to an analogous molecule comprising a homoduplex nucleotide. In some embodiments, the molecule has an increased target tissue uptake relative to an analogous molecule comprising a homoduplex nucleotide. In some embodiments, the molecule has an improved pharmacokinetics relative to an analogous molecule comprising a homoduplex nucleotide.

Disclosed herein, in certain embodiments, is a pharmaceutical composition, comprising: a molecule described above; and a pharmaceutically acceptable excipient.

Disclosed herein, in certain embodiments, is a method of treating a disease or indication, comprising: administering to a subject in need thereof a therapeutically effective amount of a molecule described above, or a pharmaceutical composition described above, thereby treating the subject. In some embodiments, the subject is a human.

### DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below. The patent application file contains at least one drawing executed in color. Copies of this patent application publication with color drawing(s) will be provided by the Office upon request and payment of the necessary fee.

FIG. 1A illustrates siRNA chemical modification pattern 1 for siRNA homoduplex.

FIG. 1B illustrates of siRNA chemical modification pattern 2 for siRNA homoduplex.

FIG. 1C illustrates siRNA chemical modification pattern 3 used on siRNA homoduplex.

FIG. 2A illustrates a 21mer duplex with 19 bases of complementarity and 3′ dinucleotide overhangs.

FIG. 2B illustrates a truncated duplex with 16 bases of complementarity and unsymmetrical 3′ overhangs.

FIG. 3A illustrates an overlaid SAX-HPLC chromatograms of EGFR mAb-SSB DAR1 and DAR2 conjugates.

FIG. 3B illustrates an overlaid SAX-HPLC chromatograms of EGFR mAb-SSB-0 PMO DAR1, DAR2 and DAR3 conjugates.

FIG. 3C illustrates an overlaid SAX-HPLC chromatograms of TfR mAb-SSB-18 PMO DAR1, and DAR2 conjugates.

FIG. 4A illustrates an analytical data table of conjugates used.

FIG. 4B illustrates in vivo study design.

FIG. 4C illustrates a graph of plasma clearance for siRNA. X axis shows time point (hours, hr) and y-axis shows percent of injected dose in plasma for EGFR-mAb-SSB DAR1 (blue solid line), EGFR-mAB-SSB DAR2 (blue hashed line), EGFR-mAB-SSB-0 PMO DAR1 (red solid line), EGFR-mAb-SSB-0 PMO DAR2 (red hashed line), EGFR mAB-SSB-18 PMO DAR1 (green solid line), and EGFR-mAB-SSB 18 PMO DAR2 (green hashed line).

FIG. 4D illustrates a graph of antibody concentration in plasma. X axis shows time point (hours, hr) and y-axis shows percent of injected dose in plasma for EGFR-mAb-SSB DAR1 (blue solid line), EGFR-mAB-SSB DAR2 (blue hashed line), EGFR-mAB-SSB-0 PMO DAR1 (red solid line), EGFR-mAb-SSB-0 PMO DAR2 (red hashed line), EGFR mAB-SSB-18 PMO DAR1 (green solid line), and EGFR-mAB-SSB 18 PMO DAR2 (green hashed line).

FIG. 4E illustrates a graph of siRNA liver concentration. X axis shows time point (hours, hr) and y-axis shows siRNA concentration in tissue (nM) for EGFR-mAb-SSB DAR1 (blue solid circles), EGFR-mAB-SSB DAR2 (blue open circles), EGFR-mAB-SSB-0 PMO DAR1 (red solid squares), EGFR-mAb-SSB-0 PMO DAR2 (red open squares), EGFR mAB-SSB-18 PMO DAR1 (green solid triangles), and EGFR-mAB-SSB 18 PMO DAR2 (green open triangles).

FIG. 5 illustrates an analytical data table of conjugates used.

FIG. 6A illustrates in vivo study design.

FIG. 6B illustrates of SSB mRNA knockdown in gastrocnemius tissue. X-axis shows dose (mg/kg) and y axis shows percentage (%) mRNA expression for TfR-mAb-SSB DAR1 (blue solid circles), TfR-mAB-SSB DAR2 (blue open circles), TfR-mAB-SSB-0 PMO DAR1 (red solid squares), TfR-mAb-SSB-0 PMO DAR2 (red open squares), TfR mAB-SSB-18 PMO DAR1 (green solid triangles), TfR-mAB-SSB 18 PMO DAR2 (green open triangles), and PBS control (black solid circles).

FIG. 6C illustrates of SSB mRNA knockdown in heart tissue. X-axis shows dose (mg/kg) and y axis shows percentage (%) mRNA expression for TfR-mAb-SSB DAR1 (blue solid circles), TfR-mAB-SSB DAR2 (blue open circles), TfR-mAB-SSB-0 PMO DAR1 (red solid squares), TfR-mAb-SSB-0 PMO DAR2 (red open squares), TfR mAB-SSB-18 PMO DAR1 (green solid triangles), TfR-mAB-SSB 18 PMO DAR2 (green open triangles), and PBS control (black solid circles).

FIG. 6D illustrates of SSB mRNA knockdown in liver tissue. X-axis shows dose (mg/kg) and y axis shows percentage (%) mRNA expression for TfR-mAb-SSB DAR1 (blue solid circles), TfR-mAB-SSB DAR2 (blue open circles), TfR-mAB-SSB-0 PMO DAR1 (red solid squares), TfR-mAb-SSB-0 PMO DAR2 (red open squares), TfR mAB-SSB-18 PMO DAR1 (green solid triangles), TfR-mAB-SSB 18 PMO DAR2 (green open triangles), and PBS control (black solid circles).

FIG. 6E illustrates of SSB guide strand accumulation in gastrocnemius tissue. X-axis shows dose (mg/kg) and y axis shows percentage (%) mRNA expression for TfR-mAb-SSB DAR1 (blue solid circles), TfR-mAB-SSB DAR2 (blue open circles), TfR-mAB-SSB-0 PMO DAR1 (red solid squares), TfR-mAb-SSB-0 PMO DAR2 (red open squares), TfR mAB-SSB-18 PMO DAR1 (green solid triangles), and TfR-mAB-SSB 18 PMO DAR2 (green open triangles).

FIG. 6F illustrates of SSB guide strand accumulation in heart tissue. X-axis shows dose (mg/kg) and y axis shows percentage (%) mRNA expression for TfR-mAb-SSB DAR1 (blue solid circles), TfR-mAB-SSB DAR2 (blue open circles), TfR-mAB-SSB-0 PMO DAR1 (red solid squares), TfR-mAb-SSB-0 PMO DAR2 (red open squares), TfR mAB-SSB-18 PMO DAR1 (green solid triangles), and TfR-mAB-SSB 18 PMO DAR2 (green open triangles).

FIG. 6G illustrates of SSB guide strand accumulation in liver tissue. X-axis shows dose (mg/kg) and y axis shows percentage (%) mRNA expression for TfR-mAb-SSB DAR1 (blue solid circles), TfR-mAB-SSB DAR2 (blue open circles), TfR-mAB-SSB-0 PMO DAR1 (red solid squares), TfR-mAb-SSB-0 PMO DAR2 (red open squares), TfR mAB-SSB-18 PMO DAR1 (green solid triangles), and TfR-mAB-SSB 18 PMO DAR2 (green open triangles).

FIG. 7 illustrates an analytical data table of conjugates used.

FIG. 8A illustrates in vivo study design.

FIG. 8B illustrates of Aha1 mRNA knockdown in gastrocnemius tissue. X-axis shows control, −24 hour, −4 hour, −1 hour, −15 minutes, and simultaneous and and y axis shows percentage (%) mRNA expression for PBS control (black bars), TfR-mAb-scramble DAR1 (orange bars), TfR-mAb-Aha1 DAR1 (blue bars), TfR-mAb-Aha1 DAR2 (red bars), PS-ASO-EON-decoy/TfR-mAb-Aha1 DAR2 (green bars), and Tfr-mAb-SSB DAR2/TfR-mAb-Aha1 DAR2 (purple bars).

FIG. 8C illustrates of Aha1 siRNA accumulation in gastrocnemius tissue. X-axis shows control, −24 hour, −4 hour, −1 hour, −15 minutes, and simultaneous and and y axis shows siRNA concentration in tissue (nM) TfR-mAb-scramble DAR1 (orange bars), TfR-mAb-Aha1 DAR1 (blue bars), TfR-mAb-Aha1 DAR2 (red bars), PS-ASO-EON-decoy/TfR-mAb-Aha1 DAR2 (green bars), and Tfr-mAb-SSB DAR2/TfR-mAb-Aha1 DAR2 (purple bars).

FIG. 8D illustrates of Aha1 mRNA knockdown in liver tissue. X-axis shows control, −24 hour, −4 hour, −1 hour, −15 minutes, and simultaneous and and y axis shows percentage (%) mRNA expression for PBS control (black bars), TfR-mAb-scramble DAR1 (orange bars), TfR-mAb-Aha1 DAR1 (blue bars), TfR-mAb-Aha1 DAR2 (red bars), PS-ASO-EON-decoy/TfR-mAb-Aha1 DAR2 (green bars), and Tfr-mAb-SSB DAR2/TfR-mAb-Aha1 DAR2 (purple bars).

FIG. 8E illustrates of Aha1 siRNA accumulation in liver tissue. X-axis shows control, −24 hour, −4 hour, −1 hour, −15 minutes, and simultaneous and and y axis shows siRNA concentration in tissue (nM) TfR-mAb-scramble DAR1 (orange bars), TfR-mAb-Aha1 DAR1 (blue bars), TfR-mAb-Aha1 DAR2 (red bars), PS-ASO-EON-decoy/TfR-mAb-Aha1 DAR2 (green bars), and Tfr-mAb-SSB DAR2/TfR-mAb-Aha1 DAR2 (purple bars).

FIG. 9 illustrates an analytical data table of conjugates used.

FIG. 10A illustrates in vivo study design.

FIG. 10B illustrates a graph of normalized siRNA plasma concentration. X-axis shows time (hours, hr) and y-axis shows normalized plasma siRNA concentration (% ID) for EGFR-mAb-HPRT DAR1 (red solid line), EGFR-mAB-HPRT DAR2 (red hashed line), EGFR-mAB-HPRT* DAR1 (blue solid line), EGFR-mAb- HPRT* DAR2 (blue hashed line), EGFR mAB-HPRT** DAR1 (green solid line), and EGFR-mAB-HPRT** DAR2 (green hashed line).

FIG. 10C illustrates a graph of siRNA concentration in liver. X-axis shows time (hours, hr) and y-axis shows siRNA concentration in liver (nM) for EGFR-mAb-HPRT DAR1 (red solid line), EGFR-mAB-HPRT DAR2 (red hashed line), EGFR-mAB-HPRT* DAR1 (blue solid line), EGFR-mAb-HPRT* DAR2 (blue hashed line), EGFR mAB-HPRT** DAR1 (green solid line), and EGFR-mAB-HPRT** DAR2 (green hashed line).

FIG. 11 illustrates percentage duplex formation and EC50 values of RNA/PMO heteroduplexes after transfection into LLC1 cells. Red base=mismatch, 0=nick and two separate passenger strands, (−)=base deletion/missing.

FIG. 12A shows % duplex formation EC50 knockdown values of PMO/RNA and PNA/RNA heteroduplexes after transfection into HCT116 cells. Red base=mismatch, ( )=nick and two separate passenger strands, (−)=base deletion/missing.

FIG. 12B illustrates SSB mRNA downregulation after RNA/PMO heteroduplexes transfection into HCT116 cells.

### DETAILED DESCRIPTION OF THE DISCLOSURE

Nucleic acid (e.g., RNAi) therapy is a targeted therapy with high selectivity and specificity. However, in some instances, nucleic acid therapy is also hindered by poor intracellular uptake, high hepatic clearance rate, limited blood stability, and non-specific off-target effect. To address these issues, various modifications of the nucleic acid composition are explored, such as for example, novel linkers for better stabilizing and/or lower toxicity, optimization of binding moiety for increased target specificity and/or target delivery, and nucleic acid polymer modifications for increased stability and/or reduced off-target effect.

Stabilins (Stabilin-1 and Stabilin-2) are class H scavenger receptors that clear negatively charged and/or sulfated carbohydrate polymer compounds from circulation. Studies have shown that Stabilins interact and internalize phosphorothioate modified antisense oligonucleotides interact and are responsible for hepatocyte uptake and clearance. See for example, Donner et al., “Co-administration of an excipient oligonucleotide helps delineate pathways of productive and nonproductive uptake of phosphorothioate antisense oligonucleotides in the liver,” Nucleic Acid Therapeutics 27(4): 209-220 (2017); and Miller et al., “Stabilin-1 and Stabilin-2 are specific receptors for the cellular internalization of phosphorothioate-modified antisense oligonucleotides (ASOs) in the liver,” Nucleic Acid Research 44(6): 2782-2794 (2016). In some instances, stabilins are further proposed to interact with nucleic acid molecules and contribute to the hepatic clearance rate.

In some embodiments, described herein are heteroduplex nucleic acid molecules, heteroduplex nucleic acid conjugates, and pharmaceutical compositions that have a reduced interaction with Stabilins (e.g., Stabilin-1 and/or Stabilin-2), relative to equivalent unmodified nucleic acid molecules. In some instances, the heteroduplex nucleic acid molecules, heteroduplex nucleic acid conjugates, and pharmaceutical compositions have improved target tissue uptake, lower hepatic clearance rate, longer blood stability, and reduced off-target effect.

In additional embodiments, further described herein are methods of using the heteroduplex nucleic acid molecules, heteroduplex nucleic acid conjugates, and pharmaceutical compositions for the treatment of a disease or indication.

### Polynucleic Acid Molecules

In some embodiments, disclosed herein is a hetero-duplex polynucleotide with one or more a greater hepatocyte stability, reduced overall charge, reduced hepatocyte uptake, or extended pharmacokinetics, compared to an analogous homoduplex nucleotide. As used herein, a hetero-duplex polynucleotide consists of a guide strand and a passenger strand, in which the guide strand comprises one or more modifications described herein, and the passenger strand comprises a plurality of phosphorodiamidate morpholino oligomers or a plurality of peptide nucleic acid-modified non-natural nucleotides. The homoduplex nucleotide consists of an equivalent guide and passenger strand, in which the nucleotides are unmodified and naturally-occurring.

In some embodiments, the guide strand comprises at least one but no more than 10 phosphorothioate-modified non-natural nucleotides. In some cases, the guide strand comprises about 2, 3, 4, 5, 6, 7, 8, or 9 phosphorothioate-modified non-natural nucleotides. In some cases, the guide strand comprises 9 phosphorothioate-modified non-natural nucleotides. In some cases, the guide strand comprises 8 phosphorothioate-modified non-natural nucleotides. In some cases, the guide strand comprises 7 phosphorothioate-modified non-natural nucleotides. In some cases, the guide strand comprises 6 phosphorothioate-modified non-natural nucleotides. In some cases, the guide strand comprises 5 phosphorothioate-modified non-natural nucleotides. In some cases, the guide strand comprises 4 phosphorothioate-modified non-natural nucleotides. In some cases, the guide strand comprises 3 phosphorothioate-modified non-natural nucleotides. In some cases, the guide strand comprises 2 phosphorothioate-modified non-natural nucleotides. In some cases, the guide strand comprises 1 phosphorothioate-modified non-natural nucleotide. In some cases, the phosphorothioate modified non-natural nucleotide is located at an internucleotide linkage of the polynucleotide.

In some cases, the guide strand further comprises at least one modified internucleotide linkage, at least one inverted abasic moiety, at least one 5′-vinylphosphonate modified non-natural nucleotide, or a combination thereof. In some instances, the at least one 5′-vinylphosphonate modified non-natural nucleotide is located at the 5′-terminus of the guide strand. In other instances, the at least one 5′-vinylphosphonate modified non-natural nucleotide is located about 1, 2, 3, 4, or 5 bases away from the 5′ terminus of the guide strand. In additional instances, the at least one 5′-vinylphosphonate modified non-natural nucleotide is further modified at the 2′-position.

In some embodiments, the guide strand comprises RNA molecules.

In some embodiments, the passenger strand comprises a plurality of phosphorodiamidate morpholino oligomers or a plurality of peptide nucleic acid-modified non-natural nucleotides, and optionally comprises at least one inverted abasic moiety. In some instances, the passenger strand comprises at least 6, 7, 8, 9, 10, 11, 12, 13, 14, 15, 16, 17, 18, 19, 20, or more phosphorodiamidate morpholino oligomer-modified non-natural nucleotides. In some instances, the passenger strand comprises 100% phosphorodiamidate morpholino oligomer-modified non-natural nucleotides. In some cases, the passenger strand is shorter in length than the guide strand, thereby generating a 5′ overhang, a 3′ overhang, or a combination thereof. In other cases, the passenger strand is equal in length to the guide strand, thereby generating a blunt end at each terminus of the hetero-duplex polynucleotide. In additional cases, the passenger strand when hybridized to the guide strand further comprises at least one, two, three, four, or more mismatches, optionally comprising at least one, two, three, four, or more internal mismatches.

In some instances, the passenger strand comprises at least 6, 7, 8, 9, 10, 11, 12, 13, 14, 15, 16, 17, 18, 19, 20, or more peptide nucleic acid-modified non-natural nucleotides. In some instances, the passenger strand comprises 100% peptide nucleic acid-modified non-natural nucleotides. In some cases, the passenger strand is shorter in length than the guide strand, thereby generating a 5′ overhang, a 3′ overhang, or a combination thereof. In other cases, the passenger strand is equal in length to the guide strand, thereby generating a blunt end at each terminus of the hetero-duplex polynucleotide. In additional cases, the passenger strand when hybridized to the guide strand further comprises at least one, two, three, four, or more mismatches, optionally comprising at least one, two, three, four, or more internal mismatches.

In some instances, the hetero-duplex polynucleotide is a phosphorodiamidate morpholino oligomer/RNA hetero-duplex.

In some instances, the hetero-duplex polynucleotide is a peptide nucleic acid/RNA hetero-duplex.

In some embodiments, the 2′ modification comprises a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety. Exemplary chemical structures of a 2′-O-methyl modification of an adenosine molecule and 2′O-methoxyethyl modification of an uridine are illustrated below.

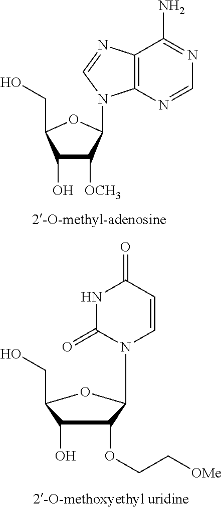

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

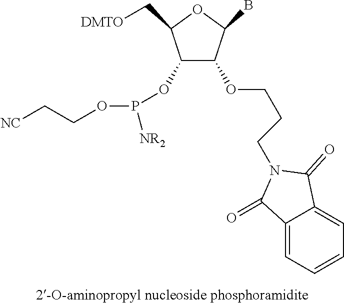

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (³E) conformation of the furanose ring of an LNA monomer.

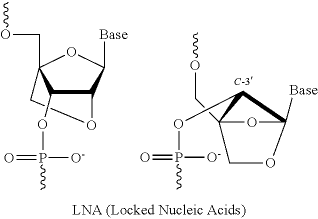

In some instances, the modification at the 2′ hydroxyl group comprises ethylene nucleic acids (ENA) such as for example 2′-4′-ethylene-bridged nucleic acid, which locks the sugar conformation into a C₃′-endo sugar puckering conformation. ENA are part of the bridged nucleic acids class of modified nucleic acids that also comprises LNA. Exemplary chemical structures of the ENA and bridged nucleic acids are illustrated below.

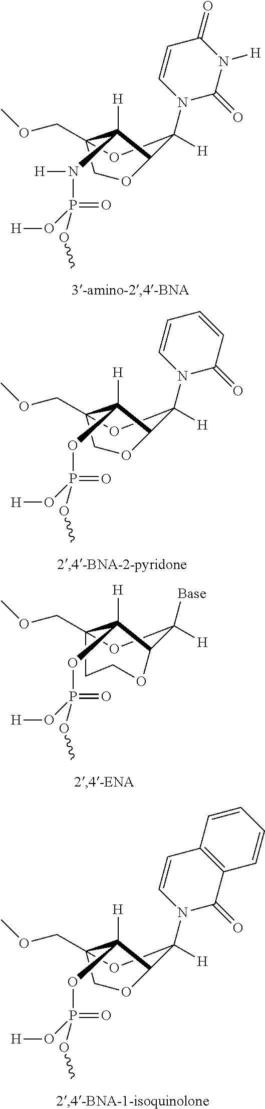

In some embodiments, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, nucleotide analogues comprise modified bases such as, but not limited to, 5-propynyluridine, 5-propynylcytidine, 6-methyladenine, 6-methylguanine, N, N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine, 4-thiouridine, and 2-thiocytidine, dihydrouridine, pseudouridine, queuosine, archaeosine, naphthyl and substituted naphthyl groups, any O- and N-alkylated purines and pyrimidines (such as N6-methyladeno sine, 5-methylcarbonylmethyluridine, uridine 5-oxyacetic acid, pyridine-4-one, or pyridine-2-one), phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, nucleotide analogues further comprise morpholinos, peptide nucleic acids (PNAs), methylphosphonate nucleotides, thiolphosphonate nucleotides, 2′-fluoro N3-P5′-phosphoramidites, 5′-anhydrohexitol nucleic acids (HNAs), or a combination thereof. Morpholino or phosphorodiamidate morpholino oligo (PMO) comprises synthetic molecules whose structure mimics natural nucleic acid structure by deviates from the normal sugar and phosphate structures. In some instances, the five member ribose ring is substituted with a six member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

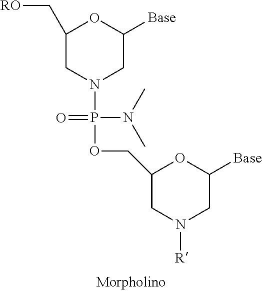

In some embodiments, peptide nucleic acid (PNA) does not contain sugar ring or phosphate linkage and the bases are attached and appropriately spaced by oligoglycine-like molecules, therefore, eliminating a backbone charge.

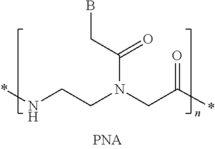

In some embodiments, one or more modifications optionally occur at the internucleotide linkage. In some instances, modified internucleotide linkage include, but is not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′linkage or 2′-5′linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisene oligonucleotides (PS ASO) are antisense oligonucleotides comprising a phosphorothioate linkage. An exemplary PS ASO is illustrated below.

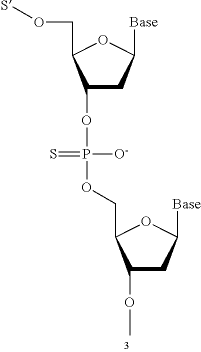

In some instances, the modification is a methyl or thiol modification such as methylphosphonate or thiolphosphonate modification. Exemplary thiolphosphonate nucleotide (left) and methylphosphonate nucleotide (right) are illustrated below.

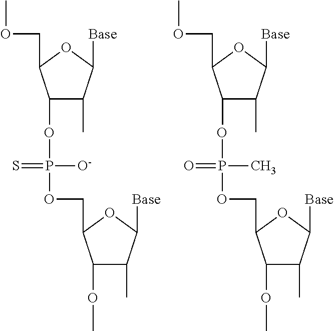

In some instances, a modified nucleotide includes, but is not limited to, 2′-fluoro N3-P5′-phosphoramidites illustrated as:

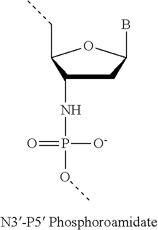

In some instances, a modified nucleotide includes, but is not limited to, hexitol nucleic acid (or 5′-anhydrohexitol nucleic acids (HNA)) illustrated as:

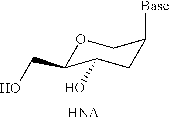

In some embodiments, a nucleotide analogue or artificial nucleotide base described above comprises a 5′-vinylphosphonate modified nucleotide nucleic acid with a modification at a 5′ hydroxyl group of the ribose moiety. In some embodiments, the 5′-vinylphosphonate modified nucleotide is selected from the nucleotide provided below, wherein X is O or S; and B is a heterocyclic base moiety.

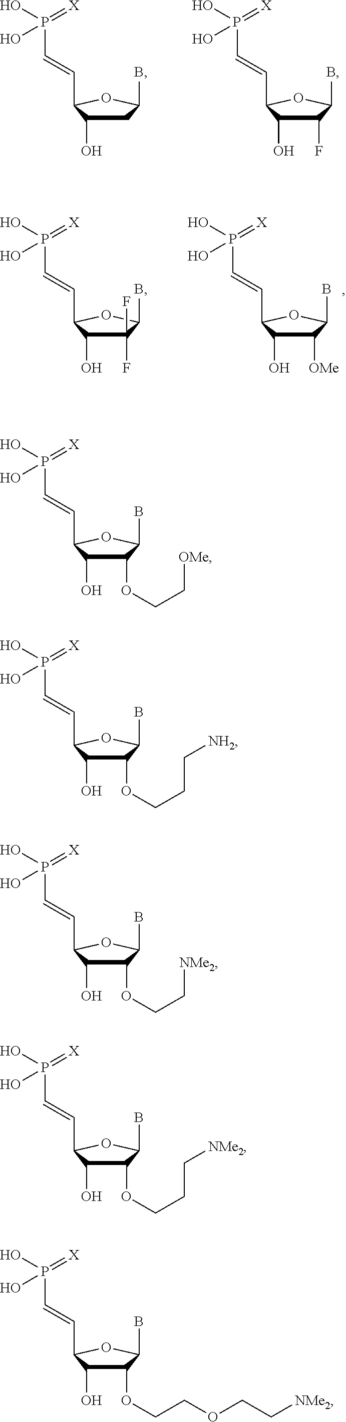

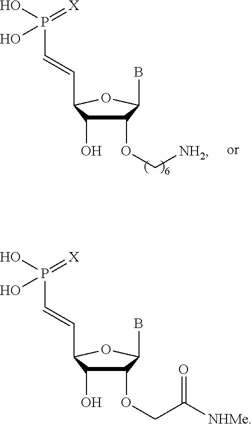

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some instances, the 5′-vinylphosphonate modified nucleotide is further modified at the 2′ hydroxyl group in a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of 5′-vinylphosphonate modified LNA are illustrated below, wherein X is O or S; B is a heterocyclic base moiety; and J is an internucleotide linking group linking to the adjacent nucleotide of the polynucleotide.

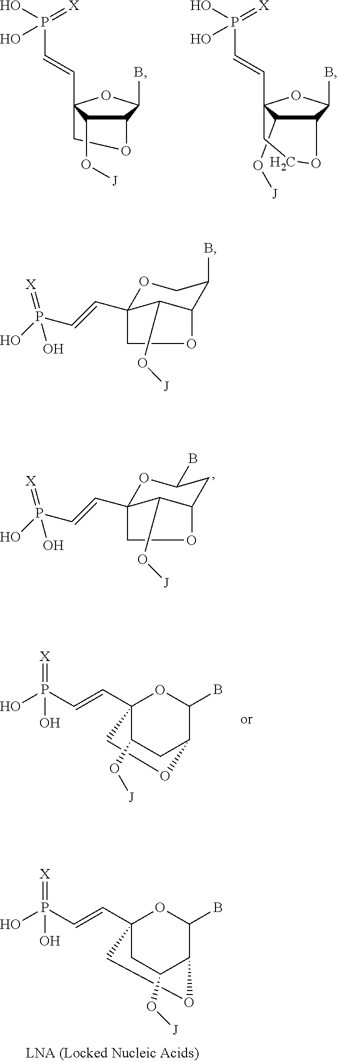

In some embodiments, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, a nucleotide analogue comprises a modified base such as, but not limited to, 5-propynyluridine, 5-propynylcytidine, 6-methyladenine, 6-methylguanine, N, N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. 5′-Vinylphosphonate modified nucleotides also include those nucleotides that are modified with respect to the sugar moiety, as well as 5′-vinylphosphonate modified nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, a 5′-vinylphosphonate modified nucleotide analogue further comprises a morpholino, a peptide nucleic acid (PNA), a methylphosphonate nucleotide, a thiolphosphonate nucleotide, a 2′-fluoro N3-P5′-phosphoramidite, or a 1′, 5′-anhydrohexitol nucleic acid (HNA). Morpholino or phosphorodiamidate morpholino oligo (PMO) comprises synthetic molecules whose structure mimics natural nucleic acid structure but deviates from the normal sugar and phosphate structures. In some instances, the five member ribose ring is substituted with a six member morpholino ring containing four carbons, one nitrogen, and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides. A non-limiting example of a 5′-vinylphosphonate modified morpholino oligonucleotide is illustrated below, wherein X is O or S; and B is a heterocyclic base moiety.

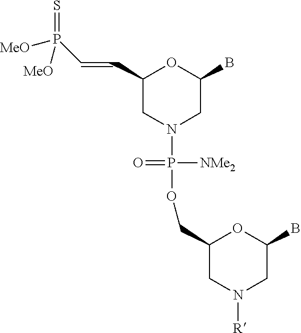

In some embodiments, a 5′-vinylphosphonate modified morpholino or PMO described above is a PMO comprising a positive or cationic charge. In some instances, the PMO is PMOplus (Sarepta). PMOplus refers to phosphorodiamidate morpholino oligomers comprising any number of (1-piperazino)phosphinylideneoxy, (1-(4-(omega-guanidino-alkanoyl))-piperazino)phosphinylideneoxy linkages (e.g., as such those described in PCT Publication No. WO2008/036127. In some cases, the PMO is a PMO described in U.S. Pat. No. 7,943,762.

In some embodiments, a morpholino or PMO described above is a PMO-X (Sarepta). In some cases, PMO-X refers to phosphorodiamidate morpholino oligomers comprising at least one linkage or at least one of the disclosed terminal modifications, such as those disclosed in PCT Publication No. WO2011/150408 and U.S. Publication No. 2012/0065169.

In some embodiments, a morpholino or PMO described above is a PMO as described in Table 5 of U.S. Publication No. 2014/0296321.

Exemplary representations of the chemical structure of 5′-vinylphosphonate modified nucleic acids are illustrated below, wherein X is O or S; B is a heterocyclic base moiety; and J is an internucleotide linkage.

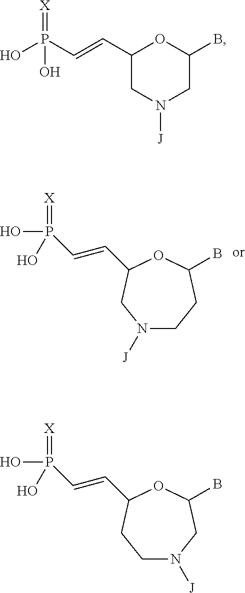

In some embodiments, peptide nucleic acid (PNA) does not contain sugar ring or phosphate linkage and the bases are attached and appropriately spaced by oligoglycine-like molecules, therefore, eliminating a backbone charge.

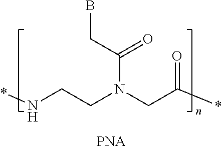

In some embodiments, one or more modifications of the 5′-vinylphosphonate modified oligonucleotide optionally occur at the internucleotide linkage. In some instances, modified internucleotide linkage includes, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3′-5′linkage or 2′-5′linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some instances, the modification is a methyl or thiol modification such as methylphosphonate or thiolphosphonate modification. Exemplary thiolphosphonate nucleotide (left), phosphorodithioates (center) and methylphosphonate nucleotide (right) are illustrated below.

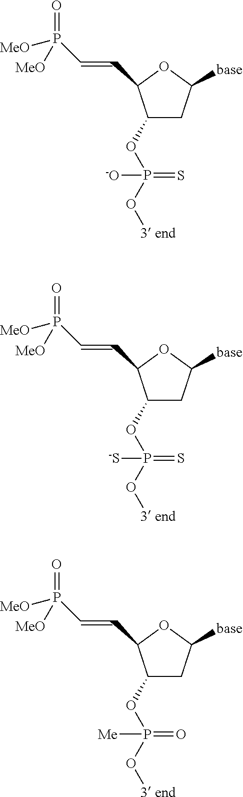

In some instances, a 5′-vinylphosphonate modified nucleotide includes, but is not limited to, phosphoramidites illustrated as:

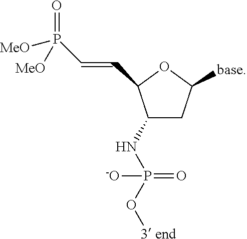

In some instances, the modified internucleotide linkage is a phosphorodiamidate linkage. A non-limiting example of a phosphorodiamidate linkage with a morpholino system is shown below.

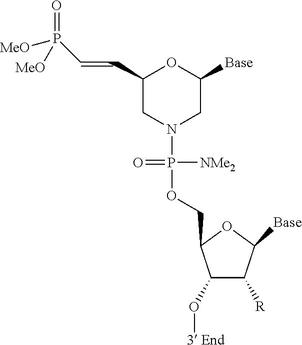

In some instances, the modified internucleotide linkage is a methylphosphonate linkage. A non-limiting example of a methylphosphonate linkage is shown below.

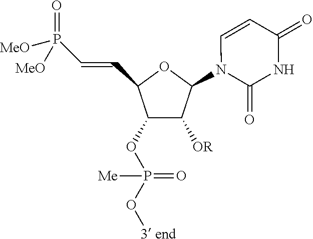

In some instances, the modified internucleotide linkage is a amide linkage. A non-limiting example of an amide linkage is shown below.

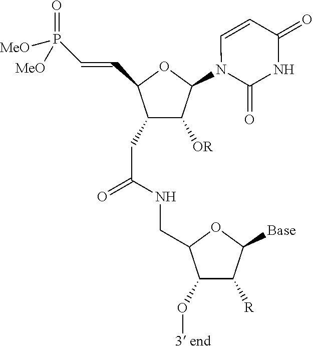

In some instances, a 5′-vinylphosphonate modified nucleotide includes, but is not limited to, the modified nucleic acid illustrated below.

In some embodiments, one or more modifications comprise a modified phosphate backbone in which the modification generates a neutral or uncharged backbone. In some instances, the phosphate backbone is modified by alkylation to generate an uncharged or neutral phosphate backbone. As used herein, alkylation includes methylation, ethylation, and propylation. In some cases, an alkyl group, as used herein in the context of alkylation, refers to a linear or branched saturated hydrocarbon group containing from 1 to 6 carbon atoms. In some instances, exemplary alkyl groups include, but are not limited to, methyl, ethyl, n-propyl, isopropyl, n-butyl, isobutyl, sec-butyl, tert-butyl, n-pentyl, isopentyl, neopentyl, hexyl, isohexyl, 1, 1-dimethylbutyl, 2,2-dimethylbutyl, 3,3-dimethylbutyl, and 2-ethylbutyl groups. In some cases, a modified phosphate is a phosphate group as described in U.S. Pat. No. 9,481,905.

In some embodiments, additional modified phosphate backbones comprise methylphosphonate, ethylphosphonate, methylthiophosphonate, or methoxyphosphonate. In some cases, the modified phosphate is methylphosphonate. In some cases, the modified phosphate is ethylphosphonate. In some cases, the modified phosphate is methylthiophosphonate. In some cases, the modified phosphate is methoxyphosphonate.

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some embodiments, the guide strand comprises about 1, 2, 3, 4, 5, 6, 7, 8, 9, 10, 11, 12, 13, 14, 15, 16, 17, 18, 20, 25, or more of the artificial nucleotide analogues described herein. In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the guide strand comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the guide strand comprises 1, 2, 3, 4, 5, 6, 7, 8, 9, 10, 11, 12, 13, 14, 15, 16, 17, 18, 20, 25, or more of 2′-O-methyl modified nucleotides. In some instances, the guide strand comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the guide strand comprises 1, 2, 3, 4, 5, 6, 7, 8, 9, 10, 11, 12, 13, 14, 15, 16, 17, 18, 20, 25, or more of thiolphosphonate nucleotides.

In some instances, the guide strand comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification.

In some cases, the guide strand comprises at least one of: from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification.

In some cases, the guide strand comprises at least one of: from about 10% to about 80% modification, from about 20% to about 80% modification, from about 30% to about 80% modification, from about 40% to about 80% modification, from about 50% to about 80% modification, from about 60% to about 80% modification, and from about 70% to about 80% modification.

In some instances, the guide strand comprises at least one of: from about 10% to about 70% modification, from about 20% to about 70% modification, from about 30% to about 70% modification, from about 40% to about 70% modification, from about 50% to about 70% modification, and from about 60% to about 70% modification.

In some instances, the guide strand comprises at least one of: from about 10% to about 60% modification, from about 20% to about 60% modification, from about 30% to about 60% modification, from about 40% to about 60% modification, and from about 50% to about 60% modification.

In some cases, the guide strand comprises at least one of: from about 10% to about 50% modification, from about 20% to about 50% modification, from about 30% to about 50% modification, and from about 40% to about 50% modification.

In some cases, the guide strand comprises at least one of: from about 10% to about 40% modification, from about 20% to about 40% modification, and from about 30% to about 40% modification.

In some cases, the guide strand comprises at least one of: from about 10% to about 30% modification, and from about 20% to about 30% modification.

In some cases, the guide strand comprises from about 10% to about 20% modification.

In some cases, the guide strand comprises from about 15% to about 90%, from about 20% to about 80%, from about 30% to about 70%, or from about 40% to about 60% modifications.

In additional cases, the guide strand comprises at least about 15%, 20%, 30%, 40%, 50%, 60%, 70%, 80%, 90%, 95%, or 99% modification.

In some embodiments, the guide strand comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modifications.

In some instances, the guide strand comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modified nucleotides.

In some instances, from about 5 to about 100% of the guide strand comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95% or 100% of the guide strand comprise the artificial nucleotide analogues described herein. In some instances, about 5% of the guide strand comprises the artificial nucleotide analogues described herein. In some instances, about 10% of the guide strand comprises the artificial nucleotide analogues described herein. In some instances, about 15% of the guide strand comprises the artificial nucleotide analogues described herein. In some instances, about 20% of the guide strand comprises the artificial nucleotide analogues described herein. In some instances, about 25% of the guide strand comprises the artificial nucleotide analogues described herein. In some instances, about 30% of the guide strand comprises the artificial nucleotide analogues described herein. In some instances, about 35% of the guide strand comprises the artificial nucleotide analogues described herein. In some instances, about 40% of the guide strand comprises the artificial nucleotide analogues described herein. In some instances, about 45% of the guide strand comprises the artificial nucleotide analogues described herein. In some instances, about 50% of the guide strand comprises the artificial nucleotide analogues described herein. In some instances, about 55% of the guide strand comprises the artificial nucleotide analogues described herein. In some instances, about 60% of the guide strand comprises the artificial nucleotide analogues described herein. In some instances, about 65% of the guide strand comprises the artificial nucleotide analogues described herein. In some instances, about 70% of the guide strand comprises the artificial nucleotide analogues described herein. In some instances, about 75% of the guide strand comprises the artificial nucleotide analogues described herein. In some instances, about 80% of the guide strand comprises the artificial nucleotide analogues described herein. In some instances, about 85% of the guide strand comprises the artificial nucleotide analogues described herein. In some instances, about 90% of the guide strand comprises the artificial nucleotide analogues described herein. In some instances, about 95% of the guide strand comprises the artificial nucleotide analogues described herein. In some instances, about 96% of the guide strand comprises the artificial nucleotide analogues described herein. In some instances, about 97% of the guide strand comprises the artificial nucleotide analogues described herein. In some instances, about 98% of the guide strand comprises the artificial nucleotide analogues described herein. In some instances, about 99% of the guide strand comprises the artificial nucleotide analogues described herein. In some instances, about 100% of the guide strand comprises the artificial nucleotide analogues described herein. In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some embodiments, the guide strand comprises from about 1 to about 25 modifications in which the modification comprises an artificial nucleotide analogues described herein. In some embodiments, the guide strand comprises about 1 modification in which the modification comprises an artificial nucleotide analogue described herein. In some embodiments, the guide strand comprises about 2 modifications in which the modifications comprise an artificial nucleotide analogue described herein. In some embodiments, the guide strand comprises about 3 modifications in which the modifications comprise an artificial nucleotide analogue described herein. In some embodiments, the guide strand comprises about 4 modifications in which the modifications comprise an artificial nucleotide analogue described herein. In some embodiments, the guide strand comprises about 5 modifications in which the modifications comprise an artificial nucleotide analogue described herein. In some embodiments, the guide strand comprises about 6 modifications in which the modifications comprise an artificial nucleotide analogue described herein. In some embodiments, the guide strand comprises about 7 modifications in which the modifications comprise an artificial nucleotide analogue described herein. In some embodiments, the guide strand comprises about 8 modifications in which the modifications comprise an artificial nucleotide analogue described herein. In some embodiments, the guide strand comprises about 9 modifications in which the modifications comprise an artificial nucleotide analogue described herein. In some embodiments, the guide strand comprises about 10 modifications in which the modifications comprise an artificial nucleotide analogue described herein. In some embodiments, the guide strand comprises about 11 modifications in which the modifications comprise an artificial nucleotide analogue described herein. In some embodiments, the guide strand comprises about 12 modifications in which the modifications comprise an artificial nucleotide analogue described herein. In some embodiments, the guide strand comprises about 13 modifications in which the modifications comprise an artificial nucleotide analogue described herein. In some embodiments, the guide strand comprises about 14 modifications in which the modifications comprise an artificial nucleotide analogue described herein. In some embodiments, the guide strand comprises about 15 modifications in which the modifications comprise an artificial nucleotide analogue described herein. In some embodiments, the guide strand comprises about 16 modifications in which the modifications comprise an artificial nucleotide analogue described herein. In some embodiments, the guide strand comprises about 17 modifications in which the modifications comprise an artificial nucleotide analogue described herein. In some embodiments, the guide strand comprises about 18 modifications in which the modifications comprise an artificial nucleotide analogue described herein. In some embodiments, the guide strand comprises about 19 modifications in which the modifications comprise an artificial nucleotide analogue described herein. In some embodiments, the guide strand comprises about 20 modifications in which the modifications comprise an artificial nucleotide analogue described herein. In some embodiments, the guide strand comprises about 21 modifications in which the modifications comprise an artificial nucleotide analogue described herein. In some embodiments, the guide strand comprises about 22 modifications in which the modifications comprise an artificial nucleotide analogue described herein. In some embodiments, the guide strand comprises about 23 modifications in which the modifications comprise an artificial nucleotide analogue described herein. In some embodiments, the guide strand comprises about 24 modifications in which the modifications comprise an artificial nucleotide analogue described herein. In some embodiments, the guide strand comprises about 25 modifications in which the modifications comprise an artificial nucleotide analogue described herein.

In some embodiments, when pyrimidine nucleotides are present in the guide strand are 2′-deoxy-2′-fluoro pyrimidine nucleotides and when purine nucleotides are present in said guide strand comprise 2′-deoxy-purine nucleotides.

In another embodiment, a guide strand described herein comprises 2′-5′ internucleotide linkages. In some instances, the 2′-5′ internucleotide linkage(s) is at the 3′-end, the 5′-end, or both of the 3′- and 5′-ends of one or both sequence strands. In addition instances, the 2′-5′ internucleotide linkage(s) is present at various other positions within the strand, for example, about 1, 2, 3, 4, 5, 6, 7, 8, 9, 10, or more including every internucleotide linkage of a pyrimidine nucleotide in the strand comprise a 2′-5′ internucleotide linkage, or about 1, 2, 3, 4, 5, 6, 7, 8, 9, 10, or more including every internucleotide linkage of a purine nucleotide in the strand comprise a 2′-5′ internucleotide linkage.

In some embodiments, the hetero-duplex polynucleotide is from about 10 to about 50 nucleotides in length. In some instances, the hetero-duplex polynucleotide is from about 10 to about 30, from about 15 to about 30, from about 18 to about 25, from about 18 to about 24, from about 19 to about 23, or from about 20 to about 22 nucleotides in length.

In some embodiments, the hetero-duplex polynucleotide is about 50 nucleotides in length. In some instances, the hetero-duplex polynucleotide is about 45 nucleotides in length. In some instances, the hetero-duplex polynucleotide is about 40 nucleotides in length. In some instances, the hetero-duplex polynucleotide is about 35 nucleotides in length. In some instances, the hetero-duplex polynucleotide is about 30 nucleotides in length. In some instances, the hetero-duplex polynucleotide is about 25 nucleotides in length. In some instances, the hetero-duplex polynucleotide is about 20 nucleotides in length. In some instances, the hetero-duplex polynucleotide is about 19 nucleotides in length. In some instances, the hetero-duplex polynucleotide is about 18 nucleotides in length. In some instances, the hetero-duplex polynucleotide is about 17 nucleotides in length. In some instances, the hetero-duplex polynucleotide is about 16 nucleotides in length. In some instances, the hetero-duplex polynucleotide is about 15 nucleotides in length. In some instances, the hetero-duplex polynucleotide is about 14 nucleotides in length. In some instances, the hetero-duplex polynucleotide is about 13 nucleotides in length. In some instances, the hetero-duplex polynucleotide is about 12 nucleotides in length. In some instances, the hetero-duplex polynucleotide is about 11 nucleotides in length. In some instances, the hetero-duplex polynucleotide is about 10 nucleotides in length. In some instances, the hetero-duplex polynucleotide is from about 10 to about 50 nucleotides in length. In some instances, the hetero-duplex polynucleotide is from about 10 to about 45 nucleotides in length. In some instances, the hetero-duplex polynucleotide is from about 10 to about 40 nucleotides in length. In some instances, the hetero-duplex polynucleotide is from about 10 to about 35 nucleotides in length. In some instances, the hetero-duplex polynucleotide is from about 10 to about 30 nucleotides in length. In some instances, the hetero-duplex polynucleotide is from about 10 to about 25 nucleotides in length. In some instances, the hetero-duplex polynucleotide is from about 10 to about 20 nucleotides in length. In some instances, the hetero-duplex polynucleotide is from about 15 to about 25 nucleotides in length. In some instances, the hetero-duplex polynucleotide is from about 15 to about 30 nucleotides in length. In some instances, the hetero-duplex polynucleotide is from about 12 to about 30 nucleotides in length.

In some embodiments, the hetero-duplex polynucleotide consists of a guide strand and a passenger strand. In some instances, the guide strand is from about 10 to about 50 nucleotides in length. In some instances, the guide strand is from about 10 to about 30, from about 15 to about 30, from about 18 to about 25, from about 18 to about 24, from about 19 to about 23, or from about 20 to about 22 nucleotides in length.

In some embodiments, the guide strand is about 50 nucleotides in length. In some instances, the guide strand is about 45 nucleotides in length. In some instances, the guide strand is about 40 nucleotides in length. In some instances, the guide strand is about 35 nucleotides in length. In some instances, the guide strand is about 30 nucleotides in length. In some instances, the guide strand is about 25 nucleotides in length. In some instances, the guide strand is about 20 nucleotides in length. In some instances, the guide strand is about 19 nucleotides in length. In some instances, the guide strand is about 18 nucleotides in length. In some instances, the guide strand is about 17 nucleotides in length. In some instances, the guide strand is about 16 nucleotides in length. In some instances, the guide strand is about 15 nucleotides in length. In some instances, the guide strand is about 14 nucleotides in length. In some instances, the guide strand is about 13 nucleotides in length. In some instances, the guide strand is about 12 nucleotides in length. In some instances, the guide strand is about 11 nucleotides in length. In some instances, the guide strand is about 10 nucleotides in length.

In some instances, the guide strand is from about 10 to about 50 nucleotides in length. In some instances, the guide strand is from about 10 to about 45 nucleotides in length. In some instances, the guide strand is from about 10 to about 40 nucleotides in length. In some instances, the guide strand is from about 10 to about 35 nucleotides in length. In some instances, the guide strand is from about 10 to about 30 nucleotides in length. In some instances, the guide strand is from about 10 to about 25 nucleotides in length. In some instances, the guide strand is from about 10 to about 20 nucleotides in length. In some instances, the guide strand is from about 12 to about 30 nucleotides in length. In some instances, the guide strand is from about 15 to about 30 nucleotides in length. In some instances, the guide strand is from about 15 to about 25 nucleotides in length. In some instances, the guide strand is from about 15 to about 24 nucleotides in length. In some instances, the guide strand is from about 15 to about 23 nucleotides in length. In some instances, the guide strand is from about 15 to about 22 nucleotides in length. In some instances, the guide strand is from about 18 to about 30 nucleotides in length. In some instances, the guide strand is from about 18 to about 25 nucleotides in length. In some instances, the guide strand is from about 18 to about 24 nucleotides in length. In some instances, the guide strand is from about 19 to about 23 nucleotides in length. In some instances, the guide strand is from about 20 to about 22 nucleotides in length.

In some embodiments, the passenger strand is about 50 nucleotides in length. In some instances, the passenger strand is about 45 nucleotides in length. In some instances, the passenger strand is about 40 nucleotides in length. In some instances, the passenger strand is about 35 nucleotides in length. In some instances, the passenger strand is about 30 nucleotides in length. In some instances, the passenger strand is about 25 nucleotides in length. In some instances, the passenger strand is about 20 nucleotides in length. In some instances, the passenger strand is about 19 nucleotides in length. In some instances, the passenger strand is about 18 nucleotides in length. In some instances, the passenger strand is about 17 nucleotides in length. In some instances, the passenger strand is about 16 nucleotides in length. In some instances, the passenger strand is about 15 nucleotides in length. In some instances, the passenger strand is about 14 nucleotides in length. In some instances, the passenger strand is about 13 nucleotides in length. In some instances, the passenger strand is about 12 nucleotides in length. In some instances, the passenger strand is about 11 nucleotides in length. In some instances, the passenger strand is about 10 nucleotides in length.

In some instances, the passenger strand is from about 10 to about 50 nucleotides in length. In some instances, the passenger strand is from about 10 to about 45 nucleotides in length. In some instances, the passenger strand is from about 10 to about 40 nucleotides in length. In some instances, the passenger strand is from about 10 to about 35 nucleotides in length. In some instances, the passenger strand is from about 10 to about 30 nucleotides in length. In some instances, the passenger strand is from about 10 to about 25 nucleotides in length. In some instances, the passenger strand is from about 10 to about 20 nucleotides in length. In some instances, the passenger strand is from about 12 to about 30 nucleotides in length. In some instances, the passenger strand is from about 15 to about 30 nucleotides in length. In some instances, the passenger strand is from about 15 to about 25 nucleotides in length. In some instances, the passenger strand is from about 15 to about 24 nucleotides in length. In some instances, the passenger strand is from about 15 to about 23 nucleotides in length. In some instances, the passenger strand is from about 15 to about 22 nucleotides in length. In some instances, the passenger strand is from about 18 to about 30 nucleotides in length. In some instances, the passenger strand is from about 18 to about 25 nucleotides in length. In some instances, the passenger strand is from about 18 to about 24 nucleotides in length. In some instances, the passenger strand is from about 19 to about 23 nucleotides in length. In some instances, the passenger strand is from about 20 to about 22 nucleotides in length.

In some instances, the hetero-duplex polynucleotide comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides.

In some embodiments, the guide strand comprises a sequence having at least 80%, 85%, 90%, 95%, 96%, 97%, 98%, 99%, or 100% sequence identity to SEQ ID NOs: 16-45, 422-1173, 1181-1184, or 1195-1242. In some instances, the guide strand comprises a sequence having at least 80% sequence identity to SEQ ID NOs: 16-45, 422-1173, 1181-1184, or 1195-1242.

In some instances, the guide strand comprises a sequence having at least 85% sequence identity to SEQ ID NOs: 16-45, 422-1173, 1181-1184, or 1195-1242. In some instances, the guide strand comprises a sequence having at least 90% sequence identity to SEQ ID NOs: 16-45, 422-1173, 1181-1184, or 1195-1242. In some instances, the guide strand comprises a sequence having at least 91% sequence identity to SEQ ID NOs: 16-45, 422-1173, 1181-1184, or 1195-1242. In some instances, the guide strand comprises a sequence having at least 92% sequence identity to SEQ ID NOs: 16-45, 422-1173, 1181-1184, or 1195-1242. In some instances, the guide strand comprises a sequence having at least 93% sequence identity to SEQ ID NOs: 16-45, 422-1173, 1181-1184, or 1195-1242. In some instances, the guide strand comprises a sequence having at least 94% sequence identity to SEQ ID NOs: 16-45, 422-1173, 1181-1184, or 1195-1242. In some instances, the guide strand comprises a sequence having at least 95% sequence identity to SEQ ID NOs: 16-45, 422-1173, 1181-1184, or 1195-1242. In some instances, the guide strand comprises a sequence having at least 96% sequence identity to SEQ ID NOs: 16-45, 422-1173, 1181-1184, or 1195-1242. In some instances, the guide strand comprises a sequence having at least 97% sequence identity to SEQ ID NOs: 16-45, 422-1173, 1181-1184, or 1195-1242. In some instances, the guide strand comprises a sequence having at least 98% sequence identity to SEQ ID NOs: 16-45, 422-1173, 1181-1184, or 1195-1242. In some instances, the guide strand comprises a sequence having at least 99% sequence identity to SEQ ID NOs: 16-45, 422-1173, 1181-1184, or 1195-1242. In some instances, the guide strand comprises a sequence having 100% sequence identity to SEQ ID NOs: 16-45, 422-1173, 1181-1184, or 1195-1242. In some instances, the guide strand consists of a sequence having 100% sequence identity to SEQ ID NOs: 16-45, 422-1173, 1181-1184, or 1195-1242.

In some instances, the passenger strand comprises a sequence having at least 85% sequence identity to SEQ ID NOs: 16-45, 422-1173, 1181-1184, or 1195-1242. In some instances, the passenger strand comprises a sequence having at least 90% sequence identity to SEQ ID NOs: 16-45, 422-1173, 1181-1184, or 1195-1242. In some instances, the passenger strand comprises a sequence having at least 91% sequence identity to SEQ ID NOs: 16-45, 422-1173, 1181-1184, or 1195-1242. In some instances, the passenger strand comprises a sequence having at least 92% sequence identity to SEQ ID NOs: 16-45, 422-1173, 1181-1184, or 1195-1242. In some instances, the passenger strand comprises a sequence having at least 93% sequence identity to SEQ ID NOs: 16-45, 422-1173, 1181-1184, or 1195-1242. In some instances, the passenger strand comprises a sequence having at least 94% sequence identity to SEQ ID NOs: 16-45, 422-1173, 1181-1184, or 1195-1242. In some instances, the passenger strand comprises a sequence having at least 95% sequence identity to SEQ ID NOs: 16-45, 422-1173, 1181-1184, or 1195-1242. In some instances, the passenger strand comprises a sequence having at least 96% sequence identity to SEQ ID NOs: 16-45, 422-1173, 1181-1184, or 1195-1242. In some instances, the passenger strand comprises a sequence having at least 97% sequence identity to SEQ ID NOs: 16-45, 422-1173, 1181-1184, or 1195-1242. In some instances, the passenger strand comprises a sequence having at least 98% sequence identity to SEQ ID NOs: 16-45, 422-1173, 1181-1184, or 1195-1242. In some instances, the passenger strand comprises a sequence having at least 99% sequence identity to SEQ ID NOs: 16-45, 422-1173, 1181-1184, or 1195-1242. In some instances, the passenger strand comprises a sequence having 100% sequence identity to SEQ ID NOs: 16-45, 422-1173, 1181-1184, or 1195-1242. In some instances, the passenger strand consists of a sequence having 100% sequence identity to SEQ ID NOs: 16-45, 422-1173, 1181-1184, or 1195-1242.

In some embodiments, the passenger strand comprises two or more polynucleotides. In some cases, each of the two or more polynucleotides hybridizes to a separate region on the guide strand, forming either a continuous strand without a gap between the termini of the two or more polynucleotides or a gap of about 1, 2, 3, or more bases between the termini of the two or more polynucleotides. In some cases, the two or more polynucleotides independently comprise at least 6, 7, 8, 9, 10, 11, 12, 13, 14, 15, 16, 17, 18, 19, 20, or more phosphorodiamidate morpholino oligomer-modified non-natural nucleotides or at least 6, 7, 8, 9, 10, 11, 12, 13, 14, 15, 16, 17, 18, 19, 20, or more peptide nucleic acid-modified non-natural nucleotides. In other cases, the two or more polynucleotides independently comprise 100% phosphorodiamidate morpholino oligomer-modified non-natural nucleotides or 100% peptide nucleic acid-modified non-natural nucleotides.

In some embodiments, the sequence of the hetero-duplex polynucleotide is at least 40%, 50%, 55%, 60%, 65%, 70%, 75%, 80%, 85%, 90%, 95%, 98%, 99%, or 99.5% complementary to a target sequence described herein. In some embodiments, the sequence of the hetero-duplex polynucleotide is at least 50% complementary to a target sequence described herein. In some embodiments, the sequence of the hetero-duplex polynucleotide is at least 60% complementary to a target sequence described herein. In some embodiments, the sequence of the hetero-duplex polynucleotide is at least 70% complementary to a target sequence described herein. In some embodiments, the sequence of the hetero-duplex polynucleotide is at least 80% complementary to a target sequence described herein. In some embodiments, the sequence of the hetero-duplex polynucleotide is at least 90% complementary to a target sequence described herein. In some embodiments, the sequence of the hetero-duplex polynucleotide is at least 95% complementary to a target sequence described herein. In some embodiments, the sequence of the hetero-duplex polynucleotide is at least 99% complementary to a target sequence described herein. In some instances, the sequence of the hetero-duplex polynucleotide is 100% complementary to a target sequence described herein.

In some embodiments, the sequence of the hetero-duplex polynucleotide has 5 or less mismatches to a target sequence described herein. In some embodiments, the sequence of the hetero-duplex polynucleotide has 4 or less mismatches to a target sequence described herein. In some instances, the sequence of the hetero-duplex polynucleotide has 3 or less mismatches to a target sequence described herein. In some cases, the sequence of the hetero-duplex polynucleotide has 2 or less mismatches to a target sequence described herein. In some cases, the sequence of the hetero-duplex polynucleotide has 1 or less mismatches to a target sequence described herein.

In some embodiments, the specificity of the hetero-duplex polynucleotide that hybridizes to a target sequence described herein is a 95%, 98%, 99%, 99.5%, or 100% sequence complementarity of the hetero-duplex polynucleotide to a target sequence. In some instances, the hybridization is a high stringent hybridization condition.

In some embodiments, the hetero-duplex polynucleotide hybridizes to at least 8, 9, 10, 11, 12, 13, 14, 15, 16, 17, 18, 19, 20, or more contiguous bases of a target sequence described herein. In some embodiments, the hetero-duplex polynucleotide hybridizes to at least 8 contiguous bases of a target sequence described herein. In some embodiments, the hetero-duplex polynucleotide hybridizes to at least 9 contiguous bases of a target sequence described herein. In some embodiments, the hetero-duplex polynucleotide hybridizes to at least 10 contiguous bases of a target sequence described herein. In some embodiments, the hetero-duplex polynucleotide hybridizes to at least 11 contiguous bases of a target sequence described herein. In some embodiments, the hetero-duplex polynucleotide hybridizes to at least 12 contiguous bases of a target sequence described herein. In some embodiments, the hetero-duplex polynucleotide hybridizes to at least 13 contiguous bases of a target sequence described herein. In some embodiments, the hetero-duplex polynucleotide hybridizes to at least 14 contiguous bases of a target sequence described herein. In some embodiments, the hetero-duplex polynucleotide hybridizes to at least 15 contiguous bases of a target sequence described herein. In some embodiments, the hetero-duplex polynucleotide hybridizes to at least 16 contiguous bases of a target sequence described herein. In some embodiments, the hetero-duplex polynucleotide hybridizes to at least 17 contiguous bases of a target sequence described herein. In some embodiments, the hetero-duplex polynucleotide hybridizes to at least 18 contiguous bases of a target sequence described herein. In some embodiments, the hetero-duplex polynucleotide hybridizes to at least 19 contiguous bases of a target sequence described herein. In some embodiments, the hetero-duplex polynucleotide hybridizes to at least 20 contiguous bases of a target sequence described herein.

In some embodiments, the hetero-duplex polynucleotide has reduced off-target effect. In some instances, “off-target” or “off-target effects” refer to any instance in which a polynucleic acid polymer directed against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the hetero-duplex polynucleotide.

In some cases, one or more of the artificial nucleotide analogues described herein are resistant toward nucleases such as for example ribonuclease such as RNase H, deoxyribunuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribunuclease such as DNase, or exonuclease such as 5′-3′ exonuclease and 3′-5′ exonuclease. In some instances, 2′-O-methyl modified polynucleic acid molecule is nuclease resistant (e.g., RNase H, DNase, 5′-3′ exonuclease or 3′-5′ exonuclease resistant). In some instances, 2′O-methoxyethyl (2′-O-MOE) modified polynucleic acid molecule is nuclease resistant (e.g., RNase H, DNase, 5′-3′ exonuclease or 3′-5′ exonuclease resistant). In some instances, 2′-O-aminopropyl modified polynucleic acid molecule is nuclease resistant (e.g., RNase H, DNase, 5′-3′ exonuclease or 3′-5′ exonuclease resistant). In some instances, 2′-deoxy modified polynucleic acid molecule is nuclease resistant (e.g., RNase H, DNase, 5′-3′ exonuclease or 3′-5′ exonuclease resistant). In some instances, T-deoxy-2′-fluoro modified polynucleic acid molecule is nuclease resistant (e.g., RNase H, DNase, 5′-3′ exonuclease or 3′-5′ exonuclease resistant). In some instances, 2′-O-aminopropyl (2′-O-AP) modified polynucleic acid molecule is nuclease resistant (e.g., RNase H, DNase, 5′-3′ exonuclease or 3′-5′ exonuclease resistant). In some instances, 2′-O-dimethylaminoethyl (2′-O-DMAOE) modified polynucleic acid molecule is nuclease resistant (e.g., RNase H, DNase, 5′-3′ exonuclease or 3′-5′ exonuclease resistant). In some instances, 2′-O-dimethylaminopropyl (2′-O-DMAP) modified polynucleic acid molecule is nuclease resistant (e.g., RNase H, DNase, 5′-3′ exonuclease or 3′-5′ exonuclease resistant). In some instances, T-O-dimethylaminoethyloxyethyl (2′-O-DMAEOE) modified polynucleic acid molecule is nuclease resistant (e.g., RNase H, DNase, 5′-3′ exonuclease or 3′-5′ exonuclease resistant). In some instances, 2′-O—N-methylacetamido (2′-O-NMA) modified polynucleic acid molecule is nuclease resistant (e.g., RNase H, DNase, 5′-3′ exonuclease or 3′-5′ exonuclease resistant). In some instances, LNA-modified polynucleic acid molecule is nuclease resistant (e.g., RNase H, DNase, 5′-3′ exonuclease or 3′-5′ exonuclease resistant). In some instances, ENA-modified polynucleic acid molecule is nuclease resistant (e.g., RNase H, DNase, 5′-3′ exonuclease or 3′-5′ exonuclease resistant). In some instances, HNA-modified polynucleic acid molecule is nuclease resistant (e.g., RNase H, DNase, 5′-3′ exonuclease or 3′-5′ exonuclease resistant). Morpholinos may be nuclease resistant (e.g., RNase H, DNase, 5′-3′ exonuclease or 3′-5′ exonuclease resistant). In some instances, PNA-modified polynucleic acid molecule is resistant to nucleases (e.g., RNase H, DNase, 5′-3′ exonuclease or 3′-5′ exonuclease resistant). In some instances, methylphosphonate nucleotide-modified polynucleic acid molecule is nuclease resistant (e.g., RNase H, DNase, 5′-3′ exonuclease or 3′-5′ exonuclease resistant). In some instances, thiolphosphonate nucleotide-modified polynucleic acid molecule is nuclease resistant (e.g., RNase H, DNase, 5′-3′ exonuclease or 3′-5′ exonuclease resistant). In some instances, polynucleic acid molecule comprising 2′-fluoro N3-P5′-phosphoramidites is nuclease resistant (e.g., RNase H, DNase, 5′-3′ exonuclease or 3′-5′ exonuclease resistant). In some instances, the 5′ conjugates described herein inhibit 5′-3′ exonucleolytic cleavage. In some instances, the 3′ conjugates described herein inhibit 3′-5′ exonucleolytic cleavage.

In some embodiments, one or more of the artificial nucleotide analogues described herein have increased binding affinity toward their mRNA target relative to an equivalent natural polynucleic acid molecule. The one or more of the artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some instances, 2′-O-methyl modified polynucleic acid molecule has increased binding affinity toward their mRNA target relative to an equivalent natural polynucleic acid molecule. In some instances, 2′-O-methoxyethyl (2′-O-MOE) modified polynucleic acid molecule has increased binding affinity toward their mRNA target relative to an equivalent natural polynucleic acid molecule. In some instances, 2′-O-aminopropyl modified polynucleic acid molecule has increased binding affinity toward their mRNA target relative to an equivalent natural polynucleic acid molecule. In some instances, 2′-deoxy modified polynucleic acid molecule has increased binding affinity toward their mRNA target relative to an equivalent natural polynucleic acid molecule. In some instances, T-deoxy-2′-fluoro modified polynucleic acid molecule has increased binding affinity toward their mRNA target relative to an equivalent natural polynucleic acid molecule. In some instances, 2′-O-aminopropyl (2′-O-AP) modified polynucleic acid molecule has increased binding affinity toward their mRNA target relative to an equivalent natural polynucleic acid molecule. In some instances, 2′-O-dimethylaminoethyl (2′-O-DMAOE) modified polynucleic acid molecule has increased binding affinity toward their mRNA target relative to an equivalent natural polynucleic acid molecule. In some instances, 2′-O-dimethylaminopropyl (2′-O-DMAP) modified polynucleic acid molecule has increased binding affinity toward their mRNA target relative to an equivalent natural polynucleic acid molecule. In some instances, T-O-dimethylaminoethyloxyethyl (2′-O-DMAEOE) modified polynucleic acid molecule has increased binding affinity toward their mRNA target relative to an equivalent natural polynucleic acid molecule. In some instances, 2′-O—N-methylacetamido (2′-O-NMA) modified polynucleic acid molecule has increased binding affinity toward their mRNA target relative to an equivalent natural polynucleic acid molecule. In some instances, LNA-modified polynucleic acid molecule has increased binding affinity toward their mRNA target relative to an equivalent natural polynucleic acid molecule. In some instances, ENA-modified polynucleic acid molecule has increased binding affinity toward their mRNA target relative to an equivalent natural polynucleic acid molecule. In some instances, PNA-modified polynucleic acid molecule has increased binding affinity toward their mRNA target relative to an equivalent natural polynucleic acid molecule. In some instances, HNA-modified polynucleic acid molecule has increased binding affinity toward their mRNA target relative to an equivalent natural polynucleic acid molecule. In some instances, morpholino-modified polynucleic acid molecule has increased binding affinity toward their mRNA target relative to an equivalent natural polynucleic acid molecule. In some instances, methylphosphonate nucleotides-modified polynucleic acid molecule has increased binding affinity toward their mRNA target relative to an equivalent natural polynucleic acid molecule. In some instances, thiolphosphonate nucleotides-modified polynucleic acid molecule has increased binding affinity toward their mRNA target relative to an equivalent natural polynucleic acid molecule. In some instances, polynucleic acid molecule comprising 2′-fluoro N3-P5′-phosphoramidites has increased binding affinity toward their mRNA target relative to an equivalent natural polynucleic acid molecule. In some cases, the increased affinity is illustrated with a lower Kd, a higher melt temperature (Tm), or a combination thereof.

In some embodiments, a hetero-duplex polynucleotide described herein is a chirally pure (or stereo pure) polynucleic acid molecule, or a polynucleic acid molecule comprising a single enantiomer. In some instances, the hetero-duplex polynucleotide comprises L-nucleotide. In some instances, the hetero-duplex polynucleotide comprises D-nucleotides. In some instance, a hetero-duplex polynucleotide composition comprises less than 30%, 25%, 20%, 15%, 10%, 5%, 4%, 3%, 2%, 1%, or less of its mirror enantiomer. In some cases, a hetero-duplex polynucleotide composition comprises less than 30%, 25%, 20%, 15%, 10%, 5%, 4%, 3%, 2%, 1%, or less of a racemic mixture. In some instances, the hetero-duplex polynucleotide is a polynucleic acid molecule described in: U.S. Patent Publication Nos: 2014/194610 and 2015/211006; and PCT Publication No.: WO2015107425.

In some embodiments, a hetero-duplex polynucleotide described herein is further modified to include an aptamer-conjugating moiety. In some instances, the aptamer-conjugating moiety is a DNA aptamer-conjugating moiety. In some instances, the aptamer-conjugating moiety is Alphamer (Centauri Therapeutics), which comprises an aptamer portion that recognizes a specific cell-surface target and a portion that presents a specific epitopes for attaching to circulating antibodies. In some instance, a hetero-duplex polynucleotide described herein is further modified to include an aptamer-conjugating moiety as described in: U.S. Pat. Nos. 8,604,184, 8,591,910, and 7,850,975.

In additional embodiments, a hetero-duplex polynucleotide described herein is modified to increase its stability. In some instances, the hetero-duplex polynucleotide is modified by one or more of the modifications described above to increase its stability. In some cases, the hetero-duplex polynucleotide is modified at the 2′ hydroxyl position, such as by 2′-O-methyl, 2′-O-methoxyethyl (2′-0-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the hetero-duplex polynucleotide is modified by 2′-O-methyl and/or 2′-O-methoxyethyl ribose. In some cases, the hetero-duplex polynucleotide also includes morpholinos, PNAs, HNA, methylphosphonate nucleotides, thiolphosphonate nucleotides, and/or 2′-fluoro N3-P5′-phosphoramidites to increase its stability. In some instances, the hetero-duplex polynucleotide is a chirally pure (or stereo pure) polynucleic acid molecule. In some instances, the chirally pure (or stereo pure) polynucleic acid molecule is modified to increase its stability. Suitable modifications to the RNA to increase stability for delivery will be apparent to the skilled person.

### Conjugation Chemistry

In some embodiments, a hetero-duplex polynucleotide is conjugated to a binding moiety. In some instances, the binding moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of binding moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some instances, the binding moiety is an antibody or binding fragment thereof. In some instances, the hetero-duplex polynucleotide is further conjugated to a polymer, and optionally an endosomolytic moiety.

In some embodiments, the hetero-duplex polynucleotide is conjugated to the binding moiety by a chemical ligation process. In some instances, the hetero-duplex polynucleotide is conjugated to the binding moiety by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some embodiments, the hetero-duplex polynucleotide is conjugated to the binding moiety either site-specifically or non-specifically via native ligation chemistry.

In some instances, the hetero-duplex polynucleotide is conjugated to the binding moiety by a site-directed method utilizing a “traceless” coupling technology (Philochem). In some instances, the “traceless” coupling technology utilizes an N-terminal 1,2-aminothiol group on the binding moiety which is then conjugate with a hetero-duplex polynucleotide containing an aldehyde group. (see Casi et al., “Site-specific traceless coupling of potent cytotoxic drugs to recombinant antibodies for pharmacodelivery,” JACS 134(13): 5887-5892 (2012))

In some instances, the hetero-duplex polynucleotide is conjugated to the binding moiety by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the hetero-duplex polynucleotide is conjugated to the binding moiety by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized hetero-duplex polynucleotide via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some instances, the enzyme-catalyzed process comprises microbial transglutaminase (mTG). In some cases, the hetero-duplex polynucleotide is conjugated to the binding moiety utilizing a microbial transglutaminze catalyzed process. In some instances, mTG catalyzes the formation of a covalent bond between the amide side chain of a glutamine within the recognition sequence and a primary amine of a functionalized hetero-duplex polynucleotide. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some instances, the hetero-duplex polynucleotide is conjugated to the binding moiety by a method as described in PCT Publication No. WO2014/140317, which utilizes a sequence-specific transpeptidase.

In some instances, the hetero-duplex polynucleotide is conjugated to the binding moiety by a method as described in U.S. Patent Publication Nos. 2015/0105539 and 2015/0105540.

### Nucleic Acid-Polypeptide Conjugate

In some embodiments, a hetero-duplex polynucleotide is further conjugated to a polypeptide A for delivery to a site of interest. In some cases, a hetero-duplex polynucleotide is conjugated to a polypeptide A and optionally a polymeric moiety.

In some instances, at least one polypeptide A is conjugated to at least one B. In some instances, the at least one polypeptide A is conjugated to the at least one B to form an A-B conjugate. In some embodiments, at least one A is conjugated to the 5′ terminus of B, the 3′ terminus of B, an internal site on B, or in any combinations thereof. In some instances, the at least one polypeptide A is conjugated to at least two B. In some instances, the at least one polypeptide A is conjugated to at least 2, 3, 4, 5, 6, 7, 8, or more B.

In some embodiments, at least one polypeptide A is conjugated at one terminus of at least one B while at least one C is conjugated at the opposite terminus of the at least one B to form an A-B-C conjugate. In some instances, at least one polypeptide A is conjugated at one terminus of the at least one B while at least one of C is conjugated at an internal site on the at least one B. In some instances, at least one polypeptide A is conjugated directly to the at least one C. In some instances, the at least one B is conjugated indirectly to the at least one polypeptide A via the at least one C to form an A-C-B conjugate.

In some instances, at least one B and/or at least one C, and optionally at least one D are conjugated to at least one polypeptide A. In some instances, the at least one B is conjugated at a terminus (e.g., a 5′ terminus or a 3′ terminus) to the at least one polypeptide A or are conjugated via an internal site to the at least one polypeptide A. In some cases, the at least one C is conjugated either directly to the at least one polypeptide A or indirectly via the at least one B. If indirectly via the at least one B, the at least one C is conjugated either at the same terminus as the at least one polypeptide A on B, at opposing terminus from the at least one polypeptide A, or independently at an internal site. In some instances, at least one additional polypeptide A is further conjugated to the at least one polypeptide A, to B, or to C. In additional instances, the at least one D is optionally conjugated either directly or indirectly to the at least one polypeptide A, to the at least one B, or to the at least one C. If directly to the at least one polypeptide A, the at least one D is also optionally conjugated to the at least one B to form an A-D-B conjugate or is optionally conjugated to the at least one B and the at least one C to form an A-D-B-C conjugate. In some instances, the at least one D is directly conjugated to the at least one polypeptide A and indirectly to the at least one B and the at least one C to form a D-A-B-C conjugate. If indirectly to the at least one polypeptide A, the at least one D is also optionally conjugated to the at least one B to form an A-B-D conjugate or is optionally conjugated to the at least one B and the at least one C to form an A-B-D-C conjugate. In some instances, at least one additional D is further conjugated to the at least one polypeptide A, to B, or to C.

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

In some embodiments, a polynucleic acid molecule conjugate comprises a construct as illustrated:

The

as illustrated above is for representation purposes only and encompasses a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof.

In some embodiments, the polynucleic acid molecule conjugate comprises a molecule of Formula (I): A-(X¹—B)ₙ, in which A comprises a binding moiety, B consists of a hetero-duplex polynucleotide consisting of a guide strand and a passenger strand, X¹ consists of a bond or first non-polymeric linker, and n is an averaged value selected from 1-12, wherein the guide strand comprises at least one but no more than 10 phosphorothioate-modified non-natural nucleotides, wherein the passenger strand comprises a plurality of phosphorodiamidate morpholino oligomers or a plurality of peptide nucleic acid-modified non-natural nucleotides, and wherein the hetero-duplex polynucleotide has one of: a greater hepatocyte stability, reduced overall charge, reduced hepatocyte uptake, or extended pharmacokinetics, compare to analogous homoduplex nucleotide. In some instances, A-X¹ is conjugated to the 5′ end of the passenger strand. In other instances, A-X¹ is conjugated to the 3′ end of the passenger strand.

In some embodiments, the polynucleic acid molecule conjugate comprises a molecule of Formula (II): A-X¹—(B—X²—C)ₙ, in which A comprises a binding moiety; B consists of a hetero-duplex polynucleotide consisting of a guide strand and a passenger strand; C consists of a polymer; X¹ consists of a bond or first non-polymeric linker; and X² consists of a bond or second non-polymeric linker; wherein A and C are not attached to B at the same terminus, wherein the guide strand comprises at least one but no more than 10 phosphorothioate-modified non-natural nucleotides, wherein the passenger strand comprises a plurality of phosphorodiamidate morpholino oligomers or a plurality of peptide nucleic acid-modified non-natural nucleotides, and wherein the hetero-duplex polynucleotide has one of: a greater hepatocyte stability, reduced overall charge, reduced hepatocyte uptake, or extended pharmacokinetics, compare to analogous homoduplex nucleotide. In some instances, C is directly conjugated to B via X². In some instances, A-X¹ is conjugated to the 5′ end of the passenger strand and X²—C is conjugated to the 3′ end of the passenger strand. In other instances, X²—C is conjugated to the 5′ end of the passenger strand and A-X¹ is conjugated to the 3′ end of the passenger strand.

### Binding Moiety

In some embodiments, the binding moiety A is a polypeptide. In some instances, the polypeptide is an antibody or its fragment thereof. In some cases, the fragment is a binding fragment. In some instances, the antibody or binding fragment thereof comprises a humanized antibody or binding fragment thereof, murine antibody or binding fragment thereof, chimeric antibody or binding fragment thereof, monoclonal antibody or binding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, camelid antibody or binding fragment thereof, bispecific antibody or biding fragment thereof, or a chemically modified derivative thereof.

In some instances, A is an antibody or binding fragment thereof. In some instances, A is a humanized antibody or binding fragment thereof, murine antibody or binding fragment thereof, chimeric antibody or binding fragment thereof, monoclonal antibody or binding fragment thereof, monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (“dsFv”), single-domain antibody (sdAb), Ig NAR, camelid antibody or binding fragment thereof, bispecific antibody or biding fragment thereof, or a chemically modified derivative thereof. In some instances, A is a humanized antibody or binding fragment thereof. In some instances, A is a murine antibody or binding fragment thereof. In some instances, A is a chimeric antibody or binding fragment thereof. In some instances, A is a monoclonal antibody or binding fragment thereof. In some instances, A is a monovalent Fab′. In some instances, A is a diavalent Fab₂. In some instances, A is a single-chain variable fragment (scFv).

In some embodiments, the binding moiety A is a bispecific antibody or binding fragment thereof. In some instances, the bispecific antibody is a trifunctional antibody or a bispecific mini-antibody. In some cases, the bispecific antibody is a trifunctional antibody. In some instances, the trifunctional antibody is a full length monoclonal antibody comprising binding sites for two different antigens. Exemplary trifunctional antibodies include catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (RO5520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), and TRBS07 (Ektomab; targets GD2/CD3; Trion Research Gmbh). Additional exemplary trifunctional antibodies include mAb² from F-star Biotechnology Ltd. In some instances, A is a bispecific trifunctional antibody. In some embodiments, A is a bispecific trifunctional antibody selected from: catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (RO5520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), TRBS07 (Ektomab; targets GD2/CD3; Trion Research Gmbh), and a mAb² from F-star Biotechnology Ltd.

In some cases, the bispecific antibody is a bispecific mini-antibody. In some instances, the bispecific mini-antibody comprises divalent Fab₂, F(ab)′₃ fragments, bis-scFv, (scFv)₂, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens. Exemplary bispecific mini-antibodies include, but are not limited to, DART (dual-affinity re-targeting platform; MacroGenics), blinatumomab (MT103 or AMG103; which targets CD19/CD3; Micromet), MT111 (targets CEA/CD3; Micromet/Amegen), MT112 (BAY2010112; targets PSMA/CD3; Micromet/Bayer), MT110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), and domain antibodies (dAbs from Domantis/GSK).

In some embodiments, the binding moiety A is a bispecific mini-antibody. In some instances, A is a bispecific Fab₂. In some instances, A is a bispecific F(ab)′₃ fragment. In some cases, A is a bispecific bis-scFv. In some cases, A is a bispecific (scFv)₂. In some embodiments, A is a bispecific diabody. In some embodiments, A is a bispecific minibody. In some embodiments, A is a bispecific triabody. In other embodiments, A is a bispecific tetrabody. In other embodiments, A is a bi-specific T-cell engager (BiTE). In additional embodiments, A is a bispecific mini-antibody selected from: DART (dual-affinity re-targeting platform; MacroGenics), blinatumomab (MT103 or AMG103; which targets CD19/CD3; Micromet), MT111 (targets CEA/CD3; Micromet/Amegen), MT112 (BAY2010112; targets PSMA/CD3; Micromet/Bayer), MT110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), and domain antibodies (dAbs from Domantis/GSK).

In some embodiments, the binding moiety A is a trispecific antibody. In some instances, the trispecific antibody comprises F(ab)′₃ fragments or a triabody. In some instances, A is a trispecific F(ab)′₃ fragment. In some cases, A is a triabody. In some embodiments, A is a trispecific antibody as described in Dimas, et al., “Development of a trispecific antibody designed to simultaneously and efficiently target three different antigens on tumor cells,” Mol. Pharmaceutics, 12(9): 3490-3501 (2015).

In some embodiments, the binding moiety A is an antibody or binding fragment thereof that recognizes a cell surface protein. In some instances, the cell surface protein is an antigen expressed by a cancerous cell. Exemplary cancer antigens include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCRS, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Ra, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) and the like.

In some instances, the cell surface protein comprises clusters of differentiation (CD) cell surface markers. Exemplary CD cell surface markers include, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), and the like.

In some instances, the binding moiety A is an antibody or binding fragment thereof that recognizes a cancer antigen. In some instances, the binding moiety A is an antibody or binding fragment thereof that recognizes alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCRS, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Rα, Immunoglobulin superfamily receptor translocation associated 2 (IRTA2), L6, Lewis Y, Lewis X, MAGE-1, MAGE-2, MAGE-3, MAGE 4, MART1, mesothelin, MCP1 (CCL2), MDP, macrophage inhibitory factor (MIF), MPF (SMR, MSLN), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) or a combination thereof.

In some instances, the binding moiety A is an antibody or binding fragment thereof that recognizes a CD cell surface marker. In some instances, the binding moiety A is an antibody or binding fragment thereof that recognizes CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), ora combination thereof.

In some embodiments, the antibody or binding fragment thereof comprises zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hu1D10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAb17-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), ficlatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C11 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-ST01, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), narnatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart MI95 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), zatuximab, and the like.

In some embodiments, the binding moiety A comprises zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hu1D10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAb17-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), ficlatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C11 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-ST01, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), narnatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart MI95 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), or zatuximab. In some embodiments, the binding moiety A is zalutumumab (HuMax-EFGr, by Genmab).

In some embodiments, the binding moiety A is conjugated according to Formula (I) to a hetero-duplex polynucleotide (B), and a polymer (C), and optionally an endosomolytic moiety (D) according to Formula (II) described herein. In some instances, the hetero-duplex polynucleotide comprises a sequence having at least 50%, 55%, 60%, 65%, 70%, 75%, 80%, 85%, 90%, 95%, 96%, 97%, 98%, 99%, or 100% sequence identity to a sequence listed in Tables 2, 4, 8, or 9. In some embodiments, the hetero-duplex polynucleotide comprises a sequence having at least 50%, 55%, 60%, 65%, 70%, 75%, 80%, 85%, 90%, 95%, 96%, 97%, 98%, 99%, or 100% sequence identity to SEQ ID NOs: 16-45, 422-1173, 1195-1214, or 1215-1242. In some instances, the hetero-duplex polynucleotide comprises a sequence selected from SEQ ID NOs: 16-45, 422-1173, 1195-1214, or 1215-1242. In some instances, the polymer C comprises polyalkylen oxide (e.g., polyethylene glycol). In some embodiments, the endosomolytic moiety D comprises INF7 or melittin, or their respective derivatives.

In some embodiments, the binding moiety A is conjugated to a hetero-duplex polynucleotide (B), and a polymer (C), and optionally an endosomolytic moiety (D). In some instances, the binding moiety A is an antibody or binding fragment thereof.

In some embodiments, the binding moiety A is conjugated to a hetero-duplex polynucleotide (B) non-specifically. In some instances, the binding moiety A is conjugated to a hetero-duplex polynucleotide (B) via a lysine residue or a cysteine residue, in a non-site specific manner. In some instances, the binding moiety A is conjugated to a hetero-duplex polynucleotide (B) via a lysine residue in a non-site specific manner. In some cases, the binding moiety A is conjugated to a hetero-duplex polynucleotide (B) via a cysteine residue in a non-site specific manner. In some instances, the binding moiety A is an antibody or binding fragment thereof.

In some embodiments, the binding moiety A is conjugated to a hetero-duplex polynucleotide (B) in a site-specific manner. In some instances, the binding moiety A is conjugated to a hetero-duplex polynucleotide (B) through a lysine residue, a cysteine residue, at the 5′-terminus, at the 3′-terminus, an unnatural amino acid, or an enzyme-modified or enzyme-catalyzed residue, via a site-specific manner. In some instances, the binding moiety A is conjugated to a hetero-duplex polynucleotide (B) through a lysine residue via a site-specific manner. In some instances, the binding moiety A is conjugated to a hetero-duplex polynucleotide (B) through a cysteine residue via a site-specific manner. In some instances, the binding moiety A is conjugated to a hetero-duplex polynucleotide (B) at the 5′-terminus via a site-specific manner. In some instances, the binding moiety A is conjugated to a hetero-duplex polynucleotide (B) at the 3′-terminus via a site-specific manner. In some instances, the binding moiety A is conjugated to a hetero-duplex polynucleotide (B) through an unnatural amino acid via a site-specific manner. In some instances, the binding moiety A is conjugated to a hetero-duplex polynucleotide (B) through an enzyme-modified or enzyme-catalyzed residue via a site-specific manner. In some instances, the binding moiety A is an antibody or binding fragment thereof.

In some embodiments, one or more regions of a binding moiety A (e.g., an antibody or binding fragment thereof) is conjugated to a hetero-duplex polynucleotide (B). In some instances, the one or more regions of a binding moiety A comprise the N-terminus, the C-terminus, in the constant region, at the hinge region, or the Fc region of the binding moiety A. In some instances, the hetero-duplex polynucleotide (B) is conjugated to the N-terminus of the binding moiety A (e.g., the N-terminus of an antibody or binding fragment thereof). In some instances, the hetero-duplex polynucleotide (B) is conjugated to the C-terminus of the binding moiety A (e.g., the N-terminus of an antibody or binding fragment thereof). In some instances, the hetero-duplex polynucleotide (B) is conjugated to the constant region of the binding moiety A (e.g., the constant region of an antibody or binding fragment thereof). In some instances, the hetero-duplex polynucleotide (B) is conjugated to the hinge region of the binding moiety A (e.g., the constant region of an antibody or binding fragment thereof). In some instances, the hetero-duplex polynucleotide (B) is conjugated to the Fc region of the binding moiety A (e.g., the constant region of an antibody or binding fragment thereof).

In some embodiments, one or more hetero-duplex polynucleotide (B) is conjugated to a binding moiety A. In some instances, about 1, 2, 3, 4, 5, 6, 7, 8, 9, 10, 11, 12, 13, 14, 15, 16, or more hetero-duplex polynucleotides are conjugated to one binding moiety A. In some instances, about 1 hetero-duplex polynucleotide is conjugated to one binding moiety A. In some instances, about 2 hetero-duplex polynucleotides are conjugated to one binding moiety A. In some instances, about 3 hetero-duplex polynucleotides are conjugated to one binding moiety A. In some instances, about 4 hetero-duplex polynucleotides are conjugated to one binding moiety A. In some instances, about 5 hetero-duplex polynucleotides are conjugated to one binding moiety A. In some instances, about 6 hetero-duplex polynucleotides are conjugated to one binding moiety A. In some instances, about 7 hetero-duplex polynucleotides are conjugated to one binding moiety A. In some instances, about 8 hetero-duplex polynucleotides are conjugated to one binding moiety A. In some instances, about 9 hetero-duplex polynucleotides are conjugated to one binding moiety A. In some instances, about 10 hetero-duplex polynucleotides are conjugated to one binding moiety A. In some instances, about 11 hetero-duplex polynucleotides are conjugated to one binding moiety A. In some instances, about 12 hetero-duplex polynucleotides are conjugated to one binding moiety A. In some instances, about 13 hetero-duplex polynucleotides are conjugated to one binding moiety A. In some instances, about 14 hetero-duplex polynucleotides are conjugated to one binding moiety A. In some instances, about 15 hetero-duplex polynucleotides are conjugated to one binding moiety A. In some instances, about 16 hetero-duplex polynucleotides are conjugated to one binding moiety A. In some cases, the one or more hetero-duplex polynucleotides are the same. In other cases, the one or more hetero-duplex polynucleotides are different. In some instances, the binding moiety A is an antibody or binding fragment thereof.

In some embodiments, the number of hetero-duplex polynucleotide (B) conjugated to a binding moiety A (e.g., an antibody or binding fragment thereof) forms a ratio. In some instances, the ratio is referred to as a DAR (drug-to-antibody) ratio, in which the drug as referred to herein is the hetero-duplex polynucleotide (B). In some instances, the DAR ratio of the hetero-duplex polynucleotide (B) to binding moiety A is about 1, 2, 3, 4, 5, 6, 7, 8, 9, 10, 11, 12, 13, 14, 15, 16, or greater. In some instances, the DAR ratio of the hetero-duplex polynucleotide (B) to binding moiety A is about 1 or greater. In some instances, the DAR ratio of the hetero-duplex polynucleotide (B) to binding moiety A is about 2 or greater. In some instances, the DAR ratio of the hetero-duplex polynucleotide (B) to binding moiety A is about 3 or greater. In some instances, the DAR ratio of the hetero-duplex polynucleotide (B) to binding moiety A is about 4 or greater. In some instances, the DAR ratio of the hetero-duplex polynucleotide (B) to binding moiety A is about 5 or greater. In some instances, the DAR ratio of the hetero-duplex polynucleotide (B) to binding moiety A is about 6 or greater. In some instances, the DAR ratio of the hetero-duplex polynucleotide (B) to binding moiety A is about 7 or greater. In some instances, the DAR ratio of the hetero-duplex polynucleotide (B) to binding moiety A is about 8 or greater. In some instances, the DAR ratio of the hetero-duplex polynucleotide (B) to binding moiety A is about 9 or greater. In some instances, the DAR ratio of the hetero-duplex polynucleotide (B) to binding moiety A is about 10 or greater. In some instances, the DAR ratio of the hetero-duplex polynucleotide (B) to binding moiety A is about 11 or greater. In some instances, the DAR ratio of the hetero-duplex polynucleotide (B) to binding moiety A is about 12 or greater.

In some instances, the DAR ratio of the hetero-duplex polynucleotide (B) to binding moiety A (e.g., an antibody or binding fragment thereof) is about 1, 2, 3, 4, 5, 6, 7, 8, 9, 10, 11, 12, 13, 14, 15, or 16. In some instances, the DAR ratio of the hetero-duplex polynucleotide (B) to binding moiety A is about 1. In some instances, the DAR ratio of the hetero-duplex polynucleotide (B) to binding moiety A is about 2. In some instances, the DAR ratio of the hetero-duplex polynucleotide (B) to binding moiety A is about 3. In some instances, the DAR ratio of the hetero-duplex polynucleotide (B) to binding moiety A is about 4. In some instances, the DAR ratio of the hetero-duplex polynucleotide (B) to binding moiety A is about 5. In some instances, the DAR ratio of the hetero-duplex polynucleotide (B) to binding moiety A is about 6. In some instances, the DAR ratio of the hetero-duplex polynucleotide (B) to binding moiety A is about 7. In some instances, the DAR ratio of the hetero-duplex polynucleotide (B) to binding moiety A is about 8. In some instances, the DAR ratio of the hetero-duplex polynucleotide (B) to binding moiety A is about 9. In some instances, the DAR ratio of the hetero-duplex polynucleotide (B) to binding moiety A is about 10. In some instances, the DAR ratio of the hetero-duplex polynucleotide (B) to binding moiety A is about 11. In some instances, the DAR ratio of the hetero-duplex polynucleotide (B) to binding moiety A is about 12. In some instances, the DAR ratio of the hetero-duplex polynucleotide (B) to binding moiety A is about 13. In some instances, the DAR ratio of the hetero-duplex polynucleotide (B) to binding moiety A is about 14. In some instances, the DAR ratio of the hetero-duplex polynucleotide (B) to binding moiety A is about 15. In some instances, the DAR ratio of the hetero-duplex polynucleotide (B) to binding moiety A is about 16.

In some instances, the DAR ratio of the hetero-duplex polynucleotide (B) to binding moiety A is 1, 2, 3, 4, 5, 6, 7, 8, 9, 10, 11, 12, 13, 14, 15, or 16. In some instances, the DAR ratio of the hetero-duplex polynucleotide (B) to binding moiety A is 1. In some instances, the DAR ratio of the hetero-duplex polynucleotide (B) to binding moiety A is 2. In some instances, the DAR ratio of the hetero-duplex polynucleotide (B) to binding moiety A is 4. In some instances, the DAR ratio of the hetero-duplex polynucleotide (B) to binding moiety A is 6. In some instances, the DAR ratio of the hetero-duplex polynucleotide (B) to binding moiety A is 8. In some instances, the DAR ratio of the hetero-duplex polynucleotide (B) to binding moiety A is 12.

In some embodiments, an antibody or its binding fragment is further modified using conventional techniques known in the art, for example, by using amino acid deletion, insertion, substitution, addition, and/or by recombination and/or any other modification (e.g. posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No. WO97/34631, which discloses amino acid residues involved in the interaction between the Fc domain and the FcRn receptor. Methods for introducing such modifications in the nucleic acid sequence underlying the amino acid sequence of an antibody or its binding fragment is well known to the person skilled in the art.

In some instances, an antibody binding fragment further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such embodiments, bi-specific single chain antibody construct is tandem bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some embodiments, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to that the antibody or its binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further embodiments, the binding is exemplified by the specificity of a “key-lock-principle”. Thus in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well in a simple binding of the site to the antigen.

In some instances, specific interaction further refers to a reduced cross-reactivity of the antibody or its binding fragment or a reduced off-target effect. For example, the antibody or its binding fragment that bind to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

### Additional Binding Moieties

In some embodiments, the binding moiety is a plasma protein. In some instances, the plasma protein comprises albumin. In some instances, the binding moiety A is albumin. In some instances, albumin is conjugated by one or more of a conjugation chemistry described herein to a hetero-duplex polynucleotide. In some instances, albumin is conjugated by native ligation chemistry to a hetero-duplex polynucleotide. In some instances, albumin is conjugated by lysine conjugation to a hetero-duplex polynucleotide.

In some instances, the binding moiety is a steroid. Exemplary steroids include cholesterol, phospholipids, di- and triacylglycerols, fatty acids, hydrocarbons that are saturated, unsaturated, comprise substitutions, or combinations thereof. In some instances, the steroid is cholesterol. In some instances, the binding moiety is cholesterol. In some instances, cholesterol is conjugated by one or more of a conjugation chemistry described herein to a hetero-duplex polynucleotide. In some instances, cholesterol is conjugated by native ligation chemistry to a hetero-duplex polynucleotide. In some instances, cholesterol is conjugated by lysine conjugation to a hetero-duplex polynucleotide.

In some instances, the binding moiety is a polymer, including but not limited to poly nucleic acid molecule aptamers that bind to specific surface markers on cells. In this instance the binding moiety is a polynucleic acid that does not hybridize to a target gene or mRNA, but instead is capable of selectively binding to a cell surface marker similarly to an antibody binding to its specific epitope of a cell surface marker.

In some cases, the binding moiety is a peptide. In some cases, the peptide comprises between about 1 and about 3 kDa. In some cases, the peptide comprises between about 1.2 and about 2.8 kDa, about 1.5 and about 2.5 kDa, or about 1.5 and about 2 kDa. In some instances, the peptide is a bicyclic peptide. In some cases, the bicyclic peptide is a constrained bicyclic peptide. In some instances, the binding moiety is a bicyclic peptide (e.g., bicycles from Bicycle Therapeutics).

In additional cases, the binding moiety is a small molecule. In some instances, the small molecule is an antibody-recruiting small molecule. In some cases, the antibody-recruiting small molecule comprises a target-binding terminus and an antibody-binding terminus, in which the target-binding terminus is capable of recognizing and interacting with a cell surface receptor. For example, in some instances, the target-binding terminus comprising a glutamate urea compound enables interaction with PSMA, thereby, enhances an antibody interaction with a cell (e.g., a cancerous cell) that expresses PSMA. In some instances, a binding moiety is a small molecule described in Zhang et al., “A remote arene-binding site on prostate specific membrane antigen revealed by antibody-recruiting small molecules,” J Am Chem Soc. 132(36): 12711-12716 (2010); or McEnaney, et al., “Antibody-recruiting molecules: an emerging paradigm for engaging immune function in treating human disease,” ACS Chem Biol. 7(7): 1139-1151 (2012).

### Production of Antibodies or Binding Fragments Thereof

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5): 155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Polymer Conjugating Moiety

In some embodiments, a polymer moiety C is further conjugated to a hetero-duplex polynucleotide described herein, a binding moiety described herein, or in combinations thereof. In some instances, a polymer moiety C is conjugated a hetero-duplex polynucleotide. In some cases, a polymer moiety C is conjugated to a binding moiety. In other cases, a polymer moiety C is conjugated to a hetero-duplex polynucleotide-binding moiety molecule. In additional cases, a polymer moiety C is conjugated, and as discussed under the Therapeutic Molecule Platform section.

In some instances, the polymer moiety C is a natural or synthetic polymer, consisting of long chains of branched or unbranched monomers, and/or cross-linked network of monomers in two or three dimensions. In some instances, the polymer moiety C includes a polysaccharide, lignin, rubber, or polyalkylen oxide (e.g., polyethylene glycol). In some instances, the at least one polymer moiety C includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the polymer moiety C comprises polyalkylene oxide. In some instances, the polymer moiety C comprises PEG. In some instances, the polymer moiety C comprises polyethylene imide (PEI) or hydroxy ethyl starch (HES).

In some instances, C is a PEG moiety. In some instances, the PEG moiety is conjugated at the 5′ terminus of the passenger strand of the hetero-duplex polynucleotide while the binding moiety is conjugated at the 3′ terminus of the passenger strand of the hetero-duplex polynucleotide. In some instances, the PEG moiety is conjugated at the 3′ terminus of the passenger strand of the hetero-duplex polynucleotide while the binding moiety is conjugated at the 5′ terminus of the passenger strand of the hetero-duplex polynucleotide. In some instances, the PEG moiety is conjugated to an internal site of the hetero-duplex polynucleotide. In some instances, the PEG moiety, the binding moiety, or a combination thereof, are conjugated to an internal site of the hetero-duplex polynucleotide. In some instances, the conjugation is a direct conjugation. In some instances, the conjugation is via native ligation.

In some embodiments, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some embodiments, C is poly- or monodispersed polyalkylene oxide (e.g., PEG) and the indicated molecular weight represents an average of the molecular weight of the polyalkylene oxide, e.g., PEG, molecules.

In some embodiments, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, C is polyalkylene oxide (e.g., PEG) and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some embodiments, C is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some instances, the molecular weight of C is about 200 Da. In some instances, the molecular weight of C is about 300 Da. In some instances, the molecular weight of C is about 400 Da. In some instances, the molecular weight of C is about 500 Da. In some instances, the molecular weight of C is about 600 Da. In some instances, the molecular weight of C is about 700 Da. In some instances, the molecular weight of C is about 800 Da. In some instances, the molecular weight of C is about 900 Da. In some instances, the molecular weight of C is about 1000 Da. In some instances, the molecular weight of C is about 1100 Da. In some instances, the molecular weight of C is about 1200 Da. In some instances, the molecular weight of C is about 1300 Da. In some instances, the molecular weight of C is about 1400 Da. In some instances, the molecular weight of C is about 1450 Da. In some instances, the molecular weight of C is about 1500 Da. In some instances, the molecular weight of C is about 1600 Da. In some instances, the molecular weight of C is about 1700 Da. In some instances, the molecular weight of C is about 1800 Da. In some instances, the molecular weight of C is about 1900 Da. In some instances, the molecular weight of C is about 2000 Da. In some instances, the molecular weight of C is about 2100 Da. In some instances, the molecular weight of C is about 2200 Da. In some instances, the molecular weight of C is about 2300 Da. In some instances, the molecular weight of C is about 2400 Da. In some instances, the molecular weight of C is about 2500 Da. In some instances, the molecular weight of C is about 2600 Da. In some instances, the molecular weight of C is about 2700 Da. In some instances, the molecular weight of C is about 2800 Da. In some instances, the molecular weight of C is about 2900 Da. In some instances, the molecular weight of C is about 3000 Da. In some instances, the molecular weight of C is about 3250 Da. In some instances, the molecular weight of C is about 3350 Da. In some instances, the molecular weight of C is about 3500 Da. In some instances, the molecular weight of C is about 3750 Da. In some instances, the molecular weight of C is about 4000 Da. In some instances, the molecular weight of C is about 4250 Da. In some instances, the molecular weight of C is about 4500 Da. In some instances, the molecular weight of C is about 4600 Da. In some instances, the molecular weight of C is about 4750 Da. In some instances, the molecular weight of C is about 5000 Da. In some instances, the molecular weight of C is about 5500 Da. In some instances, the molecular weight of C is about 6000 Da. In some instances, the molecular weight of C is about 6500 Da. In some instances, the molecular weight of C is about 7000 Da. In some instances, the molecular weight of C is about 7500 Da. In some instances, the molecular weight of C is about 8000 Da. In some instances, the molecular weight of C is about 10,000 Da. In some instances, the molecular weight of C is about 12,000 Da. In some instances, the molecular weight of C is about 20,000 Da. In some instances, the molecular weight of C is about 35,000 Da. In some instances, the molecular weight of C is about 40,000 Da. In some instances, the molecular weight of C is about 50,000 Da. In some instances, the molecular weight of C is about 60,000 Da. In some instances, the molecular weight of C is about 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some instances, a dPEG comprises about 3 or more repeating ethylene oxide units. In some instances, a dPEG comprises about 4 or more repeating ethylene oxide units. In some instances, a dPEG comprises about 5 or more repeating ethylene oxide units. In some instances, a dPEG comprises about 6 or more repeating ethylene oxide units. In some instances, a dPEG comprises about 7 or more repeating ethylene oxide units. In some instances, a dPEG comprises about 8 or more repeating ethylene oxide units. In some instances, a dPEG comprises about 9 or more repeating ethylene oxide units. In some instances, a dPEG comprises about 10 or more repeating ethylene oxide units. In some instances, a dPEG comprises about 11 or more repeating ethylene oxide units. In some instances, a dPEG comprises about 12 or more repeating ethylene oxide units. In some instances, a dPEG comprises about 13 or more repeating ethylene oxide units. In some instances, a dPEG comprises about 14 or more repeating ethylene oxide units. In some instances, a dPEG comprises about 15 or more repeating ethylene oxide units. In some instances, a dPEG comprises about 16 or more repeating ethylene oxide units. In some instances, a dPEG comprises about 17 or more repeating ethylene oxide units. In some instances, a dPEG comprises about 18 or more repeating ethylene oxide units. In some instances, a dPEG comprises about 19 or more repeating ethylene oxide units. In some instances, a dPEG comprises about 20 or more repeating ethylene oxide units. In some instances, a dPEG comprises about 22 or more repeating ethylene oxide units. In some instances, a dPEG comprises about 24 or more repeating ethylene oxide units. In some instances, a dPEG comprises about 26 or more repeating ethylene oxide units. In some instances, a dPEG comprises about 28 or more repeating ethylene oxide units. In some instances, a dPEG comprises about 30 or more repeating ethylene oxide units. In some instances, a dPEG comprises about 35 or more repeating ethylene oxide units. In some instances, a dPEG comprises about 40 or more repeating ethylene oxide units. In some instances, a dPEG comprises about 42 or more repeating ethylene oxide units. In some instances, a dPEG comprises about 48 or more repeating ethylene oxide units. In some instances, a dPEG comprises about 50 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some embodiments, the polymer moiety C comprises a cationic mucic acid-based polymer (cMAP). In some instances, cMPA comprises one or more subunit of at least one repeating subunit, and the subunit structure is represented as Formula (V):

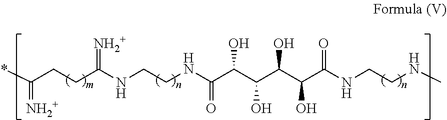

wherein m is independently at each occurrence 1, 2, 3, 4, 5, 6, 7, 8, 9, or 10, preferably 4-6 or 5; and n is independently at each occurrence 1, 2, 3, 4, or 5. In some embodiments, m and n are, for example, about 10.

In some instances, cMAP is further conjugated to a PEG moiety, generating a cMAP-PEG copolymer, an mPEG-cMAP-PEGm triblock polymer, or a cMAP-PEG-cMAP triblock polymer. In some instances, the PEG moiety is in a range of from about 500 Da to about 50,000 Da. In some instances, the PEG moiety is in a range of from about 500 Da to about 1000 Da, greater than 1000 Da to about 5000 Da, greater than 5000 Da to about 10,000 Da, greater than 10,000 to about 25,000 Da, greater than 25,000 Da to about 50,000 Da, or any combination of two or more of these ranges.

In some instances, the polymer moiety C is cMAP-PEG copolymer, an mPEG-cMAP-PEGm triblock polymer, or a cMAP-PEG-cMAP triblock polymer. In some cases, the polymer moiety C is cMAP-PEG copolymer. In other cases, the polymer moiety C is an mPEG-cMAP-PEGm triblock polymer. In additional cases, the polymer moiety C is a cMAP-PEG-cMAP triblock polymer.

In some embodiments, the polymer moiety C is conjugated to the hetero-duplex polynucleotide, the binding moiety, and optionally to the endosomolytic moiety.

### Endosomolytic Moiety

In some embodiments, a molecule of Formula (I): A-(X¹—B)ₙ or Formula (II): A-X¹—(B—X²—C)ₙ further comprises an additional conjugating moiety. In some instances, the additional conjugating moiety is an endosomolytic moiety. In some cases, the endosomolytic moiety is a cellular compartmental release component, such as a compound capable of releasing from any of the cellular compartments known in the art, such as the endosome, lysosome, endoplasmic reticulum (ER), golgi apparatus, microtubule, peroxisome, or other vesicular bodies with the cell. In some cases, the endosomolytic moiety comprises an endosomolytic polypeptide, an endosomolytic polymer, an endosomolytic lipid, or an endosomolytic small molecule. In some cases, the endosomolytic moiety comprises an endosomolytic polypeptide. In other cases, the endosomolytic moiety comprises an endosomolytic polymer.

### Endosomolytic Polypeptides

In some embodiments, a molecule of Formula (I): A-(X¹—B)ₙ or Formula (II): A-X¹—(B—X²—C)ₙ is further conjugated with an endosomolytic polypeptide. In some cases, the endosomolytic polypeptide is a pH-dependent membrane active peptide. In some cases, the endosomolytic polypeptide is an amphipathic polypeptide. In additional cases, the endosomolytic polypeptide is a peptidomimetic. In some instances, the endosomolytic polypeptide comprises INF, melittin, meucin, or their respective derivatives thereof. In some instances, the endosomolytic polypeptide comprises INF or its derivatives thereof. In other cases, the endosomolytic polypeptide comprises melittin or its derivatives thereof. In additional cases, the endosomolytic polypeptide comprises meucin or its derivatives thereof.

In some instances, INF7 is a 24 residue polypeptide those sequence comprises CGIFGEIEELIEEGLENLIDWGNA (SEQ ID NO: 1243), or GLFEAIEGFIENGWEGMIDGWYGC (SEQ ID NO: 1244). In some instances, INF7 or its derivatives comprise a sequence of: GLFEAIEGFIENGWEGMIWDYGSGSCG (SEQ ID NO: 1245), GLFEAIEGFIENGWEGMIDGWYG-(PEG)6-NH2 (SEQ ID NO: 1246), or GLFEAIEGFIENGWEGMIWDYG-SGSC-K(GalNAc)2 (SEQ ID NO: 1247).

In some cases, melittin is a 26 residue polypeptide those sequence comprises CLIGAILKVLATGLPTLISWIKNKRKQ (SEQ ID NO: 1248), or GIGAVLKVLTTGLPALISWIKRKRQQ (SEQ ID NO: 1249). In some instances, melittin comprises a polypeptide sequence as described in U.S. Pat. No. 8,501,930.

In some instances, meucin is an antimicrobial peptide (AMP) derived from the venom gland of the scorpion Mesobuthus eupeus. In some instances, meucin comprises of meucin-13 those sequence comprises IFGAIAGLLKNIF-NH₂ (SEQ ID NO: 1250) and meucin-18 those sequence comprises FFGHLFKLATKIIPSLFQ (SEQ ID NO: 1251).

In some instances, the endosomolytic polypeptide comprises a polypeptide in which its sequence is at least 50%, 60%, 70%, 80%, 90%, 95%, or 99% sequence identity to INF7 or its derivatives thereof, melittin or its derivatives thereof, or meucin or its derivatives thereof. In some instances, the endosomolytic moiety comprises INF7 or its derivatives thereof, melittin or its derivatives thereof, or meucin or its derivatives thereof.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1243-1247. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1243. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1244-1247. In some cases, the endosomolytic moiety comprises SEQ ID NO: 1243. In some cases, the endosomolytic moiety comprises SEQ ID NO: 1244-1247. In some cases, the endosomolytic moiety consists of SEQ ID NO: 1243. In some cases, the endosomolytic moiety consists of SEQ ID NO: 1244-1247.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1248 or 1249. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1248. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1249. In some cases, the endosomolytic moiety comprises SEQ ID NO: 1248. In some cases, the endosomolytic moiety comprises SEQ ID NO: 1249. In some cases, the endosomolytic moiety consists of SEQ ID NO: 1248. In some cases, the endosomolytic moiety consists of SEQ ID NO: 1249.

In some instances, the endosomolytic moiety is meucin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1250 or 1251. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1250. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1251. In some cases, the endosomolytic moiety comprises SEQ ID NO: 1250. In some cases, the endosomolytic moiety comprises SEQ ID NO: 1251. In some cases, the endosomolytic moiety consists of SEQ ID NO: 1250. In some cases, the endosomolytic moiety consists of SEQ ID NO: 1251.

In some instances, the endosomolytic moiety comprises a sequence as illustrated in Table 10.

TABLE 10

| | | | SEQ | |
|---|---|---|---|---|
| | | | ID | |
| Name | Origin | Amino Acid Sequence | NO: | Type |
| Pep-1 | NLS from | KETWWETWWTEWSQPKKKRKV | 1252 | Pri- |
| | Simian | | | mary |
| | Virus | | | amphi- |
| | 40 large | | | pathic |
| | antigen | | | |
| | and | | | |
| | Reverse | | | |
| | trans- | | | |
| | crip- | | | |
| | tase of | | | |
| | HIV | | | |
| pVEC | VE- | LLIILRRRRIRKQAHAHSK | 1253 | Pri- |
| | cadherin | | | mary |
| | | | | amphi- |
| | | | | pathic |
| VT5 | Synthe- | DPKGDPKGVTVTVTVTVTGKG | 1254 | β- |
| | tic | DPKPD | | sheet |
| | peptide | | | amphi- |
| | | | | pathic |
| C105Y | 1-anti- | CSIPPEVKFNKPFVYLI | 1255 | — |
| | trypsin | | | |
| Trans- | Galanin | GWTLNSAGYLLGKINLKALAA | 1256 | Pri- |
| portan | and | LAKKIL | | mary |
| | masto- | | | amphi- |
| | paran | | | pathic |
| TP10 | Galanin | AGYLLGKINLKALAALAKKIL | 1257 | Pri- |
| | and | | | mary |
| | masto- | | | amphi- |
| | paran | | | pathic |
| MPG | A hy- | GALFLGFLGAAGSTMGA | 1258 | β- |
| | drofobic | | | sheet |
| | domain | | | amphi- |
| | from the | | | pathic |
| | fusion | | | |
| | sequence | | | |
| | of HIV | | | |
| | gp41 and | | | |
| | NLS of | | | |
| | SV40 T | | | |
| | antigen | | | |
| gH625 | Glyco- | HGLASTLTRWAHYNALIRAF | 1259 | Secon- |
| | protein | | | dary |
| | gH of | | | amphi- |
| | HSV type | | | pathic |
| | I | | | α-hel- |
| | | | | ical |
| CADY | PPTG1 | GLWRALWRLLRSLWRLLWRA | 1260 | Secon- |
| | peptide | | | dary |
| | | | | amphi- |
| | | | | pathic |
| | | | | α-hel- |
| | | | | ical |
| GALA | Synthe- | WEAALAEALAEALAEHLAEAL | 1261 | Secon- |
| | tic | AEALEALAA | | dary |
| | peptide | | | amphi- |
| | | | | pathic |
| | | | | α-hel- |
| | | | | ical |
| INF | Influen- | GLFEAIEGFIENGWEGMIDGW | 1262 | Secon- |
| | za HA2 | YGC | | dary |
| | fusion | | | amphi- |
| | peptide | | | pathic |
| | | | | α-hel- |
| | | | | ical/ |
| | | | | pH- |
| | | | | depen- |
| | | | | dent |
| | | | | mem- |
| | | | | brane |
| | | | | active |
| | | | | pep- |
| | | | | tide |
| HA2E5- | Influen- | GLFGAIAGFIENGWEGMIDGW | 1263 | Secon- |
| TAT | za HA2 | YG | | dary |
| | subunit | | | amphi- |
| | of in- | | | pathic |
| | fluenza | | | α-hel- |
| | virus | | | ical/ |
| | X31 | | | pH- |
| | strain | | | depen- |
| | fusion | | | dent |
| | peptide | | | mem- |
| | | | | brane |
| | | | | active |
| | | | | pep- |
| | | | | tide |
| HA2- | Influen- | GLFGAIAGFIENGWEGMIDGR | 1264 | pH- |
| pene- | za HA2 | QIKIWFQNRRMKW | | depen- |
| tratin | subunit | KK-amide | | dent |
| | of in- | | | mem- |
| | fluenza | | | brane |
| | virus | | | active |
| | X31 | | | pep- |
| | strain | | | tide |
| | fusion | | | |
| | peptide | | | |
| HA-K4 | Influen- | GLFGAIAGFIENGWEGMIDG- | 1265 | pH- |
| | za HA2 | SSKKKK | | depen- |
| | subunit | | | dent |
| | of in- | | | mem- |
| | fluenza | | | brane |
| | virus | | | active |
| | X31 | | | pep- |
| | strain | | | tide |
| | fusion | | | |
| | peptide | | | |
| HA2E4 | Influen- | GLFEAIAGFIENGWEGMIDGG | 1266 | pH- |
| | za HA2 | GYC | | depen- |
| | subunit | | | dent |
| | of in- | | | mem- |
| | fluenza | | | brane |
| | virus | | | active |
| | X31 | | | pep- |
| | strain | | | tide |
| | fusion | | | |
| | peptide | | | |
| H5WYG | HA2 | GLFHAIAHFIHGGWH | 1267 | pH- |
| | analogue | GLIHGWYG | | depen- |
| | | | | dent |
| | | | | mem- |
| | | | | brane |
| | | | | active |
| | | | | pep- |
| | | | | tide |
| GALA- | INF3 | GLFEAIEGFIENGWEGLAEA | 1268 | pH- |
| INF3- | fusion | LAEALEALAA- | | depen- |
| (PEG)6- | peptide | (PEG)6-NH2 | | dent |
| NH | | | | mem |
| | | | | brane |
| | | | | active |
| | | | | pep- |
| | | | | tide |
| CM18- | Cecro- | KWKLFKKIGAVLKVLTTG- | 1269 | pH- |
| TAT11 | pin-A- | YGRKKRRQRRR | | depen- |
| | Melit- | | | dent |
| | tin₂₋₁₂ | | | mem- |
| | (CM₁₈) | | | brane |
| | fusion | | | active |
| | peptide | | | pep- |
| | | | | tide |

In some cases, the endosomolytic moiety comprises a Bak BH3 polypeptide which induces apoptosis through antagonization of suppressor targets such as Bcl-2 and/or Bcl-x_{L}. In some instances, the endosomolytic moiety comprises a Bak BH3 polypeptide described in Albarran, et al., “Efficient intracellular delivery of a pro-apoptotic peptide with a pH-responsive carrier,” Reactive & Functional Polymers 71: 261-265 (2011).

In some instances, the endosomolytic moiety comprises a polypeptide (e.g., a cell-penetrating polypeptide) as described in PCT Publication Nos. WO2013/166155 or WO2015/069587.

### Endosomolytic Polymers

In some embodiments, a molecule of Formula (I): A-(X¹—B)ₙ or Formula (II): A-X¹—(B—X²—C)ₙ is further conjugated with an endosomolytic polymer. As used herein, an endosomolytic polymer comprises a linear, a branched network, a star, a comb, or a ladder type of polymer. In some instances, an endosomolytic polymer is a homopolymer or a copolymer comprising two or more different types of monomers. In some cases, an endosomolytic polymer is a polycation polymer. In other cases, an endosomolytic polymer is a polyanion polymer.

In some instances, a polycation polymer comprises monomer units that are charge positive, charge neutral, or charge negative, with a net charge being positive. In other cases, a polycation polymer comprises a non-polymeric molecule that contains two or more positive charges. Exemplary cationic polymers include, but are not limited to, poly(L-lysine) (PLL), poly(L-arginine) (PLA), polyethyleneimine (PEI), poly[α-(4-aminobutyl)-L-glycolic acid] (PAGA), 2-(dimethylamino)ethyl methacrylate (DMAEMA), or N,N-Diethylaminoethyl Methacrylate (DEAEMA).

In some cases, a polyanion polymer comprises monomer units that are charge positive, charge neutral, or charge negative, with a net charge being negative. In other cases, a polyanion polymer comprises a non-polymeric molecule that contains two or more negative charges. Exemplary anionic polymers include p(alkylacrylates) (e.g., poly(propyl acrylic acid) (PPAA)) or poly(N-isopropylacrylamide) (NIPAM). Additional examples include PP75, a L-phenylalanine-poly(L-lysine isophthalamide) polymer described in Khormaee, et al., “Edosomolytic anionic polymer for the cytoplasmic delivery of siRNAs in localized in vivo applications,” Advanced Functional Materials 23: 565-574 (2013).

In some embodiments, an endosomolytic polymer described herein is a pH-responsive endosomolytic polymer. A pH-responsive polymer comprises a polymer that increases in size (swell) or collapses depending on the pH of the environment. Polyacrylic acid and chitosan are examples of pH-responsive polymers.

In some instances, an endosomolytic moiety described herein is a membrane-disruptive polymer. In some cases, the membrane-disruptive polymer comprises a cationic polymer, a neutral or hydrophobic polymer, or an anionic polymer. In some instances, the membrane-disruptive polymer is a hydrophilic polymer.

In some instances, an endosomolytic moiety described herein is a pH-responsive membrane-disruptive polymer. Exemplary pH-responsive membrane-disruptive polymers include p(alkylacrylic acids), poly(N-isopropylacrylamide) (NIPAM) copolymers, succinylated p(glycidols), and p(β-malic acid) polymers.

In some instances, p(alkylacrylic acids) include poly(propylacrylic acid) (polyPAA), poly(methacrylic acid) (PMAA), poly(ethylacrylic acid) (PEAA), and poly(propyl acrylic acid) (PPAA). In some instances, a p(alkylacrylic acid) include a p(alkylacrylic acid) described in Jones, et al., Biochemistry Journal 372: 65-75 (2003).

In some embodiments, a pH-responsive membrane-disruptive polymer comprises p(butyl acrylate-co-methacrylic acid). (see Bulmus, et al., Journal of Controlled Release 93: 105-120 (2003); and Yessine, et al., Biochimica et Biophysica Acta 1613: 28-38 (2003))

In some embodiments, a pH-responsive membrane-disruptive polymer comprises p(styrene-alt-maleic anhydride). (see Henry, et al., Biomacromolecules 7: 2407-2414 (2006))

In some embodiments, a pH-responsive membrane-disruptive polymer comprises pyridyldisulfide acrylate (PDSA) polymers such as poly(MAA-co-PDSA), poly(EAA-co-PDSA), poly(PAA-co-PDSA), poly(MAA-co-BA-co-PDSA), poly(EAA-co-BA-co-PDSA), or poly(PAA-co-BA-co-PDSA) polymers. (see El-Sayed, et al., “Rational design of composition and activity correlations for pH-responsive and glutathione-reactive polymer therapeutics,” Journal of Controlled Release 104: 417-427 (2005); or Flanary et al., “Antigen delivery with poly(propylacrylic acid) conjugation enhanced MHC-1 presentation and T-cell activation,” Bioconjugate Chem. 20: 241-248 (2009))

In some embodiments, a pH-responsive membrane-disruptive polymer comprises a lytic polymer comprising the base structure of:

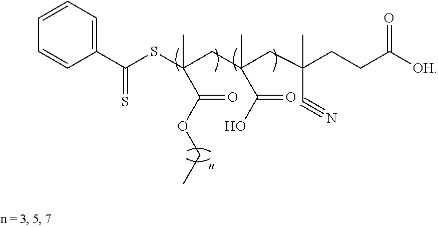

In some instances, an endosomolytic moiety described herein is further conjugated to an additional conjugate, e.g., a polymer (e.g., PEG), or a modified polymer (e.g., cholesterol-modified polymer).

In some instances, the additional conjugate comprises a detergent (e.g., Triton X-100). In some instances, an endosomolytic moiety described herein comprises a polymer (e.g., a poly(amidoamine)) conjugated with a detergent (e.g., Triton X-100). In some instances, an endosomolytic moiety described herein comprises poly(amidoamine)-Triton X-100 conjugate (Duncan, et al., “A polymer-Triton X-100 conjugate capable of pH-dependent red blood cell lysis: a model system illustrating the possibility of drug delivery within acidic intracellular compartments,” Journal of Drug Targeting 2: 341-347 (1994)).

### Endosomolytic Lipids

In some embodiments, the endosomolytic moiety is a lipid (e.g., a fusogenic lipid). In some embodiments, a molecule of Formula (I): A-(X¹—B)ₙ or Formula (II): A-X¹—(B—X²—C)ₙ is further conjugated with an endosomolytic lipid (e.g., fusogenic lipid). Exemplary fusogenic lipids include 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (XTC).

In some instances, an endosomolytic moiety is a lipid (e.g., a fusogenic lipid) described in PCT Publication No. WO09/126,933.

### Endosomolytic Small Molecules

In some embodiments, the endosomolytic moiety is a small molecule. In some embodiments, a molecule of Formula (I): A-(X¹—B)ₙ or Formula (II): A-X¹—(B—X²—C)ₙ is further conjugated with an endosomolytic small molecule. Exemplary small molecules suitable as endosomolytic moieties include, but are not limited to, quinine, chloroquine, hydroxychloroquines, amodiaquins (carnoquines), amopyroquines, primaquines, mefloquines, nivaquines, halofantrines, quinone imines, or a combination thereof. In some instances, quinoline endosomolytic moieties include, but are not limited to, 7-chloro-4-(4-diethylamino-1-methylbutyl-amino)quinoline (chloroquine); 7-chloro-4-(4-ethyl-(2-hydroxyethyl)-amino-1-methylbutyl-amino)quinoline (hydroxychloroquine); 7-fluoro-4-(4-diethylamino-1-methylbutyl-amino)quinoline; 4-(4-diethylamino-1-methylbutylamino) quinoline; 7-hydroxy-4-(4-diethyl-amino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethyl-amino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethyl-amino-1-butylamino)quinoline; 4-(1-carboxy-4-diethylamino-1-butylamino) quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethyl-amino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-fluoro-4-(4-ethyl-(2-hydroxyethyl)-amino-1-methylbutylamino)quinoline; 4-(4-ethyl-(2-hydroxy-ethyl)-amino-1-methylbutylamino-)quinoline; 7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl-1)-amino-1-butylamino)quinoline (desmethylhydroxychloroquine); 7-fluoro-4-(4-ethyl-(2-hydroxyethyl)-amino-1-butylamino)quinoline; 4-(4-ethyl-(2-hydroxyethyl)-amino-1-butylamino)quinoline; 7-hydroxy-4-(4-ethyl-(2-hydroxyethyl)-amino-1-butylamino) quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino-6-methoxydihydrochloride quinoline; 1-acetyl-1,2,3,4-tetrahydroquinoline; 8-[(4-aminopentyl)amino]-6-methoxyquinoline dihydrochloride; 1-butyryl-1,2,3,4-tetrahydroquinoline; 3-chloro-4-(4-hydroxy-alpha,alpha′-bis(2-methyl-1-pyrrolidinyl)-2,5-xylidinoquinoline, 4-[(4-diethyl-amino)-1-methylbutyl-amino]-6-methoxyquinoline; 3-fluoro-4-(4-hydroxy-alpha,alpha′-bis(2-methyl-1-pyrrolidinyl)-2,5-xylidinoquinoline, 4-[(4-diethylamino)-1-methylbutyl-amino]-6-methoxyquinoline; 4-(4-hydroxy-alpha,alpha′-bis(2-methyl-1-pyrrolidinyl)-2,5-xylidinoquinoline; 4-[(4-diethylamino)-1-methylbutyl-amino]-6-methoxyquinoline; 3,4-dihydro-1-(2H)-quinolinecarboxyaldehyde; 1,1′-pentamethylene diquinoleinium diiodide; 8-quinolinol sulfate and amino, aldehyde, carboxylic, hydroxyl, halogen, keto, sulfhydryl and vinyl derivatives or analogs thereof. In some instances, an endosomolytic moiety is a small molecule described in Naisbitt et al (1997, J Pharmacol Exp Therapy 280:884-893) and in U.S. Pat. No. 5,736,557.

### Linkers

In some embodiments, a linker described herein is a cleavable linker or a non-cleavable linker. In some instances, the linker is a cleavable linker. In other instances, the linker is a non-cleavable linker.

In some cases, the linker is a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C₅, C₄, C₃, C₂, or C₁ alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C₅, C₄, C₃, C₂, or C₁ alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

In some instances, the non-polymeric linker does not encompass a polymer that is described above. In some instances, the non-polymeric linker does not encompass a polymer encompassed by the polymer moiety C. In some cases, the non-polymeric linker does not encompass a polyalkylene oxide (e.g., PEG). In some cases, the non-polymeric linker does not encompass a PEG.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as 1-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some instances, the linker comprises a reactive functional group. In some cases, the reactive functional group comprises a nucleophilic group that is reactive to an electrophilic group present on a binding moiety. Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10): 1059-1062 (2014). In some instances, the linker comprises a self-stabilizing maleimide. In some instances, the linker is a self-stabilizing maleimide.

In some embodiments, the linker comprises a peptide moiety. In some instances, the peptide moiety comprises at least 2, 3, 4, 5, or 6 more amino acid residues. In some instances, the peptide moiety comprises at most 2, 3, 4, 5, 6, 7, or 8 amino acid residues. In some instances, the peptide moiety comprises about 2, about 3, about 4, about 5, or about 6 amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the linker comprises a peptide moiety such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the linker comprises Val-Cit. In some cases, the linker is Val-Cit.

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker is used to increase the molar ratio of polynucleotide B to the binding moiety A. In some instances, the dendritic type linker comprises PAMAM dendrimers.

In some embodiments, the linker is a traceless linker or a linker in which after cleavage does not leave behind a linker moiety (e.g., an atom or a linker group) to a binding moiety A, a polynucleotide B, a polymer C, or an endosomolytic moiety D. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

In some embodiments, X₁ and X₂ are each independently a bond or a non-polymeric linker. In some instances, X₁ and X₂ are each independently a bond. In some cases, X₁ and X₂ are each independently a non-polymeric linker.

In some instances, X¹ is a bond or a non-polymeric linker. In some instances, X¹ is a bond. In some instances, X¹ is a non-polymeric linker. In some instances, the linker is a C₁-C₆ alkyl group. In some cases, X¹ is a C₁-C₆ alkyl group, such as for example, a C₅, C₄, C₃, C₂, or C₁ alkyl group. In some cases, the C₁-C₆ alkyl group is an unsubstituted C₁-C₆ alkyl group. As used in the context of a linker, and in particular in the context of X¹, alkyl means a saturated straight or branched hydrocarbon radical containing up to six carbon atoms. In some instances, X¹ includes a homobifunctional linker or a heterobifunctional linker described supra. In some cases, X¹ includes a heterobifunctional linker. In some cases, X¹ includes sMCC. In other instances, X¹ includes a heterobifunctional linker optionally conjugated to a C₁-C₆ alkyl group. In other instances, X¹ includes sMCC optionally conjugated to a C₁-C₆ alkyl group. In additional instances, X¹ does not include a homobifunctional linker or a heterobifunctional linker described supra.

In some instances, X² is a bond or a linker. In some instances, X² is a bond. In other cases, X² is a linker. In additional cases, X² is a non-polymeric linker. In some embodiments, X² is a C₁-C₆ alkyl group. In some instances, X² is a homobifunctional linker or a heterobifunctional linker described supra. In some instances, X² is a homobifunctional linker described supra. In some instances, X² is a heterobifunctional linker described supra. In some instances, X² comprises a maleimide group, such as maleimidocaproyl (mc) or a self-stabilizing maleimide group described above. In some instances, X² comprises a peptide moiety, such as Val-Cit. In some instances, X² comprises a benzoic acid group, such as PABA. In additional instances, X² comprises a combination of a maleimide group, a peptide moiety, and/or a benzoic acid group. In additional instances, X² comprises a mc group. In additional instances, X² comprises a mc-val-cit group. In additional instances, X² comprises a val-cit-PABA group. In additional instances, X² comprises a mc-val-cit-PABA group.

### Methods of Use

In some embodiments, a composition or a pharmaceutical formulation described herein comprising a binding moiety conjugated to a hetero-duplex polynucleotide and a polymer is used for the treatment of a disease or disorder. In some instances, the disease or disorder is a cancer. In some embodiments, a composition or a pharmaceutical formulation described herein is used as an immunotherapy for the treatment of a disease or disorder. In some instances, the immunotherapy is an immuno-oncology therapy.

### Cancer

In some embodiments, a composition or a pharmaceutical formulation described herein is used for the treatment of cancer. In some instances, the cancer is a solid tumor. In some instances, the cancer is a hematologic malignancy. In some instances, the cancer is a relapsed or refractory cancer, or a metastatic cancer. In some instances, the solid tumor is a relapsed or refractory solid tumor, or a metastatic solid tumor. In some cases, the hematologic malignancy is a relapsed or refractory hematologic malignancy, or a metastatic hematologic malignancy.

In some embodiments, the cancer is a solid tumor. Exemplary solid tumor includes, but is not limited to, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, a composition or a pharmaceutical formulation described herein comprising a binding moiety conjugated to a hetero-duplex polynucleotide and a polymer is used for the treatment of a solid tumor. In some instances, a composition or a pharmaceutical formulation described herein comprising a binding moiety conjugated to a hetero-duplex polynucleotide and a polymer is used for the treatment of anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer. In some instances, the solid tumor is a relapsed or refractory solid tumor, or a metastatic solid tumor.

In some instances, the cancer is a hematologic malignancy. In some instances, the hematologic malignancy is a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), WaldenstrOm's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some instances, a composition or a pharmaceutical formulation described herein comprising a binding moiety conjugated to a hetero-duplex polynucleotide and a polymer is used for the treatment of a hematologic malignancy. In some instances, a composition or a pharmaceutical formulation described herein comprising a binding moiety conjugated to a hetero-duplex polynucleotide and a polymer is used for the treatment of a leukemia, a lymphoma, a myeloma, a non-Hodgkin's lymphoma, or a Hodgkin's lymphoma. In some instances, the hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), WaldenstrOm's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some cases, the hematologic malignancy is a relapsed or refractory hematologic malignancy, or a metastatic hematologic malignancy.

In some instances, the cancer is a KRAS-associated, EGFR-associated, AR-associated cancer, HPRT1-associated cancer, or β-catenin associated cancer. In some instances, a composition or a pharmaceutical formulation described herein comprising a binding moiety conjugated to a hetero-duplex polynucleotide and a polymer is used for the treatment of a KRAS-associated, EGFR-associated, AR-associated cancer, HPRT1-associated cancer, or β-catenin associated cancer. In some instances, a composition or a pharmaceutical formulation described herein comprising a binding moiety conjugated to a hetero-duplex polynucleotide and a polymer is used for the treatment of a KRAS-associated cancer. In some instances, a composition or a pharmaceutical formulation described herein comprising a binding moiety conjugated to a hetero-duplex polynucleotide and a polymer is used for the treatment of an EGFR-associated cancer. In some instances, a composition or a pharmaceutical formulation described herein comprising a binding moiety conjugated to a hetero-duplex polynucleotide and a polymer is used for the treatment of an AR-associated cancer. In some instances, a composition or a pharmaceutical formulation described herein comprising a binding moiety conjugated to a hetero-duplex polynucleotide and a polymer is used for the treatment of an HPRT1-associated cancer. In some instances, a composition or a pharmaceutical formulation described herein comprising a binding moiety conjugated to a hetero-duplex polynucleotide and a polymer is used for the treatment of a β-catenin associated cancer. In some instances, the cancer is a solid tumor. In some instances, the cancer is a hematologic malignancy. In some instances, the solid tumor is a relapsed or refractory solid tumor, or a metastatic solid tumor. In some cases, the hematologic malignancy is a relapsed or refractory hematologic malignancy, or a metastatic hematologic malignancy. In some instances, the cancer comprises bladder cancer, breast cancer, colorectal cancer, endometrial cancer, esophageal cancer, glioblastoma multiforme, head and neck cancer, kidney cancer, lung cancer, ovarian cancer, pancreatic cancer, prostate cancer, thyroid cancer, acute myeloid leukemia, CLL, DLBCL, or multiple myeloma. In some instances, the β-catenin associated cancer further comprises PIK3C-associated cancer and/or MYC-associated cancer.

### Immunotherapy

In some embodiments, a composition or a pharmaceutical formulation described herein is used as an immunotherapy for the treatment of a disease or disorder. In some instances, the immunotherapy is an immuno-oncology therapy. In some instances, immuno-oncology therapy is categorized into active, passive, or combinatory (active and passive) methods. In active immuno-oncology therapy method, for example, tumor-associated antigens (TAAs) are presented to the immune system to trigger an attack on cancer cells presenting these TAAs. In some instances, the active immune-oncology therapy method includes tumor-targeting and/or immune-targeting agents (e.g., checkpoint inhibitor agents such as monoclonal antibodies), and/or vaccines, such as in situ vaccination and/or cell-based or non-cell based (e.g., dendritic cell-based, tumor cell-based, antigen, anti-idiotype, DNA, or vector-based) vaccines. In some instances, the cell-based vaccines are vaccines which are generated using activated immune cells obtained from a patient's own immune system which are then activated by the patient's own cancer. In some instances, the active immune-oncology therapy is further subdivided into non-specific active immunotherapy and specific active immunotherapy. In some instances, non-specific active immunotherapy utilizes cytokines and/or other cell signaling components to induce a general immune system response. In some cases, specific active immunotherapy utilizes specific TAAs to elicite an immune response.

In some embodiments, a composition or a pharmaceutical formulation described herein is used as an active immuno-oncology therapy method for the treatment of a disease or disorder (e.g., cancer). In some embodiments, the composition or a pharmaceutical formulation described herein comprises a tumor-targeting agent. In some instances, the tumor-targeting agent is encompassed by a binding moiety A. In other instances, the tumor-targeting agent is an additional agent used in combination with a molecule of Formula (I). In some instances, the tumor-targeting agent is a tumor-directed polypeptide (e.g., a tumor-directed antibody). In some instances, the tumor-targeting agent is a tumor-directed antibody, which exerts its antitumor activity through mechanisms such as direct killing (e.g., signaling-induced apoptosis), complement-dependent cytotoxicity (CDC), and/or antibody-dependent cell-mediated cytotoxicity (ADCC). In additional instances, the tumor-targeting agent elicits an adaptive immune response, with the induction of antitumor T cells.

In some embodiments, the binding moiety A is a tumor-directed polypeptide (e.g., a tumor-directed antibody). In some instances, the binding moiety A is a tumor-directed antibody, which exerts its antitumor activity through mechanisms such as direct killing (e.g., signaling-induced apoptosis), complement-dependent cytotoxicity (CDC), and/or antibody-dependent cell-mediated cytotoxicity (ADCC). In additional instances, the binding moiety A elicits an adaptive immune response, with the induction of antitumor T cells.

In some embodiments, the composition or a pharmaceutical formulation described herein comprises an immune-targeting agent. In some instances, the immune-targeting agent is encompassed by a binding moiety A. In other instances, the immune-targeting agent is an additional agent used in combination with a molecule of Formula (I). In some instances, the immune-targeting agent comprises cytokines, checkpoint inhibitors, or a combination thereof.

In some embodiments, the immune-targeting agent is a checkpoint inhibitor. In some cases, an immune checkpoint molecule is a molecule presented on the cell surface of CD4 and/or CD8 T cells. Exemplary immune checkpoint molecules include, but are not limited to, Programmed Death-Ligand 1 (PD-L1, also known as B7-H1, CD274), Programmed Death 1 (PD-1), CTLA-4, B7H1, B7H4, OX-40, CD137, CD40, 2B4, IDOL IDO2, VISTA, CD27, CD28, PD-L2 (B7-DC, CD273), LAG3, CD80, CD86, PDL2, B7H3, HVEM, BTLA, KIR, GAL9, TIM3, A2aR, MARCO (macrophage receptor with collageneous structure), PS (phosphatidylserine), ICOS (inducible T cell costimulator), HAVCR2, CD276, VTCN1, CD70, and CD160.

In some instances, an immune checkpoint inhibitor refers to any molecule that modulates or inhibits the activity of an immune checkpoint molecule. In some instances, immune checkpoint inhibitors include antibodies, antibody-derivatives (e.g., Fab fragments, scFvs, minobodies, diabodies), antisense oligonucleotides, siRNA, aptamers, or peptides. In some embodiments, an immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDOL IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof.

In some embodiments, exemplary checkpoint inhibitors include:

PD-L1 inhibitors such as Genentech's MPDL3280A (RG7446), Anti-mouse PD-L1 antibody Clone 10F.9G2 (Cat #BE0101) from BioXcell, anti-PD-L1 monoclonal antibody MDX-1105 (BMS-936559) and BMS-935559 from Bristol-Meyer's Squibb, MSB0010718C, mouse anti-PD-L1 Clone 29E.2A3, and AstraZeneca's MEDI4736;

PD-L2 inhibitors such as GlaxoSmithKline's AMP-224 (Amplimmune), and rHIgM12B7;

PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (Opdivo®, BMS-936558, MDX1106), AstraZeneca's AMP-514 and AMP-224, and Pidilizumab (CT-011) from CureTech Ltd;

CTLA-4 inhibitors such as Bristol Meyers Squibb's anti-CTLA-4 antibody ipilimumab (also known as Yervoy®, MDX-010, BMS-734016 and MDX-101), anti-CTLA4 Antibody, clone 9H10 from Millipore, Pfizer's tremelimumab (CP-675,206, ticilimumab), and anti-CTLA4 antibody clone BNI3 from Abcam;

LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12;

B7-H3 inhibitors such as MGA271;

KIR inhibitors such as Lirilumab (IPH2101);

CD137 (41BB) inhibitors such as urelumab (BMS-663513, Bristol-Myers Squibb), PF-05082566 (anti-4-1BB, PF-2566, Pfizer), or XmAb-5592 (Xencor);

PS inhibitors such as Bavituximab;

and inhibitors such as an antibody or fragments (e.g., a monoclonal antibody, a human, humanized, or chimeric antibody) thereof, RNAi molecules, or small molecules to TIM3, CD52, CD30, CD20, CD33, CD27, OX40 (CD134), GITR, ICOS, BTLA (CD272), CD160, 2B4, LAIR1, TIGHT, LIGHT, DR3, CD226, CD2, or SLAM.

In some embodiments, a binding moiety A comprising an immune checkpoint inhibitor is used for the treatment of a disease or disorder (e.g., cancer). In some instances, the binding moiety A is a bispecific antibody or a binding fragment thereof that comprises an immune checkpoint inhibitor. In some cases, a binding moiety A comprising an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDOL IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof, is used for the treatment of a disease or disorder (e.g., cancer).

In some embodiments, a molecule of Formula (I) in combination with an immune checkpoint inhibitor is used for the treatment of a disease or disorder (e.g., cancer). In some instances, the immune checkpoint inhibitor comprises an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDOL IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some cases, a molecule of Formula (I) is used in combination with ipilimumab, tremelimumab, nivolumab, pemrolizumab, pidilizumab, MPDL3280A, MEDI4736, MSB0010718C, MK-3475, or BMS-936559, for the treatment of a disease or disorder (e.g., cancer).

In some embodiments, the immune-targeting agent is a cytokine. In some cases, cytokine is further subgrouped into chemokine, interferon, interleukin, and tumor necrosis factor. In some embodiments, chemokine plays a role as a chemoattractant to guide the migration of cells, and is classified into four subfamilies: CXC, CC, CX3C, and XC. Exemplary chemokines include chemokines from the CC subfamily: CCL1, CCL2 (MCP-1), CCL3, CCL4, CCL5 (RANTES), CCL6, CCL7, CCL8, CCL9 (or CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, and CCL28; the CXC subfamily: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, and CXCL17; the XC subfamily: XCL1 and XCL2; and the CX3C subfamily CX3CL1.

Interferon (IFNs) comprises interferon type I (e.g. IFN-α, IFN-β, IFN-ε, IFN-κ, and IFN-ω), interferon type II (e.g. IFN-γ), and interferon type III. In some embodiments, IFN-α is further classified into about 13 subtypes which include IFNA1, IFNA2IFNA4, IFNA5, IFNA6, IFNA7, IFNA8, IFNA10, IFNA13, IFNA14, IFNA16, IFNA17, and IFNA21.

Interleukin is expressed by leukocyte or white blood cell and promote the development and differentiation of T and B lymphocytes and hematopoietic cells. Exemplary interleukins include IL-1, IL-2, IL-3, IL-4, IL-S, IL-6, IL-7, IL-8 (CXCL8), IL-9, IL-10, IL-11, IL-12, IL13, IL-14, IL-15, IL-16, IL-17, IL-18, IL-19, IL-20, IL, 21, IL-22, IL-23, IL-24, IL-25, IL-26, IL-27, IL-28, IL-29, IL-30, IL-31, IL-32, IL-33, IL-35, and IL-36.

Tumor necrosis factors (INFs) are a group of cytokines that modulate apoptosis. In some instances, there are about 19 members within the TNF family, including, not limited to, TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL).

In some embodiments, a molecule of Formula (I) in combination with a cytokine is used for the treatment of a disease or disorder (e.g., cancer). In some cases, a molecule of Formula (I) in combination with a chemokine is used for the treatment of a disease or disorder (e.g., cancer). In some cases, a molecule of Formula (I) in combination with an interferon is used for the treatment of a disease or disorder (e.g., cancer). In some cases, a molecule of Formula (I) in combination with an interleukin is used for the treatment of a disease or disorder (e.g., cancer). In some cases, a molecule of Formula (I) in combination with a tumor necrosis factor is used for the treatment of a disease or disorder (e.g., cancer). In some instances, a molecule of Formula (I) in combination with IL-1β, IL-2, IL-7, IL-8, IL-15, MCP-1 (CCL2), MIP-1α, RANTES, MCP-3, MIP5, CCL19, CCL21, CXCL2, CXCL9, CXCL10, or CXCL11 is used for the treatment of a disease or disorder (e.g., cancer).

In some embodiments, the composition or a pharmaceutical formulation described herein comprises a vaccine. In some instances, the vaccine is an in situ vaccination. In some instances, the vaccine is a cell-based vaccine. In some instances, the vaccine is a non-cell based vaccine. In some instances, a molecule of Formula (I) in combination with dendritic cell-based vaccine is used for the treatment of a disease or disorder (e.g., cancer). In some instances, a molecule of Formula (I) in combination with tumor cell-based vaccine is used for the treatment of a disease or disorder (e.g., cancer). In some instances, a molecule of Formula (I) in combination with antigen vaccine is used for the treatment of a disease or disorder (e.g., cancer). In some instances, a molecule of Formula (I) in combination with anti-idiotype vaccine is used for the treatment of a disease or disorder (e.g., cancer). In some instances, a molecule of Formula (I) in combination with DNA vaccine is used for the treatment of a disease or disorder (e.g., cancer). In some instances, a molecule of Formula (I) in combination with vector-based vaccine is used for the treatment of a disease or disorder (e.g., cancer).

In some embodiments, a composition or a pharmaceutical formulation described herein is used as a passive immuno-oncology therapy method for the treatment of a disease or disorder (e.g., cancer). The passive method, in some instances, utilizes adoptive immune system components such as T cells, natural killer (NK) T cells, and/or chimeric antigen receptor (CAR) T cells generated exogenously to attack cancer cells.

In some embodiments, a molecule of Formula (I) in combination with a T-cell based therapeutic agent is used for the treatment of a disease or disorder (e.g., cancer). In some cases, the T-cell based therapeutic agent is an activated T-cell agent that recognizes one or more of a CD cell surface marker described above. In some instances, the T-cell based therapeutic agent comprises an activated T-cell agent that recognizes one or more of CD2, CD3, CD4, CD5, CD8, CD27, CD28, CD80, CD134, CD137, CD152, CD154, CD160, CD200R, CD223, CD226, CD244, CD258, CD267, CD272, CD274, CD278, CD279, or CD357. In some instances, a molecule of Formula (I) in combination with an activated T-cell agent recognizing one or more of CD2, CD3, CD4, CD5, CD8, CD27, CD28, CD80, CD134, CD137, CD152, CD154, CD160, CD200R, CD223, CD226, CD244, CD258, CD267, CD272, CD274, CD278, CD279, or CD357 is used for the treatment of a disease or disorder (e.g., cancer).

In some embodiments, a molecule of Formula (I) in combination with natural killer (NK) T cell-based therapeutic agent is used for the treatment of a disease or disorder (e.g., cancer). In some instances, the NK-based therapeutic agent is an activated NK agent that recognizes one or more of a CD cell surface marker described above. In some cases, the NK-based therapeutic agent is an activated NK agent that recognizes one or more of CD2, CD11a, CD11b, CD16, CD56, CD58, CD62L, CD85j, CD158a/b, CD158c, CD158e/f/k, CD158h/j, CD159a, CD162, CD226, CD314, CD335, CD337, CD244, or CD319. In some instances, a molecule of Formula (I) in combination with an activated NK agent recognizing one or more of CD2, CD11a, CD11b, CD16, CD56, CD58, CD62L, CD85j, CD158a/b, CD158c, CD158e/f/k, CD158h/j, CD159a, CD162, CD226, CD314, CD335, CD337, CD244, or CD319 is used for the treatment of a disease or disorder (e.g., cancer).

In some embodiments, a molecule of Formula (I) in combination with CAR-T cell-based therapeutic agent is used for the treatment of a disease or disorder (e.g., cancer).

In some embodiments, a molecule of Formula (I) in combination with an additional agent that destabilizes the endosomal membrane (or disrupts the endosomal-lysosomal membrane trafficking) is used for the treatment of a disease or disorder (e.g., cancer). In some instances, the additional agent comprises an antimitotic agent. Exemplary antimitotic agents include, but are not limited to, taxanes such as paclitaxel and docetaxel; vinca alkaloids such as vinblastine, vincristine, vindesine, and vinorelbine; cabazitaxel; colchicine; eribulin; estramustine; etoposide; ixabepilone; podophyllotoxin; teniposide; or griseofulvin. In some instances, the additional agent comprises paclitaxel, docetaxel, vinblastine, vincristine, vindesine, vinorelbine, cabazitaxel, colchicine, eribulin, estramustine, etoposide, ixabepilone, podophyllotoxin, teniposide, or griseofulvin. In some instances, the additional agent comprises taxol. In some instances, the additional agent comprises paclitaxel. In some instances, the additional agent comprises etoposide. In other instances, the additional agent comprises vitamin K3.

In some embodiments, a composition or a pharmaceutical formulation described herein is used as a combinatory method (including for both active and passive methods) in the treatment of a disease or disorder (e.g., cancer).

### Pharmaceutical Formulation

In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate-release formulations, controlled-release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a hetero-duplex polynucleotide or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, α-chymotrypsin, polylysine, polyarginine, histone, protamine, ovalbumin, dextrin, or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a hetero-duplex polynucleotide or a binding moiety described herein is conjugated either directly or indirectly to the nanoparticle. In some instances, at least 1, 5, 10, 15, 20, 30, 40, 50, 60, 70, 80, 90, 100, or more hetero-duplex polynucleotides or binding moieties described herein are conjugated either directly or indirectly to a nanoparticle.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH-adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide, and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simutaneously, sequentially, or at an interval period of time. In some embodiments, one or more pharmaceutical compositions are administered simutaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are coadministered. In some instances, the two or more different pharmaceutical compositions are coadministered simutaneously. In some cases, the two or more different pharmaceutical compositions are coadministered sequentially without a gap of time between administrations. In other cases, the two or more different pharmaceutical compositions are coadministered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, are optionally reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a molecule of Formula (I): A-X₁—B—X₂—C, optionally conjugated to an endosomolytic moiety D as disclosed herein. Such kits optionally include an identifying description or label or instructions relating to its use in the methods described herein.

A kit typically includes labels listing contents and/or instructions for use and package inserts with instructions for use. A set of instructions will also typically be included.

In one embodiment, a label is on or associated with the container. In one embodiment, a label is on a container when letters, numbers, or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that is expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### EXAMPLES

These examples are provided for illustrative purposes only and not to limit the scope of the claims provided herein.

### CHEMICAL SYNTHESIS EXAMPLES

### Example 1. Preparation of Compound 1-3, and 5-8

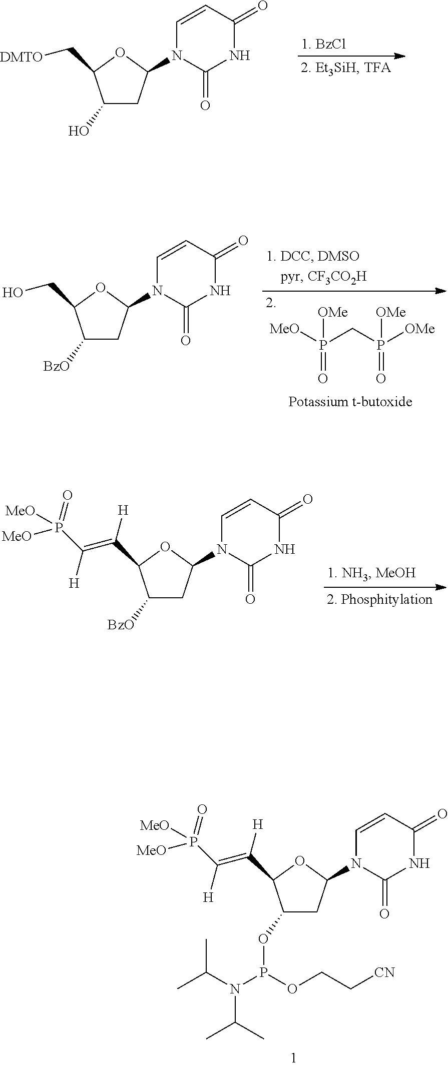

Compounds 2, 3, and 5-8 were prepared as per procedures illustrated in Example 1.

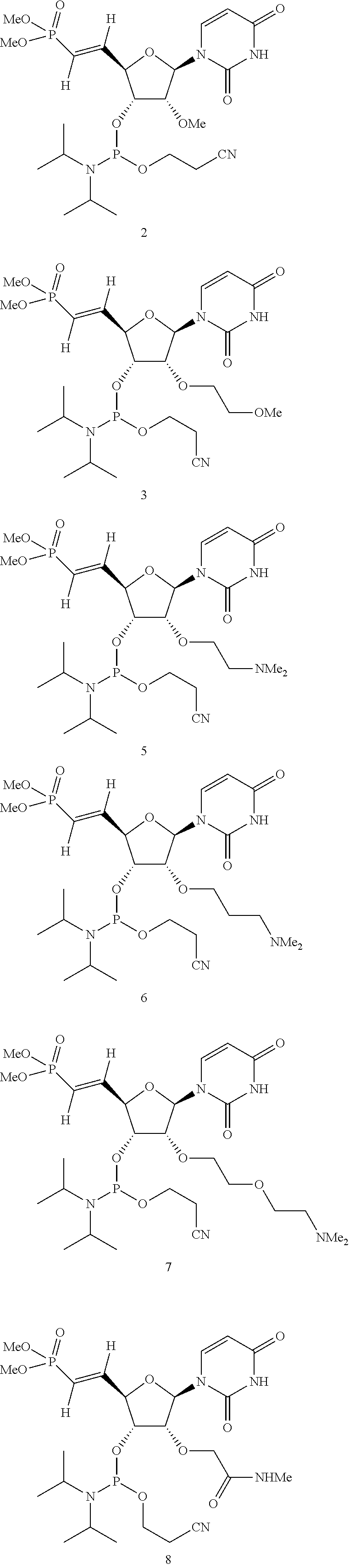

### Example 2. Preparation of Compound 4

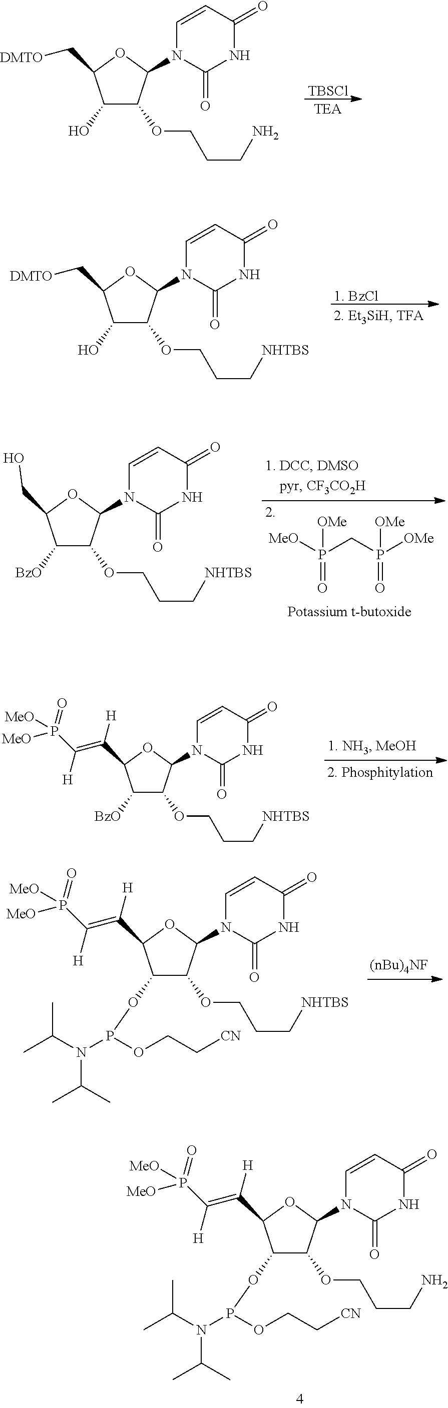

### Example 3. Preparation of Compound 9

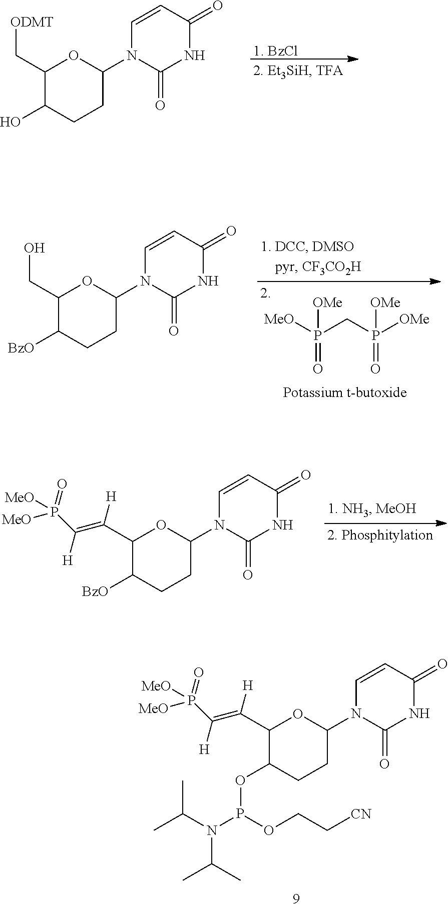

### Example 4. Preparation of Compound 10

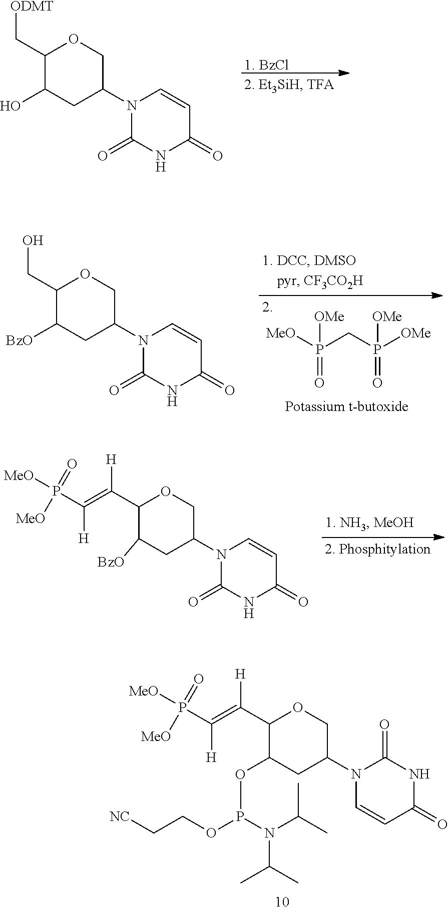

### Example 5. Preparation of Compound 11

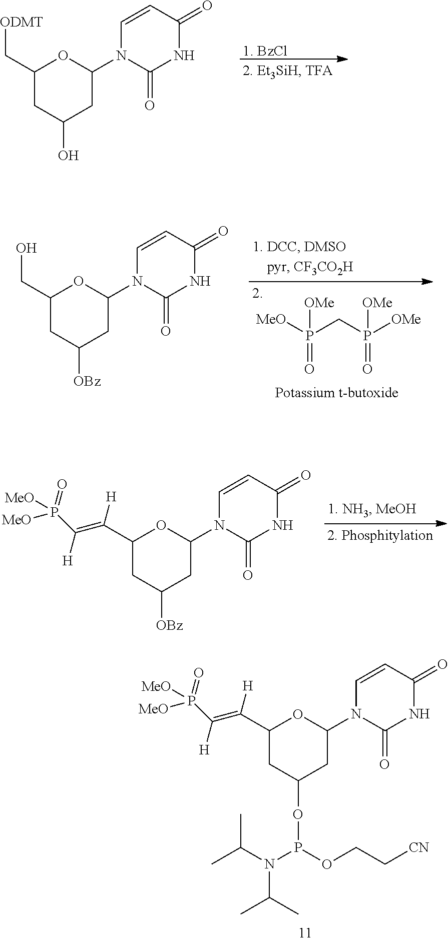

### Example 6. Preparation of Compound 12

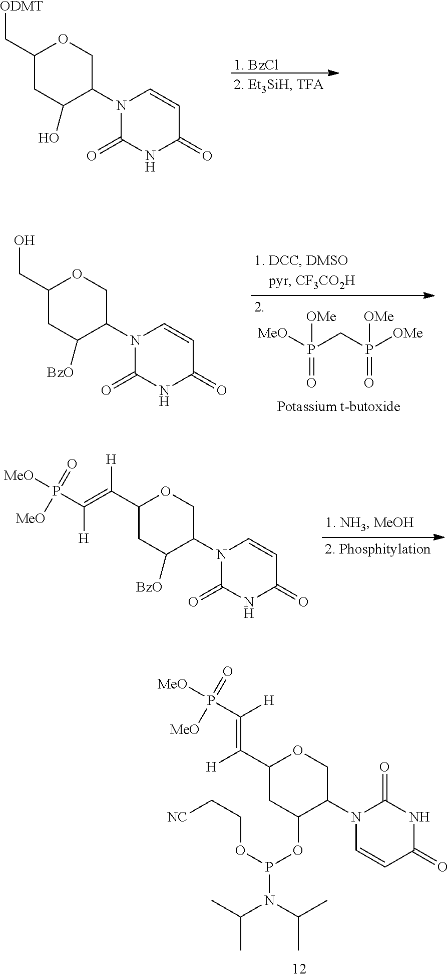

### Example 7. Preparation of Compound 13

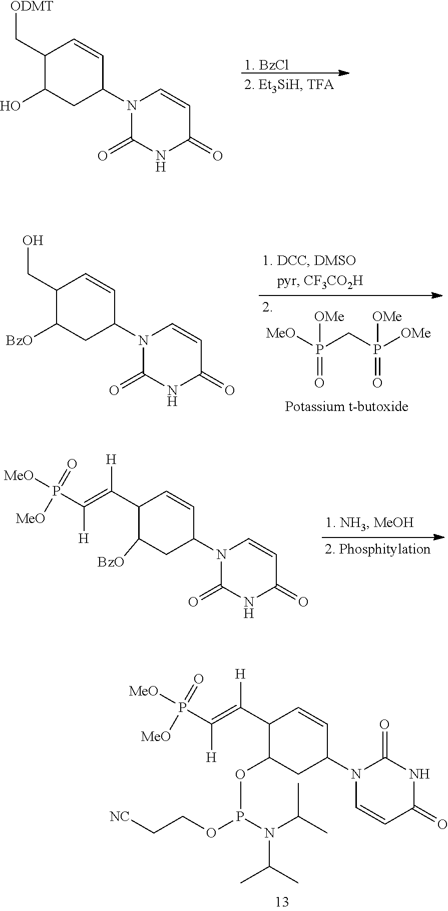

### Example 8. Preparation of Compound 14

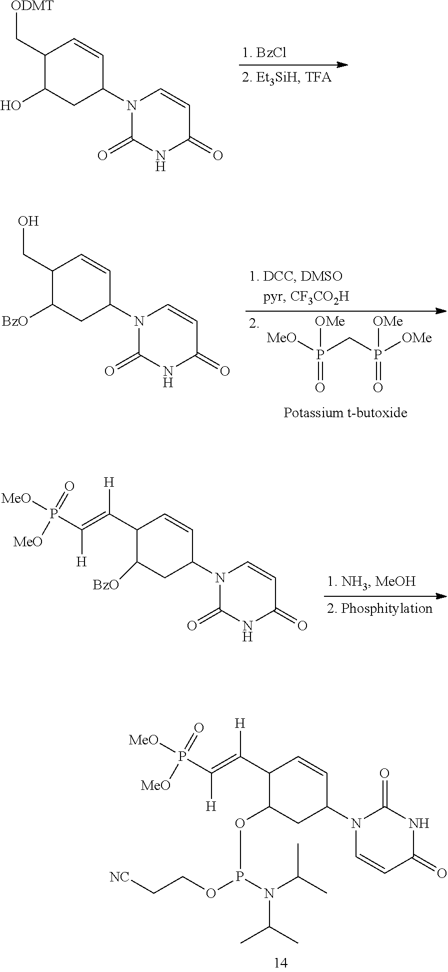

### Example 9. Preparation of Compound 15

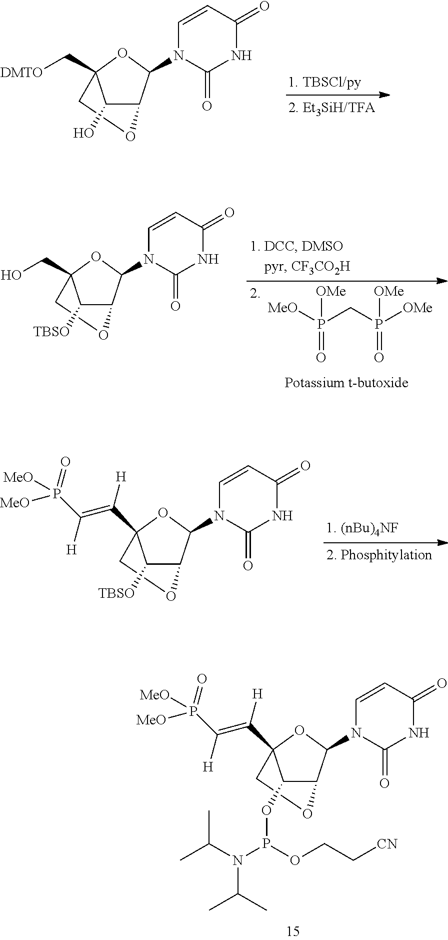

### Example 10. Preparation of Compound 16

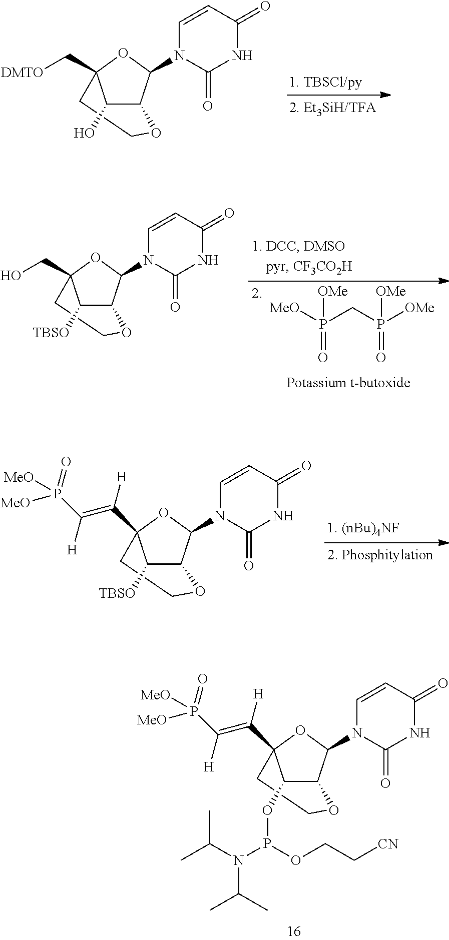

### Example 11. Preparation of Compound 17

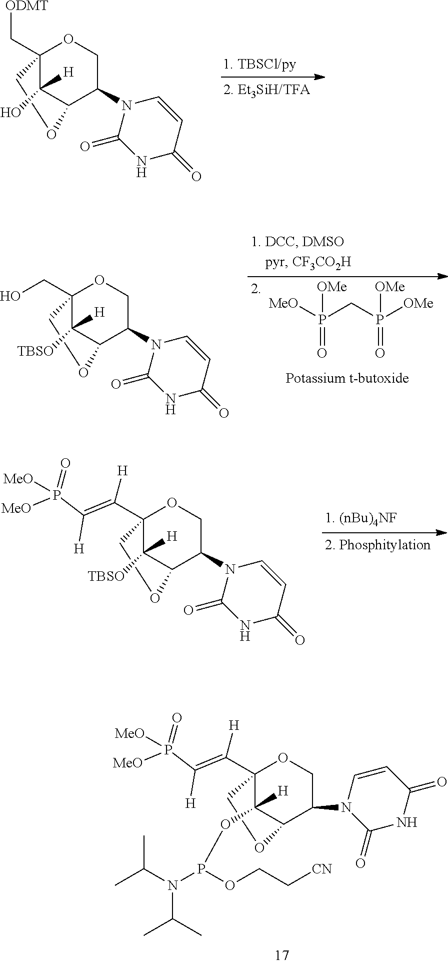

### Example 12. Preparation of Compound 18

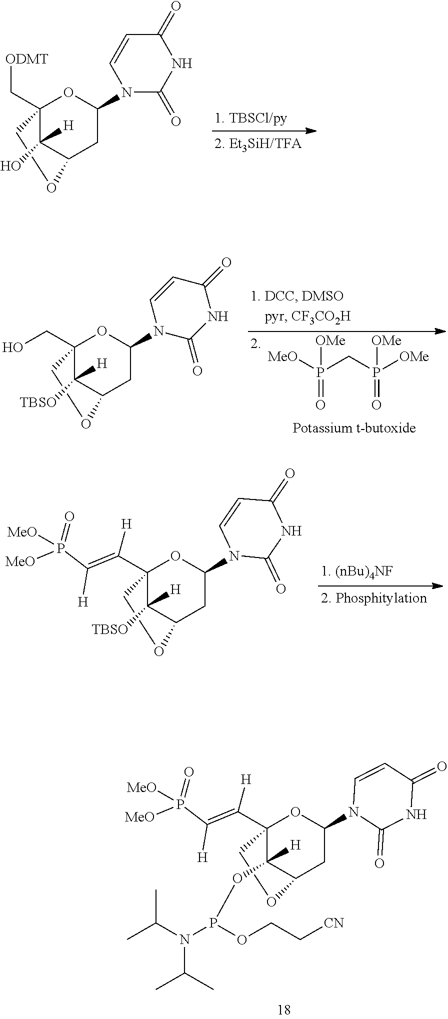

### Example 13. Preparation of Compound 19

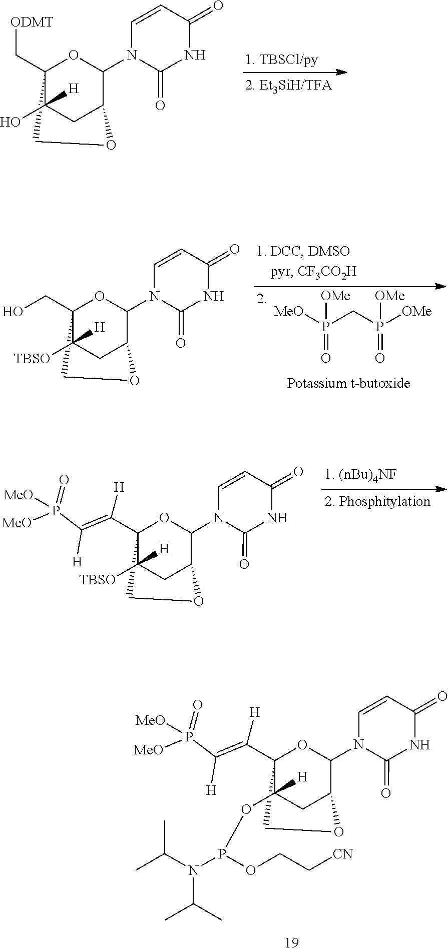

### Example 14. Preparation of Compound 20

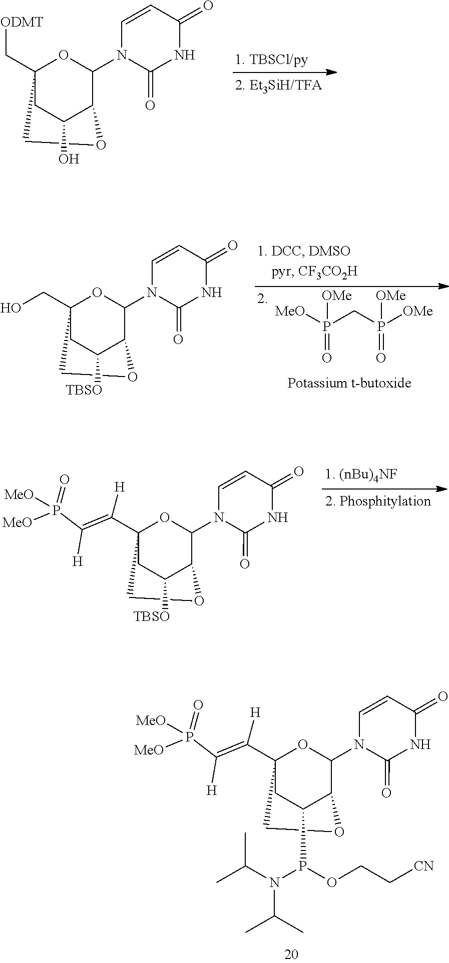

### Example 15. Preparation of Compound 21

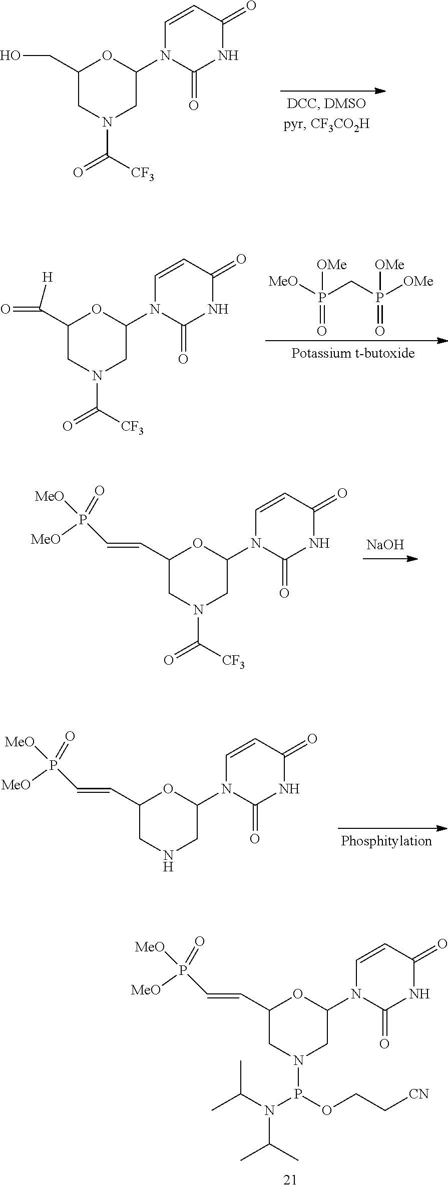

### Example 16. Preparation of Compound 22

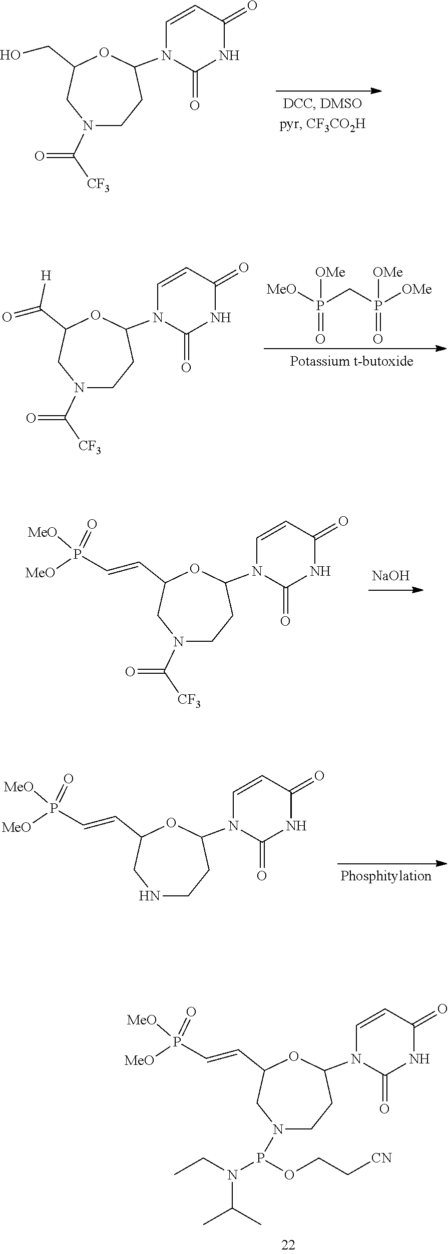

### Example 17. Preparation of Compound 23

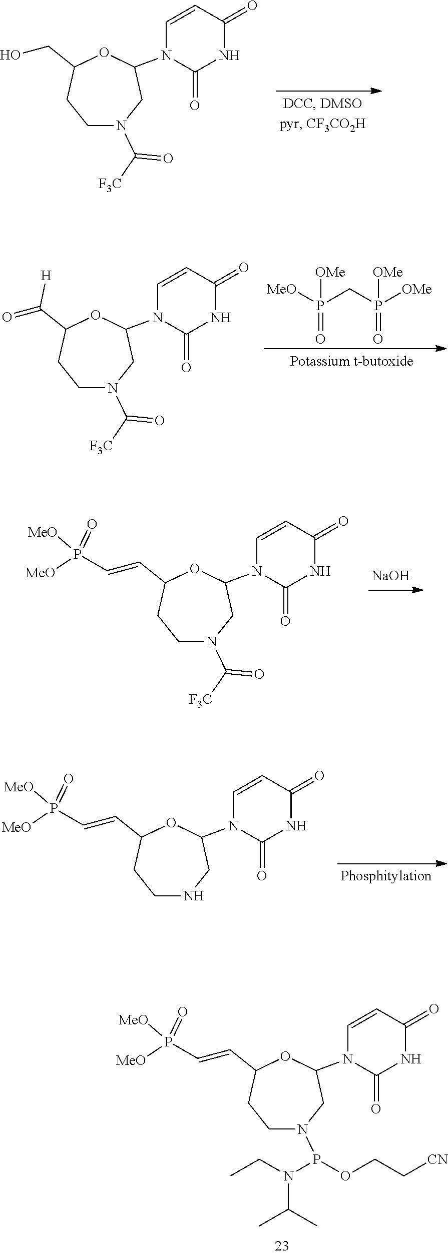

### Example 18. Preparation of Compound 24

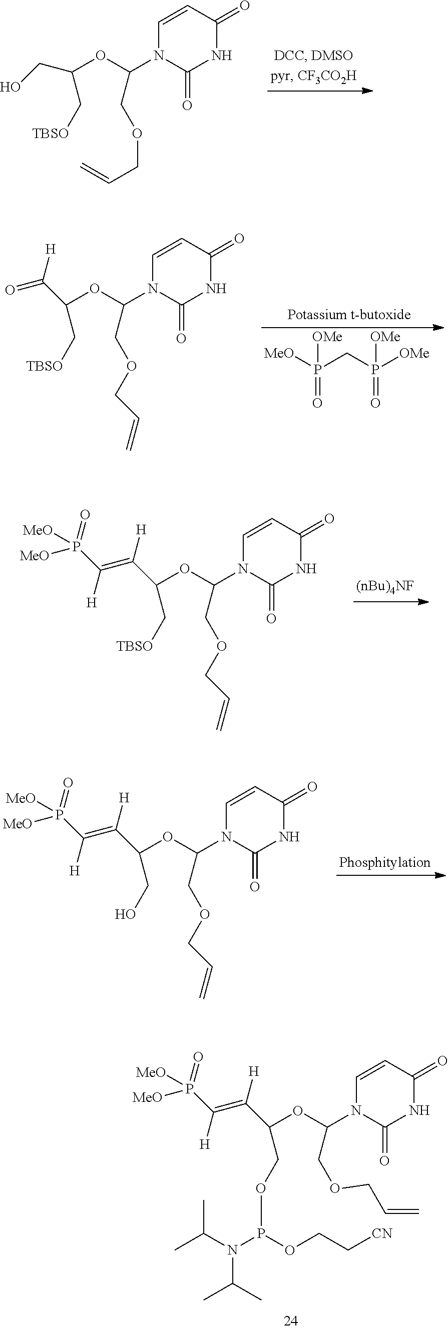 [SMILES: C=CCOCC(OC(CO)CO[Si](C)(C)C(C)(C)C)n1ccc(=O)[nH]c1=O ; C=CCOCC(OC(C=O)CO[Si](C)(C)C(C)(C)C)n1ccc(=O)[nH]c1=O ; COP(=O)(CP(=O)(OC)OC)OC ; C=CCOCC(OC(/C=C/P(=O)(OC)OC)CO[Si](C)(C)C(C)(C)C)n1ccc(=O)[nH]c1=O ; C=CCOCC(OC(/C=C/P(=O)(OC)OC)CO)n1ccc(=O)[nH]c1=O ; C=CCOCC(OC(/C=C/P(=O)(OC)OC)COP(OCCC#N)N(C(C)C)C(C)C)n1ccc(=O)[nH]c1=O]

### Example 19. Preparation of Compound 25

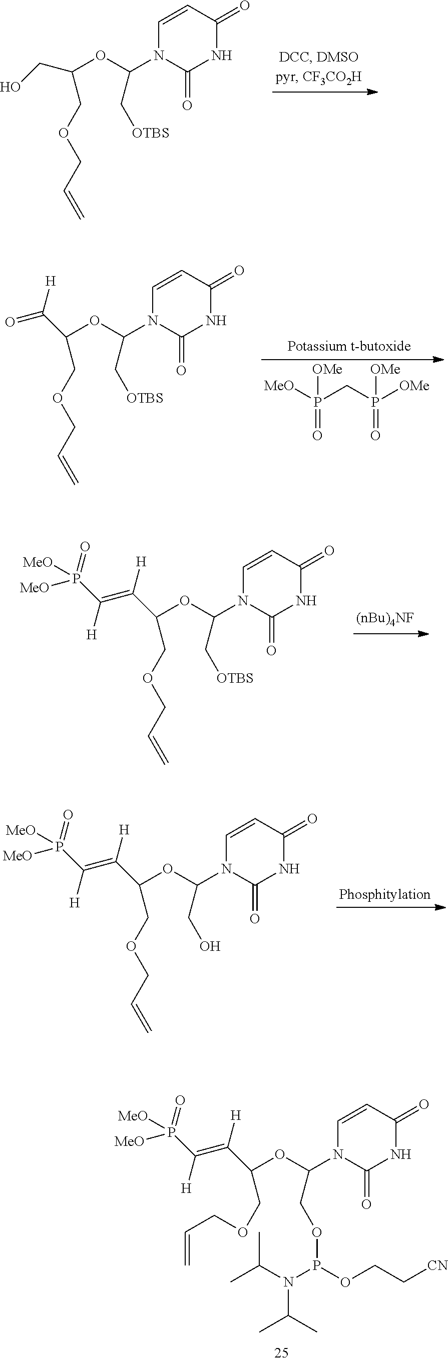 [SMILES: C=CCOCC(CO)OC(CO[Si](C)(C)C(C)(C)C)n1ccc(=O)[nH]c1=O ; C=CCOCC(C=O)OC(CO[Si](C)(C)C(C)(C)C)n1ccc(=O)[nH]c1=O ; COP(=O)(CP(=O)(OC)OC)OC ; C=CCOCC(/C=C/P(=O)(OC)OC)OC(CO[Si](C)(C)C(C)(C)C)n1ccc(=O)[nH]c1=O ; C=CCOCC(/C=C/P(=O)(OC)OC)OC(CO)n1ccc(=O)[nH]c1=O ; C=CCOCC(/C=C/P(=O)(OC)OC)OC(COP(OCCC#N)N(C(C)C)C(C)C)n1ccc(=O)[nH]c1=O]

### MOLECULAR BIOLOGY EXAMPLES

### Example 1. Sequences

Tables 1, 3, 5, 6, and 7 illustrate target sequences described herein. Tables 2, 4, 8, and 9 illustrate hetero-duplex polynucleotide sequences described herein.

TABLE 1
KRAS Target Sequences

| | sequence | | SEQ |
|---|---|---|---|
| Id | position in | target site in | ID |
| # | NM_033360.2 | NM_033360.2 | NO: |
| 182 | 182-200 | AAAUGACUGAAUAUAAACUUGUG | 1 |
| 183 | 183-201 | AAUGACUGAAUAUAAACUUGUGG | 2 |
| 197 | 197-215 | AACUUGUGGUAGUUGGAGCUGGU | 3 |
| 224 | 224-242 | UAGGCAAGAGUGCCUUGACGAUA | 4 |
| 226 | 226-244 | GGCAAGAGUGCCUUGACGAUACA | 5 |
| 227 | 227-245 | GCAAGAGUGCCUUGACGAUACAG | 6 |
| 228 | 228-246 | CAAGAGUGCCUUGACGAUACAGC | 7 |
| 232 | 232-250 | AGUGCCUUGACGAUACAGCUAAU | 8 |
| 233 | 233-251 | GUGCCUUGACGAUACAGCUAAUU | 9 |
| 236 | 236-254 | CCUUGACGAUACAGCUAAUUCAG | 10 |
| 237 | 237-255 | CUUGACGAUACAGCUAAUUCAGA | 11 |
| 245 | 245-263 | UACAGCUAAUUCAGAAUCAUUUU | 12 |
| 266 | 266-284 | UUGUGGACGAAUAUGAUCCAACA | 13 |
| 269 | 269-287 | UGGACGAAUAUGAUCCAACAAUA | 14 |
| 270 | 270-288 | GGACGAAUAUGAUCCAACAAUAG | 15 |

TABLE 2
KRAS siRNA sequences

| | sequence | | | | |
|---|---|---|---|---|---|
| | position | sense | | antisense | |
| | in NM_ | strand | SEQ | strand | SEQ |
| Id | 033360. | sequence | ID | sequence | ID |
| # | 2 | (5′-3′) | NO: | (5′-3′) | NO: |
| 182 | 182-200 | AUGACUGAAUA | 16 | CAAGUUUAUAUU | 17 |
| | | UAAACUUGTT | | CAGUCAUTT | |
| 183 | 183-201 | UGACUGAAUAU | 18 | ACAAGUUUAUAU | 19 |
| | | AAACUUGUTT | | UCAGUCATT | |
| 197 | 197-215 | CUUGUGGUAGU | 20 | CAGCUCCAACUA | 21 |
| | | UGGAGCUGTT | | CCACAAGTT | |
| 224 | 224-242 | GGCAAGAGUGC | 22 | UCGUCAAGGCAC | 23 |
| | | CUUGACGATT | | UCUUGCCTT | |
| 226 | 226-244 | CAAGAGUGCCU | 24 | UAUCGUCAAGGC | 25 |
| | | UGACGAUATT | | ACUCUUGTT | |
| 227 | 227-245 | AAGAGUGCCUU | 26 | GUAUCGUCAAGG | 27 |
| | | GACGAUACTT | | CACUCUUTT | |
| 228 | 228-246 | AGAGUGCCUUG | 28 | UGUAUCGUCAAG | 29 |
| | | ACGAUACATT | | GCACUCUTT | |
| 232 | 232-250 | UGCCUUGACGA | 30 | UAGCUGUAUCGU | 31 |
| | | UACAGCUATT | | CAAGGCATT | |
| 233 | 233-251 | GCCUUGACGAU | 32 | UUAGCUGUAUCG | 33 |
| | | ACAGCUAATT | | UCAAGGCTT | |
| 236 | 236-254 | UUGACGAUACA | 34 | GAAUUAGCUGUA | 35 |
| | | GCUAAUUCTT | | UCGUCAATT | |
| 237 | 237-255 | UGACGAUACAG | 36 | UGAAUUAGCUGU | 37 |
| | | CUAAUUCATT | | AUCGUCATT | |
| 245 | 245-263 | CAGCUAAUUCA | 38 | AAUGAUUCUGAA | 39 |
| | | GAAUCAUUTT | | UUAGCUGTT | |
| 266 | 266-284 | GUGGACGAAUA | 40 | UUGGAUCAUAUU | 41 |
| | | UGAUCCAATT | | CGUCCACTT | |
| 269 | 269-287 | GACGAAUAUGA | 42 | UUGUUGGAUCAU | 43 |
| | | UCCAACAATT | | AUUCGUCTT | |
| 270 | 270-288 | ACGAAUAUGAU | 44 | AUUGUUGGAUCA | 45 |
| | | CCAACAAUTT | | UAUUCGUTT | |

TABLE 3
EGFR Target Sequences

| | 19mer | | |
|---|---|---|---|
| hs | pos. in | sequence of | SEQ |
| Id | NM_ | total 23mer target | ID |
| # | 005228.3 | site in NM_005228.3 | NO: |
| 68 | 68-86 | GGCGGCCGGAGUCCCGAGCUAGC | 46 |
| 71 | 71-89 | GGCCGGAGUCCCGAGCUAGCCCC | 47 |
| 72 | 72-90 | GCCGGAGUCCCGAGCUAGCCCCG | 48 |
| 73 | 73-91 | CCGGAGUCCCGAGCUAGCCCCGG | 49 |
| 74 | 74-92 | CGGAGUCCCGAGCUAGCCCCGGC | 50 |
| 75 | 75-93 | GGAGUCCCGAGCUAGCCCCGGCG | 51 |
| 76 | 76-94 | GAGUCCCGAGCUAGCCCCGGCGG | 52 |
| 78 | 78-96 | GUCCCGAGCUAGCCCCGGCGGCC | 53 |
| 114 | 114-132 | CCGGACGACAGGCCACCUCGUCG | 54 |
| 115 | 115-133 | CGGACGACAGGCCACCUCGUCGG | 55 |
| 116 | 116-134 | GGACGACAGGCCACCUCGUCGGC | 56 |
| 117 | 117-135 | GACGACAGGCCACCUCGUCGGCG | 57 |
| 118 | 118-136 | ACGACAGGCCACCUCGUCGGCGU | 58 |
| 120 | 120-138 | GACAGGCCACCUCGUCGGCGUCC | 59 |
| 121 | 121-139 | ACAGGCCACCUCGUCGGCGUCCG | 60 |
| 122 | 122-140 | CAGGCCACCUCGUCGGCGUCCGC | 61 |
| 123 | 123-141 | AGGCCACCUCGUCGGCGUCCGCC | 62 |
| 124 | 124-142 | GGCCACCUCGUCGGCGUCCGCCC | 63 |
| 125 | 125-143 | GCCACCUCGUCGGCGUCCGCCCG | 64 |
| 126 | 126-144 | CCACCUCGUCGGCGUCCGCCCGA | 65 |
| 127 | 127-145 | CACCUCGUCGGCGUCCGCCCGAG | 66 |
| 128 | 128-146 | ACCUCGUCGGCGUCCGCCCGAGU | 67 |
| 129 | 129-147 | CCUCGUCGGCGUCCGCCCGAGUC | 68 |
| 130 | 130-148 | CUCGUCGGCGUCCGCCCGAGUCC | 69 |
| 131 | 131-149 | UCGUCGGCGUCCGCCCGAGUCCC | 70 |
| 132 | 132-150 | CGUCGGCGUCCGCCCGAGUCCCC | 71 |
| 135 | 135-153 | CGGCGUCCGCCCGAGUCCCCGCC | 72 |
| 136 | 136-154 | GGCGUCCGCCCGAGUCCCCGCCU | 73 |
| 141 | 141-159 | CCGCCCGAGUCCCCGCCUCGCCG | 74 |
| 164 | 164-182 | CCAACGCCACAACCACCGCGCAC | 75 |
| 165 | 165-183 | CAACGCCACAACCACCGCGCACG | 76 |
| 166 | 166-184 | AACGCCACAACCACCGCGCACGG | 77 |
| 168 | 168-186 | CGCCACAACCACCGCGCACGGCC | 78 |
| 169 | 169-187 | GCCACAACCACCGCGCACGGCCC | 79 |
| 170 | 170-188 | CCACAACCACCGCGCACGGCCCC | 80 |
| 247 | 247-265 | CGAUGCGACCCUCCGGGACGGCC | 81 |
| 248 | 248-266 | GAUGCGACCCUCCGGGACGGCCG | 82 |
| 249 | 249-267 | AUGCGACCCUCCGGGACGGCCGG | 83 |
| 251 | 251-269 | GCGACCCUCCGGGACGGCCGGGG | 84 |
| 252 | 252-270 | CGACCCUCCGGGACGGCCGGGGC | 85 |
| 254 | 254-272 | ACCCUCCGGGACGGCCGGGGCAG | 86 |
| 329 | 329-347 | AAAGAAAGUUUGCCAAGGCACGA | 87 |
| 330 | 330-348 | AAGAAAGUUUGCCAAGGCACGAG | 88 |
| 332 | 332-350 | GAAAGUUUGCCAAGGCACGAGUA | 89 |
| 333 | 333-351 | AAAGUUUGCCAAGGCACGAGUAA | 90 |
| 334 | 334-352 | AAGUUUGCCAAGGCACGAGUAAC | 91 |
| 335 | 335-353 | AGUUUGCCAAGGCACGAGUAACA | 92 |
| 336 | 336-354 | GUUUGCCAAGGCACGAGUAACAA | 93 |
| 337 | 337-355 | UUUGCCAAGGCACGAGUAACAAG | 94 |
| 338 | 338-356 | UUGCCAAGGCACGAGUAACAAGC | 95 |
| 361 | 361-379 | UCACGCAGUUGGGCACUUUUGAA | 96 |
| 362 | 362-380 | CACGCAGUUGGGCACUUUUGAAG | 97 |
| 363 | 363-381 | ACGCAGUUGGGCACUUUUGAAGA | 98 |
| 364 | 364-382 | CGCAGUUGGGCACUUUUGAAGAU | 99 |
| 365 | 365-383 | GCAGUUGGGCACUUUUGAAGAUC | 100 |
| 366 | 366-384 | CAGUUGGGCACUUUUGAAGAUCA | 101 |
| 367 | 367-385 | AGUUGGGCACUUUUGAAGAUCAU | 102 |
| 368 | 368-386 | GUUGGGCACUUUUGAAGAUCAUU | 103 |
| 369 | 369-387 | UUGGGCACUUUUGAAGAUCAUUU | 104 |
| 377 | 377-395 | UUUUGAAGAUCAUUUUCUCAGCC | 105 |
| 379 | 379-397 | UUGAAGAUCAUUUUCUCAGCCUC | 106 |
| 380 | 380-398 | UGAAGAUCAUUUUCUCAGCCUCC | 107 |
| 385 | 385-403 | AUCAUUUUCUCAGCCUCCAGAGG | 108 |
| 394 | 394-412 | UCAGCCUCCAGAGGAUGUUCAAU | 109 |
| 396 | 396-414 | AGCCUCCAGAGGAUGUUCAAUAA | 110 |
| 397 | 397-415 | GCCUCCAGAGGAUGUUCAAUAAC | 111 |
| 401 | 401-419 | CCAGAGGAUGUUCAAUAACUGUG | 112 |
| 403 | 403-421 | AGAGGAUGUUCAAUAACUGUGAG | 113 |
| 407 | 407-425 | GAUGUUCAAUAACUGUGAGGUGG | 114 |
| 409 | 409-427 | UGUUCAAUAACUGUGAGGUGGUC | 115 |
| 410 | 410-428 | GUUCAAUAACUGUGAGGUGGUCC | 116 |
| 411 | 411-429 | UUCAAUAACUGUGAGGUGGUCCU | 117 |
| 412 | 412-430 | UCAAUAACUGUGAGGUGGUCCUU | 118 |
| 413 | 413-431 | CAAUAACUGUGAGGUGGUCCUUG | 119 |
| 414 | 414-432 | AAUAACUGUGAGGUGGUCCUUGG | 120 |
| 416 | 416-434 | UAACUGUGAGGUGGUCCUUGGGA | 121 |
| 418 | 418-436 | ACUGUGAGGUGGUCCUUGGGAAU | 122 |
| 419 | 419-437 | CUGUGAGGUGGUCCUUGGGAAUU | 123 |
| 425 | 425-443 | GGUGGUCCUUGGGAAUUUGGAAA | 124 |
| 431 | 431-449 | CCUUGGGAAUUUGGAAAUUACCU | 125 |
| 432 | 432-450 | CUUGGGAAUUUGGAAAUUACCUA | 126 |
| 433 | 433-451 | UUGGGAAUUUGGAAAUUACCUAU | 127 |
| 434 | 434-452 | UGGGAAUUUGGAAAUUACCUAUG | 128 |
| 458 | 458-476 | GCAGAGGAAUUAUGAUCUUUCCU | 129 |
| 459 | 459-477 | CAGAGGAAUUAUGAUCUUUCCUU | 130 |
| 463 | 463-481 | GGAAUUAUGAUCUUUCCUUCUUA | 131 |
| 464 | 464-482 | GAAUUAUGAUCUUUCCUUCUUAA | 132 |
| 466 | 466-484 | AUUAUGAUCUUUCCUUCUUAAAG | 133 |
| 468 | 468-486 | UAUGAUCUUUCCUUCUUAAAGAC | 134 |
| 471 | 471-489 | GAUCUUUCCUUCUUAAAGACCAU | 135 |
| 476 | 476-494 | UUCCUUCUUAAAGACCAUCCAGG | 136 |
| 477 | 477-495 | UCCUUCUUAAAGACCAUCCAGGA | 137 |
| 479 | 479-497 | CUUCUUAAAGACCAUCCAGGAGG | 138 |
| 481 | 481-499 | UCUUAAAGACCAUCCAGGAGGUG | 139 |
| 482 | 482-500 | CUUAAAGACCAUCCAGGAGGUGG | 140 |
| 492 | 492-510 | AUCCAGGAGGUGGCUGGUUAUGU | 141 |
| 493 | 493-511 | UCCAGGAGGUGGCUGGUUAUGUC | 142 |
| 494 | 494-512 | CCAGGAGGUGGCUGGUUAUGUCC | 143 |
| 495 | 495-513 | CAGGAGGUGGCUGGUUAUGUCCU | 144 |
| 496 | 496-514 | AGGAGGUGGCUGGUUAUGUCCUC | 145 |
| 497 | 497-515 | GGAGGUGGCUGGUUAUGUCCUCA | 146 |
| 499 | 499-517 | AGGUGGCUGGUUAUGUCCUCAUU | 147 |
| 520 | 520-538 | UUGCCCUCAACACAGUGGAGCGA | 148 |
| 542 | 542-560 | AAUUCCUUUGGAAAACCUGCAGA | 149 |
| 543 | 543-561 | AUUCCUUUGGAAAACCUGCAGAU | 150 |
| 550 | 550-568 | UGGAAAACCUGCAGAUCAUCAGA | 151 |
| 551 | 551-569 | GGAAAACCUGCAGAUCAUCAGAG | 152 |
| 553 | 553-571 | AAAACCUGCAGAUCAUCAGAGGA | 153 |
| 556 | 556-574 | ACCUGCAGAUCAUCAGAGGAAAU | 154 |
| 586 | 586-604 | ACGAAAAUUCCUAUGCCUUAGCA | 155 |
| 587 | 587-605 | CGAAAAUUCCUAUGCCUUAGCAG | 156 |
| 589 | 589-607 | AAAAUUCCUAUGCCUUAGCAGUC | 157 |
| 592 | 592-610 | AUUCCUAUGCCUUAGCAGUCUUA | 158 |
| 593 | 593-611 | UUCCUAUGCCUUAGCAGUCUUAU | 159 |
| 594 | 594-612 | UCCUAUGCCUUAGCAGUCUUAUC | 160 |
| 596 | 596-614 | CUAUGCCUUAGCAGUCUUAUCUA | 161 |
| 597 | 597-615 | UAUGCCUUAGCAGUCUUAUCUAA | 162 |
| 598 | 598-616 | AUGCCUUAGCAGUCUUAUCUAAC | 163 |
| 599 | 599-617 | UGCCUUAGCAGUCUUAUCUAACU | 164 |
| 600 | 600-618 | GCCUUAGCAGUCUUAUCUAACUA | 165 |
| 601 | 601-619 | CCUUAGCAGUCUUAUCUAACUAU | 166 |
| 602 | 602-620 | CUUAGCAGUCUUAUCUAACUAUG | 167 |
| 603 | 603-621 | UUAGCAGUCUUAUCUAACUAUGA | 168 |
| 604 | 604-622 | UAGCAGUCUUAUCUAACUAUGAU | 169 |
| 605 | 605-623 | AGCAGUCUUAUCUAACUAUGAUG | 170 |
| 608 | 608-626 | AGUCUUAUCUAACUAUGAUGCAA | 171 |
| 609 | 609-627 | GUCUUAUCUAACUAUGAUGCAAA | 172 |
| 610 | 610-628 | UCUUAUCUAACUAUGAUGCAAAU | 173 |
| 611 | 611-629 | CUUAUCUAACUAUGAUGCAAAUA | 174 |
| 612 | 612-630 | UUAUCUAACUAUGAUGCAAAUAA | 175 |
| 613 | 613-631 | UAUCUAACUAUGAUGCAAAUAAA | 176 |
| 614 | 614-632 | AUCUAACUAUGAUGCAAAUAAAA | 177 |
| 616 | 616-634 | CUAACUAUGAUGCAAAUAAAACC | 178 |
| 622 | 622-640 | AUGAUGCAAAUAAAACCGGACUG | 179 |
| 623 | 623-641 | UGAUGCAAAUAAAACCGGACUGA | 180 |
| 624 | 624-642 | GAUGCAAAUAAAACCGGACUGAA | 181 |
| 626 | 626-644 | UGCAAAUAAAACCGGACUGAAGG | 182 |
| 627 | 627-645 | GCAAAUAAAACCGGACUGAAGGA | 183 |
| 628 | 628-646 | CAAAUAAAACCGGACUGAAGGAG | 184 |
| 630 | 630-648 | AAUAAAACCGGACUGAAGGAGCU | 185 |
| 631 | 631-649 | AUAAAACCGGACUGAAGGAGCUG | 186 |
| 632 | 632-650 | UAAAACCGGACUGAAGGAGCUGC | 187 |
| 633 | 633-651 | AAAACCGGACUGAAGGAGCUGCC | 188 |
| 644 | 644-662 | GAAGGAGCUGCCCAUGAGAAAUU | 189 |
| 665 | 665-683 | UUUACAGGAAAUCCUGCAUGGCG | 190 |
| 668 | 668-686 | ACAGGAAAUCCUGCAUGGCGCCG | 191 |
| 669 | 669-687 | CAGGAAAUCCUGCAUGGCGCCGU | 192 |
| 670 | 670-688 | AGGAAAUCCUGCAUGGCGCCGUG | 193 |
| 671 | 671-689 | GGAAAUCCUGCAUGGCGCCGUGC | 194 |
| 672 | 672-690 | GAAAUCCUGCAUGGCGCCGUGCG | 195 |
| 674 | 674-692 | AAUCCUGCAUGGCGCCGUGCGGU | 196 |
| 676 | 676-694 | UCCUGCAUGGCGCCGUGCGGUUC | 197 |
| 677 | 677-695 | CCUGCAUGGCGCCGUGCGGUUCA | 198 |
| 678 | 678-696 | CUGCAUGGCGCCGUGCGGUUCAG | 199 |
| 680 | 680-698 | GCAUGGCGCCGUGCGGUUCAGCA | 200 |
| 681 | 681-699 | CAUGGCGCCGUGCGGUUCAGCAA | 201 |
| 682 | 682-700 | AUGGCGCCGUGCGGUUCAGCAAC | 202 |
| 683 | 683-701 | UGGCGCCGUGCGGUUCAGCAACA | 203 |
| 684 | 684-702 | GGCGCCGUGCGGUUCAGCAACAA | 204 |
| 685 | 685-703 | GCGCCGUGCGGUUCAGCAACAAC | 205 |
| 686 | 686-704 | CGCCGUGCGGUUCAGCAACAACC | 206 |
| 688 | 688-706 | CCGUGCGGUUCAGCAACAACCCU | 207 |
| 690 | 690-708 | GUGCGGUUCAGCAACAACCCUGC | 208 |
| 692 | 692-710 | GCGGUUCAGCAACAACCCUGCCC | 209 |
| 698 | 698-716 | CAGCAACAACCCUGCCCUGUGCA | 210 |
| 700 | 700-718 | GCAACAACCCUGCCCUGUGCAAC | 211 |
| 719 | 719-737 | CAACGUGGAGAGCAUCCAGUGGC | 212 |
| 720 | 720-738 | AACGUGGAGAGCAUCCAGUGGCG | 213 |
| 721 | 721-739 | ACGUGGAGAGCAUCCAGUGGCGG | 214 |
| 724 | 724-742 | UGGAGAGCAUCCAGUGGCGGGAC | 215 |
| 725 | 725-743 | GGAGAGCAUCCAGUGGCGGGACA | 216 |
| 726 | 726-744 | GAGAGCAUCCAGUGGCGGGACAU | 217 |
| 733 | 733-751 | UCCAGUGGCGGGACAUAGUCAGC | 218 |
| 734 | 734-752 | CCAGUGGCGGGACAUAGUCAGCA | 219 |
| 736 | 736-754 | AGUGGCGGGACAUAGUCAGCAGU | 220 |
| 737 | 737-755 | GUGGCGGGACAUAGUCAGCAGUG | 221 |
| 763 | 763-781 | UUCUCAGCAACAUGUCGAUGGAC | 222 |
| 765 | 765-783 | CUCAGCAACAUGUCGAUGGACUU | 223 |
| 766 | 766-784 | UCAGCAACAUGUCGAUGGACUUC | 224 |
| 767 | 767-785 | CAGCAACAUGUCGAUGGACUUCC | 225 |
| 769 | 769-787 | GCAACAUGUCGAUGGACUUCCAG | 226 |
| 770 | 770-788 | CAACAUGUCGAUGGACUUCCAGA | 227 |
| 771 | 771-789 | AACAUGUCGAUGGACUUCCAGAA | 228 |
| 772 | 772-790 | ACAUGUCGAUGGACUUCCAGAAC | 229 |
| 775 | 775-793 | UGUCGAUGGACUUCCAGAACCAC | 230 |
| 789 | 789-807 | CAGAACCACCUGGGCAGCUGCCA | 231 |
| 798 | 798-816 | CUGGGCAGCUGCCAAAAGUGUGA | 232 |
| 800 | 800-818 | GGGCAGCUGCCAAAAGUGUGAUC | 233 |
| 805 | 805-823 | GCUGCCAAAAGUGUGAUCCAAGC | 234 |
| 806 | 806-824 | CUGCCAAAAGUGUGAUCCAAGCU | 235 |
| 807 | 807-825 | UGCCAAAAGUGUGAUCCAAGCUG | 236 |
| 810 | 810-828 | CAAAAGUGUGAUCCAAGCUGUCC | 237 |
| 814 | 814-832 | AGUGUGAUCCAAGCUGUCCCAAU | 238 |
| 815 | 815-833 | GUGUGAUCCAAGCUGUCCCAAUG | 239 |
| 817 | 817-835 | GUGAUCCAAGCUGUCCCAAUGGG | 240 |
| 818 | 818-836 | UGAUCCAAGCUGUCCCAAUGGGA | 241 |
| 819 | 819-837 | GAUCCAAGCUGUCCCAAUGGGAG | 242 |
| 820 | 820-838 | AUCCAAGCUGUCCCAAUGGGAGC | 243 |
| 821 | 821-839 | UCCAAGCUGUCCCAAUGGGAGCU | 244 |
| 823 | 823-841 | CAAGCUGUCCCAAUGGGAGCUGC | 245 |
| 826 | 826-844 | GCUGUCCCAAUGGGAGCUGCUGG | 246 |
| 847 | 847-865 | GGGGUGCAGGAGAGGAGAACUGC | 247 |
| 871 | 871-889 | AGAAACUGACCAAAAUCAUCUGU | 248 |
| 872 | 872-890 | GAAACUGACCAAAAUCAUCUGUG | 249 |
| 873 | 873-891 | AAACUGACCAAAAUCAUCUGUGC | 250 |
| 877 | 877-895 | UGACCAAAAUCAUCUGUGCCCAG | 251 |
| 878 | 878-896 | GACCAAAAUCAUCUGUGCCCAGC | 252 |
| 881 | 881-899 | CAAAAUCAUCUGUGCCCAGCAGU | 253 |
| 890 | 890-908 | CUGUGCCCAGCAGUGCUCCGGGC | 254 |
| 892 | 892-910 | GUGCCCAGCAGUGCUCCGGGCGC | 255 |
| 929 | 929-947 | CCCCAGUGACUGCUGCCACAACC | 256 |
| 930 | 930-948 | CCCAGUGACUGCUGCCACAACCA | 257 |
| 979 | 979-997 | GGGAGAGCGACUGCCUGGUCUGC | 258 |
| 980 | 980-998 | GGAGAGCGACUGCCUGGUCUGCC | 259 |
| 981 | 981-999 | GAGAGCGACUGCCUGGUCUGCCG | 260 |
| 982 | 982-1000 | AGAGCGACUGCCUGGUCUGCCGC | 261 |
| 983 | 983-1001 | GAGCGACUGCCUGGUCUGCCGCA | 262 |
| 984 | 984-1002 | AGCGACUGCCUGGUCUGCCGCAA | 263 |
| 989 | 989-1007 | CUGCCUGGUCUGCCGCAAAUUCC | 264 |
| 990 | 990-1008 | UGCCUGGUCUGCCGCAAAUUCCG | 265 |
| 991 | 991-1009 | GCCUGGUCUGCCGCAAAUUCCGA | 266 |
| 992 | 992-1010 | CCUGGUCUGCCGCAAAUUCCGAG | 267 |
| 994 | 994-1012 | UGGUCUGCCGCAAAUUCCGAGAC | 268 |
| 995 | 995-1013 | GGUCUGCCGCAAAUUCCGAGACG | 269 |
| 996 | 996-1014 | GUCUGCCGCAAAUUCCGAGACGA | 270 |
| 997 | 997-1015 | UCUGCCGCAAAUUCCGAGACGAA | 271 |
| 999 | 999-1017 | UGCCGCAAAUUCCGAGACGAAGC | 272 |
| 1004 | 1004-1022 | CAAAUUCCGAGACGAAGCCACGU | 273 |
| 1005 | 1005-1023 | AAAUUCCGAGACGAAGCCACGUG | 274 |
| 1006 | 1006-1024 | AAUUCCGAGACGAAGCCACGUGC | 275 |
| 1007 | 1007-1025 | AUUCCGAGACGAAGCCACGUGCA | 276 |
| 1008 | 1008-1026 | UUCCGAGACGAAGCCACGUGCAA | 277 |
| 1010 | 1010-1028 | CCGAGACGAAGCCACGUGCAAGG | 278 |
| 1013 | 1013-1031 | AGACGAAGCCACGUGCAAGGACA | 279 |
| 1014 | 1014-1032 | GACGAAGCCACGUGCAAGGACAC | 280 |
| 1015 | 1015-1033 | ACGAAGCCACGUGCAAGGACACC | 281 |
| 1016 | 1016-1034 | CGAAGCCACGUGCAAGGACACCU | 282 |
| 1040 | 1040-1058 | CCCCCCACUCAUGCUCUACAACC | 283 |
| 1042 | 1042-1060 | CCCCACUCAUGCUCUACAACCCC | 284 |
| 1044 | 1044-1062 | CCACUCAUGCUCUACAACCCCAC | 285 |
| 1047 | 1047-1065 | CUCAUGCUCUACAACCCCACCAC | 286 |
| 1071 | 1071-1089 | UACCAGAUGGAUGUGAACCCCGA | 287 |
| 1073 | 1073-1091 | CCAGAUGGAUGUGAACCCCGAGG | 288 |
| 1074 | 1074-1092 | CAGAUGGAUGUGAACCCCGAGGG | 289 |
| 1075 | 1075-1093 | AGAUGGAUGUGAACCCCGAGGGC | 290 |
| 1077 | 1077-1095 | AUGGAUGUGAACCCCGAGGGCAA | 291 |
| 1078 | 1078-1096 | UGGAUGUGAACCCCGAGGGCAAA | 292 |
| 1080 | 1080-1098 | GAUGUGAACCCCGAGGGCAAAUA | 293 |
| 1084 | 1084-1102 | UGAACCCCGAGGGCAAAUACAGC | 294 |
| 1085 | 1085-1103 | GAACCCCGAGGGCAAAUACAGCU | 295 |
| 1087 | 1087-1105 | ACCCCGAGGGCAAAUACAGCUUU | 296 |
| 1088 | 1088-1106 | CCCCGAGGGCAAAUACAGCUUUG | 297 |
| 1089 | 1089-1107 | CCCGAGGGCAAAUACAGCUUUGG | 298 |
| 1096 | 1096-1114 | GCAAAUACAGCUUUGGUGCCACC | 299 |
| 1097 | 1097-1115 | CAAAUACAGCUUUGGUGCCACCU | 300 |
| 1098 | 1098-1116 | AAAUACAGCUUUGGUGCCACCUG | 301 |
| 1104 | 1104-1122 | AGCUUUGGUGCCACCUGCGUGAA | 302 |
| 1106 | 1106-1124 | CUUUGGUGCCACCUGCGUGAAGA | 303 |
| 1112 | 1112-1130 | UGCCACCUGCGUGAAGAAGUGUC | 304 |
| 1116 | 1116-1134 | ACCUGCGUGAAGAAGUGUCCCCG | 305 |
| 1117 | 1117-1135 | CCUGCGUGAAGAAGUGUCCCCGU | 306 |
| 1118 | 1118-1136 | CUGCGUGAAGAAGUGUCCCCGUA | 307 |
| 1119 | 1119-1137 | UGCGUGAAGAAGUGUCCCCGUAA | 308 |
| 1120 | 1120-1138 | GCGUGAAGAAGUGUCCCCGUAAU | 309 |
| 1121 | 1121-1139 | CGUGAAGAAGUGUCCCCGUAAUU | 310 |
| 1122 | 1122-1140 | GUGAAGAAGUGUCCCCGUAAUUA | 311 |
| 1123 | 1123-1141 | UGAAGAAGUGUCCCCGUAAUUAU | 312 |
| 1124 | 1124-1142 | GAAGAAGUGUCCCCGUAAUUAUG | 313 |
| 1125 | 1125-1143 | AAGAAGUGUCCCCGUAAUUAUGU | 314 |
| 1126 | 1126-1144 | AGAAGUGUCCCCGUAAUUAUGUG | 315 |
| 1127 | 1127-1145 | GAAGUGUCCCCGUAAUUAUGUGG | 316 |
| 1128 | 1128-1146 | AAGUGUCCCCGUAAUUAUGUGGU | 317 |
| 1129 | 1129-1147 | AGUGUCCCCGUAAUUAUGUGGUG | 318 |
| 1130 | 1130-1148 | GUGUCCCCGUAAUUAUGUGGUGA | 319 |
| 1132 | 1132-1150 | GUCCCCGUAAUUAUGUGGUGACA | 320 |
| 1134 | 1134-1152 | CCCCGUAAUUAUGUGGUGACAGA | 321 |
| 1136 | 1136-1154 | CCGUAAUUAUGUGGUGACAGAUC | 322 |
| 1137 | 1137-1155 | CGUAAUUAUGUGGUGACAGAUCA | 323 |
| 1138 | 1138-1156 | GUAAUUAUGUGGUGACAGAUCAC | 324 |
| 1139 | 1139-1157 | UAAUUAUGUGGUGACAGAUCACG | 325 |
| 1140 | 1140-1158 | AAUUAUGUGGUGACAGAUCACGG | 326 |
| 1142 | 1142-1160 | UUAUGUGGUGACAGAUCACGGCU | 327 |
| 1145 | 1145-1163 | UGUGGUGACAGAUCACGGCUCGU | 328 |
| 1147 | 1147-1165 | UGGUGACAGAUCACGGCUCGUGC | 329 |
| 1148 | 1148-1166 | GGUGACAGAUCACGGCUCGUGCG | 330 |
| 1149 | 1149-1167 | GUGACAGAUCACGGCUCGUGCGU | 331 |
| 1150 | 1150-1168 | UGACAGAUCACGGCUCGUGCGUC | 332 |
| 1151 | 1151-1169 | GACAGAUCACGGCUCGUGCGUCC | 333 |
| 1152 | 1152-1170 | ACAGAUCACGGCUCGUGCGUCCG | 334 |
| 1153 | 1153-1171 | CAGAUCACGGCUCGUGCGUCCGA | 335 |
| 1154 | 1154-1172 | AGAUCACGGCUCGUGCGUCCGAG | 336 |
| 1155 | 1155-1173 | GAUCACGGCUCGUGCGUCCGAGC | 337 |
| 1156 | 1156-1174 | AUCACGGCUCGUGCGUCCGAGCC | 338 |
| 1157 | 1157-1175 | UCACGGCUCGUGCGUCCGAGCCU | 339 |
| 1160 | 1160-1178 | CGGCUCGUGCGUCCGAGCCUGUG | 340 |
| 1200 | 1200-1218 | AUGGAGGAAGACGGCGUCCGCAA | 341 |
| 1201 | 1201-1219 | UGGAGGAAGACGGCGUCCGCAAG | 342 |
| 1203 | 1203-1221 | GAGGAAGACGGCGUCCGCAAGUG | 343 |
| 1204 | 1204-1222 | AGGAAGACGGCGUCCGCAAGUGU | 344 |
| 1205 | 1205-1223 | GGAAGACGGCGUCCGCAAGUGUA | 345 |
| 1207 | 1207-1225 | AAGACGGCGUCCGCAAGUGUAAG | 346 |
| 1208 | 1208-1226 | AGACGGCGUCCGCAAGUGUAAGA | 347 |
| 1211 | 1211-1229 | CGGCGUCCGCAAGUGUAAGAAGU | 348 |
| 1212 | 1212-1230 | GGCGUCCGCAAGUGUAAGAAGUG | 349 |
| 1213 | 1213-1231 | GCGUCCGCAAGUGUAAGAAGUGC | 350 |
| 1214 | 1214-1232 | CGUCCGCAAGUGUAAGAAGUGCG | 351 |
| 1215 | 1215-1233 | GUCCGCAAGUGUAAGAAGUGCGA | 352 |
| 1216 | 1216-1234 | UCCGCAAGUGUAAGAAGUGCGAA | 353 |
| 1217 | 1217-1235 | CCGCAAGUGUAAGAAGUGCGAAG | 354 |
| 1219 | 1219-1237 | GCAAGUGUAAGAAGUGCGAAGGG | 355 |
| 1220 | 1220-1238 | CAAGUGUAAGAAGUGCGAAGGGC | 356 |
| 1221 | 1221-1239 | AAGUGUAAGAAGUGCGAAGGGCC | 357 |
| 1222 | 1222-1240 | AGUGUAAGAAGUGCGAAGGGCCU | 358 |
| 1223 | 1223-1241 | GUGUAAGAAGUGCGAAGGGCCUU | 359 |
| 1224 | 1224-1242 | UGUAAGAAGUGCGAAGGGCCUUG | 360 |
| 1225 | 1225-1243 | GUAAGAAGUGCGAAGGGCCUUGC | 361 |
| 1226 | 1226-1244 | UAAGAAGUGCGAAGGGCCUUGCC | 362 |
| 1229 | 1229-1247 | GAAGUGCGAAGGGCCUUGCCGCA | 363 |
| 1230 | 1230-1248 | AAGUGCGAAGGGCCUUGCCGCAA | 364 |
| 1231 | 1231-1249 | AGUGCGAAGGGCCUUGCCGCAAA | 365 |
| 1232 | 1232-1250 | GUGCGAAGGGCCUUGCCGCAAAG | 366 |
| 1233 | 1233-1251 | UGCGAAGGGCCUUGCCGCAAAGU | 367 |
| 1235 | 1235-1253 | CGAAGGGCCUUGCCGCAAAGUGU | 368 |
| 1236 | 1236-1254 | GAAGGGCCUUGCCGCAAAGUGUG | 369 |
| 1237 | 1237-1255 | AAGGGCCUUGCCGCAAAGUGUGU | 370 |
| 1238 | 1238-1256 | AGGGCCUUGCCGCAAAGUGUGUA | 371 |
| 1239 | 1239-1257 | GGGCCUUGCCGCAAAGUGUGUAA | 372 |
| 1241 | 1241-1259 | GCCUUGCCGCAAAGUGUGUAACG | 373 |
| 1261 | 1261-1279 | ACGGAAUAGGUAUUGGUGAAUUU | 374 |
| 1262 | 1262-1280 | CGGAAUAGGUAUUGGUGAAUUUA | 375 |
| 1263 | 1263-1281 | GGAAUAGGUAUUGGUGAAUUUAA | 376 |
| 1264 | 1264-1282 | GAAUAGGUAUUGGUGAAUUUAAA | 377 |
| 1266 | 1266-1284 | AUAGGUAUUGGUGAAUUUAAAGA | 378 |
| 1267 | 1267-1285 | UAGGUAUUGGUGAAUUUAAAGAC | 379 |
| 1289 | 1289-1307 | CUCACUCUCCAUAAAUGCUACGA | 380 |
| 1313 | 1313-1331 | UAUUAAACACUUCAAAAACUGCA | 381 |
| 1320 | 1320-1338 | CACUUCAAAAACUGCACCUCCAU | 382 |
| 1321 | 1321-1339 | ACUUCAAAAACUGCACCUCCAUC | 383 |
| 1322 | 1322-1340 | CUUCAAAAACUGCACCUCCAUCA | 384 |
| 1323 | 1323-1341 | UUCAAAAACUGCACCUCCAUCAG | 385 |
| 1324 | 1324-1342 | UCAAAAACUGCACCUCCAUCAGU | 386 |
| 1328 | 1328-1346 | AAACUGCACCUCCAUCAGUGGCG | 387 |
| 1332 | 1332-1350 | UGCACCUCCAUCAGUGGCGAUCU | 388 |
| 1333 | 1333-1351 | GCACCUCCAUCAGUGGCGAUCUC | 389 |
| 1335 | 1335-1353 | ACCUCCAUCAGUGGCGAUCUCCA | 390 |
| 1338 | 1338-1356 | UCCAUCAGUGGCGAUCUCCACAU | 391 |
| 1344 | 1344-1362 | AGUGGCGAUCUCCACAUCCUGCC | 392 |
| 1345 | 1345-1363 | GUGGCGAUCUCCACAUCCUGCCG | 393 |
| 1346 | 1346-1364 | UGGCGAUCUCCACAUCCUGCCGG | 394 |
| 1347 | 1347-1365 | GGCGAUCUCCACAUCCUGCCGGU | 395 |
| 1348 | 1348-1366 | GCGAUCUCCACAUCCUGCCGGUG | 396 |
| 1353 | 1353-1371 | CUCCACAUCCUGCCGGUGGCAUU | 397 |
| 1354 | 1354-1372 | UCCACAUCCUGCCGGUGGCAUUU | 398 |
| 1355 | 1355-1373 | CCACAUCCUGCCGGUGGCAUUUA | 399 |
| 1357 | 1357-1375 | ACAUCCUGCCGGUGGCAUUUAGG | 400 |
| 1360 | 1360-1378 | UCCUGCCGGUGGCAUUUAGGGGU | 401 |
| 1361 | 1361-1379 | CCUGCCGGUGGCAUUUAGGGGUG | 402 |
| 1362 | 1362-1380 | CUGCCGGUGGCAUUUAGGGGUGA | 403 |
| 1363 | 1363-1381 | UGCCGGUGGCAUUUAGGGGUGAC | 404 |
| 1366 | 1366-1384 | CGGUGGCAUUUAGGGGUGACUCC | 405 |
| 1369 | 1369-1387 | UGGCAUUUAGGGGUGACUCCUUC | 406 |
| 1370 | 1370-1388 | GGCAUUUAGGGGUGACUCCUUCA | 407 |
| 1371 | 1371-1389 | GCAUUUAGGGGUGACUCCUUCAC | 408 |
| 1372 | 1372-1390 | CAUUUAGGGGUGACUCCUUCACA | 409 |
| 1373 | 1373-1391 | AUUUAGGGGUGACUCCUUCACAC | 410 |
| 1374 | 1374-1392 | UUUAGGGGUGACUCCUUCACACA | 411 |
| 1404 | 1404-1422 | CCUCUGGAUCCACAGGAACUGGA | 412 |
| 1408 | 1408-1426 | UGGAUCCACAGGAACUGGAUAUU | 413 |
| 1409 | 1409-1427 | GGAUCCACAGGAACUGGAUAUUC | 414 |
| 1411 | 1411-1429 | AUCCACAGGAACUGGAUAUUCUG | 415 |
| 1412 | 1412-1430 | UCCACAGGAACUGGAUAUUCUGA | 416 |
| 1419 | 1419-1437 | GAACUGGAUAUUCUGAAAACCGU | 417 |
| 1426 | 1426-1444 | AUAUUCUGAAAACCGUAAAGGAA | 418 |
| 1427 | 1427-1445 | UAUUCUGAAAACCGUAAAGGAAA | 419 |
| 1430 | 1430-1448 | UCUGAAAACCGUAAAGGAAAUCA | 420 |
| 1431 | 1431-1449 | CUGAAAACCGUAAAGGAAAUCAC | 421 |

TABLE 4
EGFR siRNA Sequences

| | Sequence | | SEQ | | SEQ |
|---|---|---|---|---|---|
| hs Id | position in | sense strand sequence | ID | antisense strand | ID |
| # | NM_005228.3 | (5′-3′) | NO: | sequence (5′-3′) | NO: |
| 68 | 68-86 | CGGCCGGAGUCCCGAGCU | 422 | UAGCUCGGGACUCCGGCC | 423 |
| | | ATT | | GTT | |
| 71 | 71-89 | CCGGAGUCCCGAGCUAGC | 424 | GGCUAGCUCGGGACUCCG | 425 |
| | | CTT | | GTT | |
| 72 | 72-90 | CGGAGUCCCGAGCUAGCC | 426 | GGGCUAGCUCGGGACUCC | 427 |
| | | CTT | | GTT | |
| 73 | 73-91 | GGAGUCCCGAGCUAGCCC | 428 | GGGGCUAGCUCGGGACUC | 429 |
| | | CTT | | CTT | |
| 74 | 74-92 | GAGUCCCGAGCUAGCCCC | 430 | CGGGGCUAGCUCGGGACU | 431 |
| | | GTT | | CTT | |
| 75 | 75-93 | AGUCCCGAGCUAGCCCCG | 432 | CCGGGGCUAGCUCGGGAC | 433 |
| | | GTT | | UTT | |
| 76 | 76-94 | GUCCCGAGCUAGCCCCGG | 434 | GCCGGGGCUAGCUCGGGA | 435 |
| | | CTT | | CTT | |
| 78 | 78-96 | CCCGAGCUAGCCCCGGCG | 436 | CCGCCGGGGCUAGCUCGG | 437 |
| | | GTT | | GTT | |
| 114 | 114-132 | GGACGACAGGCCACCUCG | 438 | ACGAGGUGGCCUGUCGUC | 439 |
| | | UTT | | CTT | |
| 115 | 115-133 | GACGACAGGCCACCUCGU | 440 | GACGAGGUGGCCUGUCGU | 441 |
| | | CTT | | CTT | |
| 116 | 116-134 | ACGACAGGCCACCUCGUC | 442 | CGACGAGGUGGCCUGUCG | 443 |
| | | GTT | | UTT | |
| 117 | 117-135 | CGACAGGCCACCUCGUCG | 444 | CCGACGAGGUGGCCUGUC | 445 |
| | | GTT | | GTT | |
| 118 | 118-136 | GACAGGCCACCUCGUCGG | 446 | GCCGACGAGGUGGCCUGU | 447 |
| | | CTT | | CTT | |
| 120 | 120-138 | CAGGCCACCUCGUCGGCG | 448 | ACGCCGACGAGGUGGCCU | 449 |
| | | UTT | | GTT | |
| 121 | 121-139 | AGGCCACCUCGUCGGCGU | 450 | GACGCCGACGAGGUGGCC | 451 |
| | | CTT | | UTT | |
| 122 | 122-140 | GGCCACCUCGUCGGCGUC | 452 | GGACGCCGACGAGGUGGC | 453 |
| | | CTT | | CTT | |
| 123 | 123-141 | GCCACCUCGUCGGCGUCC | 454 | CGGACGCCGACGAGGUGG | 455 |
| | | GTT | | CTT | |
| 124 | 124-142 | CCACCUCGUCGGCGUCCG | 456 | GCGGACGCCGACGAGGUG | 457 |
| | | CTT | | GTT | |
| 125 | 125-143 | CACCUCGUCGGCGUCCGC | 458 | GGCGGACGCCGACGAGGU | 459 |
| | | CTT | | GTT | |
| 126 | 126-144 | ACCUCGUCGGCGUCCGCC | 460 | GGGCGGACGCCGACGAGG | 461 |
| | | CTT | | UTT | |
| 127 | 127-145 | CCUCGUCGGCGUCCGCCC | 462 | CGGGCGGACGCCGACGAG | 463 |
| | | GTT | | GTT | |
| 128 | 128-146 | CUCGUCGGCGUCCGCCCG | 464 | UCGGGCGGACGCCGACGA | 465 |
| | | ATT | | GTT | |
| 129 | 129-147 | UCGUCGGCGUCCGCCCGA | 466 | CUCGGGCGGACGCCGACG | 467 |
| | | GTT | | ATT | |
| 130 | 130-148 | CGUCGGCGUCCGCCCGAG | 468 | ACUCGGGCGGACGCCGAC | 469 |
| | | UTT | | GTT | |
| 131 | 131-149 | GUCGGCGUCCGCCCGAGU | 470 | GACUCGGGCGGACGCCGA | 471 |
| | | CTT | | CTT | |
| 132 | 132-150 | UCGGCGUCCGCCCGAGUC | 472 | GGACUCGGGCGGACGCCG | 473 |
| | | CTT | | ATT | |
| 135 | 135-153 | GCGUCCGCCCGAGUCCCC | 474 | CGGGGACUCGGGCGGACG | 475 |
| | | GTT | | CTT | |
| 136 | 136-154 | CGUCCGCCCGAGUCCCCG | 476 | GCGGGGACUCGGGCGGAC | 477 |
| | | CTT | | GTT | |
| 141 | 141-159 | GCCCGAGUCCCCGCCUCG | 478 | GCGAGGCGGGGACUCGGG | 479 |
| | | CTT | | CTT | |
| 164 | 164-182 | AACGCCACAACCACCGCG | 480 | GCGCGGUGGUUGUGGCGU | 481 |
| | | CTT | | UTT | |
| 165 | 165-183 | ACGCCACAACCACCGCGC | 482 | UGCGCGGUGGUUGUGGCG | 483 |
| | | ATT | | UTT | |
| 166 | 166-184 | CGCCACAACCACCGCGCA | 484 | GUGCGCGGUGGUUGUGGC | 485 |
| | | CTT | | GTT | |
| 168 | 168-186 | CCACAACCACCGCGCACG | 486 | CCGUGCGCGGUGGUUGUG | 487 |
| | | GTT | | GTT | |
| 169 | 169-187 | CACAACCACCGCGCACGG | 488 | GCCGUGCGCGGUGGUUGU | 489 |
| | | CTT | | GTT | |
| 170 | 170-188 | ACAACCACCGCGCACGGC | 490 | GGCCGUGCGCGGUGGUUG | 491 |
| | | CTT | | UTT | |
| 247 | 247-265 | AUGCGACCCUCCGGGACG | 492 | CCGUCCCGGAGGGUCGCA | 493 |
| | | GTT | | UTT | |
| 248 | 248-266 | UGCGACCCUCCGGGACGG | 494 | GCCGUCCCGGAGGGUCGC | 495 |
| | | CTT | | ATT | |
| 249 | 249-267 | GCGACCCUCCGGGACGGC | 496 | GGCCGUCCCGGAGGGUCG | 497 |
| | | CTT | | CTT | |
| 251 | 251-269 | GACCCUCCGGGACGGCCG | 498 | CCGGCCGUCCCGGAGGGU | 499 |
| | | GTT | | CTT | |
| 252 | 252-270 | ACCCUCCGGGACGGCCGG | 500 | CCCGGCCGUCCCGGAGGG | 501 |
| | | GTT | | UTT | |
| 254 | 254-272 | CCUCCGGGACGGCCGGGG | 502 | GCCCCGGCCGUCCCGGAG | 503 |
| | | CTT | | GTT | |
| 329 | 329-347 | AGAAAGUUUGCCAAGGCA | 504 | GUGCCUUGGCAAACUUUC | 505 |
| | | CTT | | UTT | |
| 330 | 330-348 | GAAAGUUUGCCAAGGCAC | 506 | CGUGCCUUGGCAAACUUU | 507 |
| | | GTT | | CTT | |
| 332 | 332-350 | AAGUUUGCCAAGGCACGA | 508 | CUCGUGCCUUGGCAAACU | 509 |
| | | GTT | | UTT | |
| 333 | 333-351 | AGUUUGCCAAGGCACGAG | 510 | ACUCGUGCCUUGGCAAAC | 511 |
| | | UTT | | UTT | |
| 334 | 334-352 | GUUUGCCAAGGCACGAGU | 512 | UACUCGUGCCUUGGCAAA | 513 |
| | | ATT | | CTT | |
| 335 | 335-353 | UUUGCCAAGGCACGAGUA | 514 | UUACUCGUGCCUUGGCAA | 515 |
| | | ATT | | ATT | |
| 336 | 336-354 | UUGCCAAGGCACGAGUAA | 516 | GUUACUCGUGCCUUGGCA | 517 |
| | | CTT | | ATT | |
| 337 | 337-355 | UGCCAAGGCACGAGUAAC | 518 | UGUUACUCGUGCCUUGGC | 519 |
| | | ATT | | ATT | |
| 338 | 338-356 | GCCAAGGCACGAGUAACA | 520 | UUGUUACUCGUGCCUUGG | 521 |
| | | ATT | | CTT | |
| 361 | 361-379 | ACGCAGUUGGGCACUUUU | 522 | CAAAAGUGCCCAACUGCG | 523 |
| | | GTT | | UTT | |
| 362 | 362-380 | CGCAGUUGGGCACUUUUG | 524 | UCAAAAGUGCCCAACUGC | 525 |
| | | ATT | | GTT | |
| 363 | 363-381 | GCAGUUGGGCACUUUUGA | 526 | UUCAAAAGUGCCCAACUG | 527 |
| | | ATT | | CTT | |
| 364 | 364-382 | CAGUUGGGCACUUUUGAA | 528 | CUUCAAAAGUGCCCAACU | 529 |
| | | GTT | | GTT | |
| 365 | 365-383 | AGUUGGGCACUUUUGAAG | 530 | UCUUCAAAAGUGCCCAAC | 531 |
| | | ATT | | UTT | |
| 366 | 366-384 | GUUGGGCACUUUUGAAGA | 532 | AUCUUCAAAAGUGCCCAA | 533 |
| | | UTT | | CTT | |
| 367 | 367-385 | UUGGGCACUUUUGAAGAU | 534 | GAUCUUCAAAAGUGCCCA | 535 |
| | | CTT | | ATT | |
| 368 | 368-386 | UGGGCACUUUUGAAGAUC | 536 | UGAUCUUCAAAAGUGCCC | 537 |
| | | ATT | | ATT | |
| 369 | 369-387 | GGGCACUUUUGAAGAUCA | 538 | AUGAUCUUCAAAAGUGCC | 539 |
| | | UTT | | CTT | |
| 377 | 377-395 | UUGAAGAUCAUUUUCUCA | 540 | CUGAGAAAAUGAUCUUCA | 541 |
| | | GTT | | ATT | |
| 379 | 379-397 | GAAGAUCAUUUUCUCAGC | 542 | GGCUGAGAAAAUGAUCUU | 543 |
| | | CTT | | CTT | |
| 380 | 380-398 | AAGAUCAUUUUCUCAGCC | 544 | AGGCUGAGAAAAUGAUCU | 545 |
| | | UTT | | UTT | |
| 385 | 385-403 | CAUUUUCUCAGCCUCCAG | 546 | UCUGGAGGCUGAGAAAAU | 547 |
| | | ATT | | GTT | |
| 394 | 394-412 | AGCCUCCAGAGGAUGUUC | 548 | UGAACAUCCUCUGGAGGC | 549 |
| | | ATT | | UTT | |
| 396 | 396-414 | CCUCCAGAGGAUGUUCAA | 550 | AUUGAACAUCCUCUGGAG | 551 |
| | | UTT | | GTT | |
| 397 | 397-415 | CUCCAGAGGAUGUUCAAU | 552 | UAUUGAACAUCCUCUGGA | 553 |
| | | ATT | | GTT | |
| 401 | 401-419 | AGAGGAUGUUCAAUAACU | 554 | CAGUUAUUGAACAUCCUC | 555 |
| | | GTT | | UTT | |
| 403 | 403-421 | AGGAUGUUCAAUAACUGU | 556 | CACAGUUAUUGAACAUCC | 557 |
| | | GTT | | UTT | |
| 407 | 407-425 | UGUUCAAUAACUGUGAGG | 558 | ACCUCACAGUUAUUGAAC | 559 |
| | | UTT | | ATT | |
| 409 | 409-427 | UUCAAUAACUGUGAGGUG | 560 | CCACCUCACAGUUAUUGA | 561 |
| | | GTT | | ATT | |
| 410 | 410-428 | UCAAUAACUGUGAGGUGG | 562 | ACCACCUCACAGUUAUUG | 563 |
| | | UTT | | ATT | |
| 411 | 411-429 | CAAUAACUGUGAGGUGGU | 564 | GACCACCUCACAGUUAUU | 565 |
| | | CTT | | GTT | |
| 412 | 412-430 | AAUAACUGUGAGGUGGUC | 566 | GGACCACCUCACAGUUAU | 567 |
| | | CTT | | UTT | |
| 413 | 413-431 | AUAACUGUGAGGUGGUCC | 568 | AGGACCACCUCACAGUUA | 569 |
| | | UTT | | UTT | |
| 414 | 414-432 | UAACUGUGAGGUGGUCCU | 570 | AAGGACCACCUCACAGUU | 571 |
| | | UTT | | ATT | |
| 416 | 416-434 | ACUGUGAGGUGGUCCUUG | 572 | CCAAGGACCACCUCACAG | 573 |
| | | GTT | | UTT | |
| 418 | 418-436 | UGUGAGGUGGUCCUUGGG | 574 | UCCCAAGGACCACCUCAC | 575 |
| | | ATT | | ATT | |
| 419 | 419-437 | GUGAGGUGGUCCUUGGGA | 576 | UUCCCAAGGACCACCUCA | 577 |
| | | ATT | | CTT | |
| 425 | 425-443 | UGGUCCUUGGGAAUUUGG | 578 | UCCAAAUUCCCAAGGACC | 579 |
| | | ATT | | ATT | |
| 431 | 431-449 | UUGGGAAUUUGGAAAUUA | 580 | GUAAUUUCCAAAUUCCCA | 581 |
| | | CTT | | ATT | |
| 432 | 432-450 | UGGGAAUUUGGAAAUUAC | 582 | GGUAAUUUCCAAAUUCCC | 583 |
| | | CTT | | ATT | |
| 433 | 433-451 | GGGAAUUUGGAAAUUACC | 584 | AGGUAAUUUCCAAAUUCC | 585 |
| | | UTT | | CTT | |
| 434 | 434-452 | GGAAUUUGGAAAUUACCU | 586 | UAGGUAAUUUCCAAAUUC | 587 |
| | | ATT | | CTT | |
| 458 | 458-476 | AGAGGAAUUAUGAUCUUU | 588 | GAAAGAUCAUAAUUCCUC | 589 |
| | | CTT | | UTT | |
| 459 | 459-477 | GAGGAAUUAUGAUCUUUC | 590 | GGAAAGAUCAUAAUUCCU | 591 |
| | | CTT | | CTT | |
| 463 | 463-481 | AAUUAUGAUCUUUCCUUC | 592 | AGAAGGAAAGAUCAUAAU | 593 |
| | | UTT | | UTT | |
| 464 | 464-482 | AUUAUGAUCUUUCCUUCU | 594 | AAGAAGGAAAGAUCAUAA | 595 |
| | | UTT | | UTT | |
| 466 | 466-484 | UAUGAUCUUUCCUUCUUA | 596 | UUAAGAAGGAAAGAUCAU | 597 |
| | | ATT | | ATT | |
| 468 | 468-486 | UGAUCUUUCCUUCUUAAA | 598 | CUUUAAGAAGGAAAGAUC | 599 |
| | | GTT | | ATT | |
| 471 | 471-489 | UCUUUCCUUCUUAAAGAC | 600 | GGUCUUUAAGAAGGAAAG | 601 |
| | | CTT | | ATT | |
| 476 | 476-494 | CCUUCUUAAAGACCAUCC | 602 | UGGAUGGUCUUUAAGAAG | 603 |
| | | ATT | | GTT | |
| 477 | 477-495 | CUUCUUAAAGACCAUCCA | 604 | CUGGAUGGUCUUUAAGAA | 605 |
| | | GTT | | GTT | |
| 479 | 479-497 | UCUUAAAGACCAUCCAGG | 606 | UCCUGGAUGGUCUUUAAG | 607 |
| | | ATT | | ATT | |
| 481 | 481-499 | UUAAAGACCAUCCAGGAG | 608 | CCUCCUGGAUGGUCUUUA | 609 |
| | | GTT | | ATT | |
| 482 | 482-500 | UAAAGACCAUCCAGGAGG | 610 | ACCUCCUGGAUGGUCUUU | 611 |
| | | UTT | | ATT | |
| 492 | 492-510 | CCAGGAGGUGGCUGGUUA | 612 | AUAACCAGCCACCUCCUG | 613 |
| | | UTT | | GTT | |
| 493 | 493-511 | CAGGAGGUGGCUGGUUAU | 614 | CAUAACCAGCCACCUCCU | 615 |
| | | GTT | | GTT | |
| 494 | 494-512 | AGGAGGUGGCUGGUUAUG | 616 | ACAUAACCAGCCACCUCC | 617 |
| | | UTT | | UTT | |
| 495 | 495-513 | GGAGGUGGCUGGUUAUGU | 618 | GACAUAACCAGCCACCUC | 619 |
| | | CTT | | CTT | |
| 496 | 496-514 | GAGGUGGCUGGUUAUGUC | 620 | GGACAUAACCAGCCACCU | 621 |
| | | CTT | | CTT | |
| 497 | 497-515 | AGGUGGCUGGUUAUGUCC | 622 | AGGACAUAACCAGCCACC | 623 |
| | | UTT | | UTT | |
| 499 | 499-517 | GUGGCUGGUUAUGUCCUC | 624 | UGAGGACAUAACCAGCCA | 625 |
| | | ATT | | CTT | |
| 520 | 520-538 | GCCCUCAACACAGUGGAG | 626 | GCUCCACUGUGUUGAGGG | 627 |
| | | CTT | | CTT | |
| 542 | 542-560 | UUCCUUUGGAAAACCUGC | 628 | UGCAGGUUUUCCAAAGGA | 629 |
| | | ATT | | ATT | |
| 543 | 543-561 | UCCUUUGGAAAACCUGCA | 630 | CUGCAGGUUUUCCAAAGG | 631 |
| | | GTT | | ATT | |
| 550 | 550-568 | GAAAACCUGCAGAUCAUC | 632 | UGAUGAUCUGCAGGUUUU | 633 |
| | | ATT | | CTT | |
| 551 | 551-569 | AAAACCUGCAGAUCAUCA | 634 | CUGAUGAUCUGCAGGUUU | 635 |
| | | GTT | | UTT | |
| 553 | 553-571 | AACCUGCAGAUCAUCAGA | 636 | CUCUGAUGAUCUGCAGGU | 637 |
| | | GTT | | UTT | |
| 556 | 556-574 | CUGCAGAUCAUCAGAGGA | 638 | UUCCUCUGAUGAUCUGCA | 639 |
| | | ATT | | GTT | |
| 586 | 586-604 | GAAAAUUCCUAUGCCUUA | 640 | CUAAGGCAUAGGAAUUUU | 641 |
| | | GTT | | CTT | |
| 587 | 587-605 | AAAAUUCCUAUGCCUUAG | 642 | GCUAAGGCAUAGGAAUUU | 643 |
| | | CTT | | UTT | |
| 589 | 589-607 | AAUUCCUAUGCCUUAGCA | 644 | CUGCUAAGGCAUAGGAAU | 645 |
| | | GTT | | UTT | |
| 592 | 592-610 | UCCUAUGCCUUAGCAGUC | 646 | AGACUGCUAAGGCAUAGG | 647 |
| | | UTT | | ATT | |
| 593 | 593-611 | CCUAUGCCUUAGCAGUCU | 648 | AAGACUGCUAAGGCAUAG | 649 |
| | | UTT | | GTT | |
| 594 | 594-612 | CUAUGCCUUAGCAGUCUU | 650 | UAAGACUGCUAAGGCAUA | 651 |
| | | ATT | | GTT | |
| 596 | 596-614 | AUGCCUUAGCAGUCUUAU | 652 | GAUAAGACUGCUAAGGCA | 653 |
| | | CTT | | UTT | |
| 597 | 597-615 | UGCCUUAGCAGUCUUAUC | 654 | AGAUAAGACUGCUAAGGC | 655 |
| | | UTT | | ATT | |
| 598 | 598-616 | GCCUUAGCAGUCUUAUCU | 656 | UAGAUAAGACUGCUAAGG | 657 |
| | | ATT | | CTT | |
| 599 | 599-617 | CCUUAGCAGUCUUAUCUA | 658 | UUAGAUAAGACUGCUAAG | 659 |
| | | ATT | | GTT | |
| 600 | 600-618 | CUUAGCAGUCUUAUCUAA | 660 | GUUAGAUAAGACUGCUAA | 661 |
| | | CTT | | GTT | |
| 601 | 601-619 | UUAGCAGUCUUAUCUAAC | 662 | AGUUAGAUAAGACUGCUA | 663 |
| | | UTT | | ATT | |
| 602 | 602-620 | UAGCAGUCUUAUCUAACU | 664 | UAGUUAGAUAAGACUGCU | 665 |
| | | ATT | | ATT | |
| 603 | 603-621 | AGCAGUCUUAUCUAACUA | 666 | AUAGUUAGAUAAGACUGC | 667 |
| | | UTT | | UTT | |
| 604 | 604-622 | GCAGUCUUAUCUAACUAU | 668 | CAUAGUUAGAUAAGACUG | 669 |
| | | GTT | | CTT | |
| 605 | 605-623 | CAGUCUUAUCUAACUAUG | 670 | UCAUAGUUAGAUAAGACU | 671 |
| | | ATT | | GTT | |
| 608 | 608-626 | UCUUAUCUAACUAUGAUG | 672 | GCAUCAUAGUUAGAUAAG | 673 |
| | | CTT | | ATT | |
| 609 | 609-627 | CUUAUCUAACUAUGAUGC | 674 | UGCAUCAUAGUUAGAUAA | 675 |
| | | ATT | | GTT | |
| 610 | 610-628 | UUAUCUAACUAUGAUGCA | 676 | UUGCAUCAUAGUUAGAUA | 677 |
| | | ATT | | ATT | |
| 611 | 611-629 | UAUCUAACUAUGAUGCAA | 678 | UUUGCAUCAUAGUUAGAU | 679 |
| | | ATT | | ATT | |
| 612 | 612-630 | AUCUAACUAUGAUGCAAA | 680 | AUUUGCAUCAUAGUUAGA | 681 |
| | | UTT | | UTT | |
| 613 | 613-631 | UCUAACUAUGAUGCAAAU | 682 | UAUUUGCAUCAUAGUUAG | 683 |
| | | ATT | | ATT | |
| 614 | 614-632 | CUAACUAUGAUGCAAAUA | 684 | UUAUUUGCAUCAUAGUUA | 685 |
| | | ATT | | GTT | |
| 616 | 616-634 | AACUAUGAUGCAAAUAAA | 686 | UUUUAUUUGCAUCAUAGU | 687 |
| | | ATT | | UTT | |
| 622 | 622-640 | GAUGCAAAUAAAACCGGA | 688 | GUCCGGUUUUAUUUGCAU | 689 |
| | | CTT | | CTT | |
| 623 | 623-641 | AUGCAAAUAAAACCGGAC | 690 | AGUCCGGUUUUAUUUGCA | 691 |
| | | UTT | | UTT | |
| 624 | 624-642 | UGCAAAUAAAACCGGACU | 692 | CAGUCCGGUUUUAUUUGC | 693 |
| | | GTT | | ATT | |
| 626 | 626-644 | CAAAUAAAACCGGACUGA | 694 | UUCAGUCCGGUUUUAUUU | 695 |
| | | ATT | | GTT | |
| 627 | 627-645 | AAAUAAAACCGGACUGAA | 696 | CUUCAGUCCGGUUUUAUU | 697 |
| | | GTT | | UTT | |
| 628 | 628-646 | AAUAAAACCGGACUGAAG | 698 | CCUUCAGUCCGGUUUUAU | 699 |
| | | GTT | | UTT | |
| 630 | 630-648 | UAAAACCGGACUGAAGGA | 700 | CUCCUUCAGUCCGGUUUU | 701 |
| | | GTT | | ATT | |
| 631 | 631-649 | AAAACCGGACUGAAGGAG | 702 | GCUCCUUCAGUCCGGUUU | 703 |
| | | CTT | | UTT | |
| 632 | 632-650 | AAACCGGACUGAAGGAGC | 704 | AGCUCCUUCAGUCCGGUU | 705 |
| | | UTT | | UTT | |
| 633 | 633-651 | AACCGGACUGAAGGAGCU | 706 | CAGCUCCUUCAGUCCGGU | 707 |
| | | GTT | | UTT | |
| 644 | 644-662 | AGGAGCUGCCCAUGAGAA | 708 | UUUCUCAUGGGCAGCUCC | 709 |
| | | ATT | | UTT | |
| 665 | 665-683 | UACAGGAAAUCCUGCAUG | 710 | CCAUGCAGGAUUUCCUGU | 711 |
| | | GTT | | ATT | |
| 668 | 668-686 | AGGAAAUCCUGCAUGGCG | 712 | GCGCCAUGCAGGAUUUCC | 713 |
| | | CTT | | UTT | |
| 669 | 669-687 | GGAAAUCCUGCAUGGCGC | 714 | GGCGCCAUGCAGGAUUUC | 715 |
| | | CTT | | CTT | |
| 670 | 670-688 | GAAAUCCUGCAUGGCGCC | 716 | CGGCGCCAUGCAGGAUUU | 717 |
| | | GTT | | CTT | |
| 671 | 671-689 | AAAUCCUGCAUGGCGCCG | 718 | ACGGCGCCAUGCAGGAUU | 719 |
| | | UTT | | UTT | |
| 672 | 672-690 | AAUCCUGCAUGGCGCCGU | 720 | CACGGCGCCAUGCAGGAU | 721 |
| | | GTT | | UTT | |
| 674 | 674-692 | UCCUGCAUGGCGCCGUGC | 722 | CGCACGGCGCCAUGCAGG | 723 |
| | | GTT | | ATT | |
| 676 | 676-694 | CUGCAUGGCGCCGUGCGG | 724 | ACCGCACGGCGCCAUGCA | 725 |
| | | UTT | | GTT | |
| 677 | 677-695 | UGCAUGGCGCCGUGCGGU | 726 | AACCGCACGGCGCCAUGC | 727 |
| | | UTT | | ATT | |
| 678 | 678-696 | GCAUGGCGCCGUGCGGUU | 728 | GAACCGCACGGCGCCAUG | 729 |
| | | CTT | | CTT | |
| 680 | 680-698 | AUGGCGCCGUGCGGUUCA | 730 | CUGAACCGCACGGCGCCA | 731 |
| | | GTT | | UTT | |
| 681 | 681-699 | UGGCGCCGUGCGGUUCAG | 732 | GCUGAACCGCACGGCGCC | 733 |
| | | CTT | | ATT | |
| 682 | 682-700 | GGCGCCGUGCGGUUCAGC | 734 | UGCUGAACCGCACGGCGC | 735 |
| | | ATT | | CTT | |
| 683 | 683-701 | GCGCCGUGCGGUUCAGCA | 736 | UUGCUGAACCGCACGGCG | 737 |
| | | ATT | | CTT | |
| 684 | 684-702 | CGCCGUGCGGUUCAGCAA | 738 | GUUGCUGAACCGCACGGC | 739 |
| | | CTT | | GTT | |
| 685 | 685-703 | GCCGUGCGGUUCAGCAAC | 740 | UGUUGCUGAACCGCACGG | 741 |
| | | ATT | | CTT | |
| 686 | 686-704 | CCGUGCGGUUCAGCAACA | 742 | UUGUUGCUGAACCGCACG | 743 |
| | | ATT | | GTT | |
| 688 | 688-706 | GUGCGGUUCAGCAACAAC | 744 | GGUUGUUGCUGAACCGCA | 745 |
| | | CTT | | CTT | |
| 690 | 690-708 | GCGGUUCAGCAACAACCC | 746 | AGGGUUGUUGCUGAACCG | 747 |
| | | UTT | | CTT | |
| 692 | 692-710 | GGUUCAGCAACAACCCUG | 748 | GCAGGGUUGUUGCUGAAC | 749 |
| | | CTT | | CTT | |
| 698 | 698-716 | GCAACAACCCUGCCCUGU | 750 | CACAGGGCAGGGUUGUUG | 751 |
| | | GTT | | CTT | |
| 700 | 700-718 | AACAACCCUGCCCUGUGC | 752 | UGCACAGGGCAGGGUUGU | 753 |
| | | ATT | | UTT | |
| 719 | 719-737 | ACGUGGAGAGCAUCCAGU | 754 | CACUGGAUGCUCUCCACG | 755 |
| | | GTT | | UTT | |
| 720 | 720-738 | CGUGGAGAGCAUCCAGUG | 756 | CCACUGGAUGCUCUCCAC | 757 |
| | | GTT | | GTT | |
| 721 | 721-739 | GUGGAGAGCAUCCAGUGG | 758 | GCCACUGGAUGCUCUCCA | 759 |
| | | CTT | | CTT | |
| 724 | 724-742 | GAGAGCAUCCAGUGGCGG | 760 | CCCGCCACUGGAUGCUCU | 761 |
| | | GTT | | CTT | |
| 725 | 725-743 | AGAGCAUCCAGUGGCGGG | 762 | UCCCGCCACUGGAUGCUC | 763 |
| | | ATT | | UTT | |
| 726 | 726-744 | GAGCAUCCAGUGGCGGGA | 764 | GUCCCGCCACUGGAUGCU | 765 |
| | | CTT | | CTT | |
| 733 | 733-751 | CAGUGGCGGGACAUAGUC | 766 | UGACUAUGUCCCGCCACU | 767 |
| | | ATT | | GTT | |
| 734 | 734-752 | AGUGGCGGGACAUAGUCA | 768 | CUGACUAUGUCCCGCCAC | 769 |
| | | GTT | | UTT | |
| 736 | 736-754 | UGGCGGGACAUAGUCAGC | 770 | UGCUGACUAUGUCCCGCC | 771 |
| | | ATT | | ATT | |
| 737 | 737-755 | GGCGGGACAUAGUCAGCA | 772 | CUGCUGACUAUGUCCCGC | 773 |
| | | GTT | | CTT | |
| 763 | 763-781 | CUCAGCAACAUGUCGAUG | 774 | CCAUCGACAUGUUGCUGA | 775 |
| | | GTT | | GTT | |
| 765 | 765-783 | CAGCAACAUGUCGAUGGA | 776 | GUCCAUCGACAUGUUGCU | 777 |
| | | CTT | | GTT | |
| 766 | 766-784 | AGCAACAUGUCGAUGGAC | 778 | AGUCCAUCGACAUGUUGC | 779 |
| | | UTT | | UTT | |
| 767 | 767-785 | GCAACAUGUCGAUGGACU | 780 | AAGUCCAUCGACAUGUUG | 781 |
| | | UTT | | CTT | |
| 769 | 769-787 | AACAUGUCGAUGGACUUC | 782 | GGAAGUCCAUCGACAUGU | 783 |
| | | CTT | | UTT | |
| 770 | 770-788 | ACAUGUCGAUGGACUUCC | 784 | UGGAAGUCCAUCGACAUG | 785 |
| | | ATT | | UTT | |
| 771 | 771-789 | CAUGUCGAUGGACUUCCA | 786 | CUGGAAGUCCAUCGACAU | 787 |
| | | GTT | | GTT | |
| 772 | 772-790 | AUGUCGAUGGACUUCCAG | 788 | UCUGGAAGUCCAUCGACA | 789 |
| | | ATT | | UTT | |
| 775 | 775-793 | UCGAUGGACUUCCAGAAC | 790 | GGUUCUGGAAGUCCAUCG | 791 |
| | | CTT | | ATT | |
| 789 | 789-807 | GAACCACCUGGGCAGCUG | 792 | GCAGCUGCCCAGGUGGUU | 793 |
| | | CTT | | CTT | |
| 798 | 798-816 | GGGCAGCUGCCAAAAGUG | 794 | ACACUUUUGGCAGCUGCC | 795 |
| | | UTT | | CTT | |
| 800 | 800-818 | GCAGCUGCCAAAAGUGUG | 796 | UCACACUUUUGGCAGCUG | 797 |
| | | ATT | | CTT | |
| 805 | 805-823 | UGCCAAAAGUGUGAUCCA | 798 | UUGGAUCACACUUUUGGC | 799 |
| | | ATT | | ATT | |
| 806 | 806-824 | GCCAAAAGUGUGAUCCAA | 800 | CUUGGAUCACACUUUUGG | 801 |
| | | GTT | | CTT | |
| 807 | 807-825 | CCAAAAGUGUGAUCCAAG | 802 | GCUUGGAUCACACUUUUG | 803 |
| | | CTT | | GTT | |
| 810 | 810-828 | AAAGUGUGAUCCAAGCUG | 804 | ACAGCUUGGAUCACACUU | 805 |
| | | UTT | | UTT | |
| 814 | 814-832 | UGUGAUCCAAGCUGUCCC | 806 | UGGGACAGCUUGGAUCAC | 807 |
| | | ATT | | ATT | |
| 815 | 815-833 | GUGAUCCAAGCUGUCCCA | 808 | UUGGGACAGCUUGGAUCA | 809 |
| | | ATT | | CTT | |
| 817 | 817-835 | GAUCCAAGCUGUCCCAAU | 810 | CAUUGGGACAGCUUGGAU | 811 |
| | | GTT | | CTT | |
| 818 | 818-836 | AUCCAAGCUGUCCCAAUG | 812 | CCAUUGGGACAGCUUGGA | 813 |
| | | GTT | | UTT | |
| 819 | 819-837 | UCCAAGCUGUCCCAAUGG | 814 | CCCAUUGGGACAGCUUGG | 815 |
| | | GTT | | ATT | |
| 820 | 820-838 | CCAAGCUGUCCCAAUGGG | 816 | UCCCAUUGGGACAGCUUG | 817 |
| | | ATT | | GTT | |
| 821 | 821-839 | CAAGCUGUCCCAAUGGGA | 818 | CUCCCAUUGGGACAGCUU | 819 |
| | | GTT | | GTT | |
| 823 | 823-841 | AGCUGUCCCAAUGGGAGC | 820 | AGCUCCCAUUGGGACAGC | 821 |
| | | UTT | | UTT | |
| 826 | 826-844 | UGUCCCAAUGGGAGCUGC | 822 | AGCAGCUCCCAUUGGGAC | 823 |
| | | UTT | | ATT | |
| 847 | 847-865 | GGUGCAGGAGAGGAGAAC | 824 | AGUUCUCCUCUCCUGCAC | 825 |
| | | UTT | | CTT | |
| 871 | 871-889 | AAACUGACCAAAAUCAUC | 826 | AGAUGAUUUUGGUCAGUU | 827 |
| | | UTT | | UTT | |
| 872 | 872-890 | AACUGACCAAAAUCAUCU | 828 | CAGAUGAUUUUGGUCAGU | 829 |
| | | GTT | | UTT | |
| 873 | 873-891 | ACUGACCAAAAUCAUCUG | 830 | ACAGAUGAUUUUGGUCAG | 831 |
| | | UTT | | UTT | |
| 877 | 877-895 | ACCAAAAUCAUCUGUGCC | 832 | GGGCACAGAUGAUUUUGG | 833 |
| | | CTT | | UTT | |
| 878 | 878-896 | CCAAAAUCAUCUGUGCCC | 834 | UGGGCACAGAUGAUUUUG | 835 |
| | | ATT | | GTT | |
| 881 | 881-899 | AAAUCAUCUGUGCCCAGC | 836 | UGCUGGGCACAGAUGAUU | 837 |
| | | ATT | | UTT | |
| 890 | 890-908 | GUGCCCAGCAGUGCUCCG | 838 | CCGGAGCACUGCUGGGCA | 839 |
| | | GTT | | CTT | |
| 892 | 892-910 | GCCCAGCAGUGCUCCGGG | 840 | GCCCGGAGCACUGCUGGG | 841 |
| | | CTT | | CTT | |
| 929 | 929-947 | CCAGUGACUGCUGCCACA | 842 | UUGUGGCAGCAGUCACUG | 843 |
| | | ATT | | GTT | |
| 930 | 930-948 | CAGUGACUGCUGCCACAA | 844 | GUUGUGGCAGCAGUCACU | 845 |
| | | CTT | | GTT | |
| 979 | 979-997 | GAGAGCGACUGCCUGGUC | 846 | AGACCAGGCAGUCGCUCU | 847 |
| | | UTT | | CTT | |
| 980 | 980-998 | AGAGCGACUGCCUGGUCU | 848 | CAGACCAGGCAGUCGCUC | 849 |
| | | GTT | | UTT | |
| 981 | 981-999 | GAGCGACUGCCUGGUCUG | 850 | GCAGACCAGGCAGUCGCU | 851 |
| | | CTT | | CTT | |
| 982 | 982-1000 | AGCGACUGCCUGGUCUGC | 852 | GGCAGACCAGGCAGUCGC | 853 |
| | | CTT | | UTT | |
| 983 | 983-1001 | GCGACUGCCUGGUCUGCC | 854 | CGGCAGACCAGGCAGUCG | 855 |
| | | GTT | | CTT | |
| 984 | 984-1002 | CGACUGCCUGGUCUGCCG | 856 | GCGGCAGACCAGGCAGUC | 857 |
| | | CTT | | GTT | |
| 989 | 989-1007 | GCCUGGUCUGCCGCAAAU | 858 | AAUUUGCGGCAGACCAGG | 859 |
| | | UTT | | CTT | |
| 990 | 990-1008 | CCUGGUCUGCCGCAAAUU | 860 | GAAUUUGCGGCAGACCAG | 861 |
| | | CTT | | GTT | |
| 991 | 991-1009 | CUGGUCUGCCGCAAAUUC | 862 | GGAAUUUGCGGCAGACCA | 863 |
| | | CTT | | GTT | |
| 992 | 992-1010 | UGGUCUGCCGCAAAUUCC | 864 | CGGAAUUUGCGGCAGACC | 865 |
| | | GTT | | ATT | |
| 994 | 994-1012 | GUCUGCCGCAAAUUCCGA | 866 | CUCGGAAUUUGCGGCAGA | 867 |
| | | GTT | | CTT | |
| 995 | 995-1013 | UCUGCCGCAAAUUCCGAG | 868 | UCUCGGAAUUUGCGGCAG | 869 |
| | | ATT | | ATT | |
| 996 | 996-1014 | CUGCCGCAAAUUCCGAGA | 870 | GUCUCGGAAUUUGCGGCA | 871 |
| | | CTT | | GTT | |
| 997 | 997-1015 | UGCCGCAAAUUCCGAGAC | 872 | CGUCUCGGAAUUUGCGGC | 873 |
| | | GTT | | ATT | |
| 999 | 999-1017 | CCGCAAAUUCCGAGACGA | 874 | UUCGUCUCGGAAUUUGCG | 875 |
| | | ATT | | GTT | |
| 1004 | 1004-1022 | AAUUCCGAGACGAAGCCA | 876 | GUGGCUUCGUCUCGGAAU | 877 |
| | | CTT | | UTT | |
| 1005 | 1005-1023 | AUUCCGAGACGAAGCCAC | 878 | CGUGGCUUCGUCUCGGAA | 879 |
| | | GTT | | UTT | |
| 1006 | 1006-1024 | UUCCGAGACGAAGCCACG | 880 | ACGUGGCUUCGUCUCGGA | 881 |
| | | UTT | | ATT | |
| 1007 | 1007-1025 | UCCGAGACGAAGCCACGU | 882 | CACGUGGCUUCGUCUCGG | 883 |
| | | GTT | | ATT | |
| 1008 | 1008-1026 | CCGAGACGAAGCCACGUG | 884 | GCACGUGGCUUCGUCUCG | 885 |
| | | CTT | | GTT | |
| 1010 | 1010-1028 | GAGACGAAGCCACGUGCA | 886 | UUGCACGUGGCUUCGUCU | 887 |
| | | ATT | | CTT | |
| 1013 | 1013-1031 | ACGAAGCCACGUGCAAGG | 888 | UCCUUGCACGUGGCUUCG | 889 |
| | | ATT | | UTT | |
| 1014 | 1014-1032 | CGAAGCCACGUGCAAGGA | 890 | GUCCUUGCACGUGGCUUC | 891 |
| | | CTT | | GTT | |
| 1015 | 1015-1033 | GAAGCCACGUGCAAGGAC | 892 | UGUCCUUGCACGUGGCUU | 893 |
| | | ATT | | CTT | |
| 1016 | 1016-1034 | AAGCCACGUGCAAGGACA | 894 | GUGUCCUUGCACGUGGCU | 895 |
| | | CTT | | UTT | |
| 1040 | 1040-1058 | CCCCACUCAUGCUCUACA | 896 | UUGUAGAGCAUGAGUGGG | 897 |
| | | ATT | | GTT | |
| 1042 | 1042-1060 | CCACUCAUGCUCUACAAC | 898 | GGUUGUAGAGCAUGAGUG | 899 |
| | | CTT | | GTT | |
| 1044 | 1044-1062 | ACUCAUGCUCUACAACCC | 900 | GGGGUUGUAGAGCAUGAG | 901 |
| | | CTT | | UTT | |
| 1047 | 1047-1065 | CAUGCUCUACAACCCCAC | 902 | GGUGGGGUUGUAGAGCAU | 903 |
| | | CTT | | GTT | |
| 1071 | 1071-1089 | CCAGAUGGAUGUGAACCC | 904 | GGGGUUCACAUCCAUCUG | 905 |
| | | CTT | | GTT | |
| 1073 | 1073-1091 | AGAUGGAUGUGAACCCCG | 906 | UCGGGGUUCACAUCCAUC | 907 |
| | | ATT | | UTT | |
| 1074 | 1074-1092 | GAUGGAUGUGAACCCCGA | 908 | CUCGGGGUUCACAUCCAU | 909 |
| | | GTT | | CTT | |
| 1075 | 1075-1093 | AUGGAUGUGAACCCCGAG | 910 | CCUCGGGGUUCACAUCCA | 911 |
| | | GTT | | UTT | |
| 1077 | 1077-1095 | GGAUGUGAACCCCGAGGG | 912 | GCCCUCGGGGUUCACAUC | 913 |
| | | CTT | | CTT | |
| 1078 | 1078-1096 | GAUGUGAACCCCGAGGGC | 914 | UGCCCUCGGGGUUCACAU | 915 |
| | | ATT | | CTT | |
| 1080 | 1080-1098 | UGUGAACCCCGAGGGCAA | 916 | UUUGCCCUCGGGGUUCAC | 917 |
| | | ATT | | ATT | |
| 1084 | 1084-1102 | AACCCCGAGGGCAAAUAC | 918 | UGUAUUUGCCCUCGGGGU | 919 |
| | | ATT | | UTT | |
| 1085 | 1085-1103 | ACCCCGAGGGCAAAUACA | 920 | CUGUAUUUGCCCUCGGGG | 921 |
| | | GTT | | UTT | |
| 1087 | 1087-1105 | CCCGAGGGCAAAUACAGC | 922 | AGCUGUAUUUGCCCUCGG | 923 |
| | | UTT | | GTT | |
| 1088 | 1088-1106 | CCGAGGGCAAAUACAGCU | 924 | AAGCUGUAUUUGCCCUCG | 925 |
| | | UTT | | GTT | |
| 1089 | 1089-1107 | CGAGGGCAAAUACAGCUU | 926 | AAAGCUGUAUUUGCCCUC | 927 |
| | | UTT | | GTT | |
| 1096 | 1096-1114 | AAAUACAGCUUUGGUGCC | 928 | UGGCACCAAAGCUGUAUU | 929 |
| | | ATT | | UTT | |
| 1097 | 1097-1115 | AAUACAGCUUUGGUGCCA | 930 | GUGGCACCAAAGCUGUAU | 931 |
| | | CTT | | UTT | |
| 1098 | 1098-1116 | AUACAGCUUUGGUGCCAC | 932 | GGUGGCACCAAAGCUGUA | 933 |
| | | CTT | | UTT | |
| 1104 | 1104-1122 | CUUUGGUGCCACCUGCGU | 934 | CACGCAGGUGGCACCAAA | 935 |
| | | GTT | | GTT | |
| 1106 | 1106-1124 | UUGGUGCCACCUGCGUGA | 936 | UUCACGCAGGUGGCACCA | 937 |
| | | ATT | | ATT | |
| 1112 | 1112-1130 | CCACCUGCGUGAAGAAGU | 938 | CACUUCUUCACGCAGGUG | 939 |
| | | GTT | | GTT | |
| 1116 | 1116-1134 | CUGCGUGAAGAAGUGUCC | 940 | GGGACACUUCUUCACGCA | 941 |
| | | CTT | | GTT | |
| 1117 | 1117-1135 | UGCGUGAAGAAGUGUCCC | 942 | GGGGACACUUCUUCACGC | 943 |
| | | CTT | | ATT | |
| 1118 | 1118-1136 | GCGUGAAGAAGUGUCCCC | 944 | CGGGGACACUUCUUCACG | 945 |
| | | GTT | | CTT | |
| 1119 | 1119-1137 | CGUGAAGAAGUGUCCCCG | 946 | ACGGGGACACUUCUUCAC | 947 |
| | | UTT | | GTT | |
| 1120 | 1120-1138 | GUGAAGAAGUGUCCCCGU | 948 | UACGGGGACACUUCUUCA | 949 |
| | | ATT | | CTT | |
| 1121 | 1121-1139 | UGAAGAAGUGUCCCCGUA | 950 | UUACGGGGACACUUCUUC | 951 |
| | | ATT | | ATT | |
| 1122 | 1122-1140 | GAAGAAGUGUCCCCGUAA | 952 | AUUACGGGGACACUUCUU | 953 |
| | | UTT | | CTT | |
| 1123 | 1123-1141 | AAGAAGUGUCCCCGUAAU | 954 | AAUUACGGGGACACUUCU | 955 |
| | | UTT | | UTT | |
| 1124 | 1124-1142 | AGAAGUGUCCCCGUAAUU | 956 | UAAUUACGGGGACACUUC | 957 |
| | | ATT | | UTT | |
| 1125 | 1125-1143 | GAAGUGUCCCCGUAAUUA | 958 | AUAAUUACGGGGACACUU | 959 |
| | | UTT | | CTT | |
| 1126 | 1126-1144 | AAGUGUCCCCGUAAUUAU | 960 | CAUAAUUACGGGGACACU | 961 |
| | | GTT | | UTT | |
| 1127 | 1127-1145 | AGUGUCCCCGUAAUUAUG | 962 | ACAUAAUUACGGGGACAC | 963 |
| | | UTT | | UTT | |
| 1128 | 1128-1146 | GUGUCCCCGUAAUUAUGU | 964 | CACAUAAUUACGGGGACA | 965 |
| | | GTT | | CTT | |
| 1129 | 1129-1147 | UGUCCCCGUAAUUAUGUG | 966 | CCACAUAAUUACGGGGAC | 967 |
| | | GTT | | ATT | |
| 1130 | 1130-1148 | GUCCCCGUAAUUAUGUGG | 968 | ACCACAUAAUUACGGGGA | 969 |
| | | UTT | | CTT | |
| 1132 | 1132-1150 | CCCCGUAAUUAUGUGGUG | 970 | UCACCACAUAAUUACGGG | 971 |
| | | ATT | | GTT | |
| 1134 | 1134-1152 | CCGUAAUUAUGUGGUGAC | 972 | UGUCACCACAUAAUUACG | 973 |
| | | ATT | | GTT | |
| 1136 | 1136-1154 | GUAAUUAUGUGGUGACAG | 974 | UCUGUCACCACAUAAUUA | 975 |
| | | ATT | | CTT | |
| 1137 | 1137-1155 | UAAUUAUGUGGUGACAGA | 976 | AUCUGUCACCACAUAAUU | 977 |
| | | UTT | | ATT | |
| 1138 | 1138-1156 | AAUUAUGUGGUGACAGAU | 978 | GAUCUGUCACCACAUAAU | 979 |
| | | CTT | | UTT | |
| 1139 | 1139-1157 | AUUAUGUGGUGACAGAUC | 980 | UGAUCUGUCACCACAUAA | 981 |
| | | ATT | | UTT | |
| 1140 | 1140-1158 | UUAUGUGGUGACAGAUCA | 982 | GUGAUCUGUCACCACAUA | 983 |
| | | CTT | | ATT | |
| 1142 | 1142-1160 | AUGUGGUGACAGAUCACG | 984 | CCGUGAUCUGUCACCACA | 985 |
| | | GTT | | UTT | |
| 1145 | 1145-1163 | UGGUGACAGAUCACGGCU | 986 | GAGCCGUGAUCUGUCACC | 987 |
| | | CTT | | ATT | |
| 1147 | 1147-1165 | GUGACAGAUCACGGCUCG | 988 | ACGAGCCGUGAUCUGUCA | 989 |
| | | UTT | | CTT | |
| 1148 | 1148-1166 | UGACAGAUCACGGCUCGU | 990 | CACGAGCCGUGAUCUGUC | 991 |
| | | GTT | | ATT | |
| 1149 | 1149-1167 | GACAGAUCACGGCUCGUG | 992 | GCACGAGCCGUGAUCUGU | 993 |
| | | CTT | | CTT | |
| 1150 | 1150-1168 | ACAGAUCACGGCUCGUGC | 994 | CGCACGAGCCGUGAUCUG | 995 |
| | | GTT | | UTT | |
| 1151 | 1151-1169 | CAGAUCACGGCUCGUGCG | 996 | ACGCACGAGCCGUGAUCU | 997 |
| | | UTT | | GTT | |
| 1152 | 1152-1170 | AGAUCACGGCUCGUGCGU | 998 | GACGCACGAGCCGUGAUC | 999 |
| | | CTT | | UTT | |
| 1153 | 1153-1171 | GAUCACGGCUCGUGCGUC | 1000 | GGACGCACGAGCCGUGAU | 1001 |
| | | CTT | | CTT | |
| 1154 | 1154-1172 | AUCACGGCUCGUGCGUCC | 1002 | CGGACGCACGAGCCGUGA | 1003 |
| | | GTT | | UTT | |
| 1155 | 1155-1173 | UCACGGCUCGUGCGUCCG | 1004 | UCGGACGCACGAGCCGUG | 1005 |
| | | ATT | | ATT | |
| 1156 | 1156-1174 | CACGGCUCGUGCGUCCGA | 1006 | CUCGGACGCACGAGCCGU | 1007 |
| | | GTT | | GTT | |
| 1157 | 1157-1175 | ACGGCUCGUGCGUCCGAG | 1008 | GCUCGGACGCACGAGCCG | 1009 |
| | | CTT | | UTT | |
| 1160 | 1160-1178 | GCUCGUGCGUCCGAGCCU | 1010 | CAGGCUCGGACGCACGAG | 1011 |
| | | GTT | | CTT | |
| 1200 | 1200-1218 | GGAGGAAGACGGCGUCCG | 1012 | GCGGACGCCGUCUUCCUC | 1013 |
| | | CTT | | CTT | |
| 1201 | 1201-1219 | GAGGAAGACGGCGUCCGC | 1014 | UGCGGACGCCGUCUUCCU | 1015 |
| | | ATT | | CTT | |
| 1203 | 1203-1221 | GGAAGACGGCGUCCGCAA | 1016 | CUUGCGGACGCCGUCUUC | 1017 |
| | | GTT | | CTT | |
| 1204 | 1204-1222 | GAAGACGGCGUCCGCAAG | 1018 | ACUUGCGGACGCCGUCUU | 1019 |
| | | UTT | | CTT | |
| 1205 | 1205-1223 | AAGACGGCGUCCGCAAGU | 1020 | CACUUGCGGACGCCGUCU | 1021 |
| | | GTT | | UTT | |
| 1207 | 1207-1225 | GACGGCGUCCGCAAGUGU | 1022 | UACACUUGCGGACGCCGU | 1023 |
| | | ATT | | CTT | |
| 1208 | 1208-1226 | ACGGCGUCCGCAAGUGUA | 1024 | UUACACUUGCGGACGCCG | 1025 |
| | | ATT | | UTT | |
| 1211 | 1211-1229 | GCGUCCGCAAGUGUAAGA | 1026 | UUCUUACACUUGCGGACG | 1027 |
| | | ATT | | CTT | |
| 1212 | 1212-1230 | CGUCCGCAAGUGUAAGAA | 1028 | CUUCUUACACUUGCGGAC | 1029 |
| | | GTT | | GTT | |
| 1213 | 1213-1231 | GUCCGCAAGUGUAAGAAG | 1030 | ACUUCUUACACUUGCGGA | 1031 |
| | | UTT | | CTT | |
| 1214 | 1214-1232 | UCCGCAAGUGUAAGAAGU | 1032 | CACUUCUUACACUUGCGG | 1033 |
| | | GTT | | ATT | |
| 1215 | 1215-1233 | CCGCAAGUGUAAGAAGUG | 1034 | GCACUUCUUACACUUGCG | 1035 |
| | | CTT | | GTT | |
| 1216 | 1216-1234 | CGCAAGUGUAAGAAGUGC | 1036 | CGCACUUCUUACACUUGC | 1037 |
| | | GTT | | GTT | |
| 1217 | 1217-1235 | GCAAGUGUAAGAAGUGCG | 1038 | UCGCACUUCUUACACUUG | 1039 |
| | | ATT | | CTT | |
| 1219 | 1219-1237 | AAGUGUAAGAAGUGCGAA | 1040 | CUUCGCACUUCUUACACU | 1041 |
| | | GTT | | UTT | |
| 1220 | 1220-1238 | AGUGUAAGAAGUGCGAAG | 1042 | CCUUCGCACUUCUUACAC | 1043 |
| | | GTT | | UTT | |
| 1221 | 1221-1239 | GUGUAAGAAGUGCGAAGG | 1044 | CCCUUCGCACUUCUUACA | 1045 |
| | | GTT | | CTT | |
| 1222 | 1222-1240 | UGUAAGAAGUGCGAAGGG | 1046 | GCCCUUCGCACUUCUUAC | 1047 |
| | | CTT | | ATT | |
| 1223 | 1223-1241 | GUAAGAAGUGCGAAGGGC | 1048 | GGCCCUUCGCACUUCUUA | 1049 |
| | | CTT | | CTT | |
| 1224 | 1224-1242 | UAAGAAGUGCGAAGGGCC | 1050 | AGGCCCUUCGCACUUCUU | 1051 |
| | | UTT | | ATT | |
| 1225 | 1225-1243 | AAGAAGUGCGAAGGGCCU | 1052 | AAGGCCCUUCGCACUUCU | 1053 |
| | | UTT | | UTT | |
| 1226 | 1226-1244 | AGAAGUGCGAAGGGCCUU | 1054 | CAAGGCCCUUCGCACUUC | 1055 |
| | | GTT | | UTT | |
| 1229 | 1229-1247 | AGUGCGAAGGGCCUUGCC | 1056 | CGGCAAGGCCCUUCGCAC | 1057 |
| | | GTT | | UTT | |
| 1230 | 1230-1248 | GUGCGAAGGGCCUUGCCG | 1058 | GCGGCAAGGCCCUUCGCA | 1059 |
| | | CTT | | CTT | |
| 1231 | 1231-1249 | UGCGAAGGGCCUUGCCGC | 1060 | UGCGGCAAGGCCCUUCGC | 1061 |
| | | ATT | | ATT | |
| 1232 | 1232-1250 | GCGAAGGGCCUUGCCGCA | 1062 | UUGCGGCAAGGCCCUUCG | 1063 |
| | | ATT | | CTT | |
| 1233 | 1233-1251 | CGAAGGGCCUUGCCGCAA | 1064 | UUUGCGGCAAGGCCCUUC | 1065 |
| | | ATT | | GTT | |
| 1235 | 1235-1253 | AAGGGCCUUGCCGCAAAG | 1066 | ACUUUGCGGCAAGGCCCU | 1067 |
| | | UTT | | UTT | |
| 1236 | 1236-1254 | AGGGCCUUGCCGCAAAGU | 1068 | CACUUUGCGGCAAGGCCC | 1069 |
| | | GTT | | UTT | |
| 1237 | 1237-1255 | GGGCCUUGCCGCAAAGUG | 1070 | ACACUUUGCGGCAAGGCC | 1071 |
| | | UTT | | CTT | |
| 1238 | 1238-1256 | GGCCUUGCCGCAAAGUGU | 1072 | CACACUUUGCGGCAAGGC | 1073 |
| | | GTT | | CTT | |
| 1239 | 1239-1257 | GCCUUGCCGCAAAGUGUG | 1074 | ACACACUUUGCGGCAAGG | 1075 |
| | | UTT | | CTT | |
| 1241 | 1241-1259 | CUUGCCGCAAAGUGUGUA | 1076 | UUACACACUUUGCGGCAA | 1077 |
| | | ATT | | GTT | |
| 1261 | 1261-1279 | GGAAUAGGUAUUGGUGAA | 1078 | AUUCACCAAUACCUAUUC | 1079 |
| | | UTT | | CTT | |
| 1262 | 1262-1280 | GAAUAGGUAUUGGUGAAU | 1080 | AAUUCACCAAUACCUAUU | 1081 |
| | | UTT | | CTT | |
| 1263 | 1263-1281 | AAUAGGUAUUGGUGAAUU | 1082 | AAAUUCACCAAUACCUAU | 1083 |
| | | UTT | | UTT | |
| 1264 | 1264-1282 | AUAGGUAUUGGUGAAUUU | 1084 | UAAAUUCACCAAUACCUA | 1085 |
| | | ATT | | UTT | |
| 1266 | 1266-1284 | AGGUAUUGGUGAAUUUAA | 1086 | UUUAAAUUCACCAAUACC | 1087 |
| | | ATT | | UTT | |
| 1267 | 1267-1285 | GGUAUUGGUGAAUUUAAA | 1088 | CUUUAAAUUCACCAAUAC | 1089 |
| | | GTT | | CTT | |
| 1289 | 1289-1307 | CACUCUCCAUAAAUGCUA | 1090 | GUAGCAUUUAUGGAGAGU | 1091 |
| | | CTT | | GTT | |
| 1313 | 1313-1331 | UUAAACACUUCAAAAACU | 1092 | CAGUUUUUGAAGUGUUUA | 1093 |
| | | GTT | | ATT | |
| 1320 | 1320-1338 | CUUCAAAAACUGCACCUC | 1094 | GGAGGUGCAGUUUUUGAA | 1095 |
| | | CTT | | GTT | |
| 1321 | 1321-1339 | UUCAAAAACUGCACCUCC | 1096 | UGGAGGUGCAGUUUUUGA | 1097 |
| | | ATT | | ATT | |
| 1322 | 1322-1340 | UCAAAAACUGCACCUCCA | 1098 | AUGGAGGUGCAGUUUUUG | 1099 |
| | | UTT | | ATT | |
| 1323 | 1323-1341 | CAAAAACUGCACCUCCAU | 1100 | GAUGGAGGUGCAGUUUUU | 1101 |
| | | CTT | | GTT | |
| 1324 | 1324-1342 | AAAAACUGCACCUCCAUC | 1102 | UGAUGGAGGUGCAGUUUU | 1103 |
| | | ATT | | UTT | |
| 1328 | 1328-1346 | ACUGCACCUCCAUCAGUG | 1104 | CCACUGAUGGAGGUGCAG | 1105 |
| | | GTT | | UTT | |
| 1332 | 1332-1350 | CACCUCCAUCAGUGGCGA | 1106 | AUCGCCACUGAUGGAGGU | 1107 |
| | | UTT | | GTT | |
| 1333 | 1333-1351 | ACCUCCAUCAGUGGCGAU | 1108 | GAUCGCCACUGAUGGAGG | 1109 |
| | | CTT | | UTT | |
| 1335 | 1335-1353 | CUCCAUCAGUGGCGAUCU | 1110 | GAGAUCGCCACUGAUGGA | 1111 |
| | | CTT | | GTT | |
| 1338 | 1338-1356 | CAUCAGUGGCGAUCUCCA | 1112 | GUGGAGAUCGCCACUGAU | 1113 |
| | | CTT | | GTT | |
| 1344 | 1344-1362 | UGGCGAUCUCCACAUCCU | 1114 | CAGGAUGUGGAGAUCGCC | 1115 |
| | | GTT | | ATT | |
| 1345 | 1345-1363 | GGCGAUCUCCACAUCCUG | 1116 | GCAGGAUGUGGAGAUCGC | 1117 |
| | | CTT | | CTT | |
| 1346 | 1346-1364 | GCGAUCUCCACAUCCUGC | 1118 | GGCAGGAUGUGGAGAUCG | 1119 |
| | | CTT | | CTT | |
| 1347 | 1347-1365 | CGAUCUCCACAUCCUGCC | 1120 | CGGCAGGAUGUGGAGAUC | 1121 |
| | | GTT | | GTT | |
| 1348 | 1348-1366 | GAUCUCCACAUCCUGCCG | 1122 | CCGGCAGGAUGUGGAGAU | 1123 |
| | | GTT | | CTT | |
| 1353 | 1353-1371 | CCACAUCCUGCCGGUGGC | 1124 | UGCCACCGGCAGGAUGUG | 1125 |
| | | ATT | | GTT | |
| 1354 | 1354-1372 | CACAUCCUGCCGGUGGCA | 1126 | AUGCCACCGGCAGGAUGU | 1127 |
| | | UTT | | GTT | |
| 1355 | 1355-1373 | ACAUCCUGCCGGUGGCAU | 1128 | AAUGCCACCGGCAGGAUG | 1129 |
| | | UTT | | UTT | |
| 1357 | 1357-1375 | AUCCUGCCGGUGGCAUUU | 1130 | UAAAUGCCACCGGCAGGA | 1131 |
| | | ATT | | UTT | |
| 1360 | 1360-1378 | CUGCCGGUGGCAUUUAGG | 1132 | CCCUAAAUGCCACCGGCA | 1133 |
| | | GTT | | GTT | |
| 1361 | 1361-1379 | UGCCGGUGGCAUUUAGGG | 1134 | CCCCUAAAUGCCACCGGC | 1135 |
| | | GTT | | ATT | |
| 1362 | 1362-1380 | GCCGGUGGCAUUUAGGGG | 1136 | ACCCCUAAAUGCCACCGG | 1137 |
| | | UTT | | CTT | |
| 1363 | 1363-1381 | CCGGUGGCAUUUAGGGGU | 1138 | CACCCCUAAAUGCCACCG | 1139 |
| | | GTT | | GTT | |
| 1366 | 1366-1384 | GUGGCAUUUAGGGGUGAC | 1140 | AGUCACCCCUAAAUGCCA | 1141 |
| | | UTT | | CTT | |
| 1369 | 1369-1387 | GCAUUUAGGGGUGACUCC | 1142 | AGGAGUCACCCCUAAAUG | 1143 |
| | | UTT | | CTT | |
| 1370 | 1370-1388 | CAUUUAGGGGUGACUCCU | 1144 | AAGGAGUCACCCCUAAAU | 1145 |
| | | UTT | | GTT | |
| 1371 | 1371-1389 | AUUUAGGGGUGACUCCUU | 1146 | GAAGGAGUCACCCCUAAA | 1147 |
| | | CTT | | UTT | |
| 1372 | 1372-1390 | UUUAGGGGUGACUCCUUC | 1148 | UGAAGGAGUCACCCCUAA | 1194 |
| | | ATT | | ATT | |
| 1373 | 1373-1391 | UUAGGGGUGACUCCUUCA | 1150 | GUGAAGGAGUCACCCCUA | 1151 |
| | | CTT | | ATT | |
| 1374 | 1374-1392 | UAGGGGUGACUCCUUCAC | 1152 | UGUGAAGGAGUCACCCCU | 1153 |
| | | ATT | | ATT | |
| 1404 | 1404-1422 | UCUGGAUCCACAGGAACU | 1154 | CAGUUCCUGUGGAUCCAG | 1155 |
| | | GTT | | ATT | |
| 1408 | 1408-1426 | GAUCCACAGGAACUGGAU | 1156 | UAUCCAGUUCCUGUGGAU | 1157 |
| | | ATT | | CTT | |
| 1409 | 1409-1427 | AUCCACAGGAACUGGAUA | 1158 | AUAUCCAGUUCCUGUGGA | 1159 |
| | | UTT | | UTT | |
| 1411 | 1411-1429 | CCACAGGAACUGGAUAUU | 1160 | GAAUAUCCAGUUCCUGUG | 1161 |
| | | CTT | | GTT | |
| 1412 | 1412-1430 | CACAGGAACUGGAUAUUC | 1162 | AGAAUAUCCAGUUCCUGU | 1163 |
| | | UTT | | GTT | |
| 1419 | 1419-1437 | ACUGGAUAUUCUGAAAAC | 1164 | GGUUUUCAGAAUAUCCAG | 1165 |
| | | CTT | | UTT | |
| 1426 | 1426-1444 | AUUCUGAAAACCGUAAAG | 1166 | CCUUUACGGUUUUCAGAA | 1167 |
| | | GTT | | UTT | |
| 1427 | 1427-1445 | UUCUGAAAACCGUAAAGG | 1168 | UCCUUUACGGUUUUCAGA | 1169 |
| | | ATT | | ATT | |
| 1430 | 1430-1448 | UGAAAACCGUAAAGGAAA | 1170 | AUUUCCUUUACGGUUUUC | 1171 |
| | | UTT | | ATT | |
| 1431 | 1431-1449 | GAAAACCGUAAAGGAAAU | 1172 | GAUUUCCUUUACGGUUUU | 1173 |
| | | CTT | | CTT | |

TABLE 5
AR Target Sequences

| | | | SEQ | | | |
|---|---|---|---|---|---|---|
| | | | ID | | | |
| ID | Code | Target Sequence | NO: | NM_000044.3 | Exon | Species |
| XD- | 17 | CAAAGGUUCUCUGCUAGACGAC | 1174 | 1987-2005 | 1 | h |
| 01817K1 | | A | | | | |
| XD- | 27 | UCUGGGUGUCACUAUGGAGCUC | 1175 | 2819-2837 | 2 | h |
| 01827K1 | | U | | | | |
| XD- | 28 | CUGGGUGUCACUAUGGAGCUCU | 1176 | 2820-2838 | 2 | h |
| 01828K1 | | C | | | | |
| XD- | 29 | GGGUGUCACUAUGGAGCUCUCA | 1177 | 2822-2840 | 2 | h |
| 01829K1 | | C | | | | |
| XD- | 21 | UACUACAACUUUCCACUGGCUCU | 1178 | 2207-2225 | 1 | h |
| 01821K1 | | | | | | |
| XD- | 25 | AAGCUUCUGGGUGUCACUAUGGA | 1179 | 2814-2832 | 2 | h, m |
| 01825K1 | | | | | | |
| XD- | 26 | CUUCUGGGUGUCACUAUGGAGCU | 1180 | 2817-2835 | 2 | h |
| 01826K1 | | | | | | |

TABLE 6
β-catenin Target Sequences

| | Generic | | | | | |
|---|---|---|---|---|---|---|
| R # | name | Gene | Target sequences | | | |
| R-1146 | 1797mfm | CTNNB1 | CUGUUGGAUUGAU | SEQ ID | UUUCGAAUCAAUCCA | SEQ ID |
| | | | UCGAAAUU | NO. | ACAGUU | NO: |
| | | | | 1181 | | 1182 |
| R-1147 | 1870mfm | CTNNB1 | ACGACUAGUUCAGU | SEQ ID | AAGCAACUGAACUAG | SEQ ID |
| | | | UGCUUUU | NO: | UCGUUU | NO. |
| | | | | 1183 | | 1184 |

TABLE 7
PIK3CA* and PIK3CB* Target Sequences

| Gene | Gene | | | SEQ ID |
|---|---|---|---|---|
| Symbol | ID | Name | Target Sequences (97-mer) | NO: |
| PIK3CA | 5290 | PIK3CA_1746 | TGCTGTTGACAGTGAGCGCCAGCTCAAAGCAATTT | 1185 |
| | | | CTACATAGTGAAGCCACAGATGTATGTAGAAATTG | |
| | | | CTTTGAGCTGTTGCCTACTGCCTCGGA | |
| PIK3CA | 5290 | PIK3CA_2328 | TGCTGTTGACAGTGAGCGAAAGGATGAAACACAA | 1186 |
| | | | AAGGTATAGTGAAGCCACAGATGTATACCTTTTGT | |
| | | | GTTTCATCCTTCTGCCTACTGCCTCGGA | |
| PIK3CA | 5290 | PIK3CA_2522 | TGCTGTTGACAGTGAGCGCCATGTCAGAGTTACTG | 1187 |
| | | | TTTCATAGTGAAGCCACAGATGTATGAAACAGTAA | |
| | | | CTCTGACATGATGCCTACTGCCTCGGA | |
| PIK3CA | 5290 | PIK3CA_3555 | TGCTGTTGACAGTGAGCGCAACTAGTTCATTTCAA | 1188 |
| | | | AATTATAGTGAAGCCACAGATGTATAATTTTGAAA | |
| | | | TGAACTAGTTTTGCCTACTGCCTCGGA | |
| PIK3CA | 5290 | PIK3CA_3484 | TGCTGTTGACAGTGAGCGCACAGCAAGAACAGAA | 1189 |
| | | | ATAAAATAGTGAAGCCACAGATGTATTTTATTTCT | |
| | | | GTTCTTGCTGTATGCCTACTGCCTCGGA | |
| PIK3CB | 5291 | PIK3CB_862 | TGCTGTTGACAGTGAGCGACAAGATCAAGAAAATG | 1190 |
| | | | TATGATAGTGAAGCCACAGATGTATCATACATTTT | |
| | | | CTTGATCTTGCTGCCTACTGCCTCGGA | |
| PIK3CB | 5291 | PIK3CB_183 | TGCTGTTGACAGTGAGCGCAGCAAGTTCACAATTA | 1191 |
| | | | CCCAATAGTGAAGCCACAGATGTATTGGGTAATTG | |
| | | | TGAACTTGCTTTGCCTACTGCCTCGGA | |
| PIK3CB | 5291 | PIK3CB_1520 | TGCTGTTGACAGTGAGCGCCCCTTCGATAAGATTA | 1192 |
| | | | TTGAATAGTGAAGCCACAGATGTATTCAATAATCT | |
| | | | TATCGAAGGGATGCCTACTGCCTCGGA | |
| PIK3CB | 5291 | PIK3CB_272 | TGCTGTTGACAGTGAGCGAGAGCTTGAAGATGAAA | 1193 |
| | | | CACGATAGTGAAGCCACAGATGTATCGTGTTTCAT | |
| | | | CTTCAAGCTCCTGCCTACTGCCTCGGA | |
| PIK3CB | 5291 | PIK3CB_948 | TGCTGTTGACAGTGAGCGACACCAAAGAAAACAC | 1194 |
| | | | GAATTATAGTGAAGCCACAGATGTATAATTCGTGT | |
| | | | TTTCTTTGGTGGTGCCTACTGCCTCGGA | |

*Species is Homo sapiens.

TABLE 8
PIK3CA and PIK3CB siRNA Sequences

| | | | | SEQ | | SEQ |
|---|---|---|---|---|---|---|
| Gene | Gene | | | ID | | ID |
| Symbol | ID | Name | siRNA Guide | NO: | siRNA passenger | NO: |
| PIK3CA | 5290 | PIK3CA_1746 | UGUAGAAAUUGCUU | 1195 | AGCUCAAAGCAAUUU | 1196 |
| | | | UGAGCUGU | | CUACAUA | |
| PIK3CA | 5290 | PIK3CA_2328 | UACCUUUUGUGUUU | 1197 | AGGAUGAAACACAAA | 1198 |
| | | | CAUCCUUC | | AGGUAUA | |
| PIK3CA | 5290 | PIK3CA_2522 | UGAAACAGUAACUC | 1199 | AUGUCAGAGUUACUG | 1200 |
| | | | UGACAUGA | | UUUCAUA | |
| PIK3CA | 5290 | PIK3CA_3555 | UAAUUUUGAAAUGA | 1201 | ACUAGUUCAUUUCAA | 1202 |
| | | | ACUAGUUU | | AAUUAUA | |
| PIK3CA | 5290 | PIK3CA_3484 | UUUUAUUUCUGUUC | 1203 | CAGCAAGAACAGAAA | 1204 |
| | | | UUGCUGUA | | UAAAAUA | |
| PIK3CB | 5291 | PIK3CB_862 | UCAUACAUUUUCUU | 1205 | AAGAUCAAGAAAAUG | 1206 |
| | | | GAUCUUGC | | UAUGAUA | |
| PIK3CB | 5291 | PIK3CB_183 | UUGGGUAAUUGUGA | 1207 | GCAAGUUCACAAUUA | 1208 |
| | | | ACUUGCUU | | CCCAAUA | |
| PIK3CB | 5291 | PIK3CB_1520 | UUCAAUAAUCUUAU | 1209 | CCUUCGAUAAGAUUA | 1210 |
| | | | CGAAGGGA | | UUGAAUA | |
| PIK3CB | 5291 | PIK3CB_272 | UCGUGUUUCAUCUU | 1211 | AGCUUGAAGAUGAAA | 1212 |
| | | | CAAGCUCC | | CACGAUA | |
| PIK3CB | 5291 | PIK3CB_948 | UAAUUCGUGUUUUC | 1213 | ACCAAAGAAAACACG | 1214 |
| | | | UUUGGUGG | | AAUUAUA | |

TABLE 9
Additional hetero-duplex polynucleotide sequences

| | Base | | SEQ | | SEQ |
|---|---|---|---|---|---|
| | start | | ID | | ID |
| | position | Guide strand | NO: | Passenger strand | NO: |
| EGFR | 333 | ACUCGUGCCUUGGCAA | 1215 | AGUUUGCCAAGGCACGA | 1216 |
| R1246 | | ACUUU | | GUUU | |
| EGFR | 333 | ACUCGUGCCUUGGCAA | 1217 | AGUUUGCCAAGGCACGA | 1218 |
| R1195 | | ACUUU | | GUUU | |
| EGFR | 333 | ACUCGUGCCUUGGCAA | 1219 | AGUUUGCCAAGGCACGA | 1220 |
| R1449 | | ACUUU | | GUUU | |
| KRAS | 237 | UGAAUUAGCUGUAUCG | 1221 | TGACGAUACAGCUAAUUC | 1222 |
| R1450 | | UCAUU | | AUU | |
| KRAS | 237 | UGAAUUAGCUGUAUCG | 1223 | UGACGAUACAGCUAAUU | 1224 |
| R1443 | | UCAUU | | CAUU | |
| KRAS | 237 | UGAAUUAGCUGUAUCG | 1225 | UGACGAUACAGCUAAUU | 1226 |
| R1194 | | UCAUU | | CAUU | |
| CTNNB1 | 1248 | UAAGUAUAGGUCCUCA | 1227 | UAAUGAGGACCUAUACU | 1228 |
| R1442 | | UUAUU | | UAUU | |
| CTNNB1 | 1797 | TUUCGAAUCAAUCCAA | 1229 | CUGUUGGAUUGAUUCGA | 1230 |
| R1404 | | CAGUU | | AAUU | |
| CTNNB1 | 1797 | UUUCGAAUCAAUCCAA | 1231 | CUGUUGGAUUGAUUCGA | 1232 |
| R1441 | | CAGUU | | AAUU | |
| CTNNB1 | 1797 | UUUCGAAUCAAUCCAA | 1233 | CUGUUGGAUUGAUUCGA | 1234 |
| R1523 | | CAGUU | | AAUU | |
| HPRT | 425 | AUAAAAUCUACAGUCA | 1235 | CUAUGACUGUAGAUUUU | 1236 |
| R1492 | | UAGUU | | AUUU | |
| HPRT | 425 | UUAAAAUCUACAGUCA | 1237 | CUAUGACUGUAGAUUUU | 1238 |
| R1526 | | UAGUU | | AAUU | |
| HPRT | 425 | UUAAAAUCUACAGUCA | 1239 | CUAUGACUGUAGAUUUU | 1240 |
| R1527 | | UAGUU | | AAUU | |
| AR | 2822 | GAGAGCUCCAUAGUGA | 1241 | GUGUCACUAUGGAGCUC | 1242 |
| R1245 | | CACUU | | UCUU | |

### Example 2. siRNA Conjugate with DAR2 or Higher

siRNA Synthesis

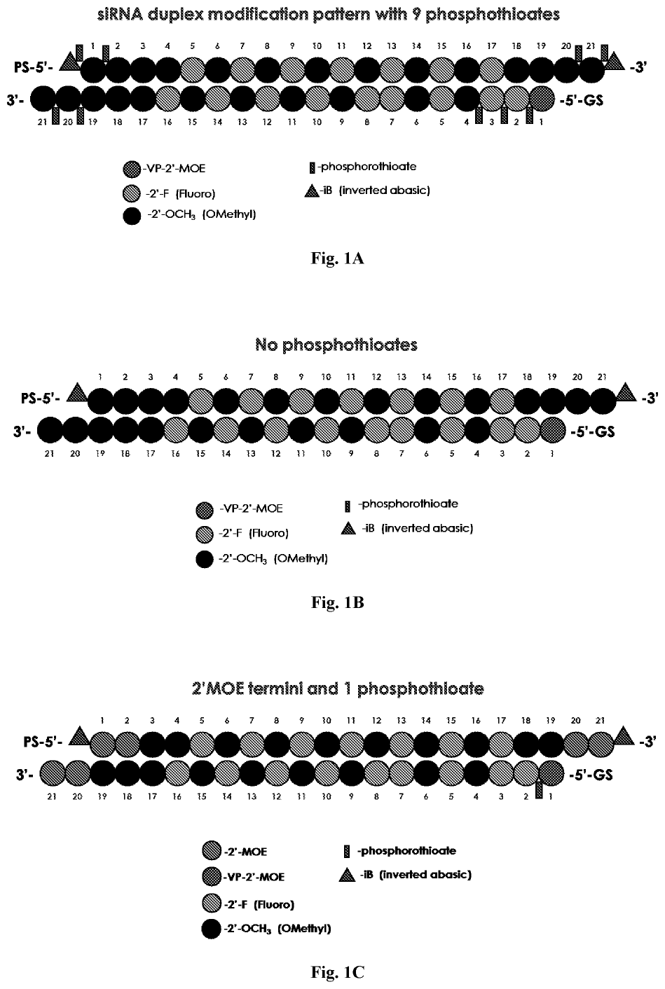

The siRNA single strands were fully assembled on solid phase using standard phosphoramidite chemistry and purified over HPLC. Purified single strands were duplexed to get the double stranded siRNA. The modification patterns used in the duplex siRNAs is shown in FIGS. 1A-1C.

The siRNA passenger strands contain conjugation handles in different formats, C6-NH₂ and/or C6-SH, one at each end of the strand. The conjugation handle or handles were connected to siRNA passenger strand via inverted abasic phosphodiester or phosphorothioate or directly attached to 3′ or 5′ end of the siRNA.

Below is a representative structure of siRNA passenger strand with C6-NH₂ conjugation handle at the 5′ end and C6-SH at 3′end.

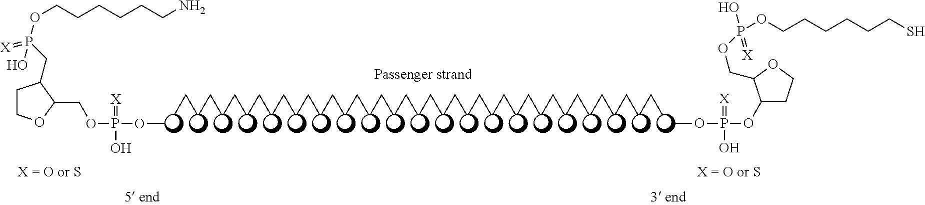

Below is a representative structure of siRNA passenger strand with C6-NH₂ conjugation handle at the 5′ end.

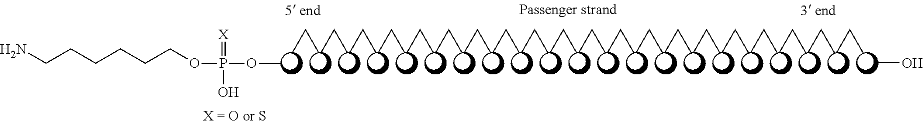

Below is a representative structure of siRNA passenger strand with C6-NH2 conjugation handle at the 3′ end.

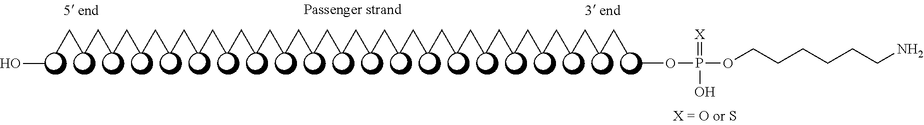

### Example 2.2. Synthesis of Phosphorodiamidate Morpholino Oligomer (PMO)

PMOs were fully assembled on solid phase using standard solid phase synthesis protocols and purified over HPLC. PMO contains an amine conjugation handle either at 5′ end or at 3′ end of the molecule for conjugation to antibodies or Fabs or other proteins.

Below is A representative structure of the PMO with 5′ amine conjugation handle

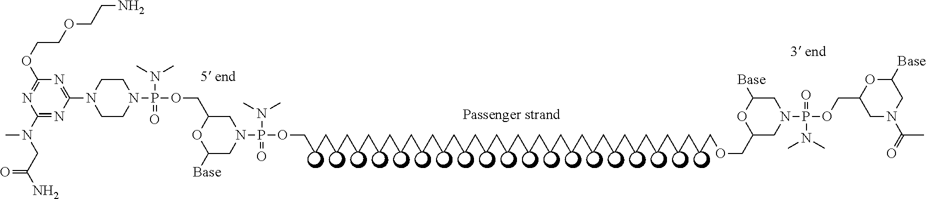

Below is a representative structure of the PMO with 3′ amine conjugation handle.

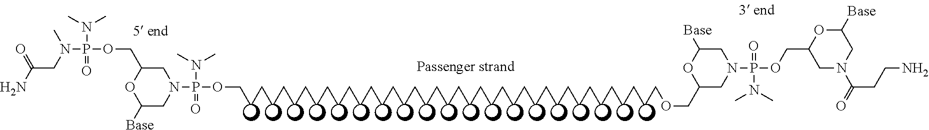

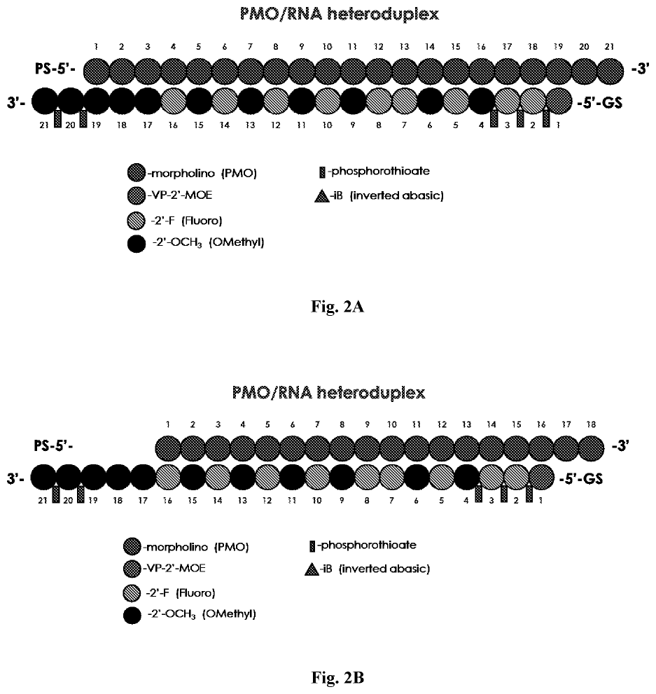

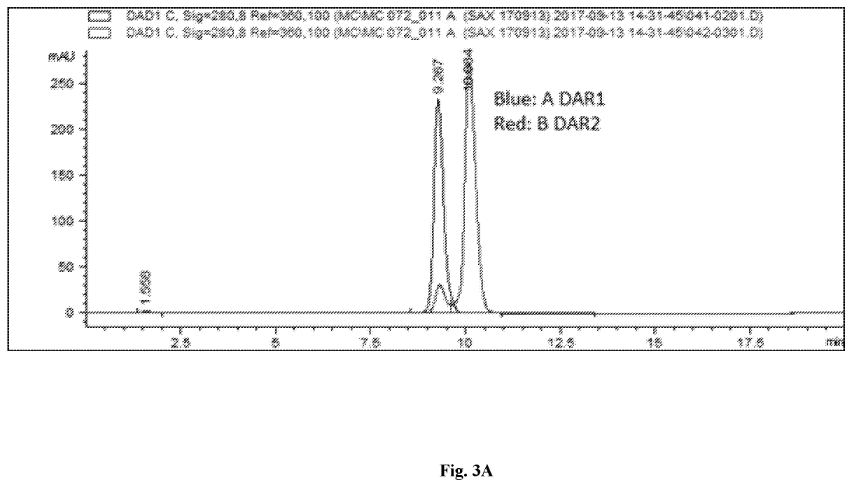

Structures of the PMO/RNA heteroduplex are seen in FIGS. 2A-2B. FIG. 2A shows a 21mer duplex with 19 bases of complementarity and 3′ dinucleotide overhangs. The guide strand was RNA and the modification pattern used is described. The 3′end or 5′ end of the PMO contained an NH₂ conjugation handle to allow attachment of the linker and antibody. FIG. 2B shows a truncated duplex with 16 bases of complementarity and unsymmetrical 3′ overhangs. The guide strand was RNA and the modification pattern used is described. The 3′end or 5′ of the PMO contained an amine conjugation handle to allow attachment of the linker and antibody.

### Example 2.3. Synthesis of the PS-ASO-EON-Decoy

The ASO decoy (PS-ASO-EON_decoy) was fully assembled on solid phase using standard phosphoramidite chemistry nd purified over HPLC.

### Example 2.4. Synthesis of Peptide Nucleic Acid (PNA)

Peptide nucleic acid was synthesized on solid phase using Fmoc chemistry. The fully assembled PNA sequence was cleaved off the solid phase and purified over HPLC before lyophilization. The PNA may contain a conjugation handle at the 5′ end of the molecule.

Structure of PNA Passenger Strand

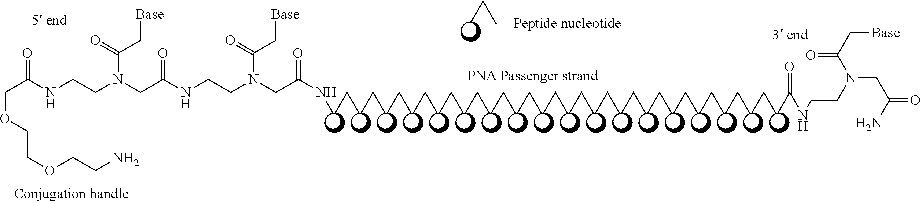

Structure of PNA/RNA Heteroduplex

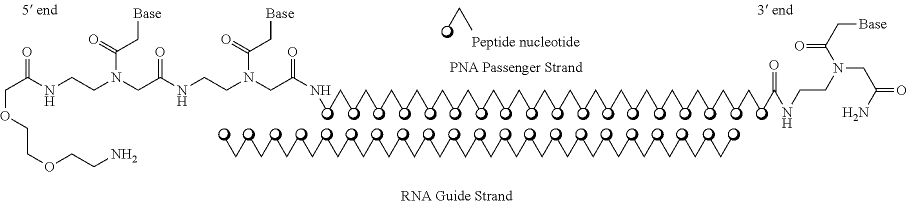

### Example 2.5. Conjugates

The architectures of the conjugates for the following experiments are described below. Details of the synthesis and purification are described in Example 4.

Architecture 1 is mAb-SMCC-3′amine-0 PMO-with the guide strand as seen below.

Architecture 2 is mAb-BisMal-3′amine-0 PMO-with the guide strand as seen below.

Architecture 3 is mAb-SMCC-5′amine-0 PMO-with the guide strand as seen below.

Architecture 4 is mAb-SMCC-5′amine-18 PMO-with the guide strand as seen below.

Architecture 5 is mAb-BisMal-5′amine-0 PMO-with the guide strand as seen below.

Architecture 6 is mAb-BisMal-5′amine-siRNA-3′-SS-dT as seen below.

Architecture 7 is mAb-BisMal-5′amine-siRNA (without inverted abasic groups) as seen below.

Architecture 8 is mAb-BisMal-3′amine-siRNA (without inverted abasic groups) as seen below.

### Example 3. General Experimental Protocol

Stem-Loop qPCR Assay for Quantification of siRNA

Plasma samples were directly diluted in TE buffer. 50 mg tissue pieces were homogenized in 1 mL of Trizol using a FastPrep-24 tissue homogenizer (MP Biomedicals) and then diluted in TE buffer. Standard curves were generated by spiking siRNA into plasma or homogenized tissue from untreated animals and then serially diluting with TE buffer. The antisense strand of the siRNA was reverse transcribed using a TaqMan MicroRNA reverse transcription kit (Applied Biosystems) with 25 nM of a sequence-specific stem-loop RT primer. The cDNA from the RT step was utilized for real-time PCR using TaqMan Fast Advanced Master Mix (Applied Biosystems) with 1.5 μM of forward primer, 0.75 μM of reverse primer, and 0.2 μM of probe. The sequences of SSB, Aha1 and HPRT siRNA antisense strands and all primers and probes used to measure them are shown in Table 11. Quantitative PCR reactions were performed using standard cycling conditions in a ViiA 7 Real-Time PCR System (Life Technologies). The Ct values were transformed into plasma or tissue concentrations using the linear equations derived from the standard curves.

TABLE 11
Sequences for siRNA antisense strands, primers,
and probes used in the stem-loop qPCR assay.

| Target | Name | Sequence (5′-3′) |
|---|---|---|
| SSB | Antisense | UUACAUUAAAGUCUGUUGUUU |
| SSB | RT primer | GTC-GTA-TCC-AGT-GCA-GGG- |
| | | TCC-GAG-GTA-TTC-GCA-CTG- |
| | | GAT-ACG-ACA-AAC-AAC |
| SSB | Forward | GGC-GGC-TTA-CAT-TAA-AGT-CTG-T |
| SSB | Reverse | AGT GCA GGG TCC GAG |
| SSB | Probe | TGG-ATA-CGA-CAA-ACA-A |
| Aha1 | Antisense | UCUAAUCUCCACUUCAUCCUU |
| Aha1 | RT primer | GTC-GTA-TCC-AGT-GCA-GGG- |
| | | TCC-GAG-GTA-TTC-GCA-CTG- |
| | | GAT-ACG-ACA-AGG-ATG |
| Aha1 | Forward | GGC-GGC-TCT-AAT-CTC-CAC-TTC |
| Aha1 | Reverse | AGT GCA GGG TCC GAG |
| Aha1 | Probe | TGG-ATA-CGA-CAA-GGA-T |
| HPRT | Antisense | UUAAAAUCUACAGUCAUAGUU |
| HPRT | RT primer | GTC-GTA-TCC-AGT-GCA-GGG- |
| | | TCC-GAG-GTA-TTC-GCA-CTG- |
| | | GAT-ACG-ACA-ACT-ATG |
| HPRT | Forward | GGC-GGC-TTA-AAA-TCT-ACA-GTC-AT |
| HPRT | Reverse | AGT GCA GGG TCC GAG |
| HPRT | Probe | TGG-ATA-CGA-CAA-CTA-TGA |

Comparative qPCR Assay for Determination of mRNA Knockdown.

Tissue samples were homogenized in Trizol as described above. Total RNA was isolated using RNeasy RNA isolation 96-well plates (Qiagen). 500 ng RNA was then reverse transcribed with a High Capacity RNA to cDNA kit (ThermoFisher). SSB, Aha1 and HPRT mRNA were quantified by TaqMan qPCR analysis performed with a ViiA 7 Real-Time PCR System. The TaqMan primers and probes were purchased from Applied Biosystems as pre-validated gene expression assays (Primer/Probe Sets: HPRT: Mm03024075_m1, PPIB: Mm00478295_m1, SSB: Mm00447374_m1, AHSA1: Mm01296842_m1). PPIB (housekeeping gene) was used as an internal RNA loading control, with all TaqMan primers and probes for PPIB purchased from Applied Biosystems as pre-validated gene expression assays. Results are calculated by the comparative Ct method, where the difference between the target gene (KRAS, CTNNB1, or EGFR) Ct value and the PPIB Ct value (ΔCt) is calculated and then further normalized relative to the PBS control group by taking a second difference (ΔΔCt).

Animals

All animal studies were conducted following protocols in accordance with the Institutional Animal Care and Use Committee (IACUC) at Explora BioLabs, which adhere to the regulations outlined in the USDA Animal Welfare Act as well as the “Guide for the Care and Use of Laboratory Animals” (National Research Council publication, 8th Ed., revised in 2011). All mice were obtained from either Charles River Laboratories or Harlan Laboratories. Wild type CD-1 mice (4-6 week old) were dosed via intravenous (iv) injection with the indicated ASCs and doses.

Anti-Transferrin Receptor Antibody

Anti-mouse transferrin receptor antibody or CD71 mAb is a rat IgG2a subclass monoclonal antibody that binds mouse CD71 or mouse transferrin receptor 1 (mTfR1). The antibody was produced by BioXcell (Catalog #BE0175).

Anti-EGFR Antibody

Anti-EGFR antibody is a fully human IgG1κ monoclonal antibody directed against the human epidermal growth factor receptor (EGFR). It is produced in the Chinese Hamster Ovary cell line DJT33, which has been derived from the CHO cell line CHO-K1SV by transfection with a GS vector carrying the antibody genes derived from a human anti-EGFR antibody producing hybridoma cell line (2F8). Standard mammalian cell culture and purification technologies are employed in the manufacturing of anti-EGFR antibody.

The theoretical molecular weight (MW) of anti-EGFR antibody without glycans is 146.6 kDa. The experimental MW of the major glycosylated isoform of the antibody is 149 kDa as determined by mass spectrometry. Using SDS-PAGE under reducing conditions the MW of the light chain was found to be approximately 25 kDa and the MW of the heavy chain to be approximately 50 kDa. The heavy chains are connected to each other by two inter-chain disulfide bonds, and one light chain is attached to each heavy chain by a single inter-chain disulfide bond. The light chain has two intra-chain disulfide bonds and the heavy chain has four intra-chain disulfide bonds. The antibody is N-linked glycosylated at Asn305 of the heavy chain with glycans composed of N-acetyl-glucosamine, mannose, fucose and galactose. The predominant glycans present are fucosylated bi-antennary structures containing zero or one terminal galactose residue.

The charged isoform pattern of the IgG1κ antibody was investigated using imaged capillary IEF, agarose IEF and analytical cation exchange HPLC. Multiple charged isoforms were found, with the main isoform having an isoelectric point of approximately 8.7.

The major mechanism of action of anti-EGFR antibody is a concentration dependent inhibition of EGF-induced EGFR phosphorylation in A431 cancer cells. Additionally, induction of antibody-dependent cell-mediated cytotoxicity (ADCC) at low antibody concentrations has been observed in pre-clinical cellular in vitro studies.

Myostatin ELISA

Myostatin protein in plasma was quantified using the GDF-8 (Myostatin) Quantikine ELISA Immunoassay (part #DGDF80) from R&D Systems according to the manufacturer's instructions.

RISC Loading Assay

Specific immunoprecipitation of the RISC from tissue lysates and quantification of small RNAs in the immunoprecipitates were determined by stem-loop PCR, using an adaptation of the assay described by Pei et al. Quantitative evaluation of siRNA delivery in vivo. RNA (2010), 16:2553-2563

### Example 4.1. Antibody PMO/RNA Heteroduplex Conjugate Synthesis Scheme

An antibody PMO/RNA heteroduplex conjugate synthesis scheme is below.

Step 1: Antibody Interchain Disulfide Reduction with TCEP

Antibody was buffer exchanged with borax buffer (pH 8) and made up to 10 mg/ml concentration. To this solution, 2 equivalents of TCEP in water was added and rotated for 2 hours at RT. The resultant reaction mixture was buffer exchanged with pH 7.4 PBS containing 5 mM EDTA and added to a solution of SMCC-PMO/RNA (1.4 equivalents) in pH 7.4 PBS containing 5 mM EDTA at RT and rotated overnight. Analysis of the reaction mixture by analytical SAX column chromatography showed antibody PMO/RNA heteroduplex conjugates along with unreacted antibody and PMO/RNA heteroduplex.

Step 2: Purification

The crude reaction mixture was purified by HPLC using anion exchange chromatography method-1 as described in “purification and analytical methods” below. Fractions containing DAR1, DAR2, DAR>2 antibody-PMO/RNA heteroduplex conjugates were separated, concentrated and buffer exchanged with pH 7.4 PBS.

Step 3: Analysis of the Purified Conjugate

The isolated conjugates were characterized by SEC, SAX chromatography and SDS-PAGE. The purity of the conjugate was assessed by analytical HPLC using either anion exchange chromatography method-2 or anion exchange chromatography method-3.

### Example 4.2. Antibody siRNA Conjugate Synthesis Scheme

An antibody siRNA conjugate synthesis scheme is seen below.

Step 1: Antibody Interchain Disulfide Reduction with TCEP

Antibody was buffer exchanged with borax buffer (pH 8) and made up to 10 mg/ml concentration. To this solution, 2 equivalents of TCEP in water was added and rotated for 2 hours at RT. The resultant reaction mixture was buffer exchanged with pH 7.4 PBS containing 5 mM EDTA and added to a solution of SMCC-C6-siRNA in pH 7.4 PBS containing 5 mM EDTA at RT and rotated overnight. Analysis of the reaction mixture by analytical SAX column chromatography showed antibody siRNA conjugate along with unreacted antibody and siRNA.

Step 2: Purification

The crude reaction mixture was purified by AKTA explorer FPLC using anion exchange chromatography method-3 as seen in the “purification and analytical methods” below. Fractions containing DAR1, DAR2 and DAR>2 antibody-siRNA conjugates were separated, concentrated and buffer exchanged with pH 7.4 PBS.

Step 3: Analysis of the Purified Conjugate

The isolated conjugates were characterized by SEC and SAX chromatography. The purity of the conjugate was assessed by analytical HPLC using either anion exchange chromatography method-2.

### Example 4.3. Purification and Analytical Methods

Table 12 depicts anion exchange chromatography method-1.

TABLE 12
Anion exchange chromatography method-1

| Column | Tosoh Biosciences TSKgel SuperQ 5PW, | | |
|---|---|---|---|
| | 7.5 mm × 7.5 cm, 10 um | | |
| Solvent A | 90% 20 mM Tris pH 7.4 + 10% EtOH; | | |
| | Solvent B: 90% 20 mM Tris with | | |
| | 1.5M NaCl, pH 7.4 + 10% EtOH | | |
| Flow rate | 1 mL/minute | | |
| Gradient | Time | % A | % B |
| | 0.0 | 92 | 8 |
| | 5.00 | 92 | 8 |
| | 40.00 | 74 | 26 |
| | 42.00 | 0 | 100 |
| | 47.00 | 0 | 100 |
| | 48.00 | 92 | 8 |
| | 53.00 | 92 | 8 |

Table 13 depicts anion exchange chromatography method-2.

TABLE 13
Anion exchange chromatography method-2

| Column | Thermo Scientific, ProPac ^{TM} SAX-10, | | |
|---|---|---|---|
| | Bio LC ^{TM}, 4 × 250 mm | | |
| Solvent A | 80% 10 mM TRIS pH 8, | | |
| | 20% ethanol; Solvent B: 80% 10 mM TRIS pH 8, | | |
| | 20% ethanol, 1.5M NaCl | | |
| Flow rate | 1.0 mL/minute | | |
| Gradient | Time | % A | % B |
| | 0.0 | 90 | 10 |
| | 3.00 | 90 | 10 |
| | 11.00 | 40 | 60 |
| | 13.00 | 40 | 60 |
| | 15.00 | 90 | 10 |
| | 20.00 | 90 | 10 |

Table 14 depicts anion exchange chromatography method-3.

TABLE 14
Anion exchange chromatography method-3

| Column | Tosoh Bioscience, TSKGel SuperQ-5PW, 21.5 mm | | |
|---|---|---|---|
| | ID × 15 cm, 13 um | | |
| Solvent A | 20 mM TRIS buffer, pH 8.0; Solvent B: 20 mM | | |
| | TRIS, 1.5M NaCl, pH 8.0 | | |
| Flow rate | 6.0 mL/minute | | |
| Gradient | Volume | % A | % B |
| | 1.00 | 100 | 0 |
| | 18.00 | 60 | 40 |
| | 2.00 | 40 | 60 |
| | 5.00 | 40 | 60 |
| | 2.00 | 0 | 100 |
| | 2.00 | 100 | 0 |

Table 15 depicts Size exclusion chromatography method-1.

TABLE 15
Size exclusion chromatography method-1

| | |
|---|---|
| Column | TOSOH Biosciences, TSKgel, G3000SW XL, |
| | 7.8 × 300 mm, 5μ |
| Mobile phase | 150 mM phosphate buffer |
| Flow rate | 1 mL/minute for 20 minutes |

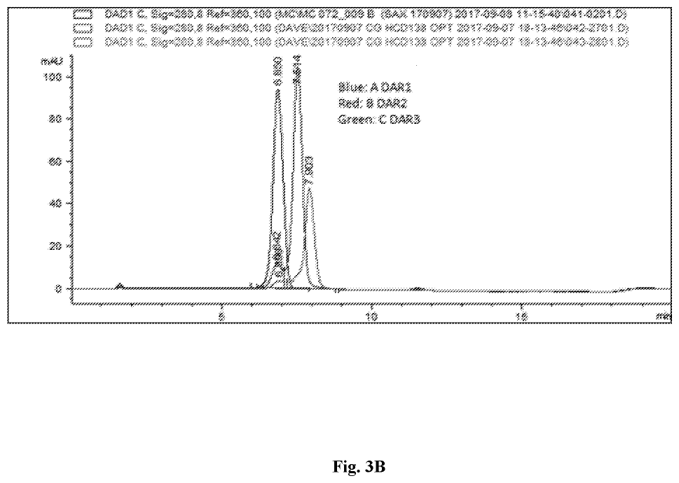

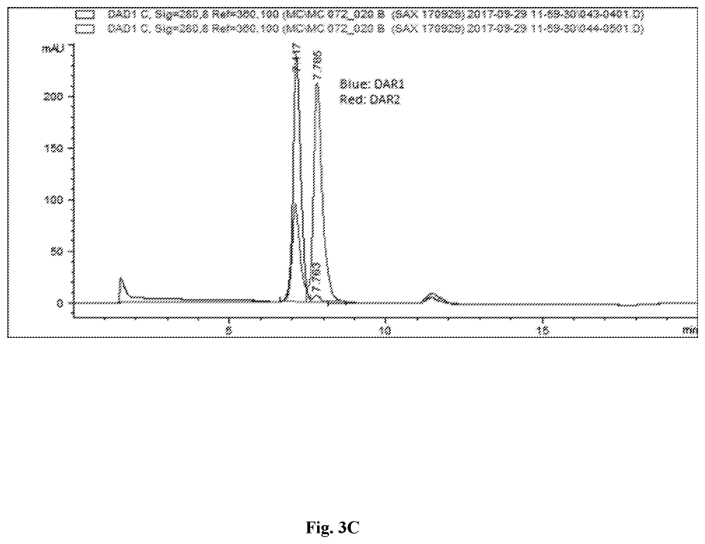

FIGS. 3A-3C depict ASC analytical chromatograms. FIG. 3A shows overlaid SAX-HPLC chromatograms of EGFR mAb-SSB DAR1 and DAR2 conjugates. FIG. 3B shows overlaid SAX-HPLC chromatograms of EGFR mAb-SSB-0 PMO DAR1, DAR2 and DAR3 conjugates. FIG. 3C shows overlaid SAX-HPLC chromatograms of TfR mAb-SSB-18 PMO DAR1, and DAR2 conjugates.

### Example 5. 2017-PK-361-WT: Plasma PK with Anti-EGFR mAb to Compare siRNA-PMO Heteroduplex (DAR1 vs DAR2)

The 21mer SSB guide strand was designed against mouse SSB. The sequence (5′ to 3′) of the guide/antisense strand was UUACAUUAAAGUCUGUUGUUU. The guide and fully complementary RNA passenger strands were assembled on solid phase using standard phospharamidite chemistry and purified over HPLC. Base, sugar and phosphate modifications were as described in Example 2, chemical modification pattern 1. Both conjugation handles were connected to siRNA passenger strand via phosphorothioate-inverted abasic-phosphorothioate linker.

21mer and 18mer complementary PMO passenger strands were fully assembled on solid phase using standard solid phase synthesis protocols and purified over HPLC. Each PMO contained a C3-NH2 conjugation handle at the 5′ end of the molecule as in Example 2. The SSB guide strand was duplexed with the RNA and two PMO guide strands to generate a siRNA homoduplex (designated as SSB) and two PMO/RNA heteroduplexes: one with the 21mer PMO (designated as SSB-0 PMO) and the other with an 18mer (designated as SSB-18 PMO).

ASC Synthesis and Characterization

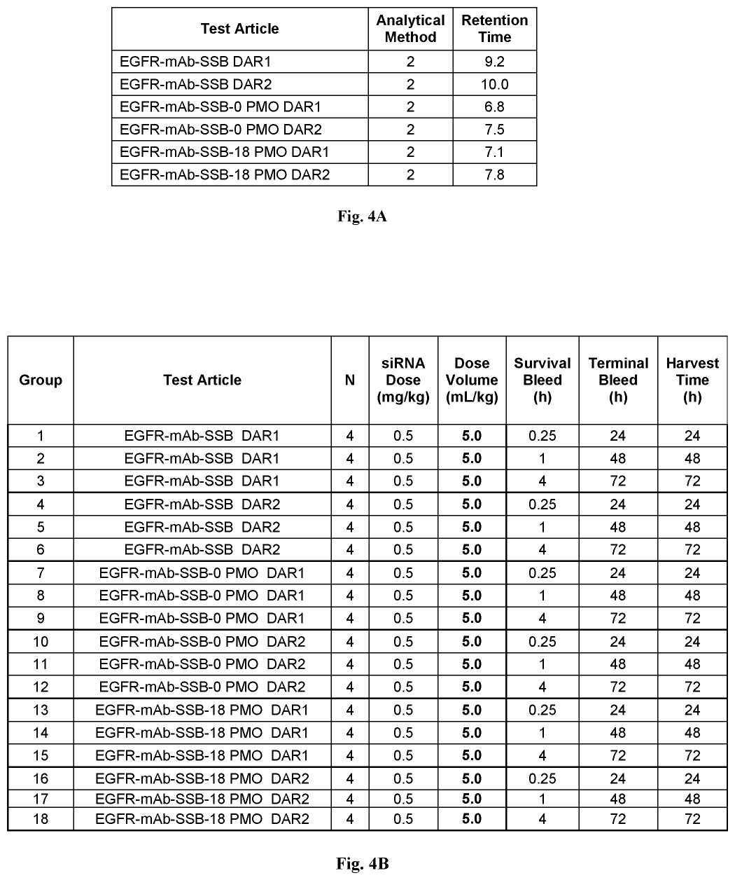

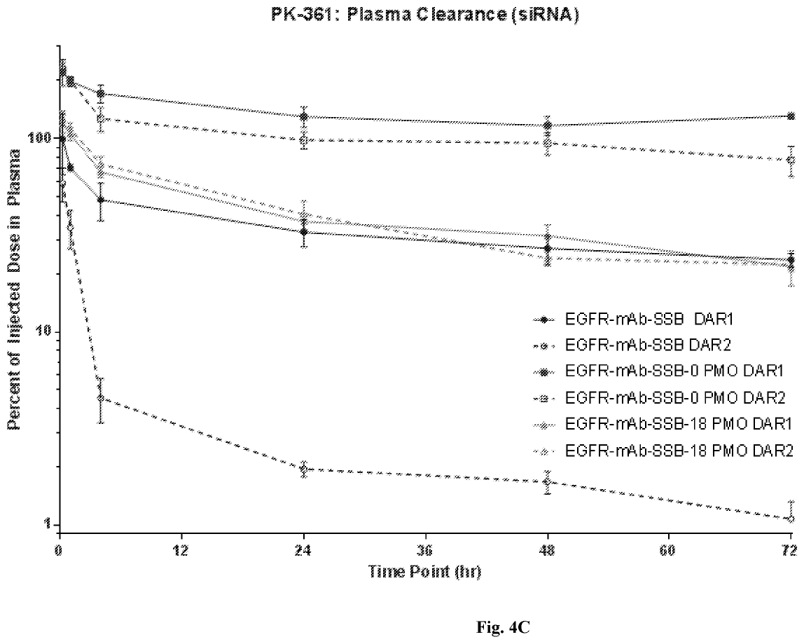

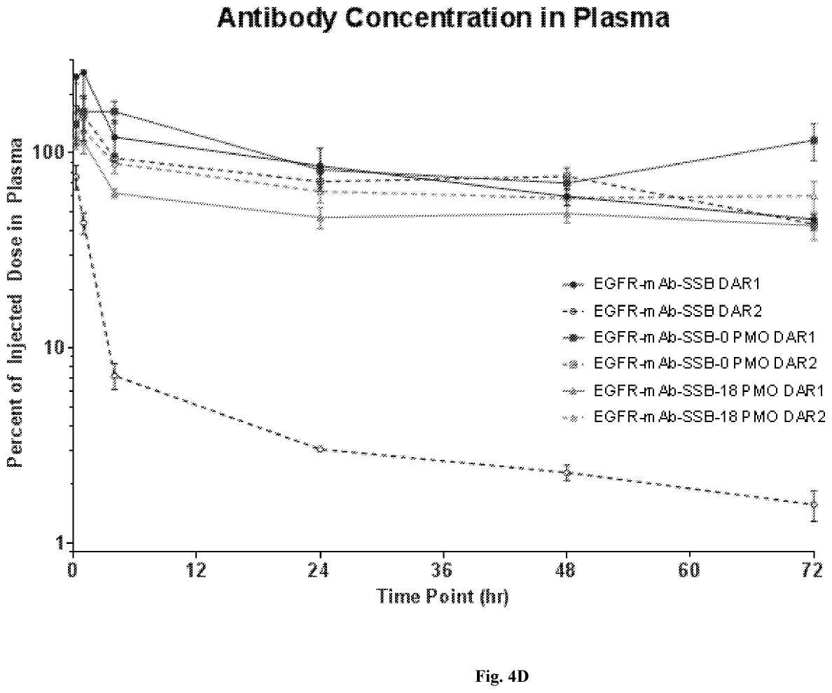

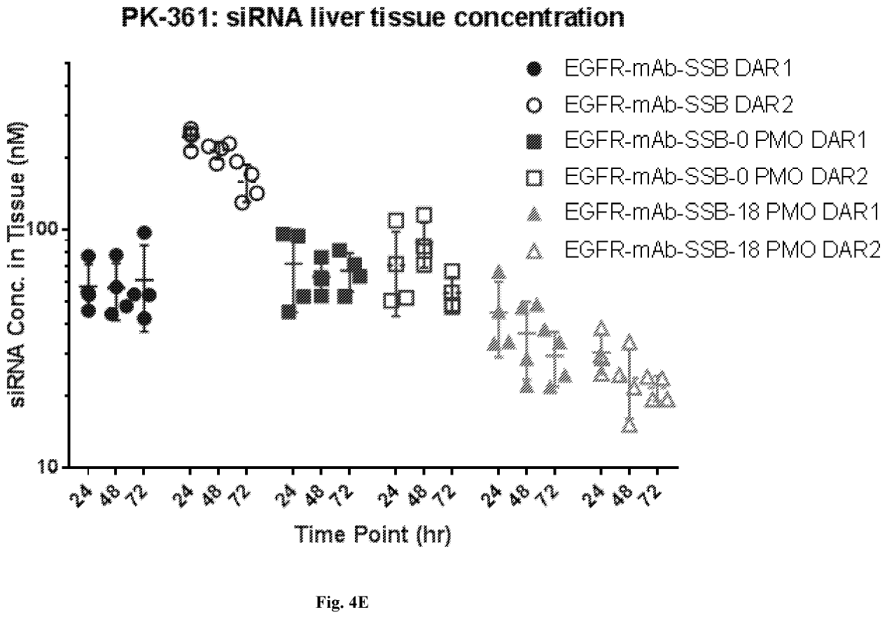

The anti-EGFR mAb-SSB DAR1 and DAR2 were synthesized and purified as described in Example 4 using a C6-NH2 conjugation handle at the 5′ end and C6-SH at 3′end of the passenger strand. The anti-EGFR mAb-SSB-0 PMO DAR1 and DAR2 were synthesized/purified as described in Example 4 using a C6-NH2 conjugation handle at the 5′end of the PMO guide strand and used architecture 5 (see Example 2). The anti-EGFR mAb-SSB-18 PMO DAR1 and DAR 2 were synthesized/purified as described in Example 2.2 using a C6-NH2 conjugation handle at the 5′end of the PMO guide strand and used architecture 4 (see Example 2). All conjugates were made through nonspecific cysteine conjugation, using a BisMal linker and were characterized chromatographically as seen in FIG. 4A. FIG. 4A shows an analytical data table of conjugates with HPLC retention time (RT) in minutes.

In Vivo Study Design

The plasma pharmacokinetics of the conjugates were assessed in vivo in wild type CD-1 mice after intravenous dosing. Mice were dosed via intravenous (iv) injection with PBS vehicle control and the indicated ASCs and doses as seen in FIG. 4B. Non-terminal blood samples (survival bleed) were collected at the indicated times via puncture of the retro-orbital plexus and centrifuged to generate plasma for PK analysis. Mice were sacrificed by CO₂ asphyxiation at (terminal bleed/harvest) at the indicated times and terminal blood samples were collected via cardiac puncture and processed to generate plasma for PK analysis. 50 mg pieces of liver were collected and snap-frozen in liquid nitrogen and total mRNA was extracted. As described in Example 3, quantitation of plasma or tissue siRNA concentrations was determined using a stem-loop qPCR assay. The antisense strand of the siRNA was reverse transcribed using a TaqMan MicroRNA reverse transcription kit using a sequence-specific stem-loop RT primer. The cDNA from the RT step was then utilized for real-time PCR and Ct values were transformed into plasma or tissue concentrations using the linear equations derived from the standard curves. Plasma concentrations of the anti-EGFR antibody were determined using an ELISA assay.

Results

For the DAR1 conjugates, use of PMO chemistry on the passenger strand of the duplex (RNA/PMO heteroduplex) resulted in a longer (EGFR-mAb-SSB-0 PMO) or equivalent (EGFR-mAb-SSB-18 PMO) plasma half-life, relative to the standard RNA/RNA homoduplex DAR1 ASC (EGFR-mAb-SSB) as seen in FIGS. 4C-4D.

For the DAR2 conjugates, use of PMO chemistry on the passenger strand of the duplex (RNA/PMO heteroduplex) resulted in a longer (EGFR-mAb-SSB-0 PMO and EGFR-mAb-SSB-18 PMO) plasma half-life, relative to the standard RNA/RNA homoduplex DAR2 ASC (EGFR-mAb-SSB) as seen in FIGS. 4C-4D. In addition, liver guide strand RNA concentrations of the DAR2 heteroduplex ASCs were much lower relative to the standard RNA/RNA homoduplex DAR2 ASC as seen in FIG. 4E.

Using PMO chemistry on the passenger strand of the duplex (RNA/PMO heteroduplex) results in improved pharmacokinetic properties of antibody conjugates.

### Example 6. 2017-PK-375-WT—CD71 mAb RNA/PMO Heteroduplex Compared to siRNA-Homoduplex (DAR1 vs DAR2)

The 21mer SSB guide strand was designed against mouse SSB. The sequence (5′ to 3′) of the guide/antisense strand was UUACAUUAAAGUCUGUUGUUU. The guide and fully complementary RNA passenger strands were assembled on solid phase using standard phospharamidite chemistry and purified over HPLC. Base, sugar and phosphate modifications were as described in Example 2, chemical modification pattern 1. Both conjugation handles were connected to siRNA passenger strand via phosphorothioate-inverted abasic-phosphorothioate linker.

21mer and 18mer complementary PMO passenger strands were fully assembled on solid phase using standard solid phase synthesis protocols and purified over HPLC. Each PMO contained a C3-NH2 conjugation handle at the 5 end of the molecule similarly described in Example 2. The SSB guide strand was duplexed with the RNA and two PMO guide strands to generate a siRNA homoduplex (designated as SSB) and two PMO/RNA heteroduplexes: one with the 21mer PMO (designated as SSB-0 PMO) and the other with an 18mer (designated as SSB-18 PMO).

ASC Synthesis and Characterization

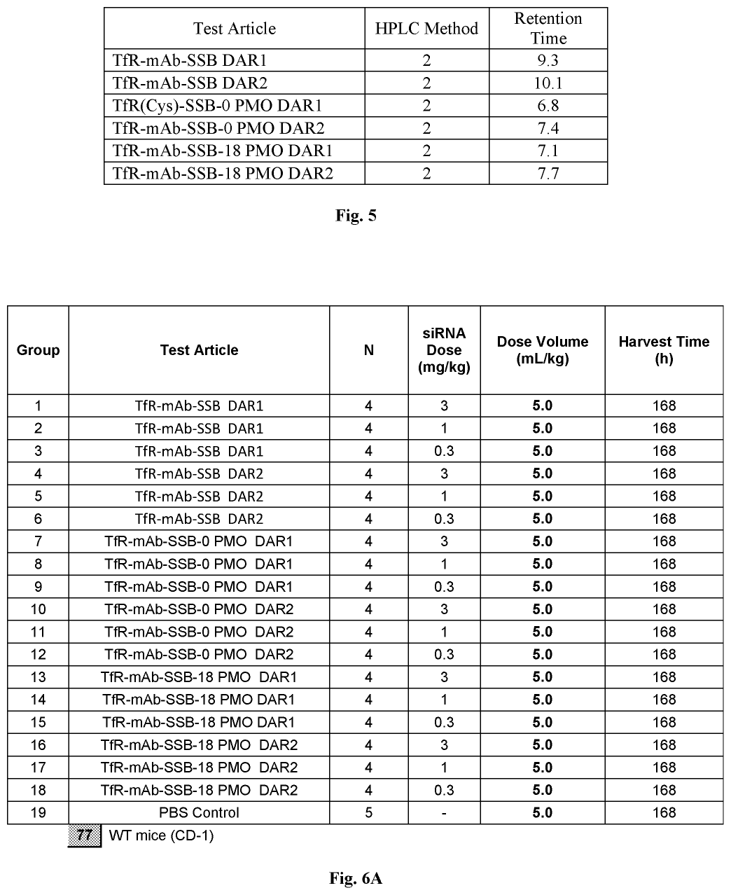

The anti-EGFR mAb-SSB DAR1 and DAR2 were synthesized as described in Example 4 using a C6-NH2 conjugation handle at the 5′ end and C6-SH at 3′end of the passenger strand. The anti-EGFR mAb-SSB-0 PMO DAR1 and DAR2 were synthesized/purified as described in Example 4 using a C6-NH2 conjugation handle at the 5′end of the PMO guide strand and used architecture 5 similarly described in Example 2). The anti-EGFR mAb-SSB-18 PMO DAR1 and DAR 2 were synthesized/purified as described in Example 2.2 using a C6-NH2 conjugation handle at the 5′end of the PMO guide strand and used architecture 4 similarly described in Example 2). All conjugates were made through nonspecific cysteine conjugation, using a BisMal linker and were characterized chromatographically as seen in FIG. 5. FIG. 5 shows analytical data table of conjugates used with HPLC retention time (RT) in minutes.

In Vivo Study Design

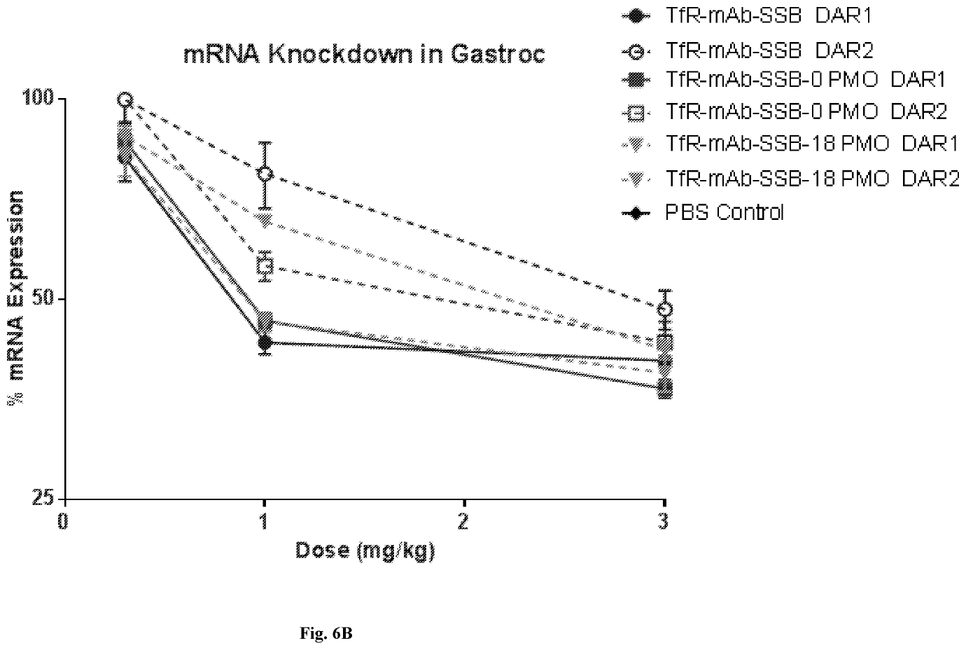

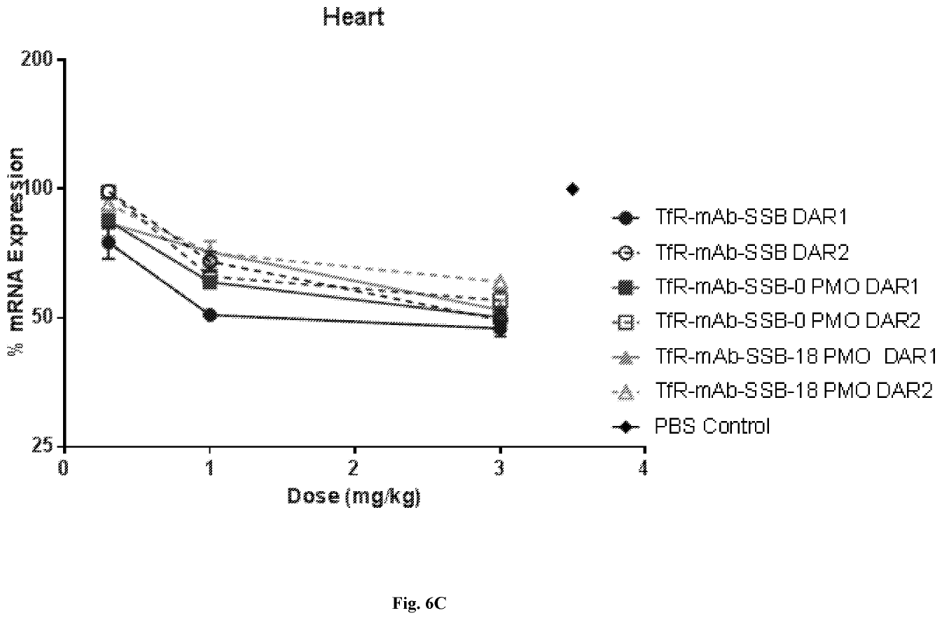

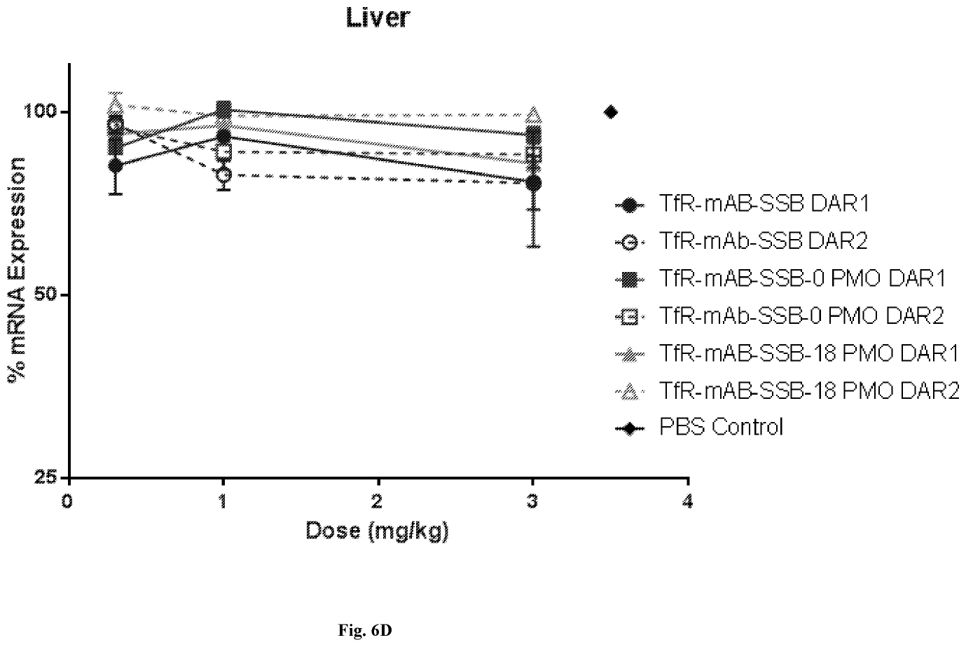

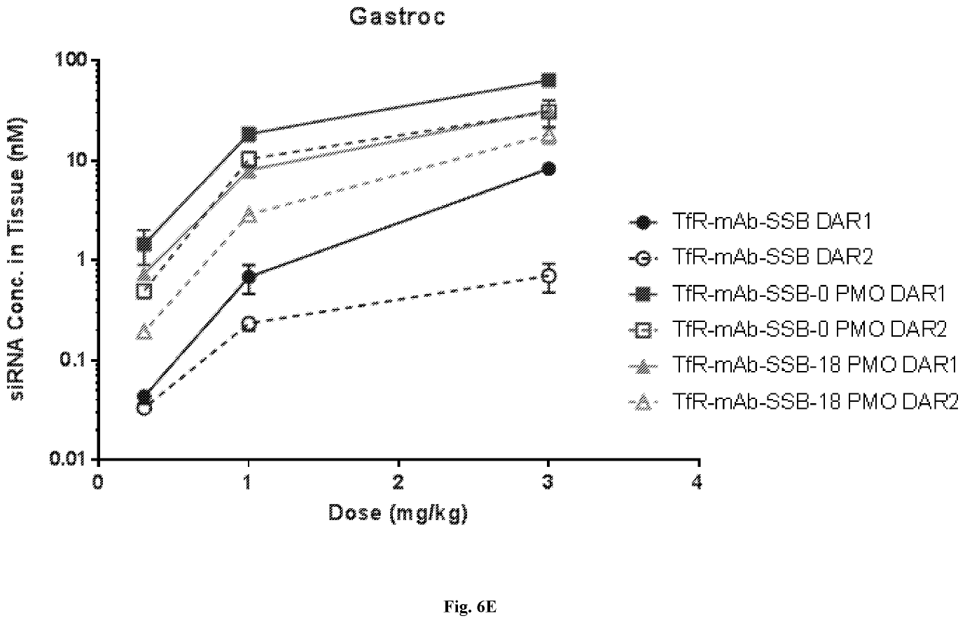

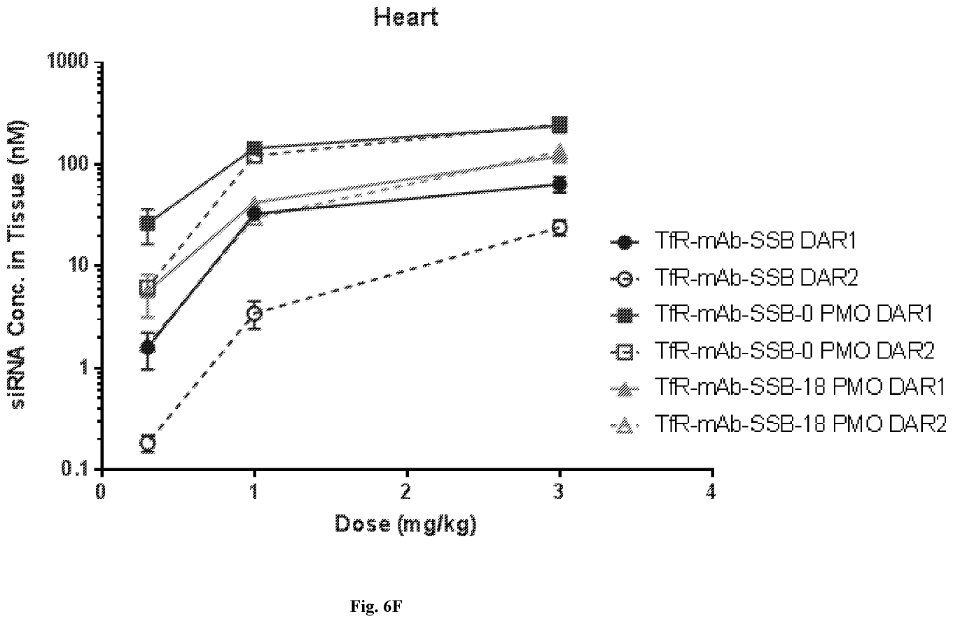

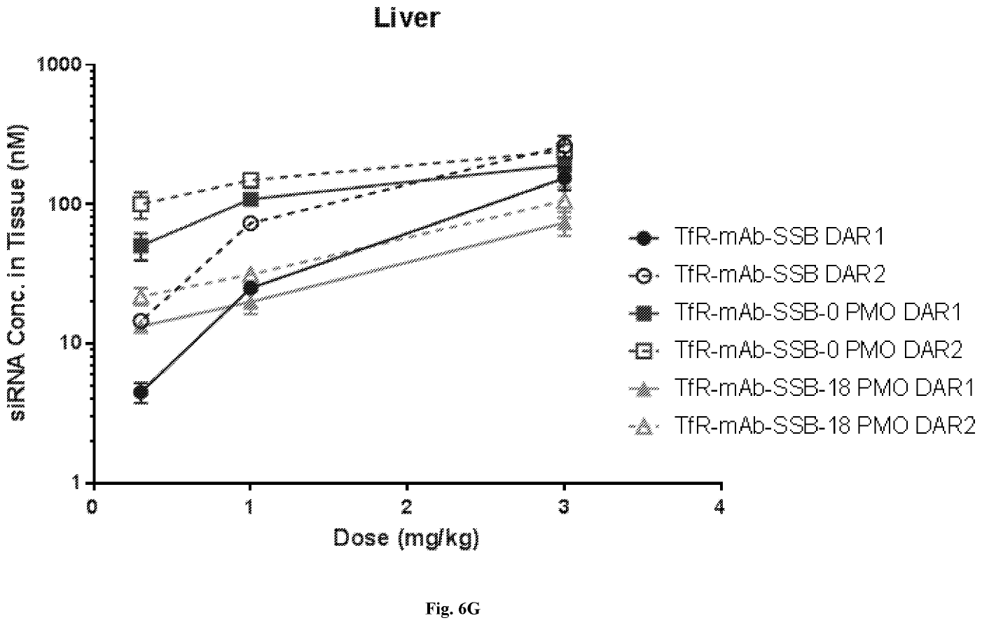

The tissue specific downregulation of the house keeping gene SSB was assessed in vivo in wild type CD-1 mice after intravenous dosing of the ASCs. Mice were dosed via intravenous (iv) injection with PBS vehicle control and the indicated ASCs and dose as seen in FIG. 6A. Non-terminal blood samples (survival bleed) were collected at the indicated times via puncture of the retro-orbital plexus and centrifuged to generate plasma for PK analysis. Mice were sacrificed by CO₂ asphyxiation at (terminal bleed/harvest) at the indicated times and terminal blood samples were collected via cardiac puncture and processed to generate plasma for PK analysis. 50 mg pieces of liver were collected and snap-frozen in liquid nitrogen and total mRNA was extracted. As described in Example 3, quantitation of plasma or tissue siRNA concentrations was determined using a stem-loop qPCR assay as described in the methods section. The antisense strand of the siRNA was reverse transcribed using a TaqMan MicroRNA reverse transcription kit using a sequence-specific stem-loop RT primer. The cDNA from the RT step was then utilized for real-time PCR and Ct values were transformed into plasma or tissue concentrations using the linear equations derived from the standard curves. Plasma concentrations of antibody were determined using an ELISA assay.

Results

The DAR1 and DAR2 conjugates, using PMO chemistry on the passenger strand of the duplex (RNA/PMO heteroduplex), resulted in measurable SSB mRNA downregulation in gastrocnemius and heart tissue as seen in FIGS. 6B-6C. In addition, in the gastrocnemius tissue mRNA downregulation was equivalent to the standard siRNA homoduplex when all the conjugates were delivered with an anti-TfR antibody. The liver tissue concentrations are seen in FIGS. 6E-6G.

This example demonstrates an accumulation of RNA/PMO heteroduplex in various muscle tissues, after a single dose, when delivered intravenously as an anti-transferrin antibody conjugate. In gastrocnemius and heart muscle, it was observed that measurable SSB mRNA downregulates with the DAR1 and DAR2 RNA/PMO heteroduplexes. Mouse gastrocnemius and heart muscle expresses the transferrin receptor and the conjugates have a mouse specific anti-transferrin antibody to target the payload, resulting in accumulation of the conjugates in muscle. Receptor mediate uptake resulted in siRNA mediated knockdown of the MSTN gene.

### Example 7. 2017-PK-376-WT—Predosing with an Excipient Oligonucleotide to Reduce Liver Accumulation of an ASC

siRNA Structure and Synthesis

For Aha1, a 21mer duplex with 19 bases of complementarity and 3′ dinucleotide overhangs was designed against mouse Aha1. The sequence (5′ to 3′) of the guide/antisense strand was UCUAAUCUCCACUUCAUCCUU. Base, sugar and phosphate modifications were as described in Example 2 for the chemical modification pattern 1. The siRNA guide and passenger strands were individually assembled on solid phase using standard phospharamidite chemistry and purified over HPLC. Purified single strands were duplexed to get the double stranded siRNA. The passenger strand contained two conjugation handles, a C6-NH2 at the 5′ end and a C6-SH at the 3′ end. Both conjugation handles were connected to siRNA passenger strand via phosphorothioate-inverted abasic-phosphorothioate linker.

For SSB, a 21mer duplex with 19 bases of complementarity and 3′ dinucleotide overhangs was designed against mouse Aha1. The sequence (5′ to 3′) of the guide/antisense strand was UUACAUUAAAGUCUGUUGUUU. Base, sugar and phosphate modifications were as described in Example 2. All siRNA single strands were fully assembled on solid phase using standard phospharamidite chemistry and purified over HPLC. Purified single strands were duplexed to get the double stranded siRNA. The passenger strand contained two conjugation handles, a C6-NH2 at the 5′ end and a C6-SH at the 3′ end. Both conjugation handles were connected to siRNA passenger strand via phosphorothioate-inverted abasic-phosphorothioate linker.

For negative control siRNA sequence (scramble), a published (Burke et al. (2014) Pharm. Res., 31(12):3445-60) 21mer duplex with 19 bases of complementarity and 3′ dinucleotide overhangs was used. The sequence (5′ to 3′) of the guide/antisense strand was UAUCGACGUGUCCAGCUAGUU. The same base, sugar and phosphate modifications that were used for the active AhA1 siRNA duplex were used in the negative control siRNA. All siRNA single strands were fully assembled on solid phase using standard phospharamidite chemistry and purified over HPLC. Purified single strands were duplexed to get the double stranded siRNA. The passenger strand contained two conjugation handles, a C6-NH2 at the 5′ end and a C6-SH at the 3′ end. Both conjugation handles were connected to siRNA passenger strand via a phosphodiester-inverted abasic-phosphodiester linker

ASC Synthesis and Characterization

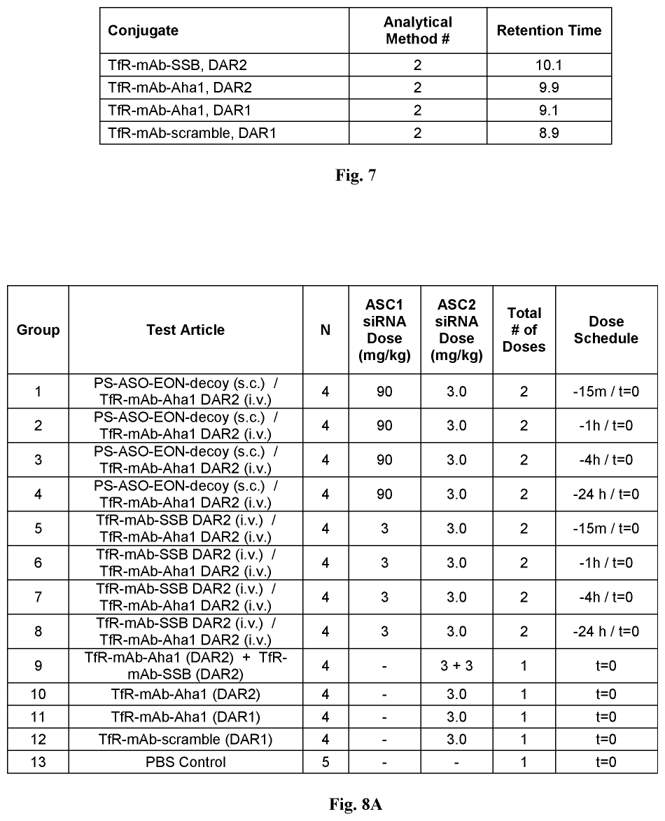

The TfR-mAb-Aha1 DAR1 and DAR 2, TfR-mAb-scramble DAR1, and TfR-mAb-SSB DAR2 were made, purified and characterized as described in Example 4. All conjugates were made through cysteine conjugation, using a BisMal linker and were characterized chromatographically as seen in FIG. 7. FIG. 7 shows an analytical data table of conjugates used with HPLC retention time (RT) in minutes. The PS-ASO-EON-decoy was synthesized as described in Example 2.3.

In Vivo Study Design

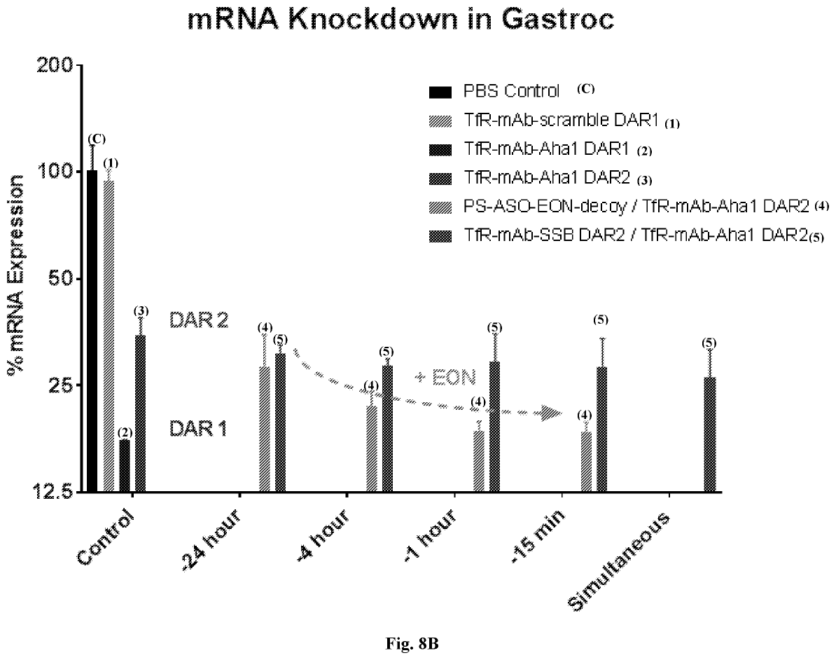

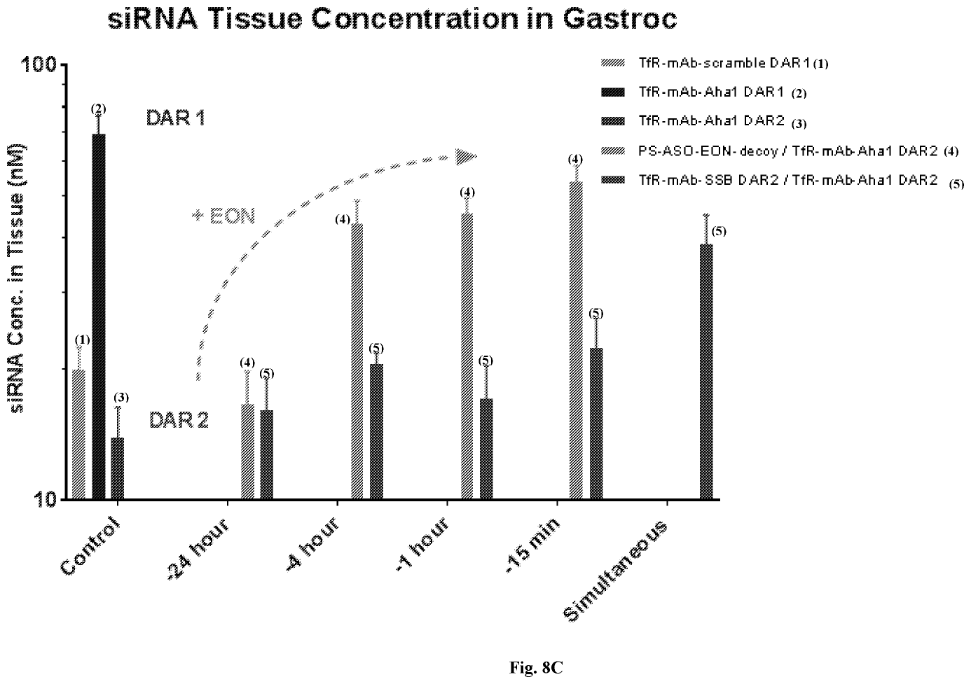

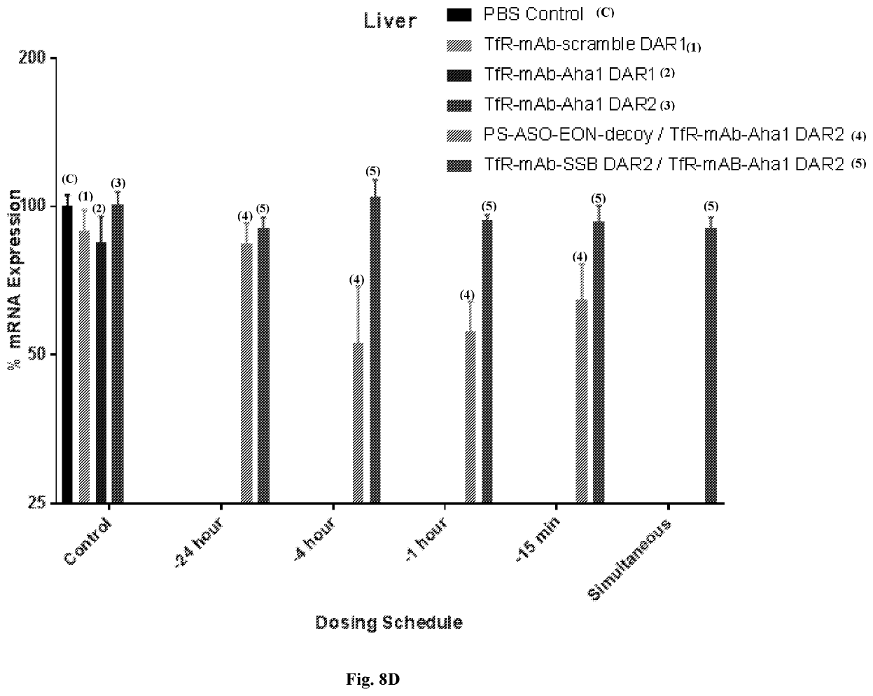

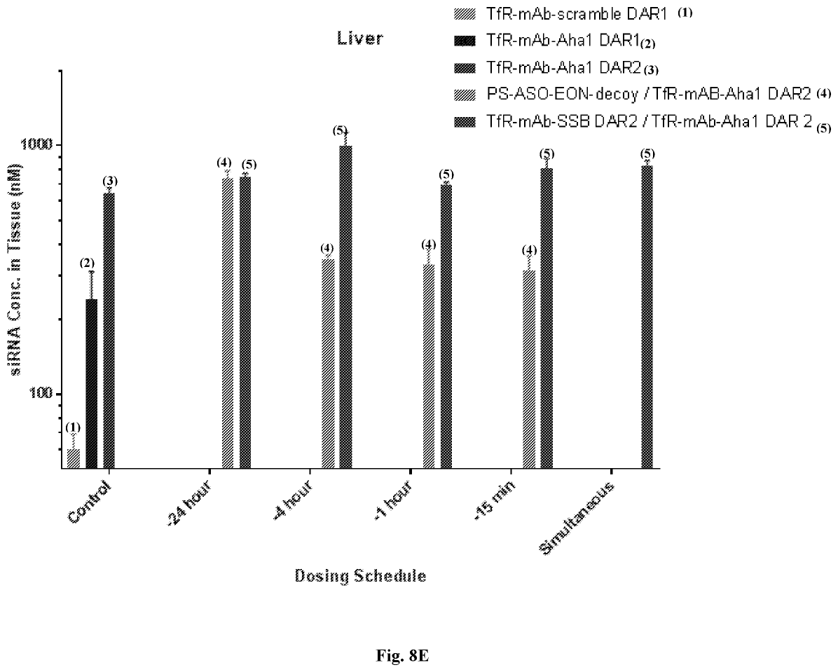

The tissue specific downregulation of the house keeping gene Aha1 was assessed in vivo in wild type CD-1 mice after intravenous dosing of the ASCs as seen in FIG. 8A. In groups 1-4, mice were predosed (s.q.) with the EON decoy (90 mg/kg) 15 minutes, 1, 4, or 24 hours before the TfR-mAb-Aha1 DAR2 conjugate. In groups 5-8, mice were predosed (i.v.) with an TfR-mAb-SSB DAR2 conjugate (3 mg/kg) 15 minutes, 1, 4, or 24 hours before the TfR-mAb-Aha1 DAR2 conjugate. In group 9, the TfR-mAb-Aha1 (DAR2) conjugate was simultaneously dosed with a TfR-mAb-SSB DAR2 conjugate. For the controls, a TfR-mAb-Aha1 DAR2 (group 10) and DAR1 (group 11), a TfR-mAb-scramble (group 12) and PBS (group 13) were used. Mice were sacrificed by CO₂ asphyxiation at (terminal bleed/harvest) 168 hours after ASC administration (t=0). 50 mg pieces of gastrocnemius, heart and liver were collected and snap-frozen in liquid nitrogen and total mRNA was extracted. As described in Example 3, quantitation of plasma or tissue siRNA concentrations was determined using a stem-loop qPCR assay as described in the methods section. The antisense strand of the siRNA was reverse transcribed using a TaqMan MicroRNA reverse transcription kit using a sequence-specific stem-loop RT primer. The cDNA from the RT step was then utilized for real-time PCR and Ct values were transformed into plasma or tissue concentrations using the linear equations derived from the standard curves.

Results

In the gastrocnemius tissue the TfR-mAb-Aha1 DAR1 control group produced significantly greater levels of Aha1 mRNA downregulation relative to the DAR2 control. The TfR-mAb-Aha1 DAR1 control group produced significantly greater siRNA tissue accumulation relative to the DAR2 control. Improvements in mRNA downregulation and siRNA tissue accumulation were observed when the PS-EON decoy was predosed s.c. at 90 mg/kg 4 h, 1 h or 15 minutes prior to administration of the TfR-mAb-Aha1 DAR2. Predosing with another siRNA (TfR-mAB-SSB DAR2) had no impact on the Aha1 mRNA downregulation produced by the TfR-mAb-Aha1 DAR2 ASC. Simultaneous dosing with another siRNA (TfR-mAB-SSB DAR2) produced a measurable increase in gasctroc muscle accumulation of the Aha1 siRNA. See FIGS. 8B-8C.

In the liver tissue the TfR-mAb-Aha1 DAR1 and DAR 2 control groups produced no significant Aha1 mRNA downregulation. The TfR-mAb-Aha1 DAR2 control group produced significantly greater siRNA tissue accumulation relative to the DAR1 control. Improvements in mRNA downregulation were observed when the PS-EON decoy was predosed 4 h, 1 h or 15 minutes prior to administration of the TfR-mAb-Aha1 DAR2. Decreased levels of Aha1 siRNA were observed when the PS-EON decoy was predosed 4 h, 1 h or 15 minutes prior to administration of the TfR-mAb-Aha1 DAR2. See FIGS. 8D-8E.

These data are consistent with the hypothesis that the phosphothioate content and negative charge of the siRNA on the ASC are modulating uptake into a nonproductive pathway in the liver and that this pathway can be saturated using a decoy molecule. Saturation of the pathway allows the DAR2 ASC to accumulate in the muscle resulting in improved mRNA target downregulation.

In this example, it was demonstrated that improvements in the performance of an ASC DAR2 was achieved by saturation of a nonproductive uptake pathway in the liver using a decoy EON.

### Example 8. 2017-PK-378-WT—Plasma PK siRNA Various Thioates DAR1 vs DAR2

siRNA Structure and Synthesis

For HPRT, a 21mer duplex with 19 bases of complementarity and 3′ dinucleotide overhangs was designed against mouse HPRT. The sequence (5′ to 3′) of the guide/antisense strand was UUAAAAUCUACAGUCAUAGUU. Base, sugar and phosphate modifications were as described in Example 2.1, chemical modification pattern 1. All siRNA single strands were fully assembled on solid phase using standard phospharamidite chemistry and purified over HPLC. Purified single strands were duplexed to get the double stranded siRNA. The passenger strand contained two conjugation handles, a C6-NH2 at the 5′ end and a C6-SH at the 3′ end. Both conjugation handles were connected to siRNA passenger strand via phosphorothioate-inverted abasic-phosphorothioate linker.

For HPRT*, a 21mer duplex with 19 bases of complementarity and 3′ dinucleotide overhangs was designed against mouse HPRT. The sequence (5′ to 3′) of the guide/antisense strand was UUAAAAUCUACAGUCAUAGUU. Base, sugar and phosphate modifications were as described in Example 2.1, chemical modification pattern 2. All siRNA single strands were fully assembled on solid phase using standard phospharamidite chemistry and purified over HPLC. Purified single strands were duplexed to get the double stranded siRNA. The passenger strand contained two conjugation handles, a C6-NH2 at the 5′ end and a C6-SH at the 3′ end. Both conjugation handles were connected to siRNA passenger strand via phosphorothioate-inverted abasic-phosphorothioate linker.

For HPRT**, a 21mer duplex with 19 bases of complementarity and 3′ dinucleotide overhangs was designed against mouse HPRT. The sequence (5′ to 3′) of the guide/antisense strand was UUAAAAUCUACAGUCAUAGUU. Base, sugar and phosphate modifications were as described in Example 2.1, chemical modification pattern 3. All siRNA single strands were fully assembled on solid phase using standard phospharamidite chemistry and purified over HPLC. Purified single strands were duplexed to get the double stranded siRNA. The passenger strand contained two conjugation handles, a C6-NH2 at the 5′ end and a C6-SH at the 3′ end. Both conjugation handles were connected to siRNA passenger strand via phosphorothioate-inverted abasic-phosphorothioate linker.

ASC Synthesis and Characterization

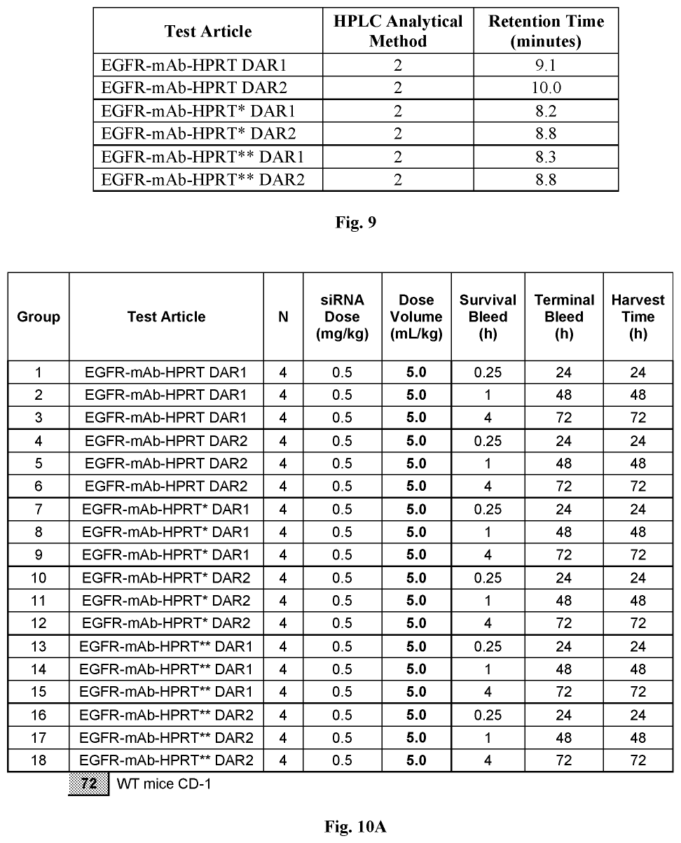

The EGFR-mAb-HPRT DAR1 and DAR 2, EGFR-mAb-HPRT* DAR1 and DAR 2 and EGFR-mAb-HPRT** DAR1 and DAR 2, were made, purified and characterized as described in Example 4. All conjugates were made through cysteine conjugation, a BisMal linker and were characterized chromatographically as seen in FIG. 9. FIG. 9 shows an analytical data table of conjugates with HPLC retention time (RT) in minutes.

In Vivo Study Design

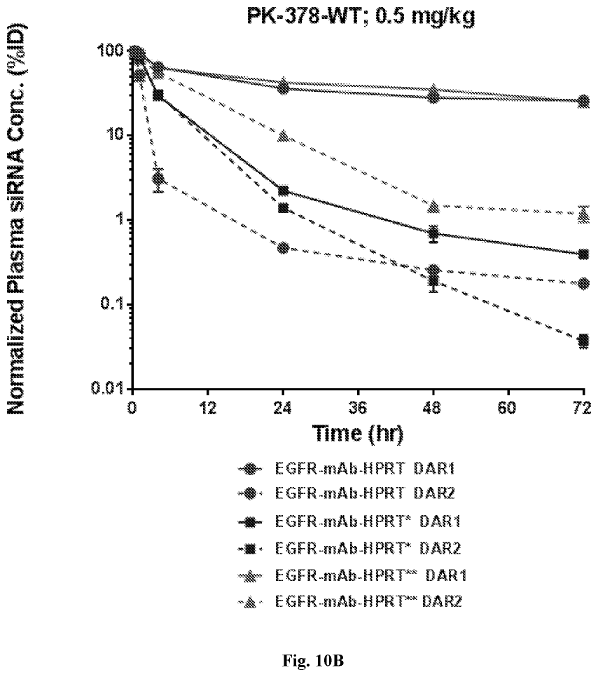

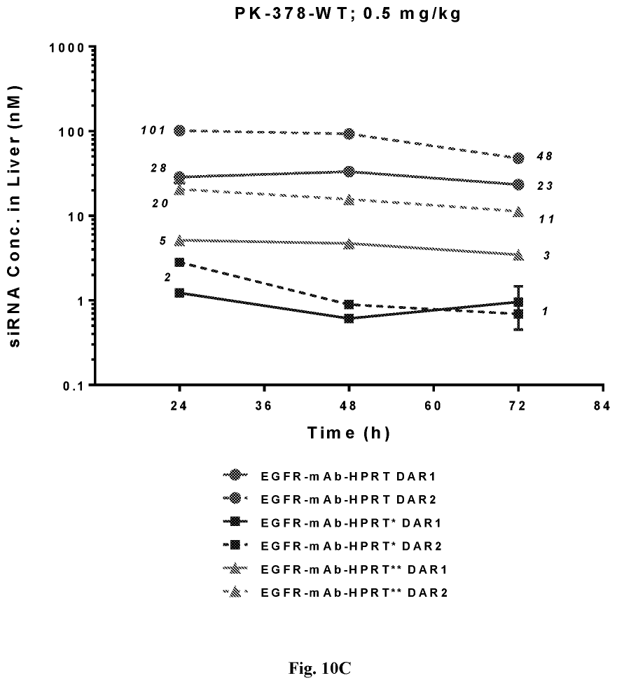

The tissue specific downregulation of the house keeping gene Aha1 was assessed in vivo in wild type CD-1 mice after intravenous dosing of the ASCs as seen in FIG. 10A. In groups 1-4, mice were predosed (s.q.) with the EON decoy (90 mg/kg) 15 minutes, 1, 4, or 24 hours before the TfR-mAb-Aha1 DAR2 conjugate. In groups 5-8, mice were predosed (i.v.) with an TfR-mAb-SSB DAR2 conjugate (3 mg/kg) 15 minutes, 1, 4, or 24 hours before the TfR-mAb-Aha1 DAR2 conjugate. In group 9, the TfR-mAb-Aha1 (DAR2) conjugate was simultaneously dosed with a TfR-mAb-SSB DAR2 conjugate. For the controls, a TfR-mAb-Aha1 DAR2 (group 10) and DAR1 (group 11), a TfR-mAb-scramble (group 12) and PBS (group 13) were used. Mice were sacrificed by CO₂ asphyxiation at (terminal bleed/harvest) 168 hours after ASC administration (t=0). 50 mg pieces of gastroc, heart and liver were collected and snap-frozen in liquid nitrogen and total mRNA was extracted. As described in Example 3, quantitation of plasma or tissue siRNA concentrations was determined using a stem-loop qPCR assay as described in the methods section. The antisense strand of the siRNA was reverse transcribed using a TaqMan MicroRNA reverse transcription kit using a sequence-specific stem-loop RT primer. The cDNA from the RT step was then utilized for real-time PCR and Ct values were transformed into plasma or tissue concentrations using the linear equations derived from the standard curves.

Results

For the DAR1 conjugates, reducing the phosphothioate content of the siRNA from 9 to 1 had no effect on the plasma PK as seen in FIG. 10B. However, it did reduce the amount of siRNA detected in the liver. For the DAR1 conjugates, reducing the phosphothioate content of the siRNA from 9 to 0 reduced the plasma half-life of the ASC as seen in FIG. 10B. This is propably instability of the siRNA duplex, since the phosphothioates provide stability to enzymatic cleavage.

For the DAR 2 conjugates, reducing the phosphothioate content of the siRNA from 9 to 1 increased the plasma half-life of the ASC as seen in FIG. 10B. In addition, it reduced the amount of siRNA detected in the liver. For the DAR2 conjugates, reducing the phosphothioate content of the siRNA from 9 to 0 reduced the plasma half-life of the ASC as seen in FIG. 10B. This is probably caused by instability of the siRNA duplex, since the phosphothioates provide stability to enzymatic cleavage. FIG. 10C shows siRNA tissue concentration in liver.

This example demonstrates improvements in the performance of an ASC DAR1 and DAR2 can be achieved by reducing the phosphorothioate content of the siRNA payload on an ASC.

These data are consistent with the hypothesis that the phosphothioate content of the siRNA payload on the ASC are modulating uptake into a nonproductive pathway in the liver and that this pathway can be avoided by reducing the phosphothioate content.

### Example 9: In Vitro Testing of the PMO/RNA Heteroduplex

RNA and PMO Structure and Synthesis

RNA single strand was held constant as the guide strand for RNAi mechanism. PMOs were generated to be fully complementary to the guide strand, or truncated, nicked, or to contain mismatched bases. RNA guide strand and PMO passenger strand were combined in equimolar ratios in water at a concentration of 1 mM to duplex. The mixture was heated to 85° C. in oil bath, incubated for 5 min, then turned off heat and cooled to RT at ˜1° C. per min. A PMO/RNA heteroduplexes was generated the house keeper gene SSB:

SSB Guide strand: vpUsUfsAfscAfuUfAfaAfgUfcUfgUfugususu

SSB siRNA passenger strand: iBsascaaCfaGfaCfuUfuAfaUfgUfaaususiB

vpN=vinyl phosphonate 2′-MOE; Upper case (N)=2′-OH (ribo); Lower case (n)=2′-O-Me (methyl); dN=2′-H (deoxy); Nf=2′-F (fluoro); s=phosphorothioate backbone modification;

iB=inverted abasic

Duplexing efficiency was assessed by size exclusion chromatography (SEC) using a Superdex 75 (10/300 GL GE) column with a flow rate of 0.75 mL/min and a mobile phase of phosphate buffered saline (PBS, pH 7.0) plus 10% acetonitrile. Signal was measured by absorbance at 260 nm.

In vitro study design: SSB Hetroduplex Transfection into LLC1 cells

LLC1 cells were transfected with RNAiMAX (Invitrogen) according to manufacturer's instructions using reverse transfection, 50,000 cells/well and incubated for 48 hours. Total RNA was extracted from the cells, reverse transcribed and mRNA levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

Results

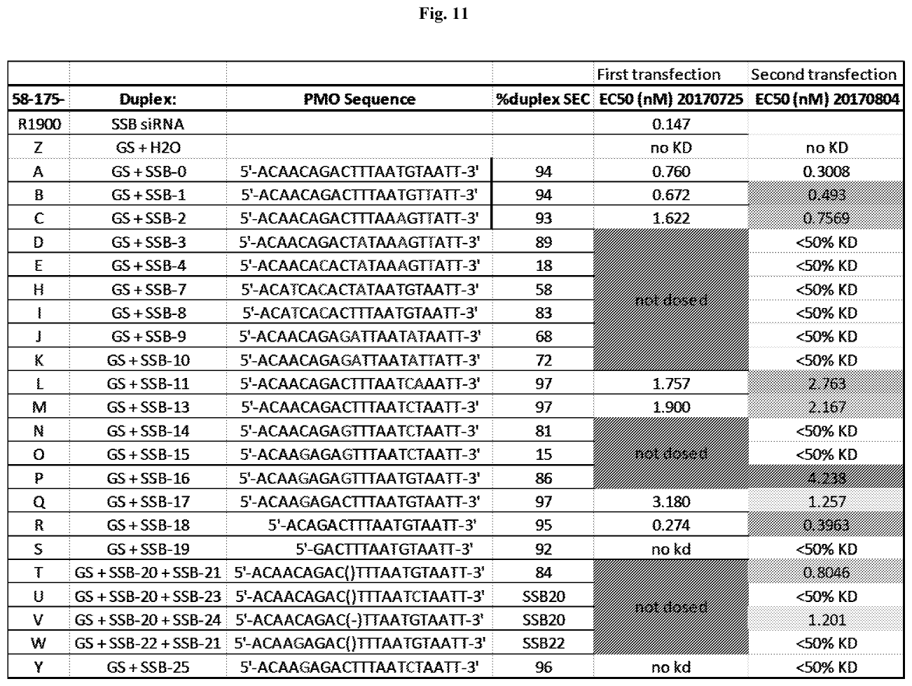

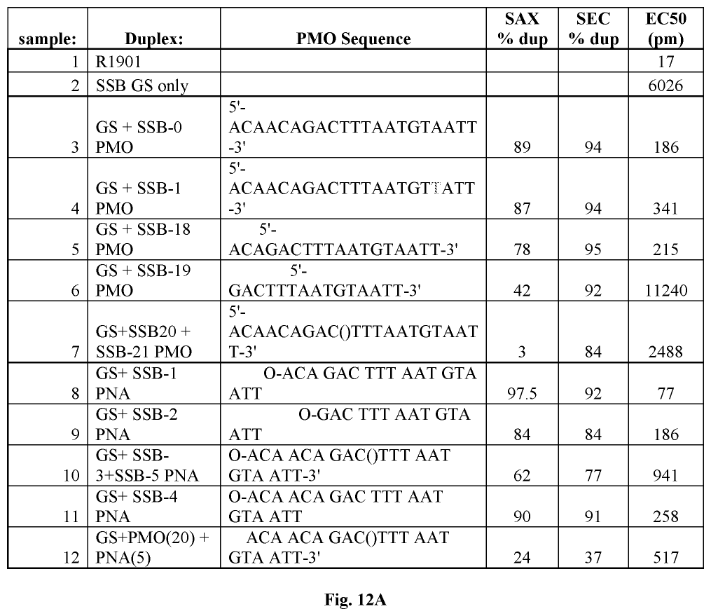

FIG. 11 describes the efficiency of duplex formation and half maximal concentrations of RNA/PMO heteroduplex (EC50) which induced mRNA downregulation halfway between the baseline and maximum at 48 hours after transfection. FIG. 11 further shows percentage duplex formation and EC50 values of RNA/PMO heteroduplexes after transfection into LLC1 cells.

Single strands of RNA and PMO, with various degrees of complementarity, formed duplexes and were able to efficiently induce gene specific mRNA downregulation after in vitro transfection.

### Example 10: In Vitro Testing of the PMO/RNA and PNA/RNA Heteroduplexes

RNA, PMO and PNA Structure and Synthesis

RNA single strand was held constant as the guide strand for RNAi mechanism. The standard siRNA duplex designed to downregulate the house keeper gene SSB had the following sequence and base modifications:

SSB Guide strand: vpUsUfsAfscAfuUfAfaAfgUfcUfgUfugususu

SSB siRNA passenger strand: iBsascaaCfaGfaCfuUfuAfaUfgUfaaususiB

vpN=vinyl phosphonate 2′-MOE; Upper case (N)=2′-OH (ribo); Lower case (n)=2′-O-Me (methyl); dN=2′-H (deoxy); Nf=2′-F (fluoro); s=phosphorothioate backbone modification;

iB=inverted abasic

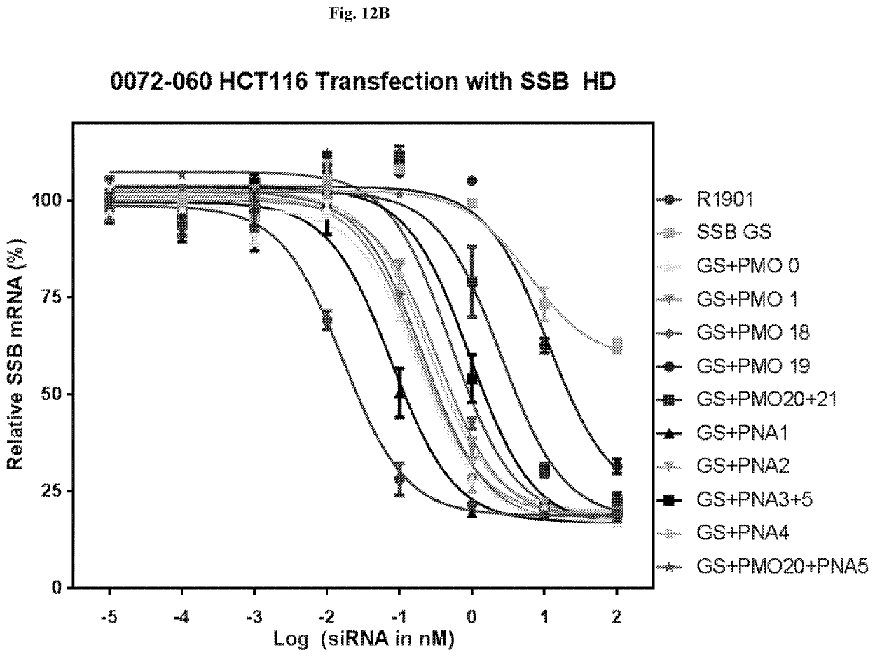

PMOs passenger strands were generated to be fully complementary to the guide strand, or truncated, nicked, or to contain mismatched bases, see FIG. 12A. RNA guide strand and PMO passenger strands were combined in equimolar ratios in water at a concentration of 1 mM to duplex. The mixture was heated to 85° C. in oil bath, incubated for 5 min, then the heat was turned off and the solution cooled to RT at ˜1° C. per min. PMO/RNA heteroduplexes were designed and generated to downregulate the house keeper gene SSB and the RNA guide strand had the sequence and base modification shown above.

PNAs passenger strands were generated to be fully complementary to the guide strand, or truncated, nicked, or to contain mismatched bases, see FIG. 12A. RNA guide strand and PNA passenger strands were combined in equimolar ratios in PBS at a concentration of 0.1 mM to duplex. The mixture was heated to 85° C. in oil bath, incubated for 5 min, then the heat was turned off and the solution cooled to RT at ˜1° C. per min. PNA/RNA heteroduplexes were designed and generated to downregulate the house keeper gene SSB and the RNA guide strand had the sequence and base modification shown above.

Duplexing efficiency was assessed using two methods:

Size exclusion chromatography (SEC) using a Superdex 75 (10/300 GL GE) column with a flow rate of 0.75 mL/min and a mobile phase of phosphate buffered saline (PBS, pH 7.0) plus 10% acetonitrile. Signal was measured by absorbance at 260 nm.

Strong anion exchange chromatography (SAX) using a ProPac™ SAX-10, Bio LC™, 4×250 mm (Thermo Scientific) column. Solvent A: 80% 10 mM TRIS pH 8, 20% ethanol; Solvent B: 80% 10 mM TRIS pH 8, 20% ethanol, 1.5 M NaCl; Flow Rate: 0.75 ml/min, using a gradient elution: 0-3 minutes (10% B), 3-11 minutes (10 to 60% B), 11-14 min (60% B), 14-15 minutes (60 to 80%). Signal was measured by absorbance at 260 nm.

In Vitro Study Design: SSB Hetroduplex Transfection intoHCT116 Cells

HCT116 cells were transfected with RNAiMAX (Invitrogen) according to manufacturer's instructions using reverse transfection, 50,000 cells/well and incubated for 48 hours. Total RNA was extracted from the cells, reverse transcribed and mRNA levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

Results

FIG. 12A describes the efficiency of duplex formation (as measured by SAX and SEC) and half maximal concentrations of RNA/PMO and RNA/PNA heteroduplex (EC50) which induced mRNA downregulation halfway between the baseline and maximum at 48 hours after transfection.

FIG. 12B illustrates SSB mRNA downregulation after RNA/PMO heteroduplexes transfection into HCT116 cells.

Single strands of RNA, PMO and PNA, with various degrees of complementarity, formed duplexes and were able to efficiently induce gene specific mRNA downregulation after in vitro transfection.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A molecule of Formula (I):
A-(X¹—B)ₙ Formula (I)
wherein,
A comprises a binding moiety;
B consists of a hetero-duplex polynucleotide consisting of a guide strand and a passenger strand;
X¹ consists of a bond or linker; and
n is an averaged value selected from 1-12;
wherein the guide strand comprises at least one but no more than 10 phosphorothioate-modified non-natural nucleotides;
wherein the passenger strand comprises a plurality of phosphorodiamidate morpholino oligomers or a plurality of peptide nucleic acid-modified non-natural nucleotides; and
wherein the hetero-duplex polynucleotide has one of: a greater hepatocyte stability, reduced overall charge, reduced hepatocyte uptake, or extended pharmacokinetics, compare to analogous homoduplex nucleotide.

2. The molecule of claim 1, wherein the passenger strand further comprises at least one inverted abasic moiety, optionally at one or both termini.

3. The molecule of claim 1, wherein the guide strand further comprises at least one modified internucleotide linkage, at least one inverted abasic moiety, at least one 5′-vinylphosphonate modified non-natural nucleotide, or a combination thereof.

4. The molecule of claim 1, wherein the guide strand comprises 1 phosphorothioate-modified non-natural nucleotide, or about 2, 3, 4, 5, 6, 7, 8, or 9 phosphorothioate-modified non-natural nucleotides.

5. The molecule of claim 1, wherein the phosphorothioate modified non-natural nucleotide is located at an internucleotide linkage of the polynucleotide.

6. The molecule of claim 3, wherein the at least one 5′-vinylphosphonate modified non-natural nucleotide is located at the 5′-terminus of the guide strand, or about 1, 2, 3, 4, or 5 bases away from the 5′ terminus of the guide strand.

7. The molecule of claim 3, wherein the at least one 5′-vinylphosphonate modified non-natural nucleotide is further modified at the 2′-position.

8. The molecule of claim 7, wherein the 2′-modification is selected from 2′-O-methyl, 2′-O-methoxyethyl (2′-O-MOE), 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified nucleotide.

9. The molecule of claim 7, wherein the at least one 5′-vinylphosphonate modified non-natural nucleotide is selected from: 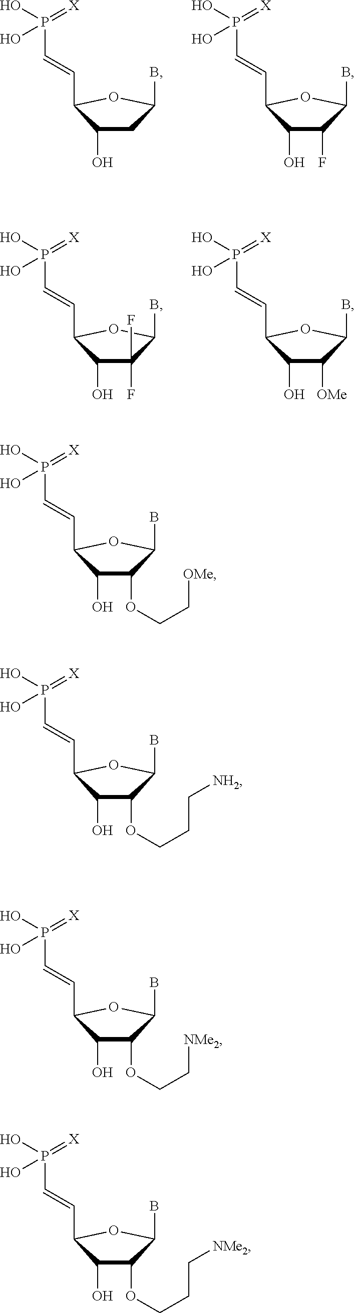 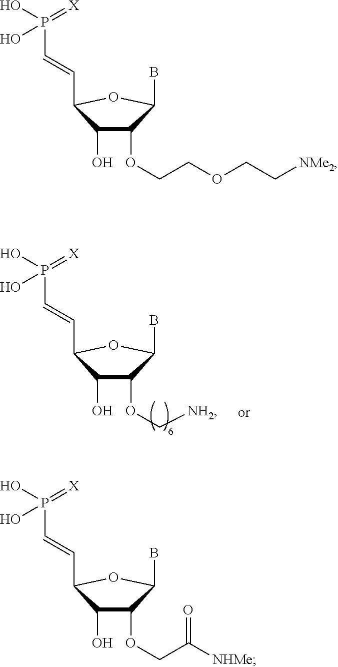
wherein X is O or S; and B is a heterocyclic base moiety.

10. The molecule of claim 7, wherein the at least one 5′-vinylphosphonate modified non-natural nucleotide is selected from: 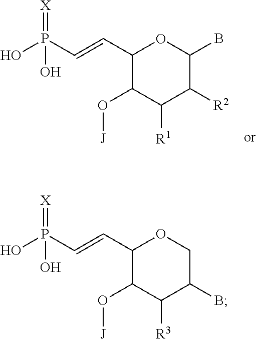
wherein X is O or S; B is a heterocyclic base moiety;
R¹, R², and R³ are independently selected from hydrogen, halogen, alkyl or alkoxy; and
J is an internucleotide linking group linking to the adjacent nucleotide of the polynucleotide.

11. The molecule of claim 3, wherein the at least one 5′-vinylphosphonate modified non-natural nucleotide is selected from: 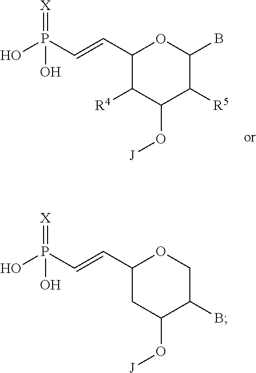
wherein X is O or S; B is a heterocyclic base moiety;
R⁴, and R⁵ are independently selected from hydrogen, halogen, alkyl or alkoxy; and
J is an internucleotide linking group linking to the adjacent nucleotide of the polynucleotide.

12. The molecule of claim 3, wherein the at least one 5′-vinylphosphonate modified non-natural nucleotide is selected from: 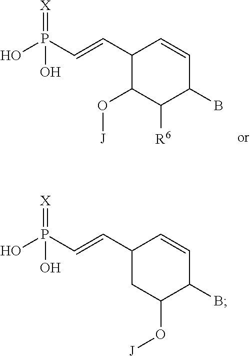
wherein X is O or S; B is a heterocyclic base moiety;
R⁶ is selected from hydrogen, halogen, alkyl or alkoxy; and
J is an internucleotide linking group linking to the adjacent nucleotide of the polynucleotide.

13. The molecule of claim 3, wherein the at least one 5′-vinylphosphonate modified non-natural nucleotide is a locked nucleic acid (LNA) or an ethylene nucleic acid (ENA).

14. The molecule of claim 3, wherein the at least one 5′-vinylphosphonate modified non-natural nucleotide is selected from: 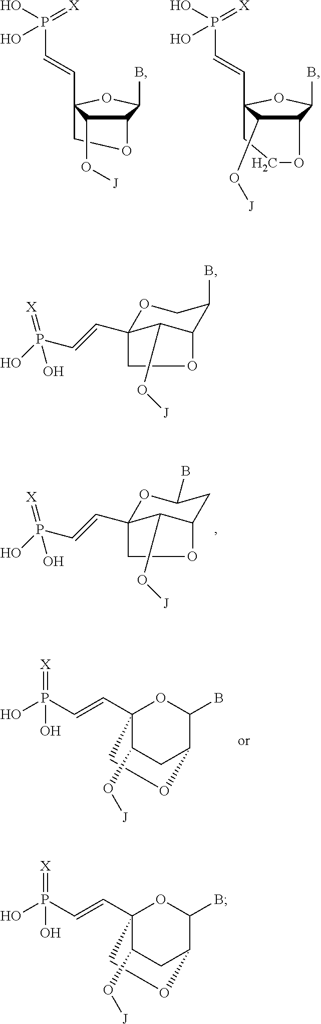
wherein X is O or S; B is a heterocyclic base moiety; and
J is an internucleotide linking group linking to the adjacent nucleotide of the polynucleotide.

15. The molecule of claim 3, wherein the at least one 5′-vinylphosphonate modified non-natural nucleotide is selected from: 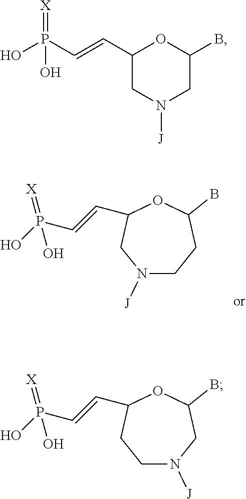
wherein X is O or S; B is a heterocyclic base moiety; and
J is an internucleotide linking group linking to the adjacent nucleotide of the polynucleotide.

16. The molecule of claim 3, wherein the at least one 5′-vinylphosphonate modified non-natural nucleotide is: 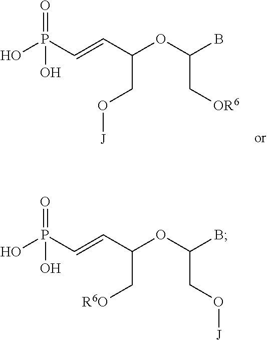
wherein X is O or S; B is a heterocyclic base moiety;
R⁶ is selected from hydrogen, halogen, alkyl or alkoxy; and
J is an internucleotide linking group linking to the adjacent nucleotide of the polynucleotide.

17. The molecule of claim 3, wherein the at least one inverted abasic moiety is at one or both termini.

18. The molecule of claim 1, wherein the guide strand comprises RNA nucleotides.

19. The molecule of claim 1, wherein the passenger strand comprises at least 6, 7, 8, 9, 10, 11, 12, 13, 14, 15, 16, 17, 18, 19, 20, or more phosphorodiamidate morpholino oligomer-modified non-natural nucleotides.

20. The molecule of claim 19, wherein the passenger strand comprises 100% phosphorodiamidate morpholino oligomer-modified non-natural nucleotides.

21. The molecule of claim 19, wherein the passenger strand is shorter in length than the guide strand, thereby generating a 5′ overhang, a 3′ overhang, or a combination thereof.

22. The molecule of claim 19, wherein the passenger strand is equal in length to the guide strand, thereby generating a blunt end at each terminus of the hetero-duplex polynucleotide.

23. The molecule of claim 19, wherein the passenger strand when hybridized to the guide strand further comprises at least one, two, three, four, or more mismatches, optionally comprising at least one, two, three, four, or more internal mismatches.

24. The molecule of claim 19, wherein the hetero-duplex polynucleotide is a phosphorodiamidate morpholino oligomer/RNA hetero-duplex.

25. The molecule of claim 1, wherein the passenger strand comprises at least 6, 7, 8, 9, 10, 11, 12, 13, 14, 15, 16, 17, 18, 19, 20, or more peptide nucleic acid-modified non-natural nucleotides.

26. The molecule of claim 25, wherein the passenger strand comprises 100% peptide nucleic acid-modified non-natural nucleotides.

27. The molecule of claim 25, wherein the passenger strand is shorter in length than the guide strand, thereby generating a 5′ overhang, a 3′ overhang, or a combination thereof.

28. The molecule of claim 25, wherein the passenger strand is equal in length to the guide strand, thereby generating a blunt end at each terminus of the hetero-duplex polynucleotide.

29. The molecule of claim 25, wherein the passenger strand when hybridized to the guide strand further comprises at least one, two, three, four, or more mismatches, optionally comprising at least one, two, three, four, or more internal mismatches.

30. The molecule of claim 25, wherein the hetero-duplex polynucleotide is a peptide nucleic acid/RNA hetero-duplex.

31. The molecule of claim 1, wherein the passenger strand is conjugated to A-X¹.

32. The molecule of claim 31, wherein A-X¹ is conjugated to the 5′ end of the passenger strand.

33. The molecule of claim 31, wherein A-X¹ is conjugated to the 3′ end of the passenger strand.

34. The molecule of claim 1, wherein the guide strand comprises a sequence having at least 80%, 85%, 90%, 95%, 96%, 97%, 98%, 99%, or 100% sequence identity to SEQ ID NOs: 16-45, 422-1173, 1181-1184, or 1195-1242.

35. The molecule of claim 1, wherein the passenger strand comprises a sequence having at least 80%, 85%, 90%, 95%, 96%, 97%, 98%, 99%, or 100% sequence identity to SEQ ID NOs: 16-45, 422-1173, 1181-1184, or 1195-1242.

36. The molecule of claim 1, wherein the passenger strand comprises two or more polynucleotides, wherein each of the two or more polynucleotides hybridizes to a separate region on the guide strand, forming either a continuous strand without a gap between the termini of the two or more polynucleotides or a gap of about 1, 2, 3, or more bases between the termini of the two or more polynucleotides.

37. The molecule of claim 36, wherein the two or more polynucleotides independently comprise at least 6, 7, 8, 9, 10, 11, 12, 13, 14, 15, 16, 17, 18, 19, 20, or more phosphorodiamidate morpholino oligomer-modified non-natural nucleotides or at least 6, 7, 8, 9, 10, 11, 12, 13, 14, 15, 16, 17, 18, 19, 20, or more peptide nucleic acid-modified non-natural nucleotides.

38. The molecule of claim 36, wherein the two or more polynucleotides independently comprise 100% phosphorodiamidate morpholino oligomer-modified non-natural nucleotides or 100% peptide nucleic acid-modified non-natural nucleotides.

39. The molecule of claim 21 or 27, wherein the overhang is about 1, 2, 3, 4, 5, 6, 7, 8, 9, 10, or more bases.

40. The molecule of claim 1, wherein X¹ is a non-polymeric linker.

41. The molecule of claim 1, wherein X¹ is a homobifuctional linker or a heterobifunctional linker, optionally conjugated to a C₁-C₆ alkyl group.

42. The molecule of claim 1, wherein the binding moiety comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof.

43. The molecule of claim 1, wherein the binding moiety comprises a peptide or small molecule.

44. The molecule of claim 1, wherein n is an averaged value selected from 2-12, 4-12, 4-8, 6-8, or 8-12.

45. The molecule of claim 1, further comprising C.

46. The molecule of claim 45, wherein C is polyethylene glycol.

47. The molecule of claim 46, wherein C has a molecular weight of about 1000 Da, 2000 Da, or 5000 Da.

48. The molecule of claim 45, wherein C is directly conjugated to B via X².

49. The molecule of claim 48, wherein X² consists of a bond or a linker, optionally a non-polymeric linker.

50. The molecule of claim 49, wherein X² is a homobifuctional linker or a heterobifunctional linker, optionally conjugated to a C₁-C₆ alkyl group.

51. The molecule of claim 48, wherein the passenger strand is conjugated to A-X¹ and X²—C.

52. The molecule of claim 51, wherein A-X¹ is conjugated to the 5′ end of the passenger strand and X²—C is conjugated to the 3′ end of the passenger strand.

53. The molecule of claim 51, wherein X²—C is conjugated to the 5′ end of the passenger strand and A-X¹ is conjugated to the 3′ end of the passenger strand.

54. The molecule of claim 1, further comprising D.

55. The molecule of claim 54, wherein D is an endosomolytic moiety.

56. The molecule of claim 1, wherein the molecule has a reduced hepatic clearance rate compare to an analogous molecule comprising a homoduplex nucleotide.

57. The molecule of claim 1, wherein the molecule has reduced uptake mediated by the Stabilin-1 or Stabilin-2 receptor relative to an analogous molecule comprising a homoduplex nucleotide.

58. The molecule of claim 1, wherein the molecule has an increased plasma half-life relative to an analogous molecule comprising a homoduplex nucleotide.

59. The molecule of claim 1, wherein the molecule has an increased target tissue uptake relative to an analogous molecule comprising a homoduplex nucleotide.

60. The molecule of claim 1, wherein the molecule has an improved pharmacokinetics relative to an analogous molecule comprising a homoduplex nucleotide.

61. A pharmaceutical composition, comprising:
a molecule of claims 1-60; and
a pharmaceutically acceptable excipient.

62. A method of treating a disease or indication, comprising:
administering to a subject in need thereof a therapeutically effective amount of a molecule of claims 1-60 or a pharmaceutical composition of claim 61, thereby treating the subject.

63. The method of claim 62, wherein the subject is a human.
